(19) United States
(12) Patent Application Publication
(10) Pub. No.: US 2005/0222027 A1
(43) Pub. Date: Oct. 6, 2005

(54) Modulation of complement to treat pain

(75) Inventors: Lillian W. Chiang, Princeton, NJ (US); Margaret E. Levin, Princeton, NJ (US)

Correspondence Address: DARBY & DARBY P.C., P. O. BOX 5257, NEW YORK, NY, 10150-5257, US

(73) Assignee: Euro-Celtique S.A., Luxembourg (LU)

(21) Appl. No.: 10/989,891

(22) Filed: Nov. 12, 2004

Related U.S. Application Data
(63) Continuation of application No. PCT/US04/23166, filed on Jul. 6, 2004.
(60) Provisional application No. 60/485,101, filed on Jul. 3, 2003.

(51) Int. Cl. A61K 38/17; A61K 38/48

(52) U.S. Cl. 514/12; 424/94.63

(57) ABSTRACT

The present invention provides compositions and methods for treating pain, including neuropathic pain, by modulating the expression or activity of one or more components of the complement pathway. The present invention further provides screening methods to identify therapeutic agents for treating pain by screening for compounds capable of modulating the expression or activity of one or more components of the complement pathway.

The present application is a continuation in part of PCT Application No. PCT/US04/23166 filed Jul. 6, 2004, which claims priority to U.S. Provisional Patent Application Ser. No. 60/485,101 filed Jul. 3, 2003. Both PCT Application No. PCT/US04/23166 and U.S. Provisional Patent Application Ser. No. 60/485,101 are incorporated herein by reference in their entirety.

### 1. FIELD OF THE INVENTION

The present invention is in the field of therapeutic agents for pain treatment, and provides compositions and methods for treating pain that act through the modulation of a component of the complement pathway.

### 2. BACKGROUND OF THE INVENTION

Pain is the most common symptom for which patients seek medical help, and can be classified as either acute or chronic. Acute pain is precipitated by immediate tissue injury (e.g., a burn or a cut), and is usually self-limited. This form of pain is a natural defense mechanism in response to immediate tissue injury, preventing further use of the injured body part, and withdrawal from the painful stimulus. It is amenable to traditional pain therapeutics, including non-steroidal anti-inflammatory drugs (NSAIDs) and opioids. In contrast, chronic pain is present for an extended period, e.g., for 3 or more months, persisting after an injury has resolved, and can lead to significant changes in a patient's life (e.g., functional ability and quality of life) (Foley, Pain, In: Cecil Textbook of Medicine, pp. 100-107, Bennett and Plum eds., 20^{th} ed., 1996).

Chronic, debilitating pain represents a significant medical dilemma. In the United States, about 40 million people suffer from chronic recurrent headaches; 35 million people suffer from persistent back pain; 20 million people suffer from osteoarthritis; 2.1 million people suffer from rheumatoid arthritis; and 5 million people suffer from cancer-related pain (Brower, Nature Biotechnology 2000; 18: 387-391). Cancer-related pain results from both inflammation and nerve damage. In addition, analgesics are often associated with debilitating side effects such as nausea, dizziness, constipation, respiratory depression and cognitive dysfunction (Brower, Nature Biotechnology 2000; 18: 387-391). Pain can be classified as either “nociceptive” or “neuropathic”, as defined below.

### 2.1. Nociceptive Pain

“Nociceptive pain” results from activation of pain-sensitive nerve fibers, either somatic or visceral. Nociceptive pain is generally a response to direct tissue damage. The initial trauma typically causes the release of several chemicals including bradykinin, serotonin, substance P, histamine, and prostaglandin. When somatic nerves are involved, the pain is typically experienced as an aching or pressure-like sensation.

Nociceptive pain has traditionally been managed by administering non-opioid analgesics. These analgesics include acetylsalicylic acid, choline magnesium trisalicylate, acetaminophen, ibuprofen, fenoprofen, diflusinal, and naproxen, among others. Opioid analgesics, such as morphine, hydromorphone, methadone, levorphanol, fentanyl, oxycodone and oxymorphone, may also be used (Foley, Pain, In: Cecil Textbook of Medicine, pp. 100-107, Bennett and Plum eds., ₂₀th ed., 1996).

### 2.2. Neuropathic Pain

The term “neuropathic pain” refers to pain that is due to injury or disease of the central or peripheral nervous system (McQuay, Acta Anaesthesiol. Scand. 1997; 41(1 Pt 2): 175-83; Portenoy, J. Clin. Oncol. 1992; 10:1830-2). In contrast to the immediate pain caused by tissue injury, neuropathic pain can develop days or months after a traumatic injury. Furthermore, while pain caused by tissue injury is usually limited in duration to the period of tissue repair, neuropathic pain frequently is long lasting or chronic. Moreover, neuropathic pain can occur spontaneously or as a result of stimulation that normally is not painful.

Neuropathic pain is associated with chronic sensory disturbances, including spontaneous pain, hyperalgesia (i.e., sensation of more pain than the stimulus would warrant), and allodynia (i.e., a condition in which ordinarily painless stimuli induce the experience of pain). In humans, prevalent symptoms include cold hyperalgesia and mechanical allodynia. Descriptors that are often used to describe such pain include “lancinating,” “burning,” or “electric”. It is estimated that about 4 million people in North America suffer from chronic neuropathic pain, and of these no more than half achieve adequate pain control (Hansson, Pain Clinical Updates 1994; 2(3)).

Examples of neuropathic pain syndromes include those resulting from disease progression, such as diabetic neuropathy, multiple sclerosis, or post-herpetic neuralgia (shingles); those initiated by injury, such as amputation (phantom-limb pain), or injuries sustained in an accident (e.g., avulsions); and those caused by nerve damage, such as from chronic alcoholism, viral infection, hypothyroidism, uremia, or vitamin deficiencies. Traumatic nerve injuries can also cause the formation of neuromas, in which pain occurs as a result of aberrant nerve regeneration. Stroke (spinal or brain) and spinal cord injury can also induce neuropathic pain. Cancer-related neuropathic pain results from tumor growth compression of adjacent nerves, brain, or spinal cord. In addition, cancer treatments, including chemotherapy and radiation therapy, can also cause nerve injury.

Unfortunately, neuropathic pain is often resistant to available drug therapies. Treatments for neuropathic pain include opioids, anti-epileptics (e.g., gabapentin, carbamazepine, valproic acid, topiramate, phenytoin), NMDA antagonists (e.g., ketamine, dextromethorphan), topical Lidocaine (for post-herpetic neuralgia), and tricyclic anti-depressants (e.g., fluoxetine (Prozac®), sertraline (Zoloft®g), amitriptyline, among others). Neuropathic pain is frequently only partially relieved by high doses of opioids, which are the most commonly used analgesics (Chemy et al., Neurology 1994; 44: 857-61.; MacDonald, Recent Results Cancer Res. 1991; 121: 24-35.; McQuay, 1997, supra). Current therapies may also have serious side effects such as cognitive changes, sedation, and nausea. Many patients suffering from neuropathic pain are elderly or have medical conditions that limit their tolerance of such side effects.

### 2.3. Inflammatory Pain

Chronic somatic pain generally results from inflammatory responses to tissue injury such as nerve entrapment, surgical procedures, cancer or arthritis (Brower, Nature Biotechnology 2000; 18: 387-391). Although many types of inflammatory pain are currently treated with NSAIDs, there is much room for improved therapies.

The inflammatory process is a complex series of biochemical and cellular events activated in response to tissue injury or the presence of foreign substances (Levine, Inflammatory Pain, In: Textbook of Pain, Wall and Melzack eds., 3rd ed., 1994). Inflammation often occurs at the site of injured tissue or foreign material, and generally contributes to the process of tissue repair and healing. The cardinal signs of inflammation include erythema (redness), heat, edema (swelling), pain and loss of function (ibid.). The majority of patients with inflammatory pain do not experience pain continually, but rather experience enhanced pain when the inflamed site is moved or touched.

Tissue injury induces the release of inflammatory mediators from damaged cells. These inflammatory mediators include ions (H⁺, K⁺), bradykinin, histamine, serotonin (5-HT), ATP and nitric oxide (NO) (Kidd and Urban, Br. J. Anaesthesia 2001, 87: 3-11). The production of prostaglandins and leukotrienes is initiated by activation of the arachidonic acid (AA) pathway. Via activation of phospholipase A2, AA is converted to prostaglandins by cyclooxygenases (Cox-1 and Cox-2), and to leukotrienes by 5-lipoxygenase. The NSAIDs exert their therapeutic action by inhibiting cyclooxygenases. Recruited immune cells release further inflammatory mediators, including cytokines and growth factors, and also activate the complement cascade. Some of these inflammatory mediators (e.g., bradykinin) activate nociceptors directly, leading to spontaneous pain. Others act indirectly via inflammatory cells, stimulating the release of additional pain-inducing (algogenic) agents. Application of inflammatory mediators (e.g., bradykinin, growth factors, prostaglandins) has been shown to produce pain, inflammation and hyperalgesia (increased responsiveness to normally noxious stimuli).

### 2.4. Genetics

Recent efforts to treat neuropathic pain have focused on identification of genes that are differentially regulated in response to pain stimuli. Using rat models of neuropathic pain, changes in gene and protein expression in the injured part of dorsal root ganglia (DRG) neurons (ipsilateral) compared with the uninjured side (contralateral) or uninjured neurons have been reported (Wang et al., Neuroscience 2002; 114: 520-46; Kim et al., NeuroReport 2001; 12: 3401-05; Xiao et al., Proc. Natl. Acad. Sci. USA 2002; 99: 8361-65; Costigan et al., BMC Neuroscience 2002; 3: 16; and Sun et al., BMC Neuroscience; 2002; 3: 11). Genes that were found to be up-regulated in injured neurons include those that encode cell-cycle and apoptosis-related proteins; genes associated with neuroinflammation and immune activation, including complement proteins; a gene encoding for calcium channel α₂δ; genes encoding transcription factors; and genes encoding structural proteins or glycoproteins involved in tissue remodeling (Wang et al., supra). Genes that were down-regulated compared with uninjured neurons include: neuropeptides such as somatostatin and Substance P; the serotonin 5HT-3 receptor; the glutamate receptor 5 (GluR5); sodium and potassium channels; calcium signaling molecules; and synaptic proteins (Wang et al., supra).

Neuronal transcription factors are also differentially regulated in injured neurons. Transcription factors determined to be differentially expressed include JunD, NGF1-A and MRGl (Xiao et al., supra; Sun et al., supra).

Despite the identification of certain genes that are differentially regulated in models of pain, there remains a need to identify other pain-related genes, and to develop more effective therapies to treat pain, particularly neuropathic pain.

### 2.5. The Complement Cascade and Its Role in Immunity

The complement system is composed of a large number of distinct plasma proteins that react with one another to opsonize pathogens and induce a series of inflammatory responses that help to fight infection. The complement system activates immune response through triggered-enzyme cascades. The components of the complement cascade include proteolytic pro-enzymes that become sequentially activated, leading to activation of complement components and amplification of the complement system. The end result of this complex pathway is the chemotaxis of immune cells, opsonization of pathogens or injured cells, and/or lysis of pathogens or injured cells. A schematic overview of the complement cascade and its consequences, including its three distinct activation pathways (i.e., the classical pathway, the mannan-binding lectin pathway, and the alternative pathway), is provided in FIG. 1. FIG. 2 shows the complement cascade with its various components. For a more detailed description of the complement cascade and its components, see Ember and Hugli, Immunopharmacology 1997, 38: 3-15; and Janeway, Immunobiology, Fifth Ed. 2001, Garland Publishing, pgs. 43-64.

The role of complement components in physiological and pathological immune and inflammatory responses has been and continues to be a major focus of study. In humans, complement has been shown to be involved in both classical inflammation conditions (such as arthritis and nephritis) as well as in reperfusion injuries (such as myocardial/cerebral infarction), arteriosclerosis, rejection of transplants, and degenerative disorders. Animal models of some of these diseases treated with complement inhibitory reagents have shown suppression of the immune and inflammatory effects of complement (reviewed and references within Morgan and Harris, Mol Immunol 2003, 40:159; Mizuno and Morgan, Inflammation and Allergy, 2004, 3:87). Animal models of neuropathies such as experimental allergic neuritis, and experimental allergic encephalitis (Vriesendorp et al., J, Neuroimmunol 1995, 58:157; Piddlesden et al., J. Immunol. 1994, 152: 5477) have also been shown to involve a complement component. Direct axonal injuries, such as nerve crush and axotomy, which lead to Wallerian degeneration of the nerve fiber along with its myelin sheath, have been shown to be accompanied by complement activation (Jonge et al., Hum Mol Gen 2004, 13: 295; Dailey et al., Hum Mol Gen 1998, 18:6713). However, even though these models of neuropathies and neuronal injuries represent painful conditions, relief of pain by complement inhibition has not been directly demonstrated.

Jinsmaa et al. (Life Science 2000, 67: 2137-2143) demonstrate that intracerebroventricular administration of C3a produces an anti-opioid effect on mice treated with morphine and U-50488H, μ- and κ-opioid receptor agonists, respectively. According to this article, the analgesic effect of morphine or U-50488H on acute pain responses as measured by tail flick or hot plate is reduced after C3a application directly to the CNS. However, this article fails to teach or make obvious whether or not the inhibition of C3a would have an “anti-anti-opioid” effect to ameliorate established chronic pain states. Jinsmaa et al. postulate that C3a antagonizes the binding of morphine and U-50488H to the μ- and κ-Opioid receptor, respectively, thus leading to a reduction in analgesia when pain is elicited acutely. During chronic pain states, it is not clear from Jinsmaa et al. what the effect would be of reducing C3a in the absence of exogenously introduced opioid receptor agonists, but rather in the presence of endogenous opioid receptor ligands. In fact, since C3a is a peptide generally expected to be incapable of crossing the blood brain barrier under normal physiological conditions, it is not clear whether the observed anti-opioid effect occurs without exogenous intervention as described. In summary, these studies suggest the possible existence of an interaction, direct or indirect, between one component of the complement pathway, C3a, and opioid-mediated analgesia occurring in the brain. However, these studies do not address a causal relationship between complement activation and maintenance of a chronic pain state, especially one in the PNS.

Chacur et al. Pain 2001, 94:231, describes development of a model of pain called sciatic inflammatory neuritis (SIN). This model is based on the observation that many pain-causing neuropathies are accompanied by inflammation and/or infection near affected nerves. In order to test the hypothesis that inflammation in close proximity to nerves can cause pain, the authors test two different pro-inflammatory agents: high mobility group-1 (HMG), a pro-inflammatory cytokine; and zymosan (yeast cell walls), whose pro-inflammatory effects are mediated through complement activation. With the injection of either pro-inflammatory reagent, the authors observed a dose-dependent shift of mechanical allodynia from unilateral (ipsilateral to the site of injection) to bilateral (both hindpaws). This is a phenomenon commonly observed in the clinic associated with neuropathies and is termed “mirror” pain. The authors specifically conclude that the allodynia is not specific to zymosan, as HMG injection in their experiments also dose-dependently induces the mirror pain. Rather they conclude that low levels of peri-sciatic acute immune activation induces unilateral allodynia, while high levels can create bilateral or mirror allodynia.

In a subsequent study, Twining et al. (Pain 2004, 110:299-309) further characterized the SIN model with respect to the effectiveness of immune inhibitors and antagonists (including the TNF binding protein, IL-6 neutralizing antibody, IL-1 receptor antagonist, reactive oxygen species scavengers, and sCR1 complement inhibitor) in alleviating the zymosan-induced pain only (not the HMG-induced pain). The authors demonstrate that perisciatic pretreatment prior to injection of zymosan with any of the above described inhibitors of inflammation was successful in preventing development of either ipsilateral or contralateral allodynia associated with the SIN model. As a result, the authors conclude that proinflammatory cytokines, reactive oxygen species, and complement are early mediators of allodynias resulting from sciatic inflammatory neuritis. While the data implicates cytokines and reactive oxygen species as downstream effectors of SIN pain induction, their interpretation with respect to complement is flawed. Since in their model, the sciatic inflammatory neuritis is specifically induced by complement activation (via zymosan injection), it should not be surprising that pretreatment with a complement inhibitor should prevent development of SIN-associated pain as the source of inflammatory neuritis itself is inhibited. In addition, the authors themselves point out that the inflammatory mediators they have identified (cytokines, reactive oxygen species, and complement) required pretreatment to prevent pain induction, and are therefore, only implicated for the creation of SIN-induced pain enhancement. Whether these same factors remain important for the prolonged maintenance of chronic allodynia was not addressed by their study. Therapeutics designed to prevent the induction of pain are of minimal utility, as it is unlikely that pain would be treated prophylactically; it is far more relevant to develop analgesics directed against mechanisms involved in the maintenance of pain, as they can be used after the establishment of the pain state. It is not obvious from this study that therapeutics directed against the complement pathway should be effective in ameliorating established chronic pain conditions.

In summary, multiple studies have previously associated complement with the development of various neuropathies. Inhibition of the immune and inflammatory effects of complement can reduce the extent of pathology associated with some of these neuropathies. However, to date, a demonstration of a causal relationship between complement cascades and chronic pain accompanying nerve injury, whether caused by physical injury or inflammation, has yet to be demonstrated. In particular, the utility of modulators of complement activity for the treatment of established chronic pain states has not been previously demonstrated.

The citation or discussion of a published reference in this section and throughout the specification is provided merely to clarify the description or context of the present invention and is not an admission that any such reference is “prior art” to the invention described herein.

### 3. SUMMARY OF THE INVENTION

The present invention provides a method for detecting a pain response in a test cell, said method comprising:
(a) determining the expression level of a complement component-encoding nucleic acid molecule in a test cell capable of expressing the nucleic acid molecule; and
(b) comparing the expression level of the complement component-encoding nucleic acid molecule in the test cell to the expression level of the nucleic acid molecule in a control cell that is not exhibiting a pain response;
   wherein a detectable difference between the expression level of the complement component-encoding nucleic acid molecule in the test cell and the expression level of the complement component-encoding nucleic acid molecule in the control cell indicates that the test cell is exhibiting a pain response.

The present invention further provides a method for detecting a pain response in a test cell, said method comprising:
(a) determining the expression level of a complement component in a test cell capable of expressing the complement component; and
(b) comparing the expression level of the complement component in the test cell to the expression level of the complement component in a control cell that is not exhibiting a pain response;
   wherein a detectable difference between the expression level of the complement component protein in the test cell and the expression level of the complement component in the control cell indicates that the test cell is exhibiting a pain response.

The present invention also provides a method for detecting a pain response in a test cell, said method comprising:
(a) determining a biological activity of a complement component in a test cell capable of expressing the complement component; and
(b) comparing the biological activity of the complement component in the test cell to the biological activity of the complement component in a control cell that is not exhibiting a pain response;
   wherein a detectable difference between the biological activity of the complement component in the test cell compared to the biological activity of the complement component in the control cell indicates that the test cell is exhibiting a pain response.

In one embodiment of any of the aforementioned methods for detecting a pain response, the complement component is a complement effector, and the detectable difference is selected from (i) an increase in the expression of the complement effector-encoding nucleic acid molecule, (ii) an increase in the expression of the complement effector, and (iii) an increase in biological activity of the complement effector. In a non-limiting embodiment, the complement effector is selected from C3, C3aR, C5aR, C5, C3 convertase, C5 convertase, Factor D, C1s, MASP-1, MASP-2, MASP-3, Factor B, C1r, and C5b-9. In a specific embodiment, the complement effector is C3 convertase.

In another embodiment of any of the aforementioned methods for detecting a pain response, the complement component is an endogenous complement inhibitor, and the detectable change is selected from (i) a decrease in the expression of the endogenous complement inhibitor-encoding nucleic acid molecule; (ii) a decrease in the expression of the endogenous complement inhibitor, and (iii) a decrease in biological activity of the endogenous complement inhibitor. In one non-limiting embodiment, the endogenous complement inhibitor is DAF, Factor H, Factor I, CRRY, CR1, clusterin, CD59, or C1 INH.

In another embodiment of any of the aforementioned methods for detecting a pain response, the type of pain detected is neuropathic pain, nociceptive pain, chronic pain, inflammatory pain, pain associated with cancer, or pain associated with rheumatic disease.

The cells used in any of the aforementioned methods for detecting a pain response can be cells that constitutively express the nucleic acid molecule encoding a complement component or express the nucleic acid molecule encoding a complement component in response to a specific stimulus. Such cells can be those that naturally express an endogenous nucleic acid molecule encoding a complement component, or cells that have been genetically modified to express or overexpress a nucleic acid molecule encoding a complement component.

Cells used in any of the aforementioned methods for detecting a pain response can be from the central nervous system (CNS) or from the peripheral nervous system (PNS). In one embodiment, such cells are from the dorsal root ganglion (DRG). In another embodiment, such cells are from an animal model of pain, such as from a mouse, rat, or from a human.

The complement component that is the focus of any of the aforementioned methods for detecting a pain response can be selected from a mammalian complement component, and preferably from a rat, mouse, or human.

The present invention provides novel methods for treating pain by modulating a component of the complement cascade. More particularly, the present invention provides a method for treating pain by modulating expression of either a complement component-encoding nucleic acid molecule or a complement component, comprising administering to a subject in need of such treatment a therapeutically effective amount of a compound that modulates expression of the complement component-encoding nucleic acid molecule or the complement component.

The present invention further provides a method for treating pain by modulating the biological activity of a complement component in a subject feeling pain, comprising administering to the subject a therapeutically effective amount of a compound that modulates a biological activity of the complement component protein, with the proviso that the compound is not cobra venom factor (CVF).

In a non-limiting embodiment of any of the aforementioned methods for treating pain, the complement component is a complement effector, and the function of the compound is selected from (i) decreasing the expression of a nucleic acid molecule having a nucleotide sequence encoding the complement effector, (ii) decreasing the expression of the complement effector; and (iii) decreasing a biological activity of the complement effector.

In another non-limiting embodiment of any of the aforementioned methods for treating pain, the complement component is a complement effector, and the function of the compound is selected from (i) inhibiting an increase in the expression of a nucleic acid molecule having a nucleotide sequence encoding the complement effector, (ii) inhibiting an increase in the expression of the complement factor, and (iii) inhibiting an increase in a biological activity of the complement effector.

In a non-limiting embodiment, the complement effector is selected from C3, C3aR, C5aR, C5, C3 convertase, C5 convertase, Factor D, C1s, MASP-1, MASP-2, MASP-3, Factor B, C1r, and C5b-9. In a specific embodiment, the complement effector is C3 convertase.

In another non-limiting embodiment of any of the aforementioned methods for treating pain, the complement component is an endogenous complement inhibitor, and the function of the compound is selected from (i) increasing the expression of a nucleic acid molecule having a nucleotide sequence encoding the endogenous complement inhibitor, (ii) increasing the expression of the endogenous complement inhibitor, and (iii) increasing a biological activity of the endogenous biological inhibitor.

In another non-limiting embodiment of any of the aforementioned methods for treating pain, the complement component is an endogenous complement inhibitor, and the function of the compound is selected from (i) inhibiting a decrease in expression of a nucleic acid molecule having a nucleotide sequence encoding an endogenous complement inhibitor, (ii) inhibiting a decrease in expression of an endogenous complement inhibitor, and (iii) inhibiting a decrease in a biological activity of an endogenous complement inhibitor.

In a non-limiting embodiment the endogenous complement inhibitor is DAF, Factor H, Factor I, CRRY, CR1, clusterin, CD59, or C1 INH.

In another embodiment of any of the aforementioned methods for treating pain, the complement component is active in at least one of the pathways selected from the group consisting of: (i) the classical pathway; (ii) the MB-lectin pathway; (iii) the alternative pathway; and (iv) the downstream shared pathway.

In any of the present methods for treating pain, the type of pain can be any type of pain, and preferably pain selected from neuropathic pain, nociceptive pain, chronic pain, pain associated with cancer, and pain associated with rheumatic disease.

The present invention further provides a method for identifying a compound capable of treating pain by modulating expression of a nucleic acid molecule having a nucleotide sequence encoding a complement component, said method comprising:
(a) contacting a first cell capable of expressing a nucleic acid molecule having a nucleotide sequence encoding a complement component with a test compound under conditions sufficient to allow the first cell to respond to said contact with the test compound;
(b) determining in the first cell the expression level of the complement component-encoding nucleic acid molecule during or after contact with the test compound; and
(c) comparing the expression level of the complement component-encoding nucleic acid molecule in the first cell determined in step (b) to the expression level of the complement component-encoding nucleic acid molecule in a second (control) cell that has not been contacted with the test compound;
   wherein a detectable difference between the expression level of the complement component-encoding nucleic acid molecule in the first cell in response to contact with the test compound and the expression level of the complement component-encoding nucleic acid molecule in the second cell indicates that the test compound modulates expression of the complement component-encoding nucleic acid molecule. A test compound that can modulate the expression of the complement component-encoding nucleic acid molecule is a candidate for a compound that can treat pain, and can be subjected to further testing and analysis.

The present invention further provides a method for identifying a compound capable of treating pain by modulating expression of a complement component, said method comprising:
(a) contacting a first cell capable of expressing a complement component with a test compound under conditions sufficient to allow the first cell to respond to said contact with the test compound;
(b) determining in the first cell the expression level of the complement component during or after contact with the test compound; and
(c) comparing the expression level of the complement component in the first cell determined in step (b) to the expression level-of the complement component in a second (control) cell that has not been contacted with the test compound;
wherein a detectable difference between the expression level of the complement component in the first cell in response to contact with the test compound and the expression level of the complement component in the second cell indicates that the test compound modulates expression of the complement component. A test compound that can modulate the expression of complement component is a candidate for a compound that can treat pain, and can be subjected to further testing and analysis.

The present invention further provides a method for identifying a compound capable of treating pain by modulating a biological activity of a complement component, said method comprising:
(a) contacting the complement component with a test compound under conditions sufficient to allow the complement component to respond to said contact with the test compound;
(b) determining a biological activity of the complement component during or after contact with the test compound; and
(c) comparing the biological activity of the complement component determined in step (b) to the biological activity of the complement component when the component has not been contacted with the test compound;
   wherein a detectable difference between the biological activity of the complement component in response to contact with the test compound and the biological activity of the complement component when the component has not been contacted with the test compound indicates that the test compound modulates the biological activity of the complement component. A test compound that can modulate a biological activity of a complement component is a candidate for a compound that can treat pain, and can be subjected to further testing and analysis.

In a non-limiting embodiment of any of the aforementioned screening methods, the complement component is a complement effector, and the function of the test compound is selected from (i) decreasing the expression of a nucleic acid molecule having a nucleotide sequence encoding the complement effector, (ii) decreasing the expression of the complement effector, and (iii) decreasing the biological activity of the complement effector.

In another non-limiting embodiment of any of the aforementioned screening methods, the complement component is a complement effector, and the function of the test compound is selected from (i) inhibiting an increase in expression of a nucleic acid molecule having a nucleotide sequence encoding the complement effector, (ii) inhibiting an increase in expression of the complement effector, and (iii) inhibiting an increase in the biological activity of the complement effector.

In a non-limiting embodiment, the complement effector that is the focus of any of the aforementioned screening methods is selected from C3, C3aR, C5aR, C5, C3 convertase, C5 convertase, Factor D, C1s, MASP-1, MASP-2, MASP-3, Factor B, C1r, and C5b-9. In a specific embodiment, the complement effector is C3 convertase.

In another non-limiting embodiment of any of the aforementioned screening methods, the complement component is an endogenous complement inhibitor, and the function of the test compound is selected from (i) increasing the expression of a nucleic acid molecule having a nucleotide sequence encoding for the endogenous complement inhibitor, (ii) increasing the expression of the endogenous complement inhibitor, and (iii) increasing the biological activity of the endogenous complement inhibitor.

In another non-limiting embodiment of any of the aforementioned screening methods, the complement component is an endogenous complement inhibitor, and the function of the test compound is selected from (i) inhibiting a decrease in expression of a nucleic acid molecule having a nucleotide sequence encoding the endogenous complement inhibitor, (ii) inhibiting a decrease in expression of the endogenous complement inhibitor, and (iii) inhibiting a decrease in biological activity of the endogenous complement inhibitor.

In a non-limiting embodiment, the endogenous complement inhibitor that is the focus of any of the aforementioned screening methods is selected from DAF, Factor H, Factor I, CRRY, CR1, clusterin, CD59, or C1 INH.

In another embodiment of any of the aforementioned screening methods, the complement component is active in at least one of the pathways selected from the group consisting of: (i) the classical pathway; (ii) the MB-lectin pathway; (iii) the alternative pathway; and (iv) the downstream shared pathway.

In one specific embodiment, the nucleic acid molecule has a nucleotide sequence encoding a mammalian complement component. In a more specific embodiment, the nucleic acid molecule has a nucleotide sequence encoding a rat, mouse or human complement component. The nucleotide sequence can be any sequence encoding said component, including a genomic sequence, a cDNA sequence, or a degenerate variant thereof.

In one specific embodiment, the complement component comprises the amino acid sequence of a mammalian complement component. In a more specific embodiment, the complement component comprises the amino acid sequence of a rat, mouse or human complement component.

In any of the aforementioned screening methods, the type of pain is selected from neuropathic pain, nociceptive pain, chronic pain, pain associated with cancer, and pain associated with rheumatic disease.

Cells used in any of the aforementioned screening methods can either constitutively express a nucleotide molecule encoding a complement component, or express a nucleotide molecule encoding a complement component in response to a specific stimulus. Such cells can be those that naturally express an endogenous nucleic acid molecule encoding a complement component, or can be cells that have been genetically modified to express or overexpress a nucleic acid molecule encoding a complement component. Cells useful in any of the aforementioned screening methods can be selected from the CNS or PNS. In certain embodiments, the cells are selected from the DRG. In certain embodiments, the cells are from an animal model of pain.

A screening method of the present invention can be performed with cells from any appropriate mammalian subject, such as a mouse, rat, guinea pig, rabbit, dog, cat, monkey or human. The cells can be from subjects used as animal models of pain.

A screening method of the present invention can further comprise the steps of:
(a) determining the degree of pain experienced by a test subject during or after contact with the test compound; and
(b) comparing the degree of pain experienced by the test subject in step (a) to the degree of pain experienced by a control subject that has not been contacted with the test compound;
   wherein a detectable difference between the degree of pain experienced by the test subject in response to contact with the test compound and the degree of pain experienced by the control subject indicates that the test compound modulates the pain experienced by the test subject. In a specific embodiment, the test compound decreases pain experienced by the test subject. Such a test compound is a candidate for a compound that can treat pain.

### 4. BRIEF DESCRIPTION OF THE DRAWINGS

FIG. 1 provides an overview of the complement cascade with its three distinct activation pathways: the classical pathway, the MB-lectin pathway, and the alternative pathway. All of these pathways generate crucial enzymatic activities that, in turn, generate the downstream effector molecules of the complement cascade. The three main known consequences of complement activation are opsonization of pathogens, the recruitment of inflammatory cells, and the direct killing of pathogens.

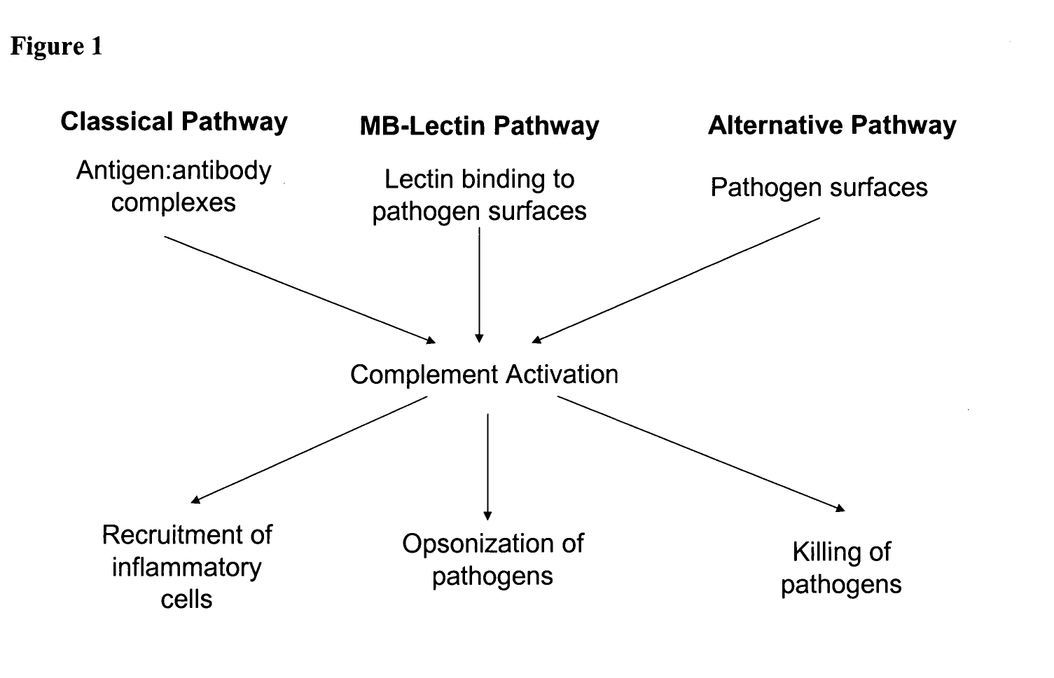

FIG. 2 is a detailed schematic of the complement cascade showing the complement components. Solid arrows show the progression of components as they are added or cleaved. Dashed arrows indicate the proteases that cleave a particular component. The classical, MB-lectin, and alternative pathways lead into the downstream shared pathway. The downstream shared pathway refers to all reactions including and downstream from the cleavage of C3 to C3b and C3a which is catalyzed by either the C3 convertase C4b2b or C3bBb. The downstream shared pathway is delineated by a bar below FIG. 2.

FIG. 3 is a summary of the experimental timeline for surgery, treatment, and testing for the spinal nerve ligation (SNL) model of neuropathic pain to identify genes that are regulated in the pain model.

FIG. 4 is a diagram showing the relationship between the brain, spinal cord, and PNS. The DRG and sciatic nerve are shown in the diagram as part of the PNS.

FIG. 5 provides TaqMan expression profiles across 20 samples from L4 DRG, L5 DRG, L6 DRG, sciatic nerve, and spinal cord from both sham and SNL animals, and from both the ipsi and contra sides for DAF, C3, and a control gene (pitpnb or phosphatidylinositol transfer protein (beta isoform)).

FIG. 6 shows in situ hybridizations of DRGs from rats subjected to either an SNL or sham surgery. The left and right panels show the presence of DAF and C3, respectively. The top and bottom panels show hybridized DRGs from sham and SNL animals, respectively. In the sham panels, DAF expression (as indicated by bright punctate dots) is restricted to a subset of small, likely nociceptive neurons (indicated by arrows), whereas C3 expression is not detected. In SNL panels, DAF expression appears to be downregulated in the neurons, while C3 (as indicated by bright punctate dots) is upregulated mostly in the cells surrounding the neurons (satellite cells as indicated by the arrows).

FIG. 7 shows immunohistochemical staining using a monoclonal antibody to DAF protein (gift from Paul Morgan, University of Wales College, Cardiff, UK) on paraformaldehyde fixed sections of DRG from sham-saline (A) and SNL-saline (B) treated animals. This staining was performed according to the techniques of Spiller et al. (Immunology 1999, 97:374-84), and Mead et al. (J. Immunol 2002, 168: 458-465). Tissue sections from DRGs of sham-saline (C) and SNL-saline (D) treated animals were stained with an antibody to ATF-3 ( Santa Cruz Biotechnology, Inc., Santa Cruz, Calif., cat #SC-188), which is a marker for neuronal injury (Tsujino et al., Mol Cell Neurosci. 2000,15:170-82). Results of immunohistochemical staining agree with results from microchip, TaqMan, and in situ hybridization experiments, i.e., DAF protein expression is down-regulated in the SNL model compared to sham animals.

FIG. 8 is a summary of the experimental timeline for SNL surgery, cobra venom factor (CVF) injections, and animal termination for the experiment testing the relationship between pain and complement inhibition

FIG. 9 compares the effect of CVF or saline treatment on the pain tolerance of rats subjected to either the SNL model of neuropathic pain or sham surgery as presented schematically in FIG. 8. Pain tolerance was determined using a test that measures mechanical hyperalgesia as quantitated by a paw withdrawal threshold (PWT).

FIG. 10 (Panels A-C) shows the activity of C3 in naïve, SNL, and sham animals, as measured in the hemolysis assay. The optical density at 540 nm, measuring hemoglobin release, increases as C3 activity increases. FIG. 10A is a CVF dosing experiment using naïve rats showing the activity of C3 in naïve rats with or without CVF treatment at 0, 3, 6, 7, 8, and 12 days after CVF treatment was given on days 0, 3, and 6. Each bar represents the average of 8 animals (n=8) with CVF treatment, or the average of 2 animals (n=2) for the naïve animals (i.e. no CVF treatment). The data shows that C3 activity decreases after CVF treatment. FIGS. 10B and 10C display C3 levels in rats, before and after SNL or sham surgery, that have been injected with CVF or saline. Each bar represents the average of 5 animals (n=5). The data shows that C3 activity decreases after CVF treatment in both SNL and sham surgery animals.

### 5. DETAILED DESCRIPTION

The present invention provides methods for detecting a pain response in a subject by determining the expression level or activity of a complement component and comparing the expression level or activity to that in a control. The present invention also provides methods for treating pain in a subject by modulating a component of the complement pathway. The present invention further provides methods of screening for compounds that modulate a component of the complement pathway and are thereby useful to treat pain in a subject.

These methods are based on a demonstration that rats with their spinal nerves ligated (SNL) in an animal model for neuropathic pain have a higher pain tolerance, as indicated by an increased withdrawal threshold to a mechanical stimulus, when treated with cobra venom factor (CVF), which is an inhibitor of the complement cascade, compared to SNL animals injected with a saline control.

### 5.1. The Complement System

Before providing a detailed description of the diagnostic, therapeutic, and screening methods of the present invention, the following paragraphs serve to describe and define the complement system, including complement components, complement effectors, and complement inhibitors. Briefly, complement components include proteins that participate in the complement system. Complement effectors are complement components that lead to or result in a consequence of the complement cascade. Complement inhibitors are compounds that inhibit or reduce a consequence of the complement system, and can be either endogenous complement components or exogenous inhibitors.

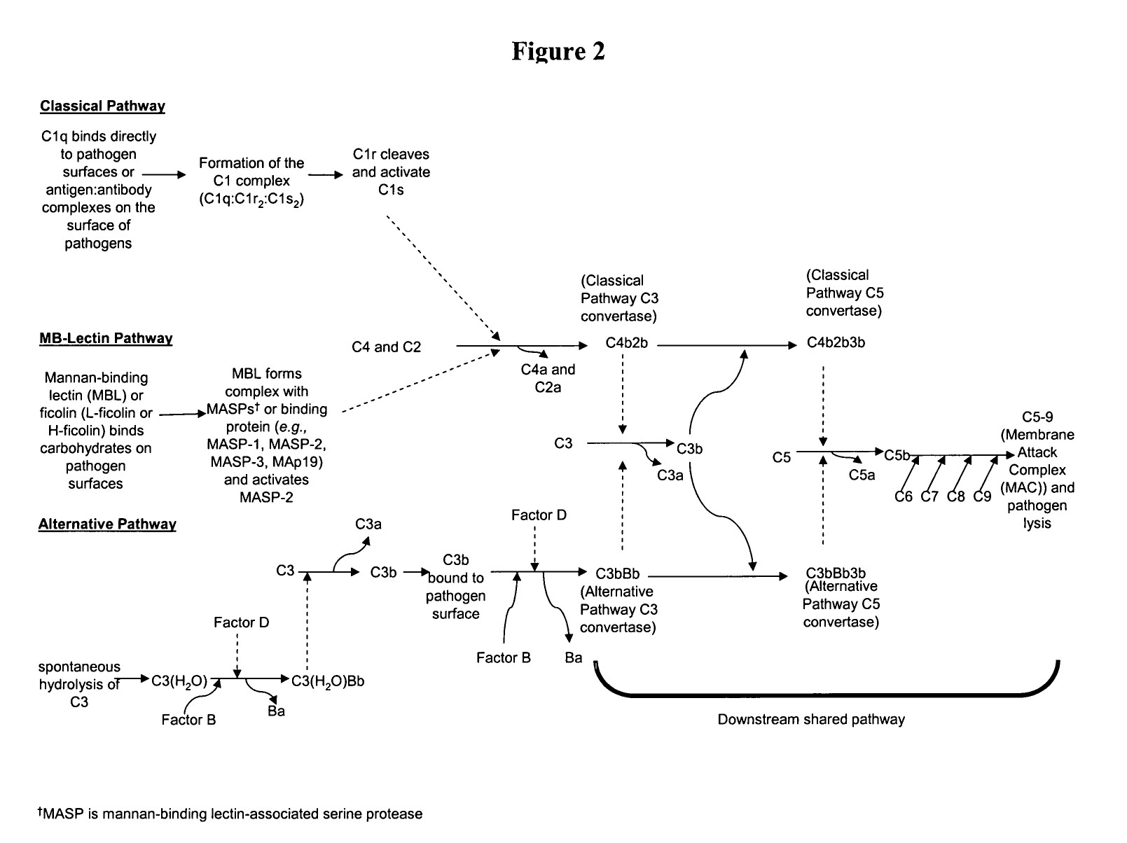

The complement system can be activated by three distinct pathways: the “classical” pathway, the “mannan binding-lectin” (or “MB-lectin”) pathway, and the “alternative” pathway, as shown in FIG. 2.

The term “classical pathway” refers to activation of the complement system triggered by the binding of the complement component C1q to an antibody:antigen complex on a pathogen surface, or by direct binding of C1q to a pathogen surface. C1q then forms the C1 complex with 2 molecules of each of C1r and C1s. Formation of the C1 complex (i.e., C1q:C1r₂:C1s₂) leads to activation of C1r, which is an autocatalytic enzyme. After activation, C1r cleaves the associated C1s to generate active C1s. Active C1s then cleaves C4 and C2 to generate C4b, C2b, C4a, and C2a. C4b and C2b then form the C4b2b complex (i.e., the “classical pathway C3 convertase”) on the pathogen surface. The term “classical pathway” refers to the steps in the complement pathway starting with C1q binding and ending with to the formation of C4b2b.

The “MB-lectin pathway” refers to activation of the complement system triggered by the binding of mannan-binding lectin (MBL) or a ficolin (e.g., L-ficolin or H-ficolin) to carbohydrates on the surface of pathogens. Following binding, MBL complexes containing MBL and mannan-binding lectin-associated serine proteases or binding proteins (e.g., MASP-1, MASP-2, MASP-3, and MAp19) are activated. For example, complex formation with MBL can result in activation of MASP-2. Subsequently, MASP-2 cleaves C4 and C2 to form C4a, C4b, C2a, and C2b. C4b and C2b then form the C4b2b complex (i.e., the “classical pathway C3 convertase”) on the pathogen surface. The MB-lectin pathway refers to the steps in the complement pathway starting with the binding of MBL to the pathogen surface and ending with the formation of the C4b2b complex.

The “alternative pathway” refers to activation of the complement system initiated by the spontaneous hydrolysis of C3 to form C3(H₂O). Following the formation of C3(H₂O), Factor B binds to C3(H₂O). Factor D then cleaves the Factor B associated with C3(H₂O) to form Bb and Ba. Bb remains bound to C3(H₂O) to form the C3(H₂O)Bb complex. The C3(H₂O)Bb complex then cleaves C3 to C3a and C3b. C3b then binds to the pathogen surface and associates with Factor B. Factor D then cleaves Factor B associated with C3b to form Bb and Ba. Bb remains bound to C3b to form the alternative pathway C3 convertase, C3bBb. The “alternative pathway” refers to steps in the complement pathway starting with the spontaneous hydrolysis of C3 and ending with formation of the C3bBb complex.

FIG. 2 provides an abbreviated schematic of the complement cascade showing some complement components. As shown in FIG. 2, each of the three pathways follows a sequence of reactions to generate a C3 convertase. The C3 convertase then cleaves C3 into C3a and C3b, and C3b subsequently binds to a C3 convertase complex to form a C5 convertase. If C3b binds to a classical pathway C3 convertase (i.e., C4b2b), a classical pathway C5 convertase is formed (i.e., C4b2b3b). If C3b binds to an alternative pathway C3 convertase (i.e., C3bBb), an alternative pathway C5 convertase is formed (i.e., C3bBb3b). Both the classical pathway C5 convertase and the alternative pathway C5 convertase cleave C5 to form C5a and C5b. C5b binds to C6, C7, C8, and C9 to form the membrane attack complex (i.e., the MAC), which induces pathogen lysis by creating a pore in the membrane of the pathogen.

The term “downstream shared pathway” refers to reactions including, and downstream from, the cleavage of C3 to C3b and C3a which is catalyzed by either the C3 convertase C4b2b or C3bBb.

### 5.2. Complement Components

As used herein, the term “complement component” refers to an endogenous component of the complement cascade. Both complement effectors (see below) and endogenous complement inhibitors (see below) are considered herein to be complement components.

Complement components include, but are not limited to, the proteolytic pro-enzymes (e.g., C2 and Factor B); proteases (e.g., C1r, C1s, C2b, Bb, Factor D, MASP-1, MASP-2, MASP-3); non-enzymatic components that form functional complexes (e.g., C1q, C4b, and C3b); regulators (e.g. properdin, decay accelerating factor (DAF), and Factor H (H)); and receptors (e.g., CR1, CR2, CR3, CR4, and CR1qR; also see below) of the complement cascade.

Complement components further include complement receptors (CRs) on phagocytes that specifically recognize and bind complement components on the surface of pathogens and which facilitate the uptake and destruction of pathogens by phagocytic cells. CR1 (i.e., CD35) binds C3b, C4b, and iC3b on the surface of pathogens. CR2 (i.e., CD21) binds C3d, iC3b, and C3dg (which is a secondary breakdown product of C3b). CR3 (i.e.,CD11b/CD18) and CR4 (i.e., gp150,95; CD11c/CD18) bind iC3b. The C5a receptor (i.e., C5aR, CD88) binds C5a. The C3a receptor (i.e., C3aR) binds C3a.

Complement components also include anaphylatoxins (e.g., C3a, C4a, and C5a) which are also known as small complement components. Anaphylatoxins act on specific receptors to produce local inflammatory responses.

### 5.2.1. Complement Effectors

A “complement effector” is a complement component that participates in the classical pathway, alternative pathway, MB-lectin pathway, or downstream shared pathway with a function that leads to or results in a consequence of the complement cascade (e.g., the recruitment of inflammatory cells, the opsonization of pathogens, or the killing of pathogens). Alternatively, a “complement effector” is a complement component that binds to a participant of the classical pathway, alternative pathway, MB-lectin pathway, or downstream shared pathway with a function that leads to or results in, a consequence of the complement cascade (e.g., the recruitment of inflammatory cells, the opsonization of pathogens, or the killing of pathogens).

Complement effectors include, but are not limited to, C1q, C1r, C1s, MBL, MASP-1, MASP-2, MASP-3, C4, C2, C4a, C2a, C3, C3a, C3b, Factor D, Factor B, Ba, Bb, C3bBb (the alternative pathway C3 convertase), C4b, C2b, C4b2b (the classical pathway C3 convertase), C4b2b3b (the classical pathway C5 convertase), C3bBb3b (the alternative pathway C5 convertase), C5, C5a, C5b, C6, C7, C8, C9, and C5-9 (or MAC) as shown in FIG. 2. Additionally, properdin (i.e., Factor P), which binds and stabilizes the C3bBb, is a complement effector.

### 5.2.2. Complement Inhibitors

A “complement inhibitor” is a compound that inhibits or reduces any consequence of the complement cascade (such as, e.g., the recruitment of inflammatory cells, the opsonization of pathogens, or the killing of a pathogen).

In one embodiment, a complement inhibitor is a molecule that inhibits or reduces the expression of a complement effector-encoding nucleic acid molecule, or the expression of a complement effector, or a biological activity of a complement effector. In a particular embodiment, a complement inhibitor leads to the reduction of complement activation and/or complement activity.

In another embodiment, a complement inhibitor is a molecule that increases, directly or indirectly, the transcription of an endogenous complement inhibitor-encoding nucleic acid molecule, or the expression of an endogenous complement inhibitor protein, or the activity of an endogenous complement inhibitor protein.

In one embodiment, the complement inhibitor is an endogenously occurring molecule (e.g., a complement regulatory protein, e.g., C1INH). In another embodiment, the complement inhibitor is a non-endogenously occurring molecule (e.g., a small molecule drug).

### 5.2.2.1. Endogenous Complement Inhibitors

In one embodiment, a complement inhibitor is an “endogenous complement inhibitor”. An endogenous complement inhibitor is a complement component that inhibits or reduces a consequence of the complement cascade (e.g., the recruitment of inflammatory cells, the opsonization of a pathogen, or the killing of a pathogen).

Endogenous complement inhibitors include, but are not limited to, the C1 inhibitor (C1 INH), the C4-binding protein (C4BP), complement receptor 1 (CR1), Factor H (H), Factor I (I), decay accelerating factor (DAF), membrane cofactor protein (MCP), CD59 (protectin), carboxypeptidase N, Protein S, and clusterin (SP-40).

C1INH binds to activated C1r:C1s and causes C1r to dissociate from C1q. C4BP binds to C4b and displaces C2b bound to C4b. C4BP is also a cofactor for I cleavage of C4b. CR1 binds C4b, which displaces C2b bound to C4b. CR1 is also a cofactor for I. Alternatively, CR1 binds C3b, which displaces CBb bound to C3b. Factor H binds C3b, which displaces Bb bound to C3b. Factor H is also a cofactor for I. Factor I is a serine protease that cleaves C3b first into iC3b and then further to C3dg. Factor I also cleaves C4b first into C4c and then to C4d. Factor H, MCP, C4BP, and CR1 are each co-factors required for optimal functioning of Factor I. DAF is a membrane protein that displaces Bb from C3b, and C2b from C4b. Membrane cofactor protein (MCP) is a membrane protein that promotes C3b and C4b inactivation by I. CD59 prevents formation of the MAC on autologous or allogenic cells-and is widely expressed on membranes. Carboxypeptidase N inactivates anaphylatoxins by removing a C-terminal arginyl residue of the anaphylatoxin. Protein S binds C5b-C7 and prevents formation of the MAC. Clusterin prevents the activity of the MAC.

In another embodiment, endogenous complement inhibitors are endogenous molecules (e.g., proteins or small molecules as described below) that upregulate the expression of an endogenous complement inhibitor-encoding nucleic acid molecule or protein and/or upregulate the activity of an endogenous complement inhibitor. In other words, endogenous upregulators of endogenous complement inhibitors are also considered herein to be endogenous complement inhibitors. These upregulators of endogenous complement inhibitors include, but are not limited to, molecules that upregulate the expression of DAF, including, e.g., estrogen (Song et al., J. Immunol. 1996, 157:4166-72); heparin-binding epidermal growth factor-like growth factor (alternatively named HB-EGF described in Young et al., J Clin Endocrinol Metab. 2002, 87:1368-75); TNFα (Zhang et al., Eur J Immunol. 1998, 28:1189-96); Interleukin (IL)-4 (Andoh et al., Gastroenterology 1996, 111:911-8); histamine (Tsuji et al., J Immunol. 1994, 152:1404-10); and nerve growth factor (NGF, described in Kendall et al., J Neurosci Res. Jul. 15, 1996; 45(2):96-103).

### 5.2.2.2. Exogenous Complement Inhibitors

Exogenous complement inhibitors include, but are not limited to, synthetic chemical compounds (e.g., small molecule inhibitors), polyionic agents, monoclonal antibodies, non-endogenous peptides, non-endogenous soluble proteins, and non-endogenous inhibitory oligonucleotides.

Examples of small molecule inhibitors include SB-290157, which is a C3aR antagonist from SmithKline Beecham Pharmaceuticals (described on the WorldWideWeb at gsk.com/about/about.htm, and referenced in Ames et al., J Immunology 2001, 166: 6341-6348, and U.S. Pat. No. 6,489,339); NGD-2000-1, which is a C5aR antagonist from Neurogen Corp., Branford, Conn. (described on the WorldWideWeb at neurogen.com/contact.htm); L-747981 (or IDDB10835), which is a C5aR antagonist from Merck, Whitehouse Station, N.J. (referenced in Laszlo et al., Bioorg. Med. Chem. Lett. 1997, 7: 213-218); PMX-53 (or AcF(OPdChaWR)), which is a CSaR antagonist from Promics Pty Ltd, St. Lucia, Queensland, Australia (referenced in Finch et al., J. Med. Chem. 1999, 42:1965-1974; PCT Publication No. WO 2004/035080, and PCT Publication No. WO 2004/035079); a C5a receptor antagonist described in Short et al. Br. J. Pharmacol 1999, 125: 551-554; C1s-INH-248 which is a C1s antagonist from BASF, Ludwigshafen, Germany, (described on the WorldWideWeb at basf.de, and referenced in Buerke et al., J. Immun. 2001, 167:5375-80); IDDB10866 which is a C1r antagonist from Pfizer, New York, N.Y., (described on the WorldWideWeb at pfizer.com, and referenced in Plummer et al., Bioorg. Med. Chem. Lett. 1999, 9:815-820; and Gilmore et al., Bioorg. Med. Chem. Lett. 1996, 6:679-682); K-76COOH (or K-76COONa), which is a C5 inhibitor from Otsuka, Tokyo, Japan, (referenced in, e.g., Fujita et al., Nephron 1999, 81:208-14); FUT-175, which is an inhibitor of C1r, C1s, Factor D, and C3/C5 convertase, from Torii Pharmaceuticals, Inc. Chuo-Ku, Japan (see U.S. Pat. No. 4,454,338; and Aoyama et al., Jap. J. Pharm. 1984, 35:203-27); and BCX-1470, which is an inhibitor of C1s and Factor D from Biocryst in Birmingham, Ala., (referenced in Szalai et al., J. Immun. 2000, 164:463-468; U.S Pat. No. 6,653,340; and PCT Publication No. WO 98/55471).

Additional small molecule complement inhibitors include inhibitors of C1s (see Subasinghe et al., Bioor. Med. Chem. Let. 2004, 14:3043-3047; and PCT Publication No. WO 00/47194); RPR120033, which is a C5a receptor antagonist, (described in Astles et al., Bioor. Med. Chem. Let. 1997, 7:907-912); and inhibitors of C5 convertase (described in Bradbury et al., J. Med. Chem. 2003, 46:2697-2705), among others. Other small molecule complement inhibitors include APT-070, soluble CR1 or CD59-Proadapin, and soluble CD59 (each available from Inflazyme Pharmaceuticals Ltd., Richmond, B.C., Canada).

Small molecule complement inhibitors also include molecules that upregulate expression of endogenous complement inhibitors. For example, upregulators of DAF expression include statins (Mason et al., Circ. Res. 2002, 91: 696-703) and phorbol-12-myristate-13-acetate (Zhang et al., Eur J Immunol. 1998, 28:1189-96).

In one embodiment, an exogenous complement inhibitor is a polyionic agent such as heparin, which is an inhibitor of C1, C3 convertase, and MAC, (see Weiler et al., J. Immunol. 1992, 148:3210-5).

In another embodiment, an exogenous complement inhibitor can be an antibody or immunospecific fragment thereof. Examples of such antibodies include anti-C5 monoclonal antibodies from Alexion-Pharmaceutical, New Haven, Conn. (referenced in published U.S. patent application No. 2003175267; U.S. Pat. No. 6,355,245: U.S. Pat. No. 5,853,722; and Thomas et al., Mol. Immun. 1997, 33:1389-1401); TNX-224 which is an anti-Factor D monoclonal antibody from Tanox, Houston, Tex. (referenced in Fung et al., J. Thor. Cardio. Sur. 2001, 122:113-22; and in Pascual et al., J. Immunological Methods 1990, 127:263-9); anti-C3a receptor antibodies from Human Genome Sciences, Inc. Rockville, Md. (referenced in PCT publication WO 2004/013287, and Zwimer et al., Immunology 1999, 97:166-172); GT-4058, which is an antibody against properdin, from Gliatech, Inc., Cleveland, Ohio, (referenced in U.S. Pat. No. 6,333,034, and in Gupta-Bansal et al., Mol. Immun. 2000, 37:191-201); and anti-C5b-9 monoclonal antibodies (as described in U.S. Pat. No. 5,135,916).

In yet another embodiment, exogenous complement inhibitors can be peptides or proteins, including, but not limited to, peptides that inhibit C1q (as described in Kozlov et al., Biokhimiia 1986, 51:707-18; and Prystowsky et al., Biochemistry 1981, 20:6349-56), or that inhibit C3 (e.g., compstatin as described in PCT Publication No. WO 99/13899; and Morikis et al., Bioch. Soc. Trans. 2004, 32: 28-32); or inhibitory peptides against serine proteases (as described, e.g., by Glover et al., Mol Immunol. 1988, 25:1261-7; Schasteen et al., Mol Immunol. 1991, 28:17-26; and Schasteen et al., Mol Immunol. 1988, 25:1269-75). Exogenous complement inhibitors also include peptides that inhibit C3 and C5 convertase activity (Sandoval et al., J. Immunol. 2000, 165:1066-1073 and Low et. al., J. Immunol. 1999, 162:6580-6588).

Cobra venom factor (CVF; available from Quidel Corp. of San Diego, Calif.) is a protein known to inhibit the complement cascade, and is also an exogenous complement inhibitor. CVF forms a stable C3 convertase, which cleaves C3, primarily in plasma, to form cleavage products C3a and C3b, which are quickly inactivated, thereby eventually depleting endogenous C3 (Cochrane et al., J. Immunology 1970, 105:55-69).

In a specific embodiment, non-endogenous complement inhibitors are soluble proteins. These soluble proteins include, but are not limited to, TP-10 and TP20 (also known as sCR1, a soluble CR1 receptor protein that targets C3b, available from Avant Immunotherapeutics, Inc., Needham, Mass., and referenced in Rittershaus et al., J. Biological Chem. 1999, 274:11237-11244); a soluble fusion of MCP and DAF, which targets C3/C5 convertase (also known as CAB-2, available from Millennium Pharmaceuticals Inc., Cambridge, Mass. and referenced in U.S. Pat. No. 5,679,546); and C1INH, which targets C1 esterase (available from Aventis Behring, Marburg, Germany and referenced in published U.S. patent application No. 2002/168352).

Non-endogenous complement inhibitors can alternatively be inhibitory oligonucleotides, such as antisense oligonucleotides, RNAi molecules, or ribozymes, as described below. Such oligonucleotides include a Factor B antisense oligonucleotide, such as that described in published U.S. patent application No. 2004038925, or antisense oligonucleotides against C3, such as those described in PCT Publication No. WO 03/066805. Such oligonucleotides are useful to inhibit the expression of complement effectors.

### 5.3. Definitions

### 5.3.1. Definitions of Pain and Related Disorders

As used herein, the term “pain” is art recognized and includes a bodily sensation elicited by noxious chemical, mechanical, or thermal stimuli, in a subject, e.g., a mammal such as a human. The term “pain” includes chronic pain such as lower back pain; pain due to arthritis, e.g., osteoarthritis; joint pain, e.g., knee pain or carpal tunnel syndrome; myofascial pain, and neuropathic pain. The term “pain” further includes acute pain, such as pain associated with muscle strains and sprains; tooth pain; headaches; pain associated with surgery; and pain associated with various forms of tissue injury, e.g., inflammation, infection, and ischemia.

“Neuropathic pain” refers to pain caused by injury or disease of the central or peripheral nervous system. In contrast to the immediate (acute) pain caused by tissue injury, neuropathic pain can develop days or months after a traumatic injury. Neuropathic pain frequently is long lasting or chronic, and is not limited in duration to the period of tissue repair. Neuropathic pain can occur spontaneously, or as a result of stimulation that normally is not painful. Neuropathic pain is caused by aberrant somatosensory processing, and is associated with chronic sensory disturbances, including spontaneous pain, hyperalgesia (i.e., sensation of more pain than the stimulus would warrant) and allodynia (i.e., a condition in which ordinarily painless stimuli induce the experience of pain). Neuropathic pain includes, but is not limited to, pain caused by peripheral nerve trauma, viral infection, diabetes mellitus, causalgia, plexus-avulsion, neuroma, limb amputation, vasculitis, nerve damage from chronic alcoholism, hypothyroidism, uremia, and vitamin deficiencies, among other causes. Neuropathic pain is one type of pain associated with cancer. Cancer pain can also be “nociceptive” or “mixed.”

“Chronic pain” can be defined as pain lasting longer than three months (Bonica, Semin. Anesth. 1986, 5:82-99), and may be characterized by unrelenting persistent pain that is not fully amenable to routine pain control methods. Chronic pain includes, but is not limited to, inflammatory pain, post-operative pain, cancer pain, osteoarthritis pain associated with metastatic cancer, trigeminal neuralgia, acute herpetic and post-herpetic neuralgia, diabetic neuropathy, pain due to arthritis, joint pain, myofascial pain, causalgia, brachial plexus avulsion, occipital neuralgia, reflex sympathetic dystrophy, fibromyalgia, gout, phantom limb pain, burn pain, pain associated with spinal cord injury, multiple sclerosis, reflex sympathetic dystrophy and lower back pain and other forms of neuralgia, neuropathic, and idiopathic pain syndromes.

“Nociceptive pain” is due to activation of pain-sensitive nerve fibers, either somatic or visceral. Nociceptive pain is generally a response to direct tissue damage. The initial trauma causes the release of several chemicals including bradykinin, serotonin, substance P, histamine, and prostaglandin. When somatic nerves are involved, the pain is typically experienced as an aching or pressure-like sensation.

In the phrase “pain and related disorders”, the term “related disorders” refers to disorders that either cause or are associated with pain, or have been shown to have similar mechanisms to pain. These disorders include addiction, seizure, stroke, ischemia, a neurodegenerative disorder, anxiety, depression, headache, asthma, rheumatic disease, osteoarthritis, retinopathy, inflammatory eye disorders, pruritis, ulcer, gastric lesions, uncontrollable urination, an inflammatory or unstable bladder disorder, inflammatory bowel disease, irritable bowel syndrome (IBS), irritable bowel disease (IBD), gastroesophageal reflux disease (GERD), functional dyspepsia, functional chest pain of presumed oesophageal origin, functional dysphagia, non-cardiac chest pain, symptomatic gastroesophageal disease, gastritis, aerophagia, functional constipation, functional diarrhea, burbulence, chronic functional abdominal pain, recurrent abdominal pain (RAP), functional abdominal bloating, functional biliary pain, functional incontinence, functional ano-rectal pain, chronic pelvic pain, pelvic floor dyssenergia, unspecified functional ano-rectal disorder, cholecystalgia, interstitial cystitis, dysmenorrhea, and dyspareunia.

### 5.3.2. Anatomical Definitions

The “dorsal root ganglion” or “DRG” is the cluster of neurons just outside the spinal cord, made of cell bodies of afferent spinal neurons that comprise the PNS. The cell bodies of sensory nerves that convey somatosensory (sense of touch) information to the brain are found in the DRG. These neurons are unipolar, where the axon splits in two, sending one branch to the sensory receptor and the other to the brain for processing.

The term “ipsilateral” (abbreviated herein as “ipsi”) refers to the side of the animal on which the injury is induced. The corresponding “ipsilateral” side in a sham-operated animal or in a naïve animal is the side that would have been injured (e.g., the left side as described in the Examples below). The term “contralateral” (abbreviated herein as “contra”) refers to the uninjured side of the animal or the side equivalent to the uninjured side in a sham-operated or naïve animal.

### 5.3.3. Definitions Related to Compounds

An “analgesic” refers to any compound (e.g., small organic molecule, polypeptide, nucleic acid molecule, etc.) that is either known or novel, and useful to treat pain. Specific categories of analgesics include but are not limited to opioids (e.g., morphine, hydromorphone, methadone, levorphanol, fentanyl, oxycodone, oxymorphone, among others), antidepressants (e.g., fluoxetine (Prozac®), sertraline (Zoloft®), amitriptyline, among others), anti-convulsants (e.g., gabapentin, carbamazepine, valproic acid, topiramate, phenytoin, among others), non-steroidal anti-inflammatory drugs (NSAIDs) and anti-pyretics (such as, e.g., acetaminophen, ibuprofen, fenoprofen, diflusinal, naproxen, aspirin and other salicylates (e.g., choline magnesium trisalicylate), among others), NMDA antagonists (e.g., ketamine, dextromethorphan, among others), and topical Lidocaine (see also Sindrup et al., Pain 1999; 83: 389-400).

The term “modulator” refers to a compound that differentially affects the expression or biological activity of a gene or gene product (i.e., a nucleic acid molecule or protein) such as, e.g., in response to a stimulus that normally activates or represses the expression or activity of that gene or gene product when compared to the expression or activity of the gene or gene product not contacted with the stimulus. In one embodiment, the gene or gene product the expression or activity of which is being modulated is a gene, cDNA molecule or mRNA transcript that encodes a mammalian complement component protein such as, e.g., from a rat, mouse, companion animal, or human. Examples of modulators of complement component-encoding nucleic acids of the present invention include, without limitation, antisense nucleic acids, ribozymes, RNAi oligonucleotides, and transcription factors. In another embodiment, the activity of a complement component is modulated where the modulator binds to the complement component and acts as either an agonist or antagonist of the complement activity. Examples of such modulators include small organic molecules and proteins (e.g., ligands, antibodies, or antibody fragments).

A “test compound” is any molecule that is tested for its ability to act as a modulator of a gene or gene product. Test compounds can be selected without limitation from small inorganic and organic molecules (i.e., those molecules of less than about 2 kD, and more preferably less than about 1 kD in molecular weight), polypeptides (including native ligands, antibodies, antibody fragments, and other immunospecific molecules), peptidomimetics, oligonucleotides, polynucleotide molecules, and derivatives thereof. In various embodiments of certain screening methods of the present invention, a test compound is screened for its ability to modulate the expression of a complement component-encoding nucleic acid molecule or complement component, or to modulate a biological activity of a complement component. A compound that modulates a nucleic acid or protein of interest can be designated as a “candidate compound” or “lead compound” suitable for further testing and development. Candidate compounds include, but are not limited to, the functional categories of agonist and antagonist.

An “agonist” is a compound that binds to and activates, or enhances the activity of, a nucleic acid molecule or protein. A “partial agonist” is a compound that binds to and only partially activates a nucleic acid molecule or protein (i.e. does not achieve as high a maximal effect as a full agonist). An “inverse agonist” is a compound that binds to and has the opposite effect of an agonist (e.g. whereas a full agonist at the mu opioid receptor reduces cellular excitability, an inverse agonist would increase cellular excitability). An “antagonist” is a compound that binds to and blocks activation by either an endogenous or exogenous agonist.

### 5.3.4. Definitions for Expression Profiling and Arrays

“Expression profile” refers to any description or measurement of one or more of the genes that are expressed by a cell, tissue, or organism under or in response to a particular condition. Expression profiles can identify genes that are up-regulated, down-regulated, or unaffected under particular conditions. Gene expression can be detected at the nucleic acid level or at the protein level. Expression profiling at the nucleic acid level can be accomplished using any available technology to measure gene transcript levels. For example, the expression profiling method can employ in situ hybridization, Northern hybridization or hybridization to a nucleic acid microarray, such as an oligonucleotide microarray, or a cDNA microarray. Alternatively, the method can employ reverse transcriptase-polymerase chain reaction (RT-PCR) such as fluorescent dye-based quantitative real time PCR (TaqMang PCR). In the Examples Section below, nucleic acid expression profiles were obtained by: (i) hybridization of labeled cRNA derived from total cellular mRNA to Affymetrix GeneChip& oligonucleotide microarrays; (ii) TaqMane PCR using gene-specific PCR primers; (iii) Northern hybridization; and (iv) in situ hybridization. Expression profiling at the protein level can be accomplished using any available technology to measure protein levels, e.g., using peptide-specific capture agent arrays (see, e.g., International PCT Publication No. WO 00/04389).

The terms “array” and “microarray” are used interchangeably and refer generally to any ordered arrangement (e.g., on a surface or substrate) of different molecules, referred to herein as “probes.” Each different probe of an array is capable of specifically recognizing and/or binding to a particular molecule, which is referred to herein as its “target,” in the context of arrays. Examples of typical target molecules that can be detected using microarrays include mRNA transcripts, cDNA molecules, cRNA molecules, and proteins. As disclosed in the Examples Section below, at least one target detectable by the Affymetrix GeneChip® microarray used as described herein is a nucleic acid molecule (such as an mRNA transcript, or a corresponding cDNA or cRNA molecule) having a nucleotide sequence encoding a complement component.

Microarrays are useful for simultaneously detecting the presence, absence and quantity of a plurality of different-target molecules in a sample (such as an mRNA preparation isolated from a relevant cell, tissue, or organism, or a corresponding cDNA or cRNA preparation). The presence and quantity of a probe's target molecule in a sample may be readily determined by analyzing whether (and how much of) a target has bound to a probe at a particular location on the surface or substrate.

In a preferred embodiment, arrays used in the present invention are “addressable arrays” where each different probe is associated with a particular “address”. For example, in a preferred embodiment where the probes are immobilized on a surface or a substrate, each different probe of the addressable array is immobilized at a particular, known location on the surface or substrate. The presence or absence of that probe's target molecule in a sample may therefore readily be determined by simply detecting whether the target has bound to that particular location on the surface or substrate.

Nucleic acid arrays are further described in the Detection Methods Section below.

### 5.3.5. Definitions related to Hybridization

The term “nucleic acid hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants (such as formamide) of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid). See Molecular Biology of the Cell, Alberts et al., 3^{rd} ed., New York and London: Garland Publ., 1994, Ch. 7.

Typically, hybridization of two strands at high stringency requires that the sequences exhibit a high degree of complementarity over an extended portion of their length. Examples of high stringency conditions include: hybridization to filter-bound DNA in 0.5 M NaHPO₄, 7% SDS, 1 mM EDTA at 65° C., followed by washingin 0.1×SSC/0.1% SDS (where 1×SSC is 0.15 M NaCl, 0.15 M Na citrate) at 68° C., or for oligonucleotide molecules washing in 6×SSC/0.5% sodium pyrophosphate at about 37° C. (for 14 nucleotide-long oligos), at about 48° C. (for about 17 nucleotide-long oligos), at about 55° C. (for 20 nucleotide-long oligos), and at about 60° C. (for 23 nucleotide-long oligos).

Conditions of intermediate or moderate stringency (such as, e.g., an aqueous solution of 2×SSC at 65° C.; alternatively, e.g., hybridization to filter-bound DNA in 0.5 M NaHPO₄, 7% SDS, 1 mM EDTA at 65° C., and washing in 0.2×SSC/0.1% SDS at 42° C.) and low stringency (such as, e.g., an aqueous solution of 2×SSC at 55° C.), require correspondingly less overall complementarity for hybridization to occur between two sequences. Specific temperature and salt conditions for any given stringency hybridization reaction depend on the concentration of the target DNA and length and base composition of the probe, and are normally determined empirically in preliminary experiments, which are routine (see Southern, J. Mol. Biol. 1975; 98: 503; Sambrook et al., Molecular Cloning: A Laboratory Manual, 2^{nd} ed., vol. 2, ch. 9.50, CSH Laboratory Press, 1989; Ausubel et al. (eds.), 1989, Current Protocols in Molecular Biology, Vol. 1, Green Publishing Associates, Inc., and John Wiley & Sons, Inc., New York, at p. 2.10.3).

As used herein, the term “standard hybridization conditions” refers to hybridization conditions that allow hybridization of two nucleotide molecules having at least 75% sequence identity. According to a specific embodiment, hybridization conditions of higher stringency may be used to allow hybridization of only sequences having at least 80% sequence identity, at least 90% sequence identity, at least 95% sequence identity, or at least 99% sequence identity.

Nucleic acid molecules that “hybridize” to any of the complement component-encoding nucleic acids of the present invention may be of any length. In one embodiment, such nucleic acid molecules are at least 10, at least 15, at least 20, at least 30, at least 40, at least 50, and at least 70 nucleotides in length. In another embodiment, nucleic acid molecules that hybridize are about the same length as a particular complement component-encoding nucleic acid.

### 5.3.6. Homology, Sequence Identity, and Orthology

The term “homologous” as used in the art commonly refers to the relationship between nucleic acid molecules or proteins possessing a “common evolutionary origin,” including nucleic acid molecules or proteins within superfamilies (e.g., the immunoglobulin superfamily) and nucleic acid molecules or proteins from different species (Reeck et al., Cell 1987; 50: 667). Such nucleic acid molecules and proteins have sequence homology, as reflected by their sequence similarity, whether in terms of substantial percent similarity or the presence of specific residues or motifs at conserved positions.

The terms “percent (%) sequence similarity”, “percent (%) sequence identity”, and the like, generally refer to the degree of identity or correspondence between the nucleotide sequences of different nucleic acid molecules or the amino acid sequences of different proteins that may or may not share a common evolutionary origin (see Reeck et al., supra). Sequence identity can be determined using any of a number of publicly available sequence comparison algorithms, such as BLAST, FASTA, DNA Strider, GCG (Genetics Computer Group, Program Manual for the GCG Package, Version 7, Madison, Wis.), etc.

To determine the percent identity between two amino acid sequences or two nucleic acid molecules, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are, or are about, of the same length. The percent identity between two sequences can be determined using techniques similar to those described below, with or without allowing gaps. In calculating percent sequence identity, exact matches are typically counted.

The determination of percent identity between two sequences can be accomplished using a mathematical algorithm. A non-limiting example of a mathematical algorithm utilized for the comparison of two sequences is the algorithm of Karlin and Altschul, Proc. Natl. Acad. Sci. USA 1990, 87:2264, modified as in Karlin and Altschul, Proc. Natl. Acad. Sci. USA 1993, 90:5873-5877. Such an algorithm is incorporated into the NBLAST and XBLAST programs of Altschul et al., J. Mol. Biol. 1990; 215: 403. BLAST nucleotide searches can be performed with the NBLAST program, score=100, wordlength=12, to obtain nucleotide sequences homologous to sequences of the invention. BLAST protein searches can be performed with the XBLAST program, score=50, wordlength=3, to obtain amino acid sequences homologous to protein sequences of the invention. To obtain gapped alignments for comparison purposes, Gapped BLAST can be utilized as described in Altschul et al., Nucleic Acids Res. 1997, 25:3389. Alternatively, PSI-Blast can be used to perform an iterated search that detects distant relationship between molecules. See Altschul et al. (1997) supra. When utilizing BLAST, Gapped BLAST, and PSI-Blast programs, the default parameters of the respective programs (e.g., XBLAST and NBLAST) can be used. See ncbi.nlm.nih.gov/BLAST/ on the WorldWideWeb. Another non-limiting example of a mathematical algorithm utilized for the comparison of sequences is the algorithm of Myers and Miller, CABIOS 1988; 4: 11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

In a preferred embodiment, the percent identity between two amino acid sequences is determined using the algorithm of Needleman and Wunsch (J. Mol. Biol. 1970, 48:444-453), which has been incorporated into the GAP program in the GCG software package (Accelrys, Burlington, Mass.; available at accelrys.com on the WorldWideWeb) using either a Blossum 62 matrix or a PAM250 matrix, a gap weight of 16, 14, 12, 10, 8, 6, or 4, and a length weight of 1, 2, 3, 4, 5, or 6. In yet another preferred embodiment, the percent identity between two nucleotide sequences is determined using the GAP program in the GCG software package using an NWSgapdna.CMP matrix, a gap weight of 40, 50, 60, 70, or 80, and a length weight of 1, 2, 3, 4, 5, or 6. A particularly preferred set of parameters (and one that can be used if the practitioner is uncertain about what parameters should be applied to determine if a molecule is a sequence identity or homology limitation of the invention) is use of a Blossum 62 scoring matrix with a gap open penalty of 12, a gap extend penalty of 4, and a frameshift gap penalty of 5.

As used herein, the term “orthologs” refers to genes in different species that apparently evolved from a common ancestral gene by speciation. Normally, orthologs retain the same function through the course of evolution. Identification of orthologs can provide reliable prediction of gene function in newly sequenced genomes. Sequence comparison algorithms that can be used to identify orthologs include without limitation BLAST, FASTA, DNA Strider, and the GCG pileup program. Orthologs often have high sequence similarity.

The present invention encompasses all orthologs of complement components. In addition to rat, mouse and human orthologs, particularly useful complement component orthologs of the present invention are monkey, porcine, canine (dog), and guinea pig orthologs. Orthologs of complement components in animal models of pain or transgenic animals are useful for the diagnostic and screening methods described herein.

### 5.3.7. Molecular Biology Definitions

“Amplification” of DNA as used herein denotes the use of exponential amplification techniques known in the art, such as the polymerase chain reaction (PCR), and non-exponential amplification techniques such as linked linear amplification, which can be used to increase the concentration of a particular DNA sequence present in a mixture of DNA sequences. For a description of PCR, see Saiki et al., Science 1988, 239:487 and U.S. Pat. No. 4,683,202. For a description of linked linear amplification, see U.S. Pat. Nos. 6,335,184 and 6,027,923; Reyes et al., Clinical Chemistry 2001; 47: 131-40; and Wu et al., Genomics 1989; 4: 560-569.

As used herein, the phrase “sequence-specific oligonucleotide” refers to an oligonucleotide that can be used to detect the presence of a specific nucleic acid molecule, or that can be used to amplify a particular segment of a specific nucleic acid molecule for which a template is present. Such oligonucleotides are also referred to as “primers” or “probes.” In a specific embodiment, “probe” is also used to refer to an oligonucleotide, for example about 25 nucleotides in length, attached to a solid support for use on “arrays” and “microarrays” described below.

The term “host cell” refers to any cell of any organism that is selected, modified, transformed, grown, used or manipulated in any way so as, e.g., to clone a recombinant vector or polynucleotide molecule that has been transformed into that cell, or to express a recombinant protein such as, e.g., a complement component protein. Host cells are useful in screening and other assays, as described below.

As used herein, the terms “transfected cell”, “transformed cell”, and “recombinantly engineered cell” refer to a host cell that has been recombinantly engineered or genetically modified to express or over-express a nucleic acid molecule encoding a specific gene product of interest such as, e.g., a complement component protein or a fragment thereof. Any eukaryotic or prokaryotic cell can be used, although eukaryotic cells are preferred, vertebrate cells are more preferred, and mammalian cells are the most preferred. In the case of multi-subunit ion channels, nucleic acids encoding the several subunits are preferably co-expressed by the transfected or transformed cell to form a functional channel. The cell may be engineered to activate an endogenous nucleic acid, e.g., the endogenous complement component-encoding gene in a rat, mouse or human cell, which cell would not normally express that gene product or would express the gene product at only a sub-optimal level. Transfected or transformed cells are suitable to conduct an assay to screen for compounds that modulate the function of the gene product. A typical “assay method” of the present invention makes use of one or more such cells, e.g., in a microwell plate or some other culture system, to screen for such compounds. The effects of a test compound can be determined on a single cell, or on a membrane fraction prepared from one or more cells, or on a collection of intact cells sufficient to allow measurement of activity.

The term “recombinantly engineered cell” refers to any prokaryotic or eukaryotic cell that has been genetically manipulated to express or over-express a nucleic acid of interest, e.g., a complement component-encoding nucleic acid of the present invention, by any appropriate method, including transfection, transformation or transduction. The term “recombinantly engineered cell” also includes a cell that has been engineered to activate an endogenous nucleic acid, e.g., the endogenous complement component-encoding gene in a rat, mouse or human cell, which cell would not normally express that gene product or would express the gene product at only a sub-optimal level. Recombinantly engineered cells expressing one or more containing complement components are useful in the diagnostic and screening methods described below.

The terms “vector”, “cloning vector” and “expression vector” refer to recombinant constructs including, e.g., plasmids, cosmids, phages, viruses, and the like, with which a nucleic acid molecule (e.g., a complement-encoding nucleic acid or an siRNA-expressing or shRNA-expressing nucleic acid) can be introduced into a host cell so as to clone the vector or express the introduced nucleic acid molecule. Vectors may further comprise one or more suitable selectable markers.

The terms “mutant”, “mutated”, “mutation”, and the like, refer to any detectable change in genetic material, (e.g., DNA), or any process, mechanism, or result of such a change. Mutations include gene mutations in which the structure (e.g., DNA sequence) of the gene is altered; any DNA or other nucleic acid molecule derived from such a mutation process; and any expression product (e.g., the encoded protein) exhibiting a non-silent modification as a result of the mutation.

The phrases “disruption of the gene”, “gene disruption”, and the like, refer to any method for achieving gene disruption, including: (i) insertion of a different or defective nucleic acid sequence into an endogenous (naturally occurring) DNA sequence, e.g., into an exon or promoter region of a gene; or (ii) deletion of a portion of an endogenous DNA sequence of a gene; or (iii) a combination of insertion and deletion, so as to decrease or prevent the expression of that gene or its gene product in the cell as compared to the expression of the endogenous gene sequence.

### 5.3.8. General Definitions

The terms “treat”, “treatment”, and the like, refer to relief from or alleviation of the perception of a pain, including the relief from or alleviation of the intensity and/or duration of a pain (e.g., burning sensation, tingling, electric-shock-like feelings, etc.) experienced by a subject in response to a given stimulus (e.g., pressure, tissue injury, cold temperature, etc.). Relief from or alleviation of the perception of pain can be any detectable decrease in the intensity or duration of pain. Treatment can occur in a subject (e.g., a human or companion animal) suffering from a pain condition or having one or more symptoms of a pain-related disorder that can be treated according to the present invention, or in an animal model of pain, such as the SNL rat model of neuropathic pain described herein, or another animal model of pain. In the context of the present invention insofar as it relates to any of the other conditions recited herein below (other than pain), the terms “treat”, “treatment”, and the like mean to relieve or alleviate at least one symptom associated with such condition, or to slow or reverse the progression of such condition.

The term “subject” as used herein refers to a mammal (e.g., a rodent such as a mouse or a rat, a pig, a primate, or a companion animal (e.g., a dog or cat)). In particular, the term refers to a human.

The term “expressed sequence tag” or “EST” refers to short (usually about 200-600 nt) single-pass sequence reads from one or both ends of a cDNA clone. Typically, ESTs are produced in large batches by performing a single, automated, sequencing read of cDNA inserts in a cDNA library using a primer based on the vector sequence. As a result, ESTs often correspond to relatively inaccurate (around 2% error) partial cDNA sequences. Since most ESTs are short, they probably will not contain the entire coding region of a large gene (exceeding 200-600 nt in ORF length). Alternatively, or in addition, ESTs may contain non-coding sequences corresponding to untranslated regions of mRNA. ESTs can provide information about the location, expression, and function of the entire gene they represent. They are useful (e.g., as hybridization probes and PCR primers) in identifying full-length genomic and coding sequences as well as in mapping exon-intron boundaries, identifying alternatively spliced transcripts, non-translated transcripts, truly unique genes, and extremely short genes. For a review, see Yuan et al., Pharmacology and Therapeutics 2001, 91:115-132. In the present application, the term “EST clone” is used to indicate the entire cloned cDNA segment of which only a portion has been initially end-sequenced to produce the “EST” or “EST sequence” which may be stored in public domain sequence databases (e.g., dbEST at NCBI, available on the WorldWideWeb at ncbi.nlm.nih.gov/dbEST/). As with other public domain DNA sequences, these ESTs or EST sequences have accession numbers, and can be analyzed by sequence comparison algorithms such as BLAST, FASTA, DNA Strider, GCG, etc. The Affymetrix GeneChip arrays used in the Examples section below include probe sets (consisting of 25 nt oligonucleotides) designed to measure mRNA levels of the gene encompassing the EST and are annotated by Affymetrix with the accession number for the relevant EST sequence. Such probe sets are referred to herein by their particular EST accession numbers.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The terms “detectable change” and “detectable difference” as used herein in relation to an expression level of a gene or gene product (e.g., a complement component) or in relation to a biological activity of a complement component means any statistically significant change or difference, respectfully, from an appropriate control or standard value. In a specific embodiment, a detectable change is at least a 1.5-fold change over an appropriate control as measured by any available technique such as hybridization or quantitative PCR.

As used herein, the term “specific binding” refers to the ability of one molecule, typically a nucleic acid molecule, a polypeptide (such as an antibody or immunospecific binding fragment thereof), or a small molecule, to bind to another specific molecule, even in the presence of many other diverse molecules. “Immunospecific binding” refers to the ability of an antibody, or immunospecific fragment thereof, to specifically bind to (or to be “specifically immunoreactive with”) its corresponding antigen.

“Endogenous” refers to any gene or gene product as it is naturally expressed or produced, respectively, inside an organism, tissue or cell.

In accordance with the present invention, there may be employed conventional molecular biology, microbiology, and recombinant DNA techniques within the skill of the art. See, e.g., Sambrook, Fritsch and Maniatis, Molecular Cloning: A Laboratory Manual, 2^{nd} ed., Cold Spring Harbor Laboratory Press, Cold Spring Harbor, New York, 1989 (herein “Sambrook et al., 1989”); DNA Cloning: A Practical Approach, Volumes I and II (Glover ed. 1985); Oligonucleotide Synthesis (Gait ed. 1984); Nucleic Acid Hybridization (Hames and Higgins eds. 1985); Transcription And Translation (Hames and Higgins eds. 1984); Animal Cell Culture (Freshney ed. 1986); Immobilized Cells And Enzymes (IRL Press, 1986); B. Perbal, A Practical Guide To Molecular Cloning (1984); Ausubel et al. eds., Current Protocols in Molecular Biology, John Wiley and Sons, Inc. 1994; among others.

### 5.4. Inhibitory Oligonucleotides

Oligonucleotides that interact (e.g., hybridize under standard conditions) with a nucleotide sequence encoding a complement component can be used to inhibit the expression of that complement component (e.g., by inhibiting transcription, splicing, transport; or translation or by promoting degradation of the corresponding mRNA). Such oligonucleotides can be antisense, RNA interference (RNAi), ribozyme, or triplex helix forming nucleotides. An oligonucleotide molecule can be used to “knock down” or “knock out” the expression of a complement component in a cell or tissue (e.g., in an animal model or in cultured cells). The Factor B antisense oligonucleotide described in U.S. patent application No. 2004038925 and the antisense oligonucleotides to C3 described in PCT Publication No. WO 03/066805 are examples of such oligonucleotides. RNAi, antisense, ribozyme, and triple helix technologies are described below.

### 5.4.1. RNA Interference (RNAi)

The present invention further provides oligonucleotides useful for inhibiting the expression of a complement component through RNA interference (RNAi), which is a process of sequence-specific post-transcriptional gene silencing by which double stranded RNA (dsRNA) homologous to a target locus specifically inactivate gene function in an organism (Hammond et al., Nature Genet. 2001; 2: 110-119; Sharp, Genes Dev. 1999; 13: 139-141). This dsRNA-induced gene silencing is mediated by short double-stranded small interfering RNAs (siRNAs) generated from longer dsRNAs by ribonuclease III cleavage (Bernstein et al., Nature 2001; 409: 363-366 and Elbashir et al., Genes Dev. 2001; 15: 188-200). RNAi-mediated gene silencing is thought to occur via sequence-specific mRNA degradation, where sequence specificity is determined by the interaction of an siRNA with its complementary sequence within a target mRNA (see, e.g., Tuschl, Chem. Biochem. 2001; 2: 239-245).

For mammalian systems, RNAi commonly involves the use of dsRNAs that are greater than 500 bp; however, it can also be activated by introduction of either siRNAs (Elbashir, et al., Nature 2001; 411: 494-498) or short hairpin RNAs (shRNAs) bearing a fold back stem-loop structure (Paddison et al., Genes Dev. 2002; 16: 948-958; Sui et al., Proc. Natl. Acad. Sci. USA 2002; 99: 5515-5520; Brummelkamp et al., Science 2002; 296: 550-553; Paul et al., Nature Biotechnol. 2002; 20: 505-508). siRNAs or shRNAs of the present invention can be 10 or more nucleotides in length and are typically 18 or more nucleotides in length. For reviews, see Bosner and Labouesse, Nature Cell Biol. 2000; 2: E3 1-E36; and Sharp and Zamore, Science 2000; 287: 2431-2433.

The siRNAs to be used in the methods of the present invention are preferably short double stranded nucleic acid duplexes comprising annealed complementary single stranded nucleic acid molecules. In one embodiment, the siRNA is a short dsRNA comprising annealed complementary single strand RNAs. In another embodiment, the siRNA comprises an annealed RNA:DNA duplex, wherein the sense strand of the duplex is a DNA molecule and the antisense strand of the duplex is a RNA molecule.

Preferably, each single stranded nucleic acid molecule of the siRNA duplex is from about 19 nucleotides to about 27 nucleotides in length. In a preferred embodiment, the duplexed siRNA has a 2 or 3 nucleotide 3′ overhang on each strand of the duplex. In one embodiment, the siRNA has 5′-phosphate and 3′-hydroxyl groups.

An RNAi molecule to be used in a method of the present invention comprises a nucleic acid sequence that is complementary to the nucleic acid sequence of a portion of the target locus. In certain embodiments, the portion of the target locus to which the RNAi molecule is complementary is at least about 15 nucleotides in length. In one embodiment, the portion of the target locus to which the RNAi molecule is complementary is at least about 19 nucleotides in length. The target locus to which an RNAi molecule is complementary may represent either a transcribed portion of a complement component-encoding gene or an untranscribed portion of a complement component-encoding gene (e.g., an intergenic region, repeat element, etc.).

The RNAi molecule may further include one or more modifications, either to the phosphate-sugar backbone or to the nucleoside. For example, the phosphodiester linkages of natural RNA may be modified to include at least one heteroatom other than oxygen, such as nitrogen or sulfur. In this case, for example, the phosphodiester linkage may be replaced by a phosphothioester linkage. Similarly, one or more bases may be modified to block the activity of adenosine deaminase. Where the RNAi molecule is produced synthetically, or by in vitro transcription, a modified ribonucleoside may be introduced during synthesis or transcription.

According to the present invention, the siRNA molecule may be introduced to a target cell as an annealed duplex siRNA, or as single stranded sense and anti-sense nucleic acid sequences that, once within the target cell, anneal to form the siRNA duplex. Alternatively, the sense and anti-sense strands of the siRNA may be encoded on an expression construct that is introduced to the target cell. Upon expression within the target cell, the transcribed sense and antisense strands may anneal to reconstitute the siRNA.

A shRNA to be used in a method of the present invention comprises a single stranded “loop” region connecting complementary inverted repeat sequences that anneal to form a double stranded “stem” region. Structural considerations for shRNA design are generally discussed, for example, in McManus et al., RNA 2002; 8: 842-850. In certain embodiments, the shRNA may be a portion of a larger RNA molecule, e.g., as part of a larger RNA that also contains U6 RNA sequences (Paul et al., supra).

In one embodiment, the loop of the shRNA is from about 1 to about 9 nucleotides in length. In another embodiment, the double stranded stem of the shRNA is from about 19 to about 33 base pairs in length. In another embodiment, the 3′ end of the shRNA stem has a 3′ overhang. In a particular embodiment, the 3′ overhang of the shRNA stem is from 1 to about 4 nucleotides in length. In another embodiment, the shRNA has 5′-phosphate and 3′-hydroxyl groups.

Although the RNAi molecules useful according to the invention preferably contain nucleotide sequences that are fully complementary to a portion of the target locus, 100% sequence complementarity between the RNAi molecule and the target locus is not necessarily required to practice the invention assuming sufficient complementarity is otherwise present.

RNAi molecules useful in a method of the present invention may, in view of the present disclosure, be chemically synthesized, for example, using appropriately protected ribonucleoside phosphoramidites and a conventional DNA/RNA synthesizer. RNAs produced by such methodologies tend to be highly pure and to anneal efficiently to form siRNA duplexes or shRNA hairpin stem-loop structures. Following chemical synthesis, single stranded RNA molecules are typically deprotected, annealed to form siRNAs or shRNAs, and purified (e.g., by gel electrophoresis or HPLC).

Alternatively, standard procedures may used for in vitro transcription of RNA from DNA templates carrying RNA polymerase promoter sequences (e.g., T7 or SP6 RNA polymerase promoter sequences). Efficient in vitro protocols for preparation of siRNAs using T7 RNA polymerase have been generally described (Donze and Picard, Nucleic Acids Res. 2002; 30: e46; and Yu et al., Proc. Natl. Acad. Sci. USA 2002; 99: 6047-6052). Similarly, an efficient in vitro protocol for preparation of shRNAs using T7 RNA polymerase has been generally described (Yu et al., supra). The sense and antisense transcripts may be synthesized in two independent reactions and subsequently annealed, or they may be synthesized simultaneously in a single reaction.

RNAi molecules may be formed within a cell by transcription of RNA from an expression construct introduced into the cell. For example, both a protocol and an expression construct for in vivo expression of siRNAs are generally described in Yu et al., supra. Similarly, protocols and expression constructs for in vivo expression of shRNAs have been described (Brummelkamp et al., supra; Sui et al., supra; Yu et al., supra; McManus et al., supra; Paul et al., supra).

Expression constructs for in vivo production of RNAi molecules comprise RNAi-encoding sequences operably linked to elements necessary for the proper transcription of the RNAi encoding sequence(s), including promoter elements and transcription termination signals. Preferred promoters for use in such expression constructs include the polymerase-III HI-RNA promoter (see, e.g., Brummelkamp et al., supra) and the U6 polymerase-III promoter (see, e.g., Sui et al., supra; Paul, et al. supra; and Yu et al., supra). The RNAi expression constructs can further comprise vector sequences that facilitate the cloning of the expression constructs. Standard vectors that maybe used in practicing the current invention are known in the art (e.g., pSilencer 2.0-U6 vector, Ambion Inc., Austin, Tex.).

### 5.4.2. Antisense Nucleic Acids

The present invention further provides antisense oligonucleotides useful for inhibiting the expression of a complement component. An “antisense” nucleic acid molecule or oligonucleotide is a single stranded nucleic acid molecule, which may be DNA, RNA, a DNA-RNA chimera, or a derivative thereof, which, upon hybridizing under physiological conditions with complementary bases in an RNA or DNA molecule of interest, inhibits the expression of the corresponding gene by inhibiting, e.g., mRNA transcription, mRNA splicing, mRNA transport, or mRNA translation or by decreasing mRNA stability. As presently used, “antisense” broadly includes RNA-RNA interactions, RNA-DNA interactions, and RNase-H mediated arrest. Antisense nucleic acid molecules can be encoded by a recombinant gene for expression in a cell (see, e.g., U.S. Pat. Nos. 5,814,500 and 5,811,234), or alternatively they can be prepared synthetically (see, e.g., U.S. Pat. No. 5,780,607). According to the present invention, a complement component involved in a pain condition may be modulated using antisense nucleic acids designed on the basis of complement component-encoding nucleic acid molecules.

An antisense oligonucleotide is typically 18 to 25 bases in length (but can be as short as 13 bases in length), and is typically designed to bind to a selected complement component-encoding mRNA transcript so as to prevent expression of the specific complement component protein. An antisense oligonucleotide will typically be at least 6 nucleotides and preferably up to about 50 nucleotides in length. In particular aspects, the antisense oligonucleotide will be at least 10 nucleotides, at least 15 nucleotides, at least 25, at least 30, at least 100 nucleotides, or at least 200 nucleotides in length.

The antisense nucleic acid oligonucleotide of the present invention can comprise a nucleotide sequence that is complementary to at least a portion of the corresponding complement component-encoding mRNA transcript. However, 100% sequence complementarity is not required so long as formation of a stable duplex (for single stranded antisense oligonucleotides) or triplex (for double stranded antisense oligonucleotides) can be achieved. The ability to hybridize will depend on both the degree of complementarity and the length of the antisense oligonucleotide. Generally, the longer the antisense oligonucleotide, the more base mismatches with the corresponding mRNA transcript can be tolerated. One skilled in the art can ascertain a tolerable degree of mismatch by use of standard procedures to determine the melting point of the hybridized complex.

The antisense oligonucleotide can be modified at the base moiety, sugar moiety, or phosphate backbone, or any combination thereof. In one non-limiting embodiment, a complement component-specific antisense oligonucleotide can comprise at least one modified base moiety selected from the group consisting of 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, and 2,6-diaminopurine.

In another embodiment, the complement component-specific antisense oligonucleotide comprises at least one modified sugar moiety, e.g., a sugar moiety selected from arabinose, 2-fluoroarabinose, xylulose, and hexose.

In yet another embodiment, the complement component-specific antisense oligonucleotide comprises at least one modified phosphate backbone selected from a phosphorothioate, a phosphorodithioate, a phosphoroamidothioate, a phosphoroamidate, a phosphorodiamidate, a methylphosphonate, an alkyl phosphotriester, and a formacetal or analog thereof.

The antisense oligonucleotide can further comprise one or more appending groups such as a peptide, or an agent facilitating transport across the cell membrane (see, e.g., Letsinger et al., Proc. Natl. Acad. Sci. USA 1989; 86: 6553-6556; Lemaitre et al., Proc. Natl. Acad. Sci. USA 1987; 84: 648-652; PCT Publication No. WO 88/09810) or across the blood-brain barrier (see, e.g., PCT Publication No. WO 89/10134), hybridization-triggered cleavage agents (see, e.g., Krol et al., BioTechniques 1988; 6: 958-976), intercalating agents (see, e.g., Zon, Pharm. Res. 1988; 5: 539-549), etc.

In another embodiment, the antisense oligonucleotide can include an α-anomeric oligonucleotide which forms a specific double-stranded hybrid with complementary RNA in which, contrary to the usual β-units, the strands run parallel to each other (Gautier et al., Nucl. Acids Res. 1987; 15: 6625-6641).

In yet another embodiment, the antisense oligonucleotide molecule can contain a morpholino antisense oligonucleotide (i.e., an oligonucleotide in which the bases are linked to 6-membered morpholine rings, which are connected to other morpholine-linked bases via non-ionic phosphorodiamidate intersubunit linkages). Morpholino oligonucleotides are resistant to nucleases and act by sterically blocking transcription of the target mRNA.

As with the above-described RNAi molecules, the antisense oligonucleotides of the invention can be synthesized by standard methods known in the art, e.g., by use of an automated synthesizer, in view of this disclosure. Antisense nucleic acid oligonucleotides of the present invention can also be produced intracellularly by transcription from an exogenous sequence. For example, a vector can be introduced in vivo such that it is taken up by a cell and the antisense RNA transcribed therein. Such a vector can remain episomal or become chromosomally integrated, so long as it can be transcribed to produce the desired antisense RNA. Such vectors can be constructed by recombinant DNA technology methods standard in the art. Vectors can be plasmid, viral, or others known in the art, used for replication and expression in mammalian cells. In another embodiment, “naked” antisense nucleic acids can be delivered to adherent cells via “scrape delivery”, whereby the antisense oligonucleotide is added to a culture of adherent cells in a culture vessel, the cells are scraped from the walls of the culture vessel, and the scraped cells are transferred to another plate where they are allowed to re-adhere. Scraping the cells from the culture vessel walls serves to pull adhesion plaques from the cell membrane, generating small holes that allow the antisense oligonucleotides to enter the cytosol.

### 5.4.3. Ribozyme Inhibition

The present invention further provides ribozyme oligonucleotides useful for inhibiting the expression of a complement component. Ribozyme molecules catalytically cleave mRNA transcripts and can prevent expression of the gene product (for a review, see Rossi, Current Biology 1994; 4: 469-471 and Cech and Bass, Annu. Rev. Biochem. 1986, 55:599-629). The mechanism of ribozyme action involves sequence-specific hybridization of the ribozyme molecule to complementary target RNA, followed by endonucleolytic cleavage of the target RNA. The composition of ribozyme molecules must include: (i) one or more sequences complementary to the target gene mRNA; and (ii) a catalytic sequence responsible for mRNA cleavage (see, e.g., U.S. Pat. No. 5,093,246). Two types of ribozymes, hammerhead and hairpin, have been described. Each has a structurally distinct catalytic center.

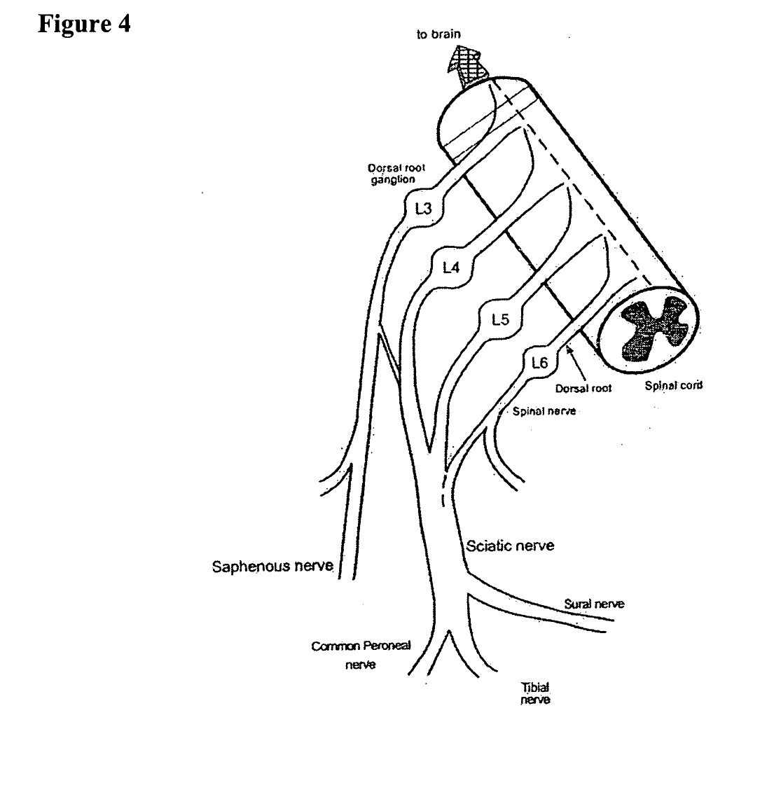

Hammerhead ribozymes cleave mRNAs at locations dictated by flanking regions that form complementary base pairs with the target mRNA. The sole requirement is that the target mRNA has the following sequence of two bases: 5′-UG-3′. The construction of hammerhead ribozymes is known in the art, and described more fully in Myers, Molecular Biology and Biotechnology: A Comprehensive Desk Reference, VCH Publishers, New York, 1995 (see especially FIG. 4, page 833) and in Haseloff and Gerlach, Nature 1988; 334: 585-591.

Ribozymes are preferably engineered so that the cleavage recognition site is located near the 5′ end of the corresponding mRNA so as to increase efficiency and minimize intracellular accumulation of non-functional mRNA transcripts.

As with RNAi and antisense oligonucleotides, ribozymes of the invention can be composed of modified oligonucleotides (e.g., to impart improved stability, targeting, etc.). Ribozymes can be delivered to mammalian cells, and preferably mouse, rat, or human cells, expressing the target complement component protein in vivo. A preferred method of delivery involves using a DNA construct “encoding” the ribozyme under the control of a strong constitutive pol III or pol II promoter, so that transfected cells will produce sufficient quantities of the ribozyme to destroy endogenous mRNA transcript encoding the protein, thereby inhibiting protein expression. Because ribozymes, unlike antisense molecules, are catalytic, a lower intracellular concentration may be required to achieve an adequate level of efficacy.

Ribozymes useful according to the present invention can be prepared by any method known in the art for the synthesis of DNA and RNA molecules, as discussed above, in view of this disclosure. Ribozyme technology is described further in Intracellular Ribozyme Applications: Principals and Protocols, Rossi and Couture eds., Horizon Scientific Press, 1999.

### 5.4.4. Triple Helix Formation

The present invention further provides triple helix-forming oligonucleotides that are useful to inhibit the expression of a complement component. Nucleic acid molecules useful to inhibit complement component gene expression via triple helix formation are preferably composed of deoxynucleotides. The base composition of these oligonucleotides is typically designed to promotetriple helix formation via Hoogsteen base pairing rules, which generally require sizeable stretches of either purines or pyrimidines to be present on one strand of a duplex. Nucleotide sequences may be pyrimidine-based, resulting in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, e.g., those containing a stretch of G residues. These molecules will typically form a triple helix with a DNA duplex that is rich in GC pairs, in which the majority of the purine residues are located on a single strand of the targeted duplex, resulting in GGC triplets across the three strands in the triplex.

Alternatively, sequences can be targeted for triple helix formation by creating a so-called “switchback” nucleic acid molecule. Switchback molecules are synthesized in an alternating 5′-3′, 3+-5′ manner, such that they base pair with one strand of a duplex and then with the other, eliminating the necessity for a sizeable stretch of either purines or pyrimidines to be present on one strand of a duplex.

As with complement component-specific RNAi, antisense oligonucleotides, and ribozymes, triple helix molecules of the invention can be prepared by any method known in the art in view of the present disclosure. These include techniques for chemically synthesizing oligodeoxyribonucleotides and oligoribonucleotides such as, e.g., solid phase phosphoramidite chemical synthesis. Alternatively, RNA molecules can be generated by in vitro or in vivo transcription of DNA sequences “encoding” the particular RNA molecule. Such DNA sequences can be incorporated into a wide variety of vectors that incorporate suitable RNA polymerase promoters such as the T7 or SP6 polymerase promoters.

### 5.5. Antibodies

The present invention further provides the use of antibodies or immunospecific antibody fragments in a diagnotistic, therapeutic, or compound screening method of the present invention. Examples of anti-complement antibodies that can be used to treat pain are provided in the Exogenous Complement Inhibitor Section, supra.

Suitable antibodies may be polyclonal, monoclonal, or recombinant. Application of gene technologies to antibody engineering has enabled the synthesis of single-chain fragment variable (scFv) antibodies-that combine within a single polypeptide chain the light and heavy chain variable domains of an antibody molecule covalently joined by a predesigned peptide linker. Examples of useful fragments include separate heavy chains, light chains, Fab, F(ab′)₂, Fabc, and Fv fragments. Fragments can be produced by enzymatic or chemical separation of intact immunoglobulins or by recombinant DNA techniques. Fragments may be expressed in the form of phage-coat fusion proteins (see, e.g. International PCT Publication Nos. WO 91/17271, WO 92/01047 and WO 92/06204). Typically, the antibodies, fragments, or similar binding agents bind a specific antigen with an affinity of at least 10⁷, 10⁸, 10⁹, or 10¹⁰ M.

In a specific embodiment, antibodies can be raised against a complement component of the invention using known methods in view of this disclosure. Various host animals selected, e.g. from pigs, cows, horses, rabbits, goats, sheep, rats, or mice, can be immunized with a partially or substantially purified complement component, or with a peptide homolog, fusion protein, peptide fragment, analog or derivative thereof. An adjuvant can be used to enhance antibody production.

Polyclonal antibodies can be obtained and isolated from the serum of an immunized animal and tested for specificity against the antigen using standard techniques. Alternatively, monoclonal antibodies can be prepared and isolated using any technique that provides for the production of antibody molecules by continuous cell lines in culture. These include but are not limited to the hybridoma technique originally described by Kohler and Milstein, Nature 1975; 256: 495-497; the human B-cell hybridoma technique (Kosbor et al., Immunology Today 1983; 4: 72; Cote et al., Proc. Natl. Acad. Sci. USA 1983; 80: 2026-2030); and the EBV-hybridoma technique (Cole et al., Monoclonal Antibodies and Cancer Therapy, Alan R. Liss, Inc., 1985, pp 77-96). Alternatively, techniques described for the production of single chain antibodies (see e.g. U.S. Pat. No. 4,946,778) can be adapted to produce specific single chain antibodies.

Antibody fragments that contain specific binding sites for a complement component are also encompassed within the present invention, and can be generated by known techniques. Such fragments include but are not limited to F(ab′)₂ fragments, which can be generated by pepsin digestion of an intact antibody molecule, and Fab fragments, which can be generated by reducing the disulfide bridges of the F(ab′)₂ fragments. Alternatively, Fab expression libraries can be constructed (Huse et al., Science 1989; 246: 1275-1281) to allow rapid identification of Fab fragments having the desired specificity to the particular protein.

Techniques for the production and isolation of monoclonal antibodies and antibody fragments are known in the art, and are generally described, among other places, in Harlow and Lane, Antibodies: A Laboratory Manual, Cold Spring Harbor Laboratory, 1988, and in Goding, Monoclonal Antibodies: Principles and Practice, Academic Press, London, 1986.

Antibodies or antibody fragments can be used in conjunctin with methods known in the art to localize and quantify a complement component, e.g. by Western blotting, in situ imaging, measuring levels thereof in appropriate physiological samples, etc. Immunoassay techniques using antibodies include radioimmunoassay, ELISA (enzyme-linked immunosorbant assay), “sandwich” immunoassays, immunoradiometric assays, gel diffusion precipitation reactions, immunodiffusion assays, in situ immunoassays (using, e.g. colloidal gold, enzyme or radioisotope labels), precipitation reactions, agglutination assays (e.g. gel agglutination assays, hemagglutination assays), complement fixation assays, immunofluorescence assays, protein A assays, and immunoelectrophoresis assays, etc. Antibodies can also be used in microarrays (see, e.g., International PCT Publication No. WO 00/04389).

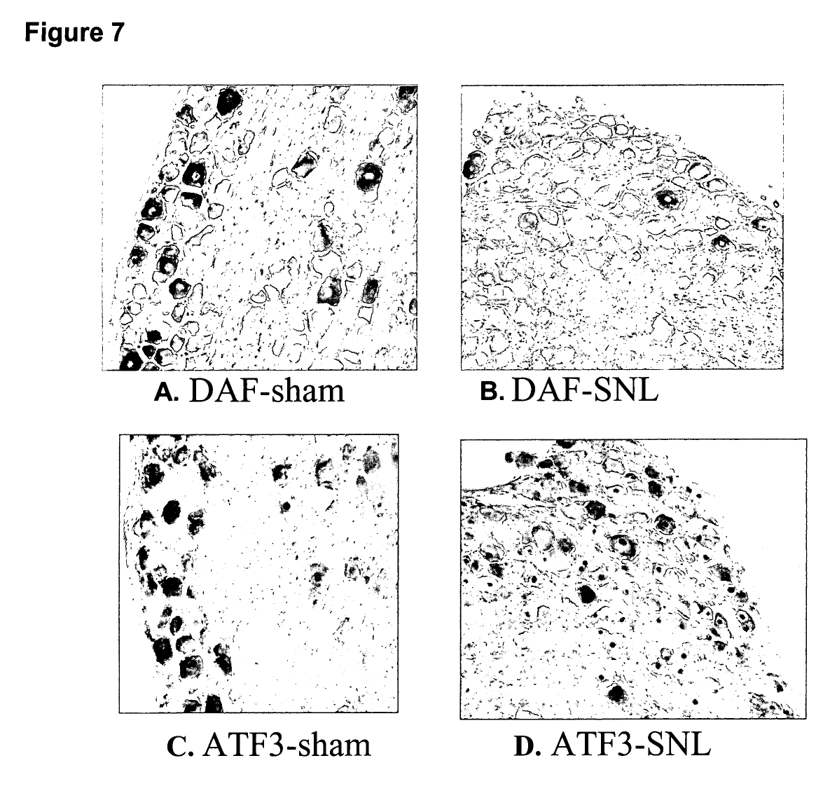

For example as shown in FIG. 7, monoclonal antibodies to DAF protein (gift from Paul Morgan, Cardiff, UK) are useful to identify DAF protein on paraformaldehyde fixed sections of DRG using immunohistochemical staining.

Recent advances in antibody engineering have allowed the genes encoding antibodies to be manipulated, so that antigen-binding molecules can be expressed within mammalian cells. Application of gene technologies to antibody engineering has enabled the synthesis of single-chain fragment variable (scFv) antibodies that combine within a molecule covalently joined by a pre-designed peptide linker. Intracellular antibodies (or intrabodies) can be used to target molecules involved in essential cellular pathways for modification or ablation of protein function. Antibody genes for intracellular expression can be derived either from murine or human monoclonal antibodies or from phage display libraries. For intracellular expression, small recombinant antibody fragments containing the antigen recognizing and binding regions can be used. Intrabodies can be directed to different intracellular compartments by targeting sequences attached to the antibody fragments.

Various methods have been developed to produce intrabodies. Techniques described for the production of single chain antibodies (U.S. Pat. Nos. 5,476,786 and 5,132,405 U.S. Pat. 4,946,778) can be adapted to produce polypeptide-specific single chain antibodies. Another method called intracellular antibody capture (IAC) is based on a genetic screening approach (Tanaka et al., Nucleic Acids Res. 2003 Mar 1; 31 (5):e23). Using this technique, consensus immunoglobulin variable frameworks are identified, which can form the basis of intrabody libraries for direct screening. The procedure comprises in vitro production of a single antibody gene fragment from oligonucleotides and diversification of CDRs of the immunoglobulin variable domain by mutagenic PCR to generate intrabody libraries. This method obviates the need for in vitro production of antigen for pre-selection of antibody fragments and also yields intrabodies with enhanced intracellular stability.

These intrabodies can be used to modulate cellular physiology and metabolism through a variety of mechanisms, including the blocking, stabilizing, or mimicking of protein-protein interactions, by altering enzyme function, or by diverting proteins from their usual intracellular compartments. Intrabodies can be directed to the relevant cellular compartments by modifying the genes that encode them to specify N- or C-terminal polypeptide extensions for providing intracellular-trafficking signals.

### 5.6. Animal Models of Pain

As specified below, the diagnostic and screening methods of the present invention can be conducted in: (i) any cell derived from a tissue of an organism experiencing pain or a pain-related condition; or (ii) any cell grown in vitro in tissue culture under specific conditions that mimic some aspect of a tissue condition in an organism experiencing pain (e.g., nerve injury, inflammation, or viral infection). Cells (especially neural cells) derived from an animal model of pain or related disorder will be particularly useful in carrying out a screening methods of the present invention. As described below, regulation of complement component genes has now been identified using a rat spinal nerve ligation (SNL) model of neuropathic pain (Kim and Chung, Pain 1992; 50: 355-363). Some of the additional useful models are described below.

### 5.6.1. FCA Injection Model

A chronic pain condition can be reproduced in mice or rats by the injection of Freund's complete adjuvant (FCA) containing heat-killed Mycobacterium into the base of the tail or into the hind footpads (Colpaert et al., Life Sci. 1980; 27: 921-928; De Castro Costa et al., Pain 1981; 10: 173-185; Larson et al., Pharmacol. Biochem Behav. 1986; 24:9-53).

For example, a chronic pain condition can be induced by intradermal injection of 50 μl of 50% FCA into one hindpaw, wherein undiluted FCA consists of 1 mg/ml heat-killed and dried Mycobacterium, each ml of vehicle contains 0.85 ml paraffin oil +0.15 ml mannide monooleate (Sigma, St. Louis, Mo.), and the FCA is then diluted 1:1 (vol:vol) with 0.9% saline prior to injection. Intradermal injection can be performed under isoflurane/O₂ inhalation anesthesia. The treated and control (e.g., given an intradermal injection of 0.9% saline) animals can be tested between 24 and 72 hours following FCA injection.

FCA injection causes an inflammation (wide-spread joint inflammation mimicking rheumatoid arthritis when injected into the base of the tail) that lasts for several days, and is evidenced by the classical signs of inflammation (erythema, edema, heat), as well as hyperalgesia (e.g., to thermal and mechanical stimuli) and allodynia (Fundytus et al., Pharmacol Biochem & Behav 2002; 73: 401-410; Binder et al., Anesthesiology 2001; 94:1034-1044). Pain sensitivity (i.e., alterations in nociceptive thresholds) can then be measured in the injected and neighboring regions by decreases in response latency (compared to control animals injected with either the same adjuvant lacking heat-killed Mycobacterium, or 0.9% saline). For example, thermal hyperalgesia can be assessed by applying focused radiant heat to the plantar surface of the hindpaw and measuring the latency for the animal to withdraw its paw from the stimulus (Hargreaves et al., Pain 1988; 32: 77-88; D'Amour and Smith, J. Pharmacol. Exp. Ther. 1941; 72: 74-79; see also the hot-plate assay described by Eddy and Leimbach, J. Pharmacol. Exp. Ther. 1953; 107: 385-393). A decrease in the paw withdrawal latency following FCA injection indicates thermal hyperalgesia. Mechanical hyperalgesia can be assessed with the paw pressure test, where the paw is placed on a small platform and weight is applied in a graded manner until the paw is completely withdrawn (Stein, Biochemistry & Behavior 1988; 31: 451-455, see also the Examples section, below). Mechanical allodynia can be also assessed by applying thin filaments (von Frey hairs) to the plantar surface of the hindpaw and determining the response threshold for paw withdrawal (see Dixon, J. Am Stat. Assoc. 1965; 60: 967-978).

### 5.6.2. Sciatic Nerve Injury Models

The first animal model of neuropathic pain to be developed involved the simple cutting of the sciatic nerve (termed “axotomy”) (Wall et al., Pain 1979; 7: 103-111). Following axotomy, neuromas form at the ends of the cut nerve. With this type of injury, self-mutilation of the injured foot (termed “autotomy”) is often observed.

In this model, a unilateral nerve injury is induced by exposing and cutting one sciatic nerve. The ends of the cut sciatic nerve are then ligated to prevent re-growth. Surgery is performed under isoflurane/O₂ anesthesia. The wound is closed with 4-0 Vicryl, dusted with antibiotic powder, and the animals are allowed to recover on a warm heating pad before being returned to their home cages. Sham-operated animals are used as a control. Sham-operation consists of exposing but not injuring the sciatic nerve. Animals are observed for up to two weeks to assess pain behaviors. Animals can be tested with the thermal and mechanical tests described above.

One of the most commonly used experimental animal models for neuropathic pain is the chronic constriction injury (CCI), where four loose ligatures are tied around the sciatic nerve (Bennett and Xie, Pain 1988; 33: 87-107). One disadvantage of this model is the introduction of foreign material into the wound causing a local inflammatory reaction, whereas hyperalgesia does not have to be associated with inflammation. Thus, a distinction between the neuropathic component and the inflammatory component of pain is difficult to discern in this model. In order to produce a pure nerve injury model without an epineurial inflammatory component due to introduction of foreign material, Lindenlaub and Sommer (Pain 2000; 89: 97-106) describe a partial sciatic nerve transection (PST) in rats. These rats developed thermal hyperalgesia and mechanical allodynia comparable to the CCI model. In both models, the thermal withdrawal thresholds of the animals are commonly assessed by response to radiant heat on the plantar surface of the hindpaw (Hargreaves et al., Pain 1988; 32: 77-88). Mechanical hypersensitivity is commonly determined by measuring the withdrawal thresholds to von Frey hairs (Dixon, J. Am Stat. Assoc. 1965; 60: 967-978).

Decosterd and Woolf (Pain 2000, 87:149-58) describe a variant of partial denervation, termed the spared nerve injury model. This model involves a lesion of two of the three terminal branches of the sciatic nerve-(tibial and common peroneal nerves), leaving the remaining sural nerve intact. The spared nerve injury model differs from the SNL, CCI and PST models in that the co-mingling of distal intact axons with degenerating axons is restricted, and permitting behavioral testing of the non-injured skin territories adjacent to the denervated areas. The spared nerve injury model results in early (i.e., less than 24 hours), prolonged (greater than 6 months), robust (all animals are responders) behavioral modifications. Mechanical sensitivity (as determined, e.g., by sensitivity to von Frey hairs and pinprick test) and thermal (hot and cold) responsiveness are increased in the ipsilateral sural, and to a lesser extent saphenous, territories, without any change in heat thermal thresholds.

Partial sciatic nerve ligation is yet another sciatic nerve injury model (Seltzer et al., Pain 1990, 43: 205-218). In mammals, e.g. rats, about half of the sciatic nerves high in the thigh are unilaterally ligated in this model. According to Seltzer et al., rats of this model develop a guarding behavior of the ipsilateral hindpaw and lick it often. These behaviors are observed within a few hours after the operation and for several months thereafter. Allodynia, thermal hyperalgesia, and mechanical hyperalgesia are each observed in this model according to Seltzer et al. The partial sciatic nerve ligation model may be used when addressing hypotheses concerning causalgiform pain disorders.

### 5.6.3. Cancer Pain Models

The models of neuropathic pain described above involve acute or sub-acute insult of the peripheral nerve, and do not necessarily reflect gradual but progressive insult of the nerve as expected to occur in such common neuropathic pain conditions as neuropathic cancer pain. However, neuropathic cancer pain can be reproduced by inoculating Meth A sarcoma cells into the immediate proximity of the sciatic nerve in BALB/c mice (Shimoyama et al., Pain 2002; 99: 167-174). The tumor grows predictably with time, gradually compressing the nerve and causing thermal hyperalgesia (as determined, e.g., by paw withdrawal latencies to radiant heat stimulation), mechanical allodynia (as determined, e.g., by sensitivity of paws to von Frey hairs), and signs of spontaneous pain (as detected, e.g., by spontaneous lifting of the paw).

A rat model of bone cancer pain was also recently described by Medhurst et al., Pain 2002; 96: 129-40. In this model, Sprague-Dawley rats receive intra-tibial injections of 3 x 10³ or 3×10⁴ syngeneic MRMT-1 rat mammary gland carcinoma cells, to produce rapidly expanding tumors within the boundaries of the tibia, thereby causing severe remodeling of the bone. Rats receiving intra-tibial injections of MRMT-1 cells develop behavioral signs indicative of pain, including the gradual development of mechanical allodynia and mechanical hyperalgesia/reduced weight bearing on the affected limb, beginning on day 12-14 or 10-12 following injection of 3×10³ or 3×10⁴ cells, respectively. These symptoms are not observed in rats receiving heat-killed cells or vehicle alone. Acute treatment with morphine produces a dose-dependent reduction in the response frequency of hind paw withdrawal to von Frey hairs, as well as reduction in the difference in hind limb weight bearing.

### 5.6.4. Incisional Model of Post-Operative Pain

Brennan and colleagues have developed an animal model of post-operative pain (Brennan et al., Pain 1996; 64: 493-501), which involves making a surgical incision on the plantar aspect of the rat hindpaw. Specifically, a 1-cm incision is made in the plantar surface of one hindpaw under isoflurane/O₂ inhalation anesthesia. The incision is closed with two sutures using 4-0 Vicryl. Rats are allowed to recover in their home cages. Naive rats are used as control animals. Mechanical and thermal sensitivity is measured 24 hours after injury, e.g., as described above. The mechanical hyperalgesia that is observed in this rat model parallels the time course of pain in post-operative patients, and is alleviated by systemic and intrathecal (i.t.) morphine (Zahn et al., Anesthesiology 1997; 86: 1066-1077).

### 5.7. Genetically Modified Animals

Genetically modified animals, particularly genetically modified mammals, may be used for diagnosing pain states, including neuropathic, inflammatory and cancer pain, and for evaluating compounds to treat such pain. Non-human genetically modified mammals are a specific embodiment of genetically modified animals. The use of non-human genetically modified mammals in diagnostic and screening methods allows a researcher to perform a wider variety of experiments than is possible with human subjects.

As used herein, the term “genetically modified animal” encompasses any animal into which an exogenous genetic material has been introduced and/or whose endogenous genetic material has been manipulated. Examples of genetically modified animals include, without limitation, e.g., “knock-in” animals, “knockout” animals, transgenic animals, and animals containing cells harboring a non-integrated nucleic acid construct (e.g., viral-based vector, antisense oligonucleotide, shRNA, siRNA, ribozyme, etc.). Animals containing cells harboring a non-integrated nucleic acid construct include animals wherein the expression of an endogenous gene has been modulated (e.g., increased or decreased) due to the presence of such construct.

### 5.7.1. Knock-In Animals

A “knock-in animal” is a genetically modified animal (e.g., a mammal such as a mouse or a rat) in which an endogenous gene has been substituted in part or in total with a heterologous gene (i.e., a gene that is not endogenous to the locus in question; see Roamer et al., New Biol. 1991, 3:331), an orthologous gene from another species, or a mutated gene. This can be achieved by homologous recombination (see “knockout animal” below), transposition (Westphal and Leder, Curr. Biol. 1997; 7: 530), use of mutated recombination sites (Araki et al., Nucleic Acids Res. 1997; 25: 868), PCR (Zhang and Henderson, Biotechniques 1998; 25: 784), or any other technique known in the art. The heterologous gene may be, e.g., a reporter gene linked to the appropriate (e.g., endogenous) promoter, which may be used to evaluate the expression or function of the endogenous gene (see, e.g., Elegant et al., Proc. Natl. Acad. Sci. USA 1998; 95: 11897).

### 5.7.2. Knockout Animals

A “knockout animal” is a genetically modified animal (e.g., a mammal such as a mouse or a rat) that has had a specific gene in its genome partially or completely inactivated by gene targeting (see, e.g., U.S. Pat. Nos. 5,777,195 and 5,616,491). A knockout animal can be a heterozygous knockout (i.e., with one defective allele and one wild type allele) or a homozygous knockout (i.e., with both alleles rendered defective). In particular embodiments, knockout animals can be naturally occurring or prepared from a naïve animal.

Preparation of a knockout animal typically requires first introducing a nucleic acid construct (a “knockout construct”), that will be used to decrease or eliminate expression of a particular gene, into an undifferentiated cell type termed an embryonic stem (ES) cell. The knockout construct is typically comprised of: (i) DNA from a portion (e.g., an exon sequence, intron sequence, promoter sequence, or some combination thereof) of a gene to be knocked out; and (ii) a selectable marker sequence used to identify the presence of the knockout construct in the ES cell. The knockout construct is typically introduced (e.g., electroporated) into ES cells so that it can homologously recombine with the genomic DNA of the cell in a double crossover event. This recombined ES cell can be identified (e.g., by Southern hybridization or PCR reactions that show the genomic alteration) and is then injected into a mammalian embryo at the blastocyst stage. In a preferred embodiment where the knockout animal is a mammal, a mammalian embryo with integrated ES cells is then implanted into a foster mother for the duration of gestation (see, e.g., Zhou et al., Genes and Dev. 1995; 9: 2623-34).

Regulated knockout animals can be prepared using various systems, such as the tet-repressor system (see U.S. Pat. No. 5,654,168), or the Cre-Lox system (see U.S. Pat. Nos. 4,959,317 and 5,801,030).

Particularly useful knockout animals of the present invention include C3, C4, and C5 knockouts which are available from Jackson Laboratory (Bar Harbor, Me.). Further information on the C4 and C3 knockout animals can also be found in Wessels et al. (Proc Natl Acad Sci USA. 1995, 92:11490-4). Other particularly useful knockout animals include C5a receptor knockout mice (Hopken et al., Nature 1996, 383:86-9), C3a receptor knockout mice (Kildsgaard et al., J Immunol. 2000, 165:5406-9), C6 deficient rats (Qian et al., J Heart Lung Transplant 1998, 17:470-8), Factor D knockout mice (Xu et al., Proc Natl Acad Sci USA. 2001, 98:14577-82), Factor B knockout mice (Matsumoto et al., Proc Natl Acad Sci USA 1997, 94:8720-5), and Factor C1q knockout mice (Botto et al., Nat Genet. 1998, 19:56-9).

Included within the scope of the present invention is an animal, preferably a mammal (e.g., a mouse or rat), in which one, two or more neuropathic pain-associated genes identified according to the present invention have been knocked out or knocked in. For example, multiple knockout animals can be generated by repeating the procedures for generating each knockout construct, or by breeding two animals, each with a different knocked out gene, to each other, and screening for those animals with the double knockout genotype.

### 5.7.3. Transgenic Animals

As used herein, a “transgenic animal” is a non-human genetically modified animal, preferably a mammal, more preferably a rodent such as a rat or mouse, in which one or more of the cells of the animal include a transgene. A “transgene” is exogenous DNA that has been integrated into the genome of a cell from which a transgenic animal develops, and which remains in the genome of the mature animal directing the expression of an encoded gene product in one or more cell types or tissues of the transgenic animal. Examples of transgenic animals include non-human primates, sheep, dogs, pigs, cows, goats, chickens, amphibians, etc.

Transgenic animals can be created in which: (i) a human counterpart of a gene is stably inserted into the genome of the target animal; and/or (ii) an endogenous gene is inactivated and replaced with its human counterparts (see, e.g., Coffman, Semin. Nephrol. 1997, 17:404; Esther et al., Lab. Invest. 1996, 74:953; Murakami et al., Blood Press. Suppl. 1996, 2:36). In one embodiment, a human ortholog of a gene inserted into a transgenic animal is a wild-type gene. In another aspect, the human gene inserted into the transgenic animal is a mutated or variant form of the human gene. In one embodiment, the mutation is associated with neuropathic pain.

### 5.8. Neuronal Cell Cultures

Neuronal cell cultures can be used in the diagnostic and screening methods of the present invention.

DRG neuronal cultures can be produced using ordinary techniques known in the art. The cells are preferably neurons or neuronal cells. In another embodiment, transformed neuronal cell lines, such as those created with tetracarcinoma cell lines, can also be used.

Cultured post-mitotic or neuronal precursors can be obtained using various methods. As one example, primary neurons or neural progenitor cells are extracted and cultured according to methods known in the art (see, e.g., U.S. Pat. No. 5,654,189). Examples of neurons useful in methods of the present invention include neurons in brain tissue collected from mammals, and neuronal cell lines in which nerve projections are extended by addition of growth factors such as NGF (nerve growth factor; neurotrophic factor) and IGF (insulin-like growth factor). For example, DRG neurons from rats can-be dissociated (Caldero et al., J. Neurosci. 1998; 18: 356-370), and placed on tissue-culture dishes or microwells coated, e.g., with omithine-laminin, medium supplemented with glutamine, fetal bovine serum (FBS), putrescine, sodium selenite, progesterone and antibiotics (see, for example, Baudet et al., Development 2000; 127: 4335-4344). Growth factors such as NGF, FGF (fibroblast growth factor), EGF (epidermal growth factor), interleukin 6, etc. (Ann. Rev. Pharmacol. Toxicol. 1991; 31:205-228); IGF (The Journal of Cell Biology 1986; 102:1949-1954) and those disclosed in Cell Culture in the Neurosciences, New York: Plenum Press, pages 95-123 (1955), can also be included. Alternatively, clonal cell lines may be isolated from a conditionally-immortalized neural precursor cell line (See, e.g., U.S. Pat. No. 6,255,122). In one embodiment, the neural cells are primary cultures of neurons. A skilled artisan will readily appreciate that cells or cell cultures used in the methods of the present invention should be carefully controlled for parameters such as cell passage number, cell density, the methods by which the cells are dispensed, and growth time after dispensing, so as to optimize the use of these cells or cell cultures in the diagnostic and screening methods of the present invention.

### 5.9. Determining Nucleic Acid Expression Levels Protein Expression Levels, and Protein Activity

This section describes techniques for determining the expression levels of nucleic acid molecules that encode complement components, the expression levels of complement components (i.e., protein), and the biological activity of complement components.

### 5.9.1. Determining Nucleic Acid Expression Levels

Diagnostic and screening methods of the present invention can include the step of determining the expression level of a complement component-encoding nucleic acid. Assays for determining the expression levels of a complement component-encoding nucleic acid are known in the art. These assays include quantitative hybridization (e.g., quantitative in situ hybridization, Northern blot analysis or microarray hybridization) or quantitative PCR (e.g., TaqMang) using complement component-specific nucleic acids as hybridization probes and PCR primers, respectively. Microarray, PCR-based, in situ, and Northern Blot detection methods are further described, infra. These assays can also be adapted for high-throughput screening.

### 5.9.1.1. Nucleic Acid Microarrays

Nucleic acid arrays (also referred to herein as “transcript arrays” or “hybridization arrays”) can be used to determine the expression level of a nucleic acid molecule. These arrays are comprised of a plurality of nucleic acid probes immobilized on a surface or substrate. The different nucleic acid probes are complementary to, and therefore can hybridize to, different target nucleic acid molecules in a sample. Thus, such probes can be used to simultaneously detect the presence and quantity of a plurality of different nucleic acid molecules in a sample, to determine the expression level of a plurality of different genes, e.g., the presence and abundance of different mRNA molecules, or of nucleic acid molecules derived therefrom (for example, cDNA or cRNA).

There are two major types of microarray technology; spotted cDNA arrays and manufactured oligonucleotide arrays. The Examples Section below describes the use of high density oligonucleotide Affymetrix GeneChipe arrays.

The arrays are preferably reproducible, allowing multiple copies of a given array to be produced and the results from each array easily compared to others. Preferably the microarrays are small, usually smaller than 5 cm², and are made from materials that are stable under binding (e.g., nucleic acid hybridization) conditions. A given binding site or unique set of binding sites in the microarray will specifically bind the target (e.g., the mRNA of a single gene in the cell). Although there may be more than one physical binding site (hereinafter “site”) per specific target, for the sake of clarity the discussion below will assume that there is a single site. It will be appreciated that when cDNA complementary to the RNA of a cell is made and hybridized to a microarray under suitable hybridization conditions, the level or degree of hybridization to the site in the array corresponding to any particular gene will reflect the prevalence in the cell of mRNA transcribed from that gene. For example, when detectably labeled (e.g., with a fluorophore) cDNA complementary to the total cellular mRNA is hybridized to a microarray, any site on the array corresponding to a gene (i.e., capable of specifically binding a nucleic acid product of the gene) that is not transcribed in the cell will have little or no signal, while a gene for which the encoded mRNA is highly prevalent will have a relatively strong signal.

By way of example, GeneChip expression analysis (Affymetrix; Santa Clara, Calif.) generates data for the assessment of gene expression profiles and other biological assays. Oligonucleotide expression arrays simultaneously and quantitatively “interrogate” thousands of iRNA transcripts (genes or ESTs), simplifying large genomic studies. Each transcript can be represented on a probe array by multiple probe pairs to differentiate among closely related members of gene families. Each probe set contains millions of copies of a specific oligonucleotide probe, permitting the accurate and sensitive detection of even low-intensity mRNA hybridization patterns. After hybridization intensity data is captured, e.g., using optical detection systems (e.g., a scanner), software can be used to automatically calculate intensity values for each probe cell. Probe cell intensities can be used to calculate an average intensity for each gene, which correlates with mRNA abundance levels. Expression data can be quickly sorted based on any analysis parameter and displayed in a variety of graphical formats for any selected subset of genes. Gene expression detection technologies include, among others, the research products manufactured and sold by Hewlett-Packard, Perkin-Elmer and Gene Logic.

### 5.9.1.2. PCR-Based Assays

In PCR-based assays, gene expression can be measured after extraction of cellular mRNA and preparation of cDNA by reverse transcription (RT). A sequence within the cDNA can then be used as a template for a nucleic acid amplification reaction. A nucleic acid molecule encoding a specific complement component can be used to design specific RT and PCR oligonucleotide primers (such as, e.g., SEQ ID NOS: 157, 158, 160, 161, 163, 164, 166, and 167, see Table 5, below). Preferably, the oligonucleotide primers are at least about 9 to about 30 nucleotides in length. The amplification can be performed using, e.g., radioactively labeled or fluorescently labeled nucleotides for detection. Alternatively, enough amplified product may be made such that the product can be visualized simply by standard ethidium bromide or other staining methods.

A preferred PCR-based detection method useful in carrying out a method of the present invention is quantitative real time PCR (e.g., TaqMan® technology, Applied Biosystems, Foster City, Calif.). This method is based on the observation that there is a quantitative relationship between the amount of the starting target molecule and the amount of PCR product produced at any given cycle number. Real time PCR detects-the accumulation of amplified product during the reaction by detecting a fluorescent signal produced proportionally during the amplification of a PCR product. The method takes advantage of the properties of Taq DNA polymerases having 5′ exo-nuclease activity (e.g., AmpliTaq®) and Fluorescent Resonant Energy Transfer (FRET) method for detection in real time. The 5′ exo-nuclease activity of the Taq DNA polymerase acts upon the surface of the template to remove obstacles downstream of the growing amplicon that may interfere with its generation. FRET is based on the observation that when a high-energy dye is in close proximity to a low-energy dye, a transfer of energy from high to low will typically occur. The real time PCR probe is designed with a high-energy dye termed a “reporter” at the 5′ end, and a low-energy molecule termed a “quencher” at the 3′ end. When this probe is intact and excited by a light source, the reporter dye's emission is suppressed by the quencher dye as a result of the close proximity of the dyes. When the probe is cleaved by the 5′ nuclease activity of the Taq enzyme, the distance between the reporter and the quencher increases, causing the transfer of energy to stop, resulting in an increase of fluorescent emissions of the reporter, and a decrease in the fluorescent emissions of the quencher. The increase in reporter signal is captured by the Sequence Detection instrument and displayed. The amount of reporter signal increase is proportional to the amount of product being produced for a given sample. According to this method, the data is preferably measured at the exponential phase of the PCR reaction.

Specifically, a fluorogenic probe complementary to the target sequence is designed to anneal to the target sequence between the traditional forward and reverse primers. The probe is labeled at the 5′ end with a reporter fluorochrome (e.g., 6-carboxyfluorescein (6-FAM)). A quencher fluorochrome (e.g., 6-carboxy-tetramethyl-rhodamine (TAMRA)) is added at any T position or at the 3′ end. The probe is designed to have a higher melting temperature (Tₘ) than the primers, and during the extension phase the probe must be 100% hybridized for success of the assay. As long as both fluorochromes are on the probe, the quencher molecule stops all fluorescence by the reporter. However, as Taq polymerase extends the primer, the intrinsic 5′ nuclease activity of Taq degrades the probe, releasing the reporter fluorochrome and resulting in an increase in the fluorescence intensity of the reporter dye. The amount of fluorescence released during the amplification cycle is proportional to the amount of product generated in each cycle. This process occurs in every cycle and does not interfere with the accumulation of PCR product.

In a high throughput setting, to induce fluorescence during PCR, laser light is distributed to 96 sample wells via a multiplexed array of optical fibers. The resulting fluorescent emission returns via the fibers and is directed to a spectrograph with a charge-coupled device (CCD) camera. Emissions sent through the fiber to the CCD camera are analyzed by the software's algorithms. Collected data are subsequently sent to the computer. Emissions are measured, e.g., every 7 seconds. The sensitivity of detection allows acquisition of data when PCR amplification is still in the exponential phase and makes real time PCR more reliable than end-point measurements of accumulated PCR products used by traditional PCR methods.

Some of the preferred parameters of the quantitative real time PCR reactions of the present invention include: (i) designing the probe so that its Tₘ is 10° C. higher than for the PCR primers, (ii) having primer Tₘ's between 58° C. and 60° C., (iii) having amplicon sizes between 50 and 150 bases, and (iv) avoiding 5′ Gs. However, other parameters can be used (e.g., determined using Primer Express® software, Applied Biosystems, Foster City, Calif.). For example, the best design for primers and probes to use for the quantitation of mRNA expression involves positioning of a primer or probe over an intron.

For more details on quantitative real time PCR, see Gibson et al., Genome Res. 1996; 6: 995-1001; Heid et al., Genome Res. 1996; 6: 986-994; Livak et al., PCR Methods Appl. 1995; 4: 357-362; Holland et al., Proc. Natl. Acad. Sci. USA 1991; 88: 7276-7280. Also see the Examples section presented herein below.

SYBR Green Dye PCR (Molecular Probes, Inc., Eugene, Oreg.), competitive PCR as well as other quantitative PCR techniques can also be used to quantify complement component gene expression according to the present invention.

### 5.9.1.3. In Situ Hybridization and Northern Analysis

Complement component gene expression detection assays of the invention can also be performed in situ (e.g., directly upon sections of fixed or frozen tissue collected from a subject, thereby eliminating the need for nucleic acid purification). Complement component-encoding nucleic acid molecules or portions thereof can be used as labeled probes or primers for such in situ procedures (see, e.g., Example 1 below; see also, e.g., Nuovo, PCR in situ Hybridization: Protocols And Application, Raven Press, New York, 1992). Alternatively, if a sufficient quantity of the appropriate cells can be obtained, standard quantitative Northern analysis can be performed to determine the level of gene expression using the nucleic acid molecules of the invention or portions thereof as labeled probes.

### 5.9.2. Determining Protein Expression Levels

Diagnostic and screening methods of the present invention can include the step of determining the expression level of a complement component. Various techniques can be used to measure the levels of a complement component in a sample, including the use of anti-complement component antibodies or antibody fragments. For example, anti-complement component antibodies or antibody fragments can be used to detect the presence of a complement component by, e.g., immunofluorescence techniques employing a fluorescently labeled antibody coupled with light microscopic, flow cytometric or fluorimetric detection methods. Such techniques are particularly preferred for detecting the presence of a complement component on the surface of cells.

In addition, protein isolation methods such as those described by Harlow and Lane (Antibodies: A Laboratory Manual, Cold Spring Harbor Laboratory Press, Cold Spring Harbor, N.Y., 1988) can be employed to measure the levels of a complement component in a sample.

Antibodies or antigen-binding fragments may also be employed histologically, e.g., in immunofluorescence or immunoelectron microscopy techniques, for in situ detection of a complement component. In situ detection may be accomplished by, e.g., removing an appropriate fluid, cell, or tissue sample from a subject and applying to the sample a detectably labeled antibody or antibody fragment specific to a complement component. This procedure can be used to detect the presence, quantity, and tissue distribution of a complement component. Such assays described above may be modified for high-throughput.

Complement component protein levels can be determined as described by Reinhard Würzner (“Immunochemical measurement of complement components and activation products”, pp 103-112) and Antti Väkevä and Seppo Meri (“Complement Deposition in Tissues”, pg 113-121) in Methods in Molecular Biology, vol 150: Complement Methods and Protocols edited by B. P. Morgan (Humana Press Inc., Totowa, N.J.). Levels of complement component proteins can also be determined using ELISA kits available from Quidel Corporation (San Diego, Calif.) and BD Biosciences (San Diego, Calif.).

### 5.9.3. Determining Protein Activity

Diagnostic and screening methods of the present invention can include the step of determining a biological activity level of a complement component. Complement components useful for diagnostic and screening purposes can be obtained from a variety of sources (e.g., cell-based expression systems, purification from natural sources (such as serum), production in vitro by cell-free translation systems, and synthetic methods for peptides). For example, a complement component can be obtained using a protein expression system in host cells (which cells may or may not express an endogenous complement component). The complement component can be isolated and purified using techniques known in the art. Alternatively, cells or tissues that express a complement component can be used in these assays. Protein fragments (e.g., proteolytic fragments or synthetic fragments) of a complement component protein may be used in the assay described below.

### 5.9.3.1. Assaying Protein-Ligand Binding

Determining a biological activity of a complement component may include the step of determining the binding of a compound (e.g., a ligand) to a complement component. For example, a ligand (or binding partner) of a complement component can be determined by the following procedure. First, a standard complement component preparation is prepared by suspending cells or membranes containing a complement component in a buffer appropriate for use in the determination method. Any buffer can be used so long as it will not inhibit the ligand-complement component binding. Such a buffer can be, e.g., a phosphate buffer or a Tris-HCl buffer having pH of 4 to 10 (preferably pH of 6 to 8). To minimize non-specific binding, a surfactant such as CHAPS, Tween-80™ (manufactured by Kao-Atlas Inc.), digitonin or deoxycholate, and various proteins such as bovine serum albumin or gelatin, may optionally be added to the buffer. To suppress degradation of the complement component or ligand by proteases, a protease inhibitor such as PMSF, leupeptin, E-64 (manufactured by Peptide Institute, Inc.) and pepstatin can be added.

Next, a given amount (e.g., 5,000 to 500,000 cpm) of the test compound labeled with [³H], [¹²¹I], [¹⁴C], [³⁵ S] or the like can be added to about 0.01 ml to 10 ml of the solution containing the complement component. To determine the amount of non-specific binding (NSB), a reaction tube containing an unlabeled test compound in large excess is also prepared. The reaction is carried out at about 0 to 50° C., preferably about 4 to 37° C. for about 20 minutes to about 24 hours, preferably about 30 minutes to about 3 hours.

After completion of the reaction, the cells or membranes containing bound ligand are separated, e.g., by filtering the reaction mixture through glass fiber filter paper and washing with an appropriate volume of the same buffer. The residual radioactivity on the glass fiber filter paper can be measured by means of a liquid scintillation counter or gamma (γ)- or beta (β)-counter. A test compound exceeding 0 cpm obtained by subtracting NSB from the total binding (B) (B minus NSB) may be selected as a ligand or binding partner of a complement component.

Protein-ligand binding assays can also include competition binding assays to determine the binding affinity of a test compound compared to a known binding compound. In this type of assay, the complement component is incubated with a detectably labeled compound (e.g., a peptide or antibody) known to bind to the complement component. Following or during incubation with the known binding compound, an unlabeled test compound is introduced to the complement component. The unlabeled test compound competes with the known binding compound for the complement component. Following incubation, the complement component and any bound test compound or bound known binding compound are then separated from the unbound test compound and unbound known binding compound using, e.g., filteration or another techniques known in the art. The amount of labeled known binding compound associated with the complement component is then determined. The binding of different test compounds can be compared to each other by comparing their abilities to compete the known binding compound from the complement component.

Additionally, if the ligand or binding partner of the complement component is a protein, any of a variety of known methods for detecting protein-protein interactions may be used to detect and/or identify the protein that binds to the complement component. For example, co-immunoprecipitation, chemical cross-linking and yeast two-hybrid systems may be employed. In one non-limiting example, Western blotting or mass spectroscopy can be performed on co-immunoprecipitated proteins to identify these proteins and their stoichiometries. In another example in a yeast two-hybrid assay, a host cell harbors a first construct that expresses a complement component fused to a DNA binding domain and a second construct that expresses a potential binding partner fused to an activation domain. The host cell also includes a reporter gene that will be expressed in response to binding of the complement component-partner complex, which complex is formed as a result of binding of the binding partner to the complement component, to an expression control sequence operatively associated with the reporter gene. Reporter genes for useful in the yeast two-hybrid assay, typically encode detectable proteins, including, but not limited to, chloramphenicol transferase (CAT), β-galactosidase (β-gal), luciferase, green fluorescent protein (GFP), alkaline phosphatase, and other genes that can be detected, e.g., immunologically (by antibody assay). See the Mammalian MATCHMAKER Two-Hybrid Assay Kit User Manual from Clontech (Palo Alto, Calif.) for further details on mammalian two-hybrid methods.

Alternatively or in addition, protein arrays can be used to determine complement component-ligand binding. Protein arrays are a type of high-throughput screening, as described, infra. These arrays are solid-phase, ligand binding assay systems using immobilized proteins on surfaces which include glass, membranes, microtiter wells, mass spectrometer plates, and beads or other particles. The assays are highly parallel and often miniaturized. Their advantages include being rapid and automatable, capable of high sensitivity, economical on reagents, and producing an abundance of data from a single experiment.

Automated multi-well formats are the best developed high-throughput screening systems. Automated 96-well plate-based screening systems are the most widely used. The current trend in plate based screening systems is to reduce the volume of the reaction wells further, thereby increasing the density of the wells per plate (96-well to 384-, and up to 1536-wells per plate). The reduction in reaction volumes results in increased throughput, dramatically decreased bioreagent costs, and a decrease in the number of plates that need to be managed by automation. For a description of protein arrays that can be used for high-throughput screening, see U.S. Pat. Nos. 6,475,809; 6,406,921; and 6,197,599; and PCT Publication Nos. WO 00/04389 and WO 00/07024 herein incorporated by reference.

The immobilization method used should be reproducible, applicable to proteins of different properties (size, hydrophilic, hydrophobic), amenable to high throughput and automation, and compatible with retention of fully functional protein activity. Both covalent and noncovalent methods of protein immobilization are used. Substrates for covalent attachment include glass slides coated with amino- or aldehyde-containing silane reagents (Telechem). In the Versalinx™ system (Prolinx), reversible covalent coupling is achieved by interaction between the protein derivatized with phenyldiboronic acid, and salicylhydroxamic acid immobilized on the support surface. Covalent coupling methods providing a stable linkage can be applied to a range of proteins. Noncovalent binding of unmodified protein occurs within porous structures such as HydroGel™ (PerkinElmer), based on a 3-dimensional polyacrylamide gel.

Detection of ligand binding to protein arrays and protein-ligand binding is also described in the Detection Section below.

### 5.9.3.2. Assaying for Protein Activity

A variety of methods well-known in the art can be used to determine at least one activity of a complement component. As described in the Examples Section below, the hemolysis assay can be used to measure the activity of C3 in the serum from blood samples. In the hemolysis assay, erythrocytes are sensitized by coating these erythrocytes with antibodies against red blood cells. Next, the sensitized erythrocytes, C3-depleted serum, and a blood sample to be tested for C3 activity are combined and incubated. During incubation, the complement pathway proceeds on the surface of the erythorcytes using complement components from the C3-depleted serum and C3 from the blood sample. This pathway can result in the formation of a sufficient number of MAC pores to induce erythorcyte lysis and hemoglobin release. The optical density at 540 run is then measured to determine the quantity of free hemoglobin in solution as a result of erythrocyte lysis. Since erythrocyte lysis is a result of complement activation and the presence of C3, the optical density at 540 nm is a measure of the activity of C3 in the blood sample.

The hemolysis assay can also be used to measure the activity of C2, C5, C6, C7, C8, C9, Factor B, C4, and C1q by using sera depleted of each of these complement components in the place of C3-depleted sera. These depleted serums are available from Quidel Corporation (San Diego, Calif.), as well as other commercial and non-commercial sources. Additionally, the hemolysis assay can be adapted to high throughput screening as described, infra.

Variations of the hemolysis assay are also used as techniques to measure complement activity. In some of these variations, complement component activity is measured by quantitating the release of a non-endogenous substance from a cell or quantitating the entry of an endogenous substance during MAC pore formation and cell lysis. For example, nucleated cells can be loaded with calcein AM, which fluoresces in the green wavelength range. Upon MAC formation and cell lysis, calcein is release and measured to determine complement activity (see Spiller, O. B., Measurement of Complement Lysis of Nucleated cells., p73-81, in Complement Methods and Protocols, Ed. By B. Paul Morgan, Humana Press, Totowa, N.J.: 2000). Nucleated cells can also be loaded with a calcium sensitive dye, such as fura-2 acetooxymethyl ester. Upon MAC formation, calcium enters the cell and activates the calcium sensitive dye. The activated dye can be measured using fluorimetry (Berger et al., AM J. Physiol. 1993, 265 (1 Pt 2): H267-72). Fluo-4 AM (available from Molecular Devices, Sunnyvale, Calif.) can also be used to measure calcium mobilization and Fluor-4 AM fluorescence can be measured using a fluorescence plate reader (available from Molecular Probes, Sunnyvale, Calif.) (see Valenzano et al, Journal of Pharmacology and Experimental Therapeutics 2003, 306: 377-386). Other references for the use of calcium dyes to measure calcium influx or mobilization include Chapter 20 of the “Handbook of Fluorescent Probes” published by Molecular Probes, Eugene, Oreg.

Other variations of the hemolysis assay include replacing the cells used in the hemolysis assay with liposomes containing a detectable substance. These liposomes are synthesized with dinitrophenyl (DNP) on their surfaces to allow anti-DNP antibodies to attach to the liposome surface. These antibody-covered liposomes can activate the complement-pathway which can induce MAC formation, liposome lysis, and release of the interior contents of the liposomes. Liposomes can be loaded with a variety of detectable substances. In one example, liposomes contain glucose-6-phosphate dehydrogenase. Upon release, glucose-6-phosphate dehydrogenase binds NAD and glucose-6-phosphate and catalyzes the reduction of NAD to NADH. The absorbance of NADH can then be measured at 340 nm. Kits using liposomes to determine complement activity as described are available from Wako Chemicals USA, Inc. (Richmond, Va.; catalog number: 991-40803). Additionally, the use of liposomes to determine complement activity as described can be adapted to high throughput screening according to Yamamoto et al. (Clin Chem. 1995, 41:586-90). Any of the variations above can be adapted to high throughput screening as described, infra.

Complement deposition on the surface of cells can also be used to measure a biological activity of a complement component. In this immunohistochemical (IHC) method, paraformaldehyde fixed tissue sections are contacted with antibodies that can distinguish the activated (cleaved) forms of a complement component. Alternatively, antibodies that recognize both precursor and cleaved forms of a complement component are contacted with tissue. If the antibodies bind to the tissue, it may be concluded that the complement component of interest is active since only the activated complement component will be deposited on the surface of the cells or tissue. Antibodies to various complement components (e.g., C5, C6, C7, C8, and C9) are available from Quidel Corporation.

The activity of complement components can also be measured using ELISA (enzyme-linked immunosorbent assay). The activity of proteolytic enzymes of the complement system (e.g., Factor D or C3 covertase) can be measured by detecting the cleavage products in reactions catalyzed by these proteolytic enzymes using ELISAs. For example, ELISA detection of Bb and Ba suggest that Factor D is active. Additionally, ELISA detection of C3a and C3b suggest that at least one of the C3 convertases is active. The ELISA technique can be adapted to high throughput screening as described, infra.

For complement components that are serine proteases (e.g., Factor D and C1s), their activity can be measured using serine protease assays. For example, their activity can be assessed by a standard in vitro serine protease assay (see, for example, Stief and Heimburger, U.S. Pat. No. 5,057,414 (1991)). Those of skill in the art are aware of a variety of substrates suitable for in vitro assays, such as Suc-Ala-Ala-Pro-Phe-pNA, Bz-Val-Gly-Arg-pNA-AcOH, fluorescein mono-p-guanidinobenzoate hydrochloride, benzyloxycarbonyl-L-Arginyl-S-benzylester, Nalpha-Benzoyl-L-arginine ethyl ester hydrochloride, and the like. Substrates for serine proteases of the complement pathway are cited by Sim and Tsiftsoglou (Biochem Soc Trans. 2004, 32(Pt 1):21-7).

In addition, protease assay kits are available from commercial sources, such as Calbiochem.RTM. (San Diego, Calif.). For general references, see Barrett (Ed.), Methods in Enzymology, Proteolytic Enzymes: Serine and Cysteine Peptidases (Academic Press Inc. 1994), and Barrett et al., (Eds.), Handbook of Proteolytic Enzymes (Academic Press Inc. 1998).

For complement components that are G-protein coupled receptors (GPCRs), activity can be measured using assays for GPCRs. GPCRs of the complement cascade include C3aR and C5aR which transduce signals via G_{αi} and G_{α16}, respectively, in leukocytes. These assays can be based upon the ability of GPCR family proteins to modulate G protein-activated second messenger signal transduction pathways. In one non-limiting embodiment of this invention, biological activity of a GPCR of the complement pathway can be tested by monitoring the activity of adenylate cyclase, an enzyme that is known to be part of the downstream signaling pathway of many GPCRs (Voet and Voet, Biochemistry, 2^{nd} edition, New York 1995). Adenylate cyclase catalyzes the conversion of ATP to cAMP (Voet and Voet, Biochemistry, 2^{nd} edition, New York 1995). Thus, assays that detect cAMP (e.g., in the presence or absence of a test compound) can be used to monitor GPCR activity (see, e.g., Gaudin et al., J. Biol. Chem. 1998; 273:4990-4996). For example, a plasmid encoding a full-length GPCR can be transfected into a mammalian cell line (e.g., Chinese hamster ovary (CHO) or human embryonic kidney (HEK-293) cell lines) using methods well-known in the art. Transfected cells can be grown in 12-well trays in culture medium for 48 hours, then the culture medium is discarded and the attached cells are gently washed with PBS. The cells can then be incubated in culture medium with or without a test compound for 30 minutes, the medium removed and the cells lysed by treatment with 1M perchloric acid. The cAMP levels in the lysate can be measured by radioimmunoassay using known methods. Changes in the levels of cAMP in the lysate from cells exposed to a test compound compared to those without test compound are proportional to the amount of GPCR present in the transfected cells.

In yet another non-limiting embodiment of this invention, the biological activity of a GPCR of the present invention can be tested by monitoring the activity of phospholipase C, another enzyme that responds to signals from some GPCRs. Phospholipase C hydrolyzes the phospholipid, PIP₂, releasing two intracellular messengers: diacylglycerol (DAG) and inositol-1,4,5-triphosphate (IP₃) (Voet and Voet, Biochemistry, 2^{nd} edition, New York 1995). Accordingly, assays that detect DAG and/or IP₃ accumulation (e.g., in the presence or absence of a test compound) can be used to monitor the activity of a GPCR.

For example, to measure changes in inositol phosphate levels, the cells are grown in 24-well plates containing 1×10⁵ cells/well and incubated with inositol-free media and [³H]myoinositol, 2 mCi/well, for 48 hr. The culture medium is removed, and the cells are washed with buffer containing 10 mM LiCl followed by addition of a test compound. The reaction is stopped by addition of perchloric acid. Inositol phosphates are extracted and separated on Dowex AG1-X8 (Bio-Rad) anion exchange resin; and the total labeled inositol phosphates are counted by liquid scintillation. Changes in the levels of labeled inositol phosphate from cells exposed to ligand compared to those without ligand are proportional to the amount of GPCR present in the transfected cells.

The biological activity of a GPCR may be also tested by measuring calcium mobilization, MAP kinase activity, or GTPγS binding.

It is recognized in the art that agonist-bound GPCRs can form ternary complexes with other ligands or “accessory” proteins and display altered binding and/or signaling properties in relation to the binary agonist-receptor complex. Accordingly, allosteric sites on GPCR proteins represent novel modulator targets and potential drug targets since allosteric modulators possess a number of theoretical advantages over classic orthosteric ligands, such as a ceiling level to the allosteric effect and a potential for greater GPCR subtype-selectivity. Because of the noncompetitive nature of allosteric phenomena, the detection and quantification of such effects often rely on a combination of equilibrium binding, nonequilibrium kinetic, and functional signaling assays. For review see, e.g., Christopoulos and Kenakin, Pharmacological Reviews, 2002, 54: 323-74.

For additional information on complement component GPCRs and assays to detect their activity, see “Complement Anaphylatoxins (C3a, C4a, C5a) and their Receptors (C3aR, C5aR/CD88) as Therapeutic Targets in Inflammation” (Contemporary Immunology: Therapeutic Intervention in the Complement System edited by John D. Lambris and V. Micael Holers; Humana Press, Totowa, N.J. 2000 ).

References detailing assays to determine complement activity include “Evaluation of complement inhibitors.” by P. C. Giclas (pg. 225-236 in Contemporary Immunology: Therapeutic interventions in the complement system, ed. By J. D. Lambris and V. M. Holers). The following references detail assays that can be adapted to high throughput screening to find complement inhibitors: “Measurement of Complement hemolytic activity, generation of complement-depleted sera, and production of hemolytic intermediates” by B. P. Morgan and “Measurement of Complement lysis of nucleated cells” by B. Spiller (pg. 61-71 and pg. 73-81, respectively in Methods in molecular biology vol 150: Complement Methods and protocols, edited by B. P. Morgan, Humana Press Inc., Totowa, N.J.).

### 5.9.4. Detection in Assays

The diagnostic and screening assays of the present invention allow for the detection of molecules.

A molecule (e.g., antibody or polynucleotide probe) can be detectably labeled with an atom (such as a radionuclide), or a molecule (such as fluorescein) that signals its presence. Alternatively, a molecule may be covalently bound to a “reporter” molecule (e.g., an enzyme) that acts on a substrate to produce a detectable product. Detectable labels or other detectable products suitable for use in the present invention include any composition detectable by spectroscopic, photochemical, biochemical, immunochemical, electrical, optical or chemical means. Labels useful in the present invention include biotin for staining with labeled avidin or streptavidin conjugate, magnetic beads (e.g., Dynabeads™), fluorescent dyes (e.g., fluorescein, fluorescein-isothiocyanate (FITC), Texas red, rhodamine, green fluorescent protein, enhanced green fluorescent protein, lissamine, phycoerythrin, Cy2, Cy3, Cy3.5, Cy5, Cy5.5, Cy7, FluorX [Amersham], SyBR Green I & II [Molecular Probes], and the like), radiolabels (e.g., ³H, 125I, ³⁵S, ¹⁴C, or ³²P), enzymes (e.g., hydrolases, particularly phosphatases such as alkaline phosphatase, esterases and glycosidases, or oxidoreductases, particularly peroxidases such as horse radish peroxidase, and the like), substrates, cofactors, inhibitors, chemilluminescent groups, chromogenic agents, and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are known in the art. Thus, for example, chemilluminescent and radioactive labels may be detected using photographic film or scintillation counters, and fluorescent markers may be detected using a photodetector to detect emitted light (e.g., as in fluorescence-activated cell sorting). Enzymatic labels are typically detected by providing the enzyme with a substrate and detecting a colored reaction product produced by the action of the enzyme on the substrate. Colorimetric labels are detected by simply visualizing the colored label. Thus, for example, where the label is a radioactive label, means for detection include a scintillation counter, photographic film as in autoradiography, or storage phosphor imaging. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrate to the enzyme and detecting the resulting reaction product. Also, simple colorimetric labels may be detected by observing the color associated with the label. Fluorescence resonance energy transfer has been adapted to detect binding of unlabeled ligands, which may be useful on arrays.

### 5.9.5. High-Throughput Assays

Generally, high-throughput screens can be used to determine the expression of complement component-encoding nucleic acids, the expression of a complement component, or a biological activity of a complement component. High-throughput assays include cell-based and cell-free assays against individual protein targets. It will be appreciated that various assays can be used to detect different types of agents. Several methods of automated assays have been developed in recent years to enable the screening of tens of thousands of compounds in a short period of time (see, e.g., U.S. Pat. Nos. 5,585,277; 5,679,582; and 6,020,141).

High-throughput cell-based arrays combine the technique of cell culture with the use of fluidic devices for (i) measurement of cell response to analytes (i.e., test compounds) in a sample of interest, (ii) screening of samples for identifying molecules or organisms that induce a desired effect in cultured cells, and (iii)selection and identification of cell populations with novel and desired characteristics. High-throughput screens can be performed either on fixed cells using fluorescently labeled antibodies, biological ligands, and/or nucleic acid hybridization probes, or on live cells using multicolor fluorescent indicators and biosensors. The choice of fixed or live cell screens depends on the specific cell-based assay utilized.

There are numerous single- and multi-cell-based array techniques known in the art. Recently developed techniques such as micro-patterned arrays (described in WO 97/45730, WO 98/38490) and microfluidic arrays provide valuable tools for comparative cell-based analysis. Transfected cell microarrays are a complementary technique in which array features comprise clusters of cells overexpressing defined cDNAs. Complementary DNAs cloned in expression vectors are printed on microscope slides, which become “living arrays” after the addition of a lipid transfection reagent and adherent mammalian cells (Bailey et al., Drug Discov. Today 2002; 7 (18 Suppl.): S113-8). Cell-based arrays are described in detail in, e.g., Beske, Drug Discov. Today 2002;7 (18 Suppl.) :S131-5; Sundberg et al., Curr. Opin. Biotechnol. 2000; 11(1):47-53; Johnston et al., Drug Discov. Today 2002; 7 (6):353-63; U.S. Pat. Nos. 6,406,840 and 6,103,479, and U.S. published patent application 2002/0197656. For cell-based assays specifically used to screen for modulators of ligand-gated ion channels, see Mattheakis et al., Curr. Opin. Drug Discov. Devel. 2001; (1):124-34 and Baxter et al., J. Biomol. Screen. 2002; 7(1):79-85.

### 5.10. Diagnostic Methods

The present invention further provides a method for detecting a pain response in a test cell, said method comprising:
(a) determining the expression level of a nucleic acid molecule encoding a complement component in a test cell; and
(b) comparing the expression level of the complement component-encoding nucleic acid molecule in the test cell to the expression level of the same nucleic acid molecule in a control cell that is not exhibiting a pain response;
   wherein a detectable difference between the expression level of the complement component-encoding nucleic acid molecule in the test cell and the expression level of the complement component-encoding nucleic acid molecule in the control cell indicates that the test cell is exhibiting a pain response.

The present invention further provides a method for detecting a pain response in a test cell, said method comprising:
(a) determining the expression level of a complement component in a test cell; and
(b) comparing the expression level of the complement component in the test cell to the expression level of the same complement component in a control cell that is not exhibiting a pain response;
   wherein a detectable difference between the expression level of the complement component in the test cell and the expression level of the complement component in the control cell indicates that the test cell is exhibiting a pain response.

The present invention further provides a method for detecting a pain response in a test cell, said method comprising:
(a) determining a biological activity of a complement component in the test cell; and
(b) comparing the biological activity of the complement component in the test cell to the biological activity of the same complement component in a control cell that is not exhibiting a pain response;
   wherein a detectable difference between the biological activity of the complement component in the test cell and the biological activity of the complement component in the control cell indicates that the test cell is exhibiting a pain response.

### 5.10.1. Test and Control Cells

Test and control cells are preferably the same type of cells from the same species and tissue, and can be any cells useful for conducting this type of assay where a meaningful result can be obtained. If the method focuses on complement component-encoding nucleic acids, any cell type may be used in which a complement component-encoding nucleic acid molecule is ordinarily expressed, or in which a complement component-encoding nucleic acid is expressed in connection with pain or a related treatment or stimulus. If the method focuses on complement component protein expression or biological activity, any cell type may be used in which a complement component is ordinarily expressed, or in which a complement component is expressed in connection with pain or a related treatment or stimulus.

The test cell, for example, can be any cell derived from a tissue of an organism experiencing pain or an associated disorder. Alternatively, the test cell can be any cell grown in vitro under defined conditions. When the test cell is derived from a tissue of an organism experiencing a feeling of pain or associated disorder, it may or may not be known to be located in the region associated with the feeling of pain.

In one embodiment, the test and control cells are cells from the central nervous system (CNS) or peripheral nervous system (PNS). Preferably, the test and control cells are neuronal cells from the DRG, the sciatic nerve, or the spinal cord. The test and control cells can be derived from any appropriate organism, but are preferably human, rat or mouse cells. For example, the test and control cells can be derived from any appropriate organism during a biopsy or by withdrawing blood or spinal fluid.

In a specific embodiment, the test and control cells are from an animal model of pain (e.g., a rat SNL model of neuropathic pain) or any related disorder, and may or may not be isolated from that animal model. Both the test cell and the control cell must have the ability to express the complement component of interest.

The control cell can be any cell that has not been subjected to any treatment or stimulus associated with pain, or which otherwise is not exhibiting a pain response. Preferably, the control cell is otherwise similar and treated in an identical manner to the test cell. For example, when the test cell is derived from a tissue of an animal experiencing pain or associated disorder, the control cell can be derived from an identical tissue or body part of a different animal from the same species which animal is not experiencing pain or associated disorder. Alternatively, the control cell can be derived from an identical tissue or body part of the same animal from which the test cell is derived. However, if this is the case, the identical tissue or body part should not have been subjected to any treatment or stimulus associated with pain within a relevant time frame. When the test cell is a cell grown in vitro under specific conditions, the control cell can be a similar cell grown in vitro under identical conditions but in the absence of the pain-associated treatment or stimulus.

In one embodiment, the test cell has been exposed to a treatment or stimulus that is, or that simulates or mimics, a pain condition prior to determining: (i) the expression level of the nucleic acid molecule encoding a complement component protein, (ii) the expression level of a complement component protein, or (iii) a biological activity of a complement component. The control cell is useful as an appropriate comparator cell to allow a determination of whether or not the test cell is exhibiting a pain response. For example, where the test cell has been exposed to a treatment or stimulus that is, or that simulates or mimics, a pain condition, the control cell has not been exposed to such a treatment or stimulus. In another embodiment, the test cell has been exposed to a compound that is being tested to determine whether it simulates or mimics a pain condition.

### 5.10.2. Determining Nucleic Acid Expression, Protein Expression, or Protein Activity

Any appropriate technique can be used to determine the expression level of a nucleic acid molecule encoding a complement component, or the expression level of a complement component, or the level of biological activity of a complement component protein.

### 5.10.3. Comparing the Nucleic Acid Expression, Protein Expression, or Protein Activity of the Test and Control Cells

A detectable change, as defined supra, indicating that a test cell is exhibiting a pain response can be selected from:
(i) an increase in expression of a nucleic acid molecule encoding a complement effector in the test cell relative to the expression of the nucleic acid in a control cell;
(ii) a decrease in expression of a nucleic acid molecule encoding an endogenous complement inhibitor in the test cell relative to the expression of the nucleic acid in a control cell;
(iii) an increase in expression of a complement effector in a test cell relative to the expression of the effector in a control cell;
(iv) a decrease in expression of an endogenous complement inhibitor in a test cell relative to the expression of the endogenous inhibitor in a control cell;
(v) an increase in activity of a complement effector in a test cell relative to the activity of the effector in a control cell; and
(vi) a decrease in activity of an endogenous complement inhibitor in a test cell relative to the activity of the endogenous inhibitor in a control cell.

### 5.11. Methods of Inhibiting Complement to Treat Pain

The present invention further provides methods for treating pain or related disorders by modulating expression of a complement component-encoding nucleic acid molecule or a complement component comprising administering to a subject in need of such treatment a therapeutically effective amount of a compound that modulates expression of a complement component-encoding nucleic acid molecule or a complement component.

The present invention further provides methods for treating pain or related disorders by modulating a biological activity of a complement component, comprising administering to a subject in need of such treatment a therapeutically effective amount of a compound that modulates a biological activity of a complement component protein.

Treating pain can require the modulation of: (i) the expression of one or more nucleic acids encoding one or more complement components; (ii) the expression of one or more complement components; or (iii) one or more activities of one or more complement components, or a combination thereof.

Conditions that can be treated using any of the methods herein disclosed include a pain condition or a pain-related disorder selected without limitation from chronic pain, nociceptive pain, neuropathic pain (including all types of hyperalgesia and allodynia), and cancer pain. In a preferred embodiment, a condition treated by a method of the present invention is chronic pain. In another preferred embodiment, a condition treated by a method of the present invention is neuropathic pain.

### 5.11.1. Modulation of Complement Effectors

In one embodiment of this method, the complement component is a complement effector. In another specific embodiment, the expression of a complement effector-encoding nucleic acid, or the expression of a complement effector, is decreased by administering a complement inhibitor (e.g., an antisense oligonucleotide that targets a specific complement effector). In another specific embodiment, the activity of a complement effector is decreased by administering a complement inhibitor (e.g., a small molecule, polyionic agent, antibody, peptide, or protein). Alternatively, the complement inhibitor can inhibit an increase in the expression or biological activity of a complement effector.

### 5.11.2. Modulation of Endogenous Complement Inhibitors

In one embodiment of this method, the complement component is an endogenous complement inhibitor. In a specific embodiment, the expression (i) of a nucleic acid molecule having a nucleotide sequence encoding an endogenous complement inhibitor, or (ii) of an endogenous complement inhibitor is increased by administering a molecule that stimulates expression of the nucleic acid molecule or protein, respectively (e.g., a statin, HB-EGF, TNFα, estrogen, IL4, NFG, histamine, or phorbol-12-myristate-13-acetate).

In another embodiment, the activity of an endogenous complement inhibitor is increased by administering a compound that increases the activity of an endogenous complement inhibitor. Alternatively, a compound is administered that inhibits a decrease in the expression or activity of an endogenous complement inhibitor.

### 5.11.3. Inhibition of Specific Portions of the Complement Cascade

In yet another embodiment, a complement component is modulated such that only a specific portion of the complement cascade is affected. Modulating a complement component may affect complement components that are downstream of the modulated component, but leave the upstream components unaffected. In one non-limiting embodiment, the complement effectors, C5b-9, are inhibited by binding of a monoclonal antibody to C5 (see U.S. Pat. No. 5,135,916) and, as a result, the MAC is unable to lyse pathogens. However, in this example, the complement cascade upstream of C5b-9 remains unaffected.

A complement component specific to the classical pathway (e.g., C1q, C1r, or C1s), or the MB-lectin pathway (e.g., MBL, MASP-1, or MASP-2), or the alternative pathway (e.g., Factor D or Factor B), can be modulated. In one non-limiting example, inhibition of C1s by C1s-1NH-248 (Buerke et al., J. Immun. 2001, 167:5375-80) blocks the classical pathway of the complement cascade, but presumably (although it has not been directly tested in the MB-lectin pathway assay) leaves both the MB-lectin pathway and the alternative pathway uninhibited. Modulating complement components of different pathways could effectively reduce pain while leaving intact complement-mediated surveillance of the immune system.

### 5.11.4. Formulations and Dosages

According to the present invention, a therapeutically effective amount of a compound that modulates a complement component can be administered to a subject to treat pain.

The term “therapeutically effective amount” is used here to refer to an amount or dose of a compound sufficient: (i) to detectably change the level of expression of a complement component-encoding nucleic acid or a complement component in a subject; or (ii) to detectably change the level of a biological activity of a complement component in a subject; or (iii) to cause a detectable improvement in a clinically significant symptom or condition (e.g., amelioration of pain) in a subject.

A compound useful in carrying out a therapeutic method of the present invention is advantageously formulated in a pharmaceutical composition in combination with a pharmaceutically acceptable carrier. The amount of compound in the pharmaceutical composition depends on the desired dosage and route of administration, as discussed below. In one embodiment, suitable dose ranges of the active ingredient are from about 0.01 mg/kg to about 1500 mg/kg of body weight taken at necessary intervals (e.g., daily, every 12 hours, etc.). In another embodiment, a suitable dosage range of the active ingredient is from about 0.1 mg/kg to about 150 mg/kg of body weight taken at necessary intervals. In another embodiment, a suitable dosage range of the active ingredient is from about 1 mg/kg to about 15 mg/kg of body weight taken at necessary intervals.

In one embodiment, the dosage and administration are such that the complement cascade is only partially inhibited so as to avoid any unacceptably deleterious effects of reducing complement immunity.

A therapeutically effective compound can be provided to the patient in a standard formulation that includes one or more pharmaceutically acceptable additives, such as excipients, lubricants, diluents, flavorants, colorants, buffers, and disintegrants. The formulation may be produced in unit dosage form for administration by oral, parenteral, transmucosal, intranasal, rectal, vaginal, or transdermal routes. Parental routes include intravenous, intra-arteriole, intramuscular, intradermal, subcutaneous, intraperitoneal, intraventricular, intrathecal, and intracranial administration.

The pharmaceutical composition may also include one or more other biologically active substances in combination with the complement-modulating compound. Such substances include but are not limited to opioids, non-steroidal anti-inflammatory drugs (NSAIDs), and other analgesics.

The pharmaceutical composition can be added to a retained physiological fluid such as blood or synovial fluid. In one embodiment for CNS administration, a variety of techniques are available for promoting transfer of the therapeutic agent across the blood brain barrier, or to gain entry into an appropriate cell, including disruption by surgery or injection, co-administration of a drug that transiently opens adhesion contacts between CNS vasculature endothelial cells, and co-administration of a substance that facilitates translocation through such cells. In another embodiment, for example, to target the peripheral nervous system (PNS), the pharmaceutical composition has a restricted ability to cross the blood brain barrier and can be administered using techniques known in the art.

In yet another embodiment, the complement-modulating compound is delivered in a vesicle, particularly a liposome. In one embodiment, the complement-modulating compound is delivered topically (e.g., in a cream) to the site of pain (or related disorder) to avoid the systemic effects of inhibiting complement in non-target cells or tissues.

In another embodiment, the therapeutic agent is delivered in a controlled release manner. For example, a therapeutic agent can be administered using intravenous infusion with a continuous pump, or in a polymer matrix such as poly-lactic/glutamic acid (PLGA), or in a pellet containing a mixture of cholesterol and the active ingredient (SilasticR™; Dow Coming, Midland, Mich.; see U.S. Pat. No. 5,554,601), or by subcutaneous implantation, or by transdermal patch.

In one embodiment, an inhibitory RNA oligonucleotide or an antisense oligonucleotide that can inhibit expression of a complement component or a nucleic acid molecule encoding a complement inhibitor is delivered to a subject by administration of an appropriately constructed vector. Delivery of a nucleic acid can be performed using a viral vector or, alternatively, a nucleic acid can be introduced through direct introduction of DNA.

The formulation and dosage for a therapeutic agent according to a method of the present invention will depend on the severity of the disease condition being treated, whether other drugs are being administered, whether other actions are taken (such as diet modification), the weight, age, and sex of the subject, and other criteria. The skilled medical practitioner will be able to select the appropriate formulation and dosage in view of these criteria and based on the results of published clinical trials.

### 5.12. Screening Methods

The present invention further provides a method to identify compounds that modulate the complement cascade for use as therapeutics to treat pain. The pain can be any type of pain such as, but not limited to inflammatory pain, cancer-related pain, or neuropathic pain.

In one embodiment, the present invention provides a method for identifying a compound capable of treating pain by modulating expression of a complement component-encoding nucleic acid molecule, said method comprising:
(a) contacting a first cell capable of expressing a complement component-encoding nucleic acid molecule with a test compound under conditions sufficient to allow the cell to respond to said contact with the test compound;
(b) determining in the cell of step (a) the expression level of the complement component-encoding nucleic acid molecule during or after contact with the test compound; and
(c) comparing the expression level of the complement component-encoding nucleic acid molecule determined in step (b) to the expression level of the complement component-encoding nucleic acid molecule in a control cell that has not been contacted with the test compound;
wherein a detectable difference between the expression level of the complement component-encoding nucleic acid molecule in the first cell in response to contact with the test compound and the expression level of the complement component-encoding nucleic acid molecule in the control cell that has not been contacted with the test compound indicates that the test compound modulates the expression of the complement component-encoding nucleic acid. Such a test compound can be considered a candidate compound, and subjected to further testing and analysis.

In another embodiment, the present invention provides a method for identifying a compound capable of treating pain by modulating expression of a complement component, said method comprising:
(a) contacting a first cell capable of expressing a complement component with a test compound under conditions to allow the cell to respond to said contact with the test compound;
(b) determining in the cell of step (a) the expression level of the complement component during or after contact with the test compound; and
(c) comparing the expression level of the complement component determined in step (b) to the expression level of the complement component in a control cell that has not been contacted with the test compound;
   wherein a detectable difference between the expression level of the complement component in the first cell in response to contact with the test compound and the expression level of the complement component in the control cell that has not been contacted with the test compound indicates that the test compound modulates the expression of the complement component. Such a test compound can be considered a candidate compound, and subjected to further testing and analysis.

In another embodiment, the present invention provides a method for identifying a compound capable of treating pain by modulating a biological activity of a complement component, said method comprising:
(a) contacting a complement component with a test compound under conditions to allow the complement component to respond to said contact with the test compound;
(b) determining a biological activity of the complement component during or after contact with the test compound; and
(c) comparing the biological activity of the complement component determined in step (b) to the biological activity of the complement component when the protein has not been contacted with the test compound;
   wherein a detectable difference between the activity of the complement component in response to contact with the test compound and the activity of the complement component when the complement component has not been contacted with the test compound indicates that the test compound modulates a biological activity of the complement component. Such a test compound can be considered a candidate compound, and subjected to further testing and analysis.

In vitro and cell-based assays can be used to screen compounds for their ability to modulate a component of the complement cascade and to treat pain. In vivo assays can also be used to screen compounds for their ability to modulate a component of the complement cascade and to treat pain. In one embodiment, in vitro and/or cell-based assays are used to identify “candidate compounds” having the ability to modulate a component of the complement pathway. These candidate compounds can be further tested in an in vivo assay to confirm their ability to treat pain.

### 5.12.1.Cells Used in Screening Methods

In any of the aforementioned screens for compounds that modulate the expression of a complement component-encoding nucleic acid, any appropriate cell type may be used which can express the complement component-encoding nucleic acid molecule of interest. If the screening method identifies compounds that modulate complement component expression or a biological activity thereof, any appropriate cell type may be used which can express the complement component of interest. Such a cell can be derived from a tissue of an organism, cultured in vitro under defined conditions, or engineered to recombinantly express or overexpress the nucleic acid molecule or complement component of interest. (For further description of cells that recombinantly express complement components, see below.) In one embodiment, the cells are from the CNS or PNS. In a specific embodiment, the cells are neuronal cells from the DRG, the sciatic nerve, or the spinal cord. Cells can be derived from any appropriate mammal, such as human, rat and mouse. For example, the cells can be derived from an appropriate organism during a biopsy or by withdrawing an appropriate fluid sample, such as blood or spinal fluid.

In a specific embodiment, the cells are from an animal model of pain (e.g., a rat SNL model of neuropathic pain) or an animal model of a pain-related disorder, and may or may not be isolated from that animal model. In another embodiment, the cells are from a subject, such as a human or companion animal. The cells may or may not be isolated from the subject being tested.

### 5.12.1.1. Cells Engineered to Express a Complement Component

A cell used in the screening methods described above can be a cell that has been recombinantly engineered to express or overexpress a nucleic acid molecule encoding a complement component. Such cells can be made by the transformation of host cells with a vector capable of expressing a complement component, and by the subsequent expression of the complement component. This section describes expression vectors, transformation methods, and expression methods that can be used in the formation of a cell that has been recombinantly engineered to express nucleic acid molecules and proteins. Table 2 provides examples of nucleic acid molecules encoding complement components that can be expressed.

### 5.12.1.2. Expression Vectors

Expression vectors can be constructed comprising the coding sequence for a complement component in operative association with one or more regulatory elements necessary for transcription and translation of the coding sequence to produce a polypeptide. As used herein, the term “regulatory element” includes but is not limited to nucleotide sequences that encode inducible and non-inducible promoters, enhancers, operators and other elements known in the art that serve to drive and/or regulate expression of polynucleotide coding sequences. Also, as used herein, the coding sequence is in operative association with one or more regulatory elements where the regulatory elements effectively regulate and allow for the transcription of the coding sequence or the translation of its mRNA, or both.

The regulatory elements of these and other vectors can vary in their strength and specificities. Depending on the host/vector system utilized, any of a number of suitable transcription and translation elements can be used. For instance, when cloning in mammalian cell systems, promoters isolated from the genome of mammalian cells, e.g., mouse metallothionein promoter, or from viruses that grow in these cells, e.g., vaccinia virus 7.5 K promoter or Maloney murine sarcoma virus long terminal repeat, can be used. Promoters obtained by recombinant DNA or synthetic techniques can also be used to provide for transcription of the inserted sequence. In addition, expression from certain promoters can be elevated in the presence of particular inducers, e.g., zinc and cadmium ions for metallothionein promoters. Non-limiting examples of transcriptional regulatory regions or promoters include for bacteria, the β-gal promoter, the T7 promoter, the TAC promoter, λ left and right promoters, trp and lac promoters, trp-lac fusion promoters, etc.; for yeast, glycolytic enzyme promoters, such as ADH-I and -II promoters, GPK promoter, PGI promoter, TRP promoter, etc.; and for mammalian cells, SV40 early and late promoters, and adenovirus major late promoters, among others.

Specific initiation signals are also required for sufficient translation of inserted coding sequences. These signals typically include an ATG initiation codon and adjacent sequences. In cases where the nucleic acid molecule, including its own initiation codon and adjacent sequences, is inserted into the appropriate expression vector, no additional translation control signals may be needed. However, in cases where only a portion of a coding sequence is inserted, exogenous translational control signals, including the ATG initiation codon, may be required. These exogenous translational control signals and initiation codons can be obtained from a variety of sources, both natural and synthetic. Furthermore, the initiation codon must be in-phase with the reading frame of the coding regions to ensure in-frame translation of the entire insert.

Methods are known in the art for constructing recombinant vectors containing particular coding sequences in operative association with appropriate regulatory elements, and these can be used to practice the present invention. These methods include in vitro recombinant techniques, synthetic techniques, and in vivo genetic recombination. See, e.g., the techniques described in Ausubel et al., 1989, above; Sambrook et al., 1989, above; Saiki et al., 1988, above; Reyes et al., 2001, above; Wu et al., 1989, above; U.S. Pat. Nos. 4,683,202; 6,335,184 and 6,027,923.

A variety of expression vectors are known in the art that can be utilized to express a nucleic acid molecule encoding a complement component, including recombinant bacteriophage DNA, plasmid DNA, and cosmid DNA expression vectors containing the particular coding sequences. Typical prokaryotic expression vector plasmids that can be engineered to contain a polynucleotide molecule include pUC8, pUC9, pBR322 and pBR329 (Biorad Laboratories, Richmond, Calif.), pPL and pKK223 (Pharmacia, Piscataway, N.J.), pQE50 (Qiagen, Chatsworth, Calif.), and pGEM-T EASY (Promega, Madison, Wis.), pcDNA6.2/V5-DEST and pcDNA3.2NV5DEST (Invitrogen, Carlsbad, Calif.) among many others. Typical eukaryotic expression vectors that can be engineered to contain a polynucleotide molecule include an ecdysone-inducible mammalian expression system (Invitrogen, Carlsbad, Calif.), cytomegalovirus promoter-enhancer-based systems (Promega, Madison, Wis.; Stratagene, La Jolla, Calif.; Invitrogen), and baculovirus-based expression systems (Promega), among many others.

Expression vectors can also be constructed that will express a fusion protein comprising a complement component. Such fusion proteins can be used, e.g., to study the biochemical properties, to aid in the identification or purification, or to improve the stability, of a recombinantly-expressed complement component. Possible fusion protein expression vectors include but are not limited to vectors incorporating sequences that encode β-galactosidase and trpE fusions, maltose-binding protein fusions, glutathione-S-transferase fusions, polyhistidine fusions (carrier regions), V5, HA, myc, and HIS. Methods known in the art can be used to construct expression vectors encoding these and other fusion proteins.

A signal sequence upstream from, and in reading frame with, the complement component coding sequence can be engineered into the expression vector by known methods to direct the trafficking and secretion of the expressed protein. Non-limiting examples of signal sequences include those from α-factor, immunoglobulins, outer membrane proteins, penicillinase, and T-cell receptors, among others. Other examples of the signal sequences that can be used are PhoA signal sequence, OmpA signal sequence, etc., in the case of using bacteria of the genus Escherichia as the host; α-amylase signal sequence, subtilisin signal sequence, etc., in the case of using bacteria of the genus Bacillus as the host; MFα signal sequence, SUC2 signal sequence, etc., in the case of using yeast as the host; and insulin signal sequence, α-interferon signal sequence, antibody molecule signal sequence, etc., in the case of using animal cells as the host.

To aid in the selection of host cells transformed or transfected with a recombinant vector, the vector can be engineered to further comprise a coding sequence for a reporter gene product or other selectable marker. Such a coding sequence is preferably in operative association with the regulatory elements, as described above. Reporter genes that are useful in practicing the invention are known in the art, and include those encoding chloramphenicol acetyltransferase (CAT), green fluorescent protein, firefly luciferase, and human growth hormone, among others. Nucleotide sequences encoding selectable markers are known in the art, and include those that encode gene products conferring resistance to antibiotics or anti-metabolites, or that supply an auxotrophic requirement. Examples of such sequences include those that encode thymidine kinase activity, or resistance to methotrexate, ampicillin, kanamycin, chloramphenicol, zeocin, pyrimethamine, aminoglycosides, hygromycin, blasticidine, or neomycin, among others.

### 5.12.1.3. Transformation Methods

A transformed host cell comprising a polynucleotide molecule or recombinant vector encoding a complement component is useful for expressing a complement component. Such transformed host cells include but are not limited to microorganisms, such as bacteria transformed with recombinant bacteriophage DNA, plasmid DNA or cosmid DNA vectors, or yeast transformed with a recombinant vector, or animal cells, such as insect cells infected with a recombinant virus vector, e.g., baculovirus, or mammalian cells infected with a recombinant virus vector, e.g., adenovirus, vaccinia virus, lentivirus, adeno-associated virus (AAV), or herpesvirus, among others. For example, a strain of E. coli can be used such as, e.g., the DH5α strain available from the ATCC, Manassas, Va., USA (Accession No. 31343), or from Stratagene (La Jolla, Calif.). Eukaryotic host cells include yeast cells, although mammalian cells, e.g., from a mouse, rat, hamster, cow, monkey, or human cell line, among others, can also be utilized effectively. Examples of eukaryotic host cells that can be used to express a recombinant protein of the invention include Chinese hamster ovary (CHO) cells (e.g., ATCC Accession No. CCL-61), NIH Swiss mouse embryo cells NIH/3T3 (e.g., ATCC Accession No. CRL-1658), human epithelial kidney cells HEK 293 (e.g., ATCC Accession No. CRL-1573), and Madin-Darby bovine kidney (MDBK) cells (ATCC Accession No. CCL-22).

As described above, the present invention provides mammalian cells infected with a virus containing a recombinant viral vector. For example, an overview and instructions concerning the infection of mammalian cells with adenovirus using the AdEasy™ Adenoviral Vector System is given in the Instructions Manual for this system from Stratagene (La Jolla, Calif.). As another example, an overview and instructions concerning the infection of mammalian cells with AAV using the AAV Helper-Free System is given in the Instructions Manual for this system from Strategene (La Jolla, Calif.).

The recombinant vector of the present invention is preferably transformed or transfected into one or more host cells of a substantially homogeneous culture of cells. The vector is generally introduced into host cells in accordance with known techniques, such as, e.g., by protoplast transformation, calcium phosphate precipitation, calcium chloride treatment, microinjection, electroporation, transfection by contact with a recombined virus, liposome-mediated transfection, DEAE-dextran transfection, transduction, conjugation, or microprojectile bombardment, among others. Selection of transformants can be conducted by standard procedures, such as by selecting for cells expressing a selectable marker, e.g., antibiotic resistance, associated with the recombinant expression vector.

Once an expression vector is introduced into the host cell, the presence of the nucleic acid molecule of the present invention, either integrated into the host cell genome or maintained episomally, can be confirmed by standard techniques, e.g., by DNA-DNA, DNA-RNA, or RNA-antisense RNA hybridization analysis, restriction enzyme analysis, PCR analysis including reverse transcriptase PCR (RT-PCR), detecting the presence of a “marker” gene function, or by immunological or functional assay to detect the expected protein product.

### 5.12.1.4. Expression Methods

Once a nucleic acid molecule encoding a complement component has been stably introduced into an appropriate host cell, the transformed host cell is clonally propagated, and the resulting cells can be grown under conditions conducive to the efficient production (i.e., expression or overexpression) of the encoded complement component. Where the expression vector comprises an inducible promoter, appropriate induction conditions such as, e.g., temperature shift, exhaustion of nutrients, addition of gratuitous inducers (e.g., analogs of carbohydrates, such as isopropyl-β-D-thiogalactopyranoside (IPTG)), accumulation of excess metabolic by-products, or the like, are employed as needed to induce expression.

### 5.12.2. Proteins Used in Screening

In any of the aforementioned methods to screen for compounds that modulate the activity of a complement component, the activity of the complement component can be measured in a subject, in a tissue, in a cell, or in isolation. Cells used in such screening methods have been described, supra. The complement component can be isolated by purification from a cell expressing the complement component. In additional embodiments, complement components can be produced by in vitro translation of a nucleic acid molecule that encodes the complement component, by chemical synthesis (e.g., solid phase peptide synthesis), or by any other suitable method.

### 5.12.2.1. Purification of Complement Component from Cells

Where the polypeptide is retained inside the host cells or contained in a cell membrane, the cells are harvested and lysed, and the product is substantially purified or isolated from the lysate or membrane fraction under extraction conditions known in the art to minimize protein degradation such as, e.g., at 4° C., or in the presence of protease inhibitors, or both. Where the polypeptide is secreted from the host cells, the exhausted nutrient medium can simply be collected and the polypeptide substantially purified or isolated therefrom.

The polypeptide can be substantially purified or isolated from cell lysates, membrane fractions, or culture medium, as necessary, using standard methods, including but not limited to one or more of the following methods: ammonium sulfate precipitation, size fractionation, ion exchange chromatography, HPLC, density centrifugation, affinity chromatography, ethanol precipitation, and chromatofocusing. During purification, the polypeptide can be detected based, e.g., on size, or reactivity with a polypeptide-specific antibody, or by detecting the presence of a fusion tag.

According to the present invention, the recombinantly expressed full-length complement component protein may be associated with the cellular membrane as a transmembrane protein. Such protein can be isolated from membrane fractions of host cells. The cell membrane fraction refers to a fraction abundant in cell membrane obtained by cell disruption and subsequent fractionation by any of the known methods. Useful cell disruption methods include, e.g., cell squashing using a Potter-Elvehjem homogenizer, disruption using a Waring blender or Polytron (manufactured by Kinematica Inc.), disruption by ultrasonication, and disruption by cell spraying through thin nozzles under an increased pressure using a French press or the like. Cell membrane fractionation is effected mainly by fractionation using a centrifugal force, such as centrifugation for fractionation and density gradient centrifugation. For example, cell disruption fluid can be centrifuged at a low speed (500 rpm to 3,000 rpm) for a short period of time (normally about 1 to about 10 minutes), the resulting supernatant is then centrifuged at a higher speed (15,000 rpm to 30,000 rpm) normally for 30 minutes to 2 hours. The precipitate thus obtained can be used as the membrane fraction. The membrane fraction is rich in membrane components such as cell-derived phospholipids and transmembrane and membrane-associated proteins. In yet other embodiments, the membrane fraction may be further solubilized with a detergent. Detergents that may be used with the present invention include without limitation Triton X-100, β-octyl glucoside, and CHAPS (see also Langridge et al., Biochim. Biophys. Acts. 1983; 751: 318).

A preferred method for isolating transmembrane proteins is a technique that uses 2-D gel electrophoresis as described, for example, in the instructions for “2-D Sample Prep for Membrane Proteins” from Pierce Biotechnology, Inc. (Rockford, Ill.).

Upon isolation of the membrane fraction, the peripheral proteins of these membranes can be removed by extraction with high salt concentrations, high pH or chaotropic agents such as lithium diiodosalicylate. The integral proteins can then be solubilized using a detergent such as Triton X-100, β-octyl glucoside, CHAPS, or other compounds of similar action (see, e.g., Beros et al., J. Biol. Chem. 1987; 262: 10613). A combination of several standard chromatographic steps (e.g., ion exchange chromatography, gel permeation chromatography, adsorption chromatography or isoelectric focusing) and/or a single purification step involving immuno-affinity chromatography using immobilized antibodies (or antibody fragments) to the protein and/or preparative polyacrylamide gel electrophoresis using instrumentation such as the Applied Biosystems “230A EPEC System” can be then used to purify the protein and remove it from other integral proteins of the detergent-stabilized mixture. It is recognized that the hydrophobic nature of the transmembrane protein may necessitate the inclusion of amphiphillic compounds such as detergents and other surfactants (see bud Kar and Maloney, J. Biol. Chem. 1986; 261: 10079) during handling.

For use in practicing the present invention, the polypeptide can be in an unpurified state as secreted into the culture fluid or as present in a cell lysate or membrane fraction. Alternatively, the polypeptide may be purified therefrom. Once a polypeptide of the present invention of sufficient purity has been obtained, it can be characterized by standard methods, including by SDS-PAGE, size exclusion chromatography, amino acid sequence analysis, immunological activity, biological activity, etc. The polypeptide can be further characterized using hydrophilicity analysis (see, e.g., Hopp and Woods, Proc. Natl. Acad. Sci. USA 1981; 78: 3824), or analogous software algorithms, to identify hydrophobic and hydrophilic regions. Structural analysis can be carried out to identify regions of the polypeptide that assume specific secondary structures. Biophysical methods such as X-ray crystallography (Engstrom, Biochem. Exp. Biol. 1974; 11: 7-13), computer modeling (Fletterick and Zoller eds., In: Current Communications in Molecular Biology, Cold Spring Harbor Laboratory, Cold Spring Harbor, N.Y., 1986), and nuclear magnetic resonance (NMR) can be used to map and study potential sites of interaction between the polypeptide and other putative interacting proteins/receptors/molecules. Information obtained from these studies can be used to design deletion mutants, and to design or select therapeutic compounds that can specifically modulate the biological function of the complement component protein in vivo.

The fusion protein can be useful to aid in purification of the expressed protein. In non-limiting embodiments, e.g., a complement component-maltose-binding fusion protein can be purified using amylose resin; a complement component-glutathione-S-transferase fusion protein can be purified using glutathione-agarose beads; and a complement component-polyhistidine fusion protein can be purified using divalent nickel resin. Alternatively, antibodies against a carrier protein or peptide can be used for affinity chromatography purification of the fusion protein. For example, a nucleotide sequence coding for the target epitope of a monoclonal antibody can be engineered into the expression vector in operative association with the regulatory elements and situated so that the expressed epitope is fused to a complement component protein of the present invention. In a non-limiting embodiment, a nucleotide sequence coding for the FLAG™ epitope tag (International Biotechnologies Inc.), which is a hydrophilic marker peptide, can be inserted by standard techniques into the expression vector at a point corresponding, e.g., to the amino or carboxyl terminus of the complement component protein. The expressed complement component protein-FLAG™ epitope fusion product can then be detected and affinity-purified using commercially available anti-FLAG™ antibodies. The expression vector can also be engineered to contain polylinker sequences that encode specific protease cleavage sites so that the expressed complement component protein can be released from a carrier region or fusion partner by treatment with a specific protease. For example, the fusion protein vector can include a nucleotide sequence encoding a thrombin or factor Xa cleavage site, among others.

### 5.12.3. Compounds Used for Screening

A compound that can be screened according to a method of the present invention can be any compound having a potential therapeutic ability to treat pain. Examples of such compounds include: (i) small inorganic molecules; (ii) small organic molecules (including natural product compounds); (iii) peptides, peptide analogs, and mimetics; (iv) antibodies (including recombinant humanized antibodies) and immunospecific fragments of antibodies; and (v) soluble proteins (such as recombinantly produced endogenous complement inhibitors (e.g. soluble DAF and CR1)). Small inorganic and organic molecules are less than about 2 kDa in molecular weight, and more preferably less than about 1 kDa in molecular weight. In one embodiment, compounds that remain extracellullar and/or bind to the cell surface are selected. Compounds can also be selected that can cross the blood-brain barrier or gain entry into an appropriate cell to affect the expression of the complement component-encoding gene or a biological activity of the complement component. Compounds identified by these screening assays may also be selected from polypeptides, such as soluble peptides, fusion peptides, antibodies, members of combinatorial libraries (such as those described by Lam et al., Nature 1991, 354:82-84; and by Houghten et al., Nature 1991, 354:84-86); members of libraries derived by combinatorial chemistry, such as molecular libraries of D- and/or L-configuration amino acids; phosphopeptides, such as members of random or partially degenerate, directed phosphopeptide libraries (see, e.g., Songyang et al., Cell 1993, 72:767-778); peptide libraries derived from the “phage method” (Scott and Smith, Science 1990, 249:386-390; Cwirla, et al., Proc. Natl. Acad. Sci. USA 1990, 87:6378-6382; Devlin et al., Science 1990, 49:404-406); chemicals from other chemical libraries (Geysen et al., Molecular Immunology 1986, 23:709-715; Geysen et al., J. Immunologic Methods 1987, 102:259-274; Fodor et al., Science 1991, 251:767-773;. Furka et al., 14th International Congress of Biochemistry 1988, Volume #5, Abstract FR:013; Furka, Int. J. Peptide Protein Res. 1991, 37:487-493; U.S. Pat. No. 4,631,211; U.S. Pat. No. 5,010,175;Needels et al., Proc. Natl. Acad. Sci. USA 1993, 90:10700-4; Ohlmeyer et al., Proc. Natl. Acad. Sci. USA 1993, 90:10922-10926; PCT Publication No. WO 92/00252; and PCT Publication No. WO 94/28028); and large libraries of synthetic or natural compounds available from a variety of sources, including Maybridge Chemical Co. (Trevillet, Cornwall, UK), Comgenex (Princeton, N.J.), Brandon Associates (Merrimack, N.H.), Microsource (New Milford, Conn.), Aldrich (Milwaukee, Wis.), Pan Laboratories (Bothell, Wash.), and MycoSearch (NC) (see, e.g., Blondelle et al., TIBTech 1996, 14:60).

One skilled in the art can appreciate that a plurality of compounds can be screened simultaneously in a single screening assay. Screening more than a single compound at a time allows for the possibility that, although a single compound may be insufficient to create an effect, a combination of compounds may produce the desired effect.

### 5.12.4. Determining Nucleic Acid Expression Levels, Protein Expression Levels, and Protein Activity Levels

Screening methods of the present invention can include the step of determining the expression level of a complement component-encoding nucleic acid during or after contact with a test compound. Screening methods of the present invention can alternatively or additionally include the step of determining the expression level of a complement component during or after contact with a test compound. Screening methods of the present invention can alternatively or additionally include the step of determining a biological activity of a complement component during or after contact with a test compound. Determining a biological activity of a complement component may include determining the binding of a complement component to a compound.

Any of the techniques described in the “Determining Nucleic Acid Expression Levels, Protein Expression Levels, and Protein Activity” Section, supra, can be used.

### 5.12.5. Testing the Effectiveness of Candidate Agents in Treating Pain In vivo

Screening for compounds that treat pain and related disorders by modulating a complement component can be accomplished using in vivo methods as described below. In vivo methods of the present invention can be used in conjunction with the assays described above, or can be used independently of the above methods. In one embodiment, in vitro and/or cell-based methods are performed to identify candidate compounds that can be further tested in one or more in vivo assays to determine the ability of the compounds to treat pain.

These screening methods can further comprise the in vivo steps of:
(a) determining the degree of pain experienced by a test subject during or after contact with the test compound; and
(b) comparing the degree of pain experienced by the test subject in step (a) to the degree of pain experienced by a control subject that has not been contacted with the test compound;
   wherein a detectable difference between the degree of pain experienced by the test subject in response to contact with the test compound and the degree of pain experienced by the control subject indicates that the test compound modulates pain.

Test and control subjects used in these in vivo methods can include transgenic animals and animals models of pain, both of which are described herein above. For example, animal test subjects from an appropriate pain model can be administered a test compound that inhibits a complement component. The subject animals can then be tested to determine their sensitivity to pain (see, e.g., the paw withdrawal threshold test described in the Examples Section 6 below or an assay described in the Animal Models of Pain Section). The pain threshold of an animal treated with a test compound can be compared with the pain threshold of a control animal that was not treated with the test compound to determine the effect of the compound on pain. Alternatively, the pain threshold of an animal treated with a test compound can be compared with the pain threshold of the same animal before treatment with the test compound to determine the effect of the compound on pain. In a preferred embodiment, the candidate compound decreases pain. In a specific embodiment, the test and control subjects are mice, rats, companion animals, or humans.

In conjunction with an assay to test pain, an assay to determine complement activity (e.g., the hemolysis assay) can also be performed to determine if the compound is modulating activity of a complement component in vivo, as demonstrated in the Examples Section below. An assay to determine the expression level of a complement component-encoding nucleic acid molecule or complement component can be performed to determine if the compound is modulating complement expression in vivo.

In another embodiment of in vivo methods, known analgesics can be administered to an animal. The pain threshold and complement activity of the animal can then be tested. This method is useful to determine the mechanism of action for known analgesics. Alternatively, if a known analgesic targets the complement pathway, in vivo methods are useful to determine the effectiveness of that analgesic (see “Evaluation of complement inhibitors” by P. C. Giclas on pg 225-236 in Therapeutic interventions in the complement system, ed. by J. D. Lambris and V. M. Holers).

Also in conjunction with an assay to test pain in vivo, an assay to independently determine the effectiveness of a complement inhibitor on a complement-mediated pathology other than pain can be used to correlate or confirm that pain relief occurs through complement inhibition. Examples of such assays include various inflammation models such as heterologous passive cutaneous anaphylaxis; systemic Forssman reactions; passive Arthus reactions; delayed (contact) sensitivity reactions; endotoxin shock;, and experimental autoimmune myasthenia gravis (Himoti et al., Int Arch Allergy Appl Immunol 1982, 69:262-7; Sato et al., Jpn J Pharmacol. 1986, 42:587-9; and Piddlesden et al., J Neuroimmunol. 1996, 71:173-7).

The present invention is further described by way of the following examples. The use of these and other examples anywhere in the specification is illustrative only and not intended to limit the scope and meaning of the invention or of any exemplified term. Likewise, it is not intended that the invention be limited to any particular preferred embodiments described here. Indeed, many modifications and variations of the invention may be apparent to those skilled in the art upon reading this specification, and such variations can be made without departing the invention in spirit or in scope. The invention is therefore to be limited only by the terms of the appended claims along with the full scope of equivalents to which those claims are entitled.

### 6. EXAMPLES

### 6.1. Example 1

### GeneChip, Taqman, and in situ Analysis of Complement Effectors and Inhibitors in a Neuropathic Pain Model

The present example provides GeneChip® (Affymetrix, Santa Clara, Calif.), Taqman® (Applied Biosystems, Foster City, Calif.), in situ analysis, and immunohistochemistry data indicating that the expression of many complement effectors increase and the expression of one specific endogenous complement inhibitor decreases in an animal experiencing pain.

### 6.1.1. GeneChip® Analysis

### 6.1.1.1. Methods: Preparation of Neuropathic Pain Model

Rats having the L5-L6 spinal nerves ligated (SNL) according to the method of Kim and Chung, Pain 1992; 50:355-63 were used in this experiment. Briefly, nerve injury was induced by tight ligation of the left L5 and L6 spinal nerves, producing symptoms of neuropathic pain as described below. The advantage of this model is that it allows the investigation of dorsal root ganglia that are injured (L5 and L6) versus dorsal root ganglia that are not injured (L4). Thus, it is possible to see changes in gene expression specifically in response to nerve injury.

Surgery was performed under isoflurane/O₂ inhalation anesthesia. Following induction of anesthesia, a 3 cm incision was made just lateral to the spinal vertebrae. The left paraspinal muscles were separated from the spinous process at the L4-S2 levels. The L6 transverse process was carefully removed with a pair of small rongeurs to visually identify the L4-L6 spinal nerves. The left L5 and L6 spinal nerves were isolated and tightly ligated with 7-0 silk suture. A complete hemostasis was confirmed, and the wound was sutured using non-absorbable sutures, such as 4-0 Vicryl.

Both naïve and sham-operated animals were used as controls. Sham-operation consisted of exposing the spinal nerves without ligation or manipulation. After surgery, animals were weighed and administered a subcutaneous (s.c.) injection of Ringers lactate solution. Following injection, the wound area was dusted with antibiotic powder and the animals were kept on a warm pad until recovery from anesthesia. Animals were then returned to their home cages until behavioral testing. The naïve control group consisted of rats that were not operated on (naïve). Eight to twelve rats in each group were evaluated.

Some rats from the SNL and naïve groups were also treated with gabapentin (GPN) as described below. Gabapentin (GPN), an anti-convulsant, has been shown in the clinic to be effective for treating neuropathic pain (Mellegers et al., Clin. J Pain 2001; 17: 284-295; Rose and Kam, Anaesthesia 2002; 57: 451-462).

The L4, L5 and L6 DRGs and the sciatic nerve from the SNL model of neuropathic pain were used to identify genes involved in mediating and responding to pain (including genes affected by GPN treatment) by using expression profiling. Expression profiling is based on identifying probes on a “genome-scale” microarray that are differentially expressed in SNL DRGs and sciatic nerves as compared to DRGs and sciatic nerves of naïve and sham-operated animals.

TABLE 1
summarizes five experimental groups consisting of sham surgery, naïve
or SNL surgery with or without GPN treatment:

| | Experimental | | |
|---|---|---|---|
| Group Number | Group Name | Surgery | Drug Treatment |
| 1 | naïve + vehicle | none performed | vehicle |
| 2 | naïve + GPN | none performed | gabapentin |
| 3 | sham + vehicle | sham | vehicle |
| 4 | SNL + vehicle | SNL | vehicle |
| 5 | SNL + GPN | SNL | gabapentin |

### 6.1.1.2. Methods: Behavioral Testing

Mechanical sensitivity was assessed using the paw pressure test. This test measures mechanical hyperalgesia. Hind paw withdrawal thresholds (“PWT”) (measured in grams) in response to a noxious mechanical stimulus were determined using an analgesymeter (Model 7200, commercially available from Ugo Basile of Italy), as described in Stein, Biochemistry & Behavior 1988; 31: 451-455. The rat's paw was placed on a small platform, and weight was applied in a graded manner up to a maximum of 250 grams. The endpoint was taken as the weight at which the paw was completely withdrawn. PWT was determined once for each rat at each time point, and only the injured ipsilateral paw (i.e., the hind paw on the same side of the animal as the ligation in SNL animals, or the side of the animal where the nerve was exposed but not injured in sham-operated animals) was used in the test. For naïve animals, the left paw or the side that “would have been” subjected to surgery (herein also referred to as “ipsilateral”) was used for the test.

Rats were tested prior to injury (SNL or sham surgery; naïve rats were tested at the same time) to determine a baseline, or normal, PWT. To verify that the surgical procedure was successful, rats were again tested at 12-14 days after surgery. At that time, the observed pain behavior was attributed to neuropathic pain, and inflammation is presumed to have been resolved, since NSAIDs no longer had an effect on pain behavior. Rats with an SNL injury at this time should exhibit a significantly reduced PWT compared to their baseline PWT, while sham-operated and naïve rats should have PWT that is not significantly different from their baseline PWT. Only rats that met these criteria were included in further behavioral testing and the gene expression study.

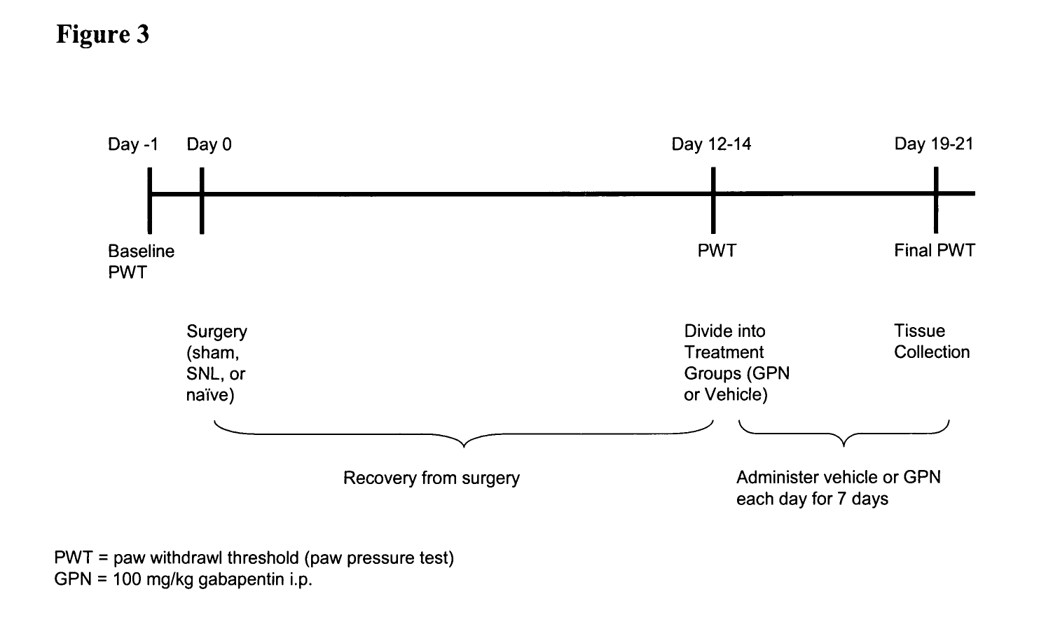

Rats that met the behavior criteria were divided into the treatment groups (described above): 1) naïve+vehicle; 2) naïve+GPN; 3) sham+vehicle; 4) SNL+vehicle; 5) SNL+GPN (Table 1). Vehicle (0.9% saline) and GPN (dissolved in 0.9% saline) were administered intraperitoneally (i.p.) in a volume of 2 ml/kg. The dose of GPN was 100 mg/kg. The rats in the above treatment groups were treated each day for 7 days (with either vehicle or GPN as per their group), and on the last (7^{th}) treatment day (corresponding to 19-21 days post surgery), rats were again assessed for mechanical sensitivity using the paw pressure test described above, in particular to confirm the reversal of neuropathic pain with GPN treatment. Similar to the 12-14 day testing, the observed pain behavior at this time is attributed to neuropathic pain rather than inflammatory pain because NSAIDs no longer have an effect on pain behavior. Following testing, tissues were collected as described below. See FIG. 3 for a summary of the experimental timeline for surgery, treatment, and testing.

### 6.1.1.3. Methods: Determining Gene Expression Profiles in the SNL Model—Tissue Collection and RNA Preparation

Eight to twelve rats meeting behavioral criteria for the five experimental groups described above were sacrificed, and the following tissues were collected separately: brain, hemisected spinal cord cut into ipsilateral (same side) to injury and contralateral (opposite side) to injury, mid-thigh sciatic nerve, and L4, L5 and L6 dorsal root ganglia (DRG), both ipsilateral and contralateral to injury. Samples were rapidly frozen on dry ice. Next, for each experimental group and tissue (5 groups×6 tissues=30 total), the samples were separated into two pools (Pool 1 and Pool 2), consisting of half or 4-6 animals each.

In addition, a separate experiment was conducted with the following samples obtained from naïve animals: adrenal, aorta, fetal brain, kidney, liver, quadriceps muscle, spleen, submaxillary gland, and testis. Samples were rapidly frozen on dry ice. Next, for each experimental group and tissue, the samples were separated into two pools (Pool 1 and Pool 2), consisting of half or 4-6 animals each.

Total RNA from each tissue sample pool was prepared using Tri-Reagent (Sigma, St. Louis, Mo.). Total RNA was quantified by measuring absorption at 260 nm. RNA quality was assessed by measuring absorption at 260 nm/280 nm and by capillary electrophoresis on an RNA Lab-on-chip using Bioanalyzer 2100 (Agilent, Palo Alto, Calif.) to ensure that the ratio of 260 nm/280 nm exceeded 2.0, and that the ratio of 28S rRNA to 18S rRNA exceeded 1.0 for each sample. Pool 1 total RNA was used for the Affymetrix microarray hybridization, and Pool 2 total RNA was used for validation of gene expression profiles by TaqMan® analysis.

### 6.1.1.4. Methods: Determining Gene Expression Profiles in the SNL Model—Microarray Analysis

GeneChip® (Affymetrix, Santa Clara, Calif.) technology allows comparative analysis of the relative expression of thousands of known genes annotated in the public domain (herein, referred to as simply “known genes”), and genes encompassing ESTs (herein, referred to as simply “ESTs”), under multiple experimental conditions. Each gene is represented by a “probeset” consisting of multiple pairs of oligonucleotides (25 nt in length) with sequence complementary to the gene sequence or EST sequence of interest, and the same oligonucleotide sequence with a one base-pair mismatch. These probeset pairs allow for the detection of gene-specific nucleic acid hybridization signals as described below. The Affymetrix Rat U34 A, B and C arrays used for the described analysis contain probesets representing about 26,000 genes including 1200 genes of known relevance to the field of neurobiology. For example, these arrays include probesets specific for detecting the mRNA for kinases, cell surface receptors, cytokines, growth factors and oncogenes.

Hybridization probes were prepared according to the Affymetrix Technical Manual (available on the WorldWideWeb at affymetrix.com/support/technical/manual/expression_manual.affx). First-strand cDNA synthesis was primed for each total RNA sample (10 μg), using 5 mM of oligonucleotide primer encoding the T7 RNA polymerase promoter linked to oligo-dT₂₄ primer. cDNA synthesis reactions were carried out at 42° C. using Superscript II-reverse transcriptase (Invitrogen, Carlsbad, Calif.). Second-strand cDNA synthesis was carried out using DNA polymerase I and T4 DNA ligase. Each double-stranded cDNA sample was purified by sequential Phase Lock Gels (Brinkman Instrument, Westbury, N.Y.) and extracted with a 1:1 mixture of phenol to chloroform (Ambion Inc., Austin, Tex.). Half of each cDNA sample was transcribed in vitro into copy RNA (cRNA) labeled with biotin-UTP and biotin-CTP using the BioArray High Yield RNA Transcript Labeling Kit (Enzo Biochemicals, New York, N.Y.). These cRNA transcripts were purified using RNeasy™ columns (Qiagen, Hilden Germany), and quantified by measuring absorption at 260 nm/280 nm. Aliquots (15 μg) of each cRNA sample were fragmented at 95° C. for 35 min in 40 mM Tris-acetate, pH 8.0, 100 mM KOAc, and 30 mM MgOAc to a mean size of about 50 to 150 nucleotides. Hybridization buffer (0.1 M MES, pH 6.7, 1M NaCl, 0.01% Triton, 0.5 mg/ml BSA, 0.1 mg/ml H. sperm DNA, 50 pM control oligo B2, and 1× eukaryotic hybridization control (Affymetrix, Santa Clara, Calif.) was added to each sample.

Samples were then hybridized to RG-U34 A, B, and C microarrays (Affymetrix) at 45° C. for 16 h. Microarrays were washed and sequentially incubated with streptavidin phycoerythrin (Molecular Probes, Inc., Eugene, Oreg.), biotinylated anti-streptavidin antibody (Vector Laboratories, Inc., Burlingame, Calif.), and streptavidin phycoerythrin on the Affymetrix Fluidic Station. Finally, the microarrays were scanned with a gene array scanner (Hewlett Packard Instruments, Tex.) to capture the fluorescence image of each hybridization. Microarray Suite 5.0 software (Affymetrix) was used to extract gene expression intensity signal from the scanned array images for each probeset under each experimental condition.

### 6.1.1.5. Methods: Determining Gene Expression Profiles in the SNL Model—Statistical Criteria

Based on cumulative historical statistical analysis of replicate sample data (not shown), it was determined that the reproducibility of GeneChip data is dependent on the intensity of the signal. For intensities above 130, the reproducibility exhibits a coefficient of variation (CV; standard deviation divided by the average intensity) of 0.2 or better. Below 130, the reproducibility quickly falls off to CVs approaching infinity. Therefore, for genes having a gene expression intensity greater than 130, there is a high confidence of greater than two standard deviations for apparent fold-changes of three-fold or more.

As has been observed by others (Wang et al., Neuroscience 2002; 114: 529-546), the apparent gene regulation in L5 and L6 was much more robust than in L4. In order to optimize filtering criteria to reduce the about 26,000 rat genes represented on the GeneChip to those most relevant for pain, multiple filtering criteria were applied based on different threshold detection limits, and fold-regulation in various tissues and conditions. The best criteria that captured the most genes known to be molecular substrates of pain, and most likely to be reproducibly regulated by the SNL model in L4, L5 or L6, are listed below.

For L4, it was required that:
1. The maximum value between L4 sham (ipsilateral), SNL (ipsilateral), and SNL (contralateral) be at least 130, AND
2. that the L4 SNL (ipsilateral) compared to L4 sham (ipsilateral) exhibit at least three-fold regulation, AND
3. that the L4 SNL (ipsilateral) compared to L4 SNL (contralateral) exhibit at least three-fold regulation.

For L5 and L6, it was required that:
1. The maximum value between L5 sham (ipsilateral), L5 SNL (ipsilateral), L6 sham (ipsilateral), and L6 SNL (ipsilateral) be 130, AND
2. that the L5 SNL (ipsilateral) compared to L5 sham (ipsilateral) exhibit at least three-fold regulation, AND
3. that the L6 SNL (ipsilateral) compared to L6 sham (ipsilateral) exhibit at least three-fold regulation.

Probesets representing 249 known genes and 87 ESTs were selected based on the above criteria. Thirteen genes known to be molecular mediators of pain captured by the filtering criteria included the vanilloid receptor (VR-1), voltage-gated sodium channels NaN and SNS/PN3/Nav1.8, serotonin receptor (5HT3), glutamate receptor (iGluR5), regulator of G protein signaling (RGS4), nicotinic acetylcholine receptor alpha 3 subunit, transcription factor DREAM, galanin receptor type 2, somatostatin, galanin, vasoactive intestinal peptide, and neuropeptide Y.

To further characterize the 336 genes (249 known plus 87 ESTs) regulated by SNL according to the stringent criteria described above, hierarchical clustering algorithms with a standard correlation distance measure available in GeneSpring software (Silicon Genetics, Redwood City, Calif.) were used to order the 336 genes based on their gene expression profiles. The experimental samples used for the hierarchical clustering analysis included: L4 naïve ipsi, L4 naïve contra, L4 sham ipsi, L4 SNL ipsi, L4 SNL contra, L4 GPN ipsi, L5 naïve ipsi, L5 sham ipsi, L5 SNL ipsi, L5 SNL contra, L5 SNL+GPN ipsi, L6 naïve ipsi, L6 sham ipsi, L6 SNL ipsi, L6 SNL contra, L6 SNL+GPN ipsi, sciatic nerve, spinal cord, brain, adrenal, aorta, fetal brain, kidney, liver, quadriceps muscle, spleen, submaxillary gland, and testis. The sciatic nerve, spinal cord, brain, adrenal, aorta, fetal brain, kidney, liver, quadriceps muscle, spleen, submaxillary gland, and testis samples were from naïve animals. Using the results of hierarchical clustering and determining the functional annotations of grouped genes, nine transcript regulation classes were determined and designated as: (1) known and novel DRG-specific pain targets; (2) neuronal cellular signal transduction proteins; (3) neuronal markers; (4) cellular signal transduction proteins; (5) known and novel neuropeptides or secreted molecules; (6) inflammatory response genes A; (7) inflammatory response genes B; (8) markers of muscle tissue; and (9) unknown. See PCT Application No. PCT/US04/23166, herein incorporated by reference in its entirety.

### 6.1.1.6. Results: Complement Components Regulated in the SNL Model of Neuropathic Pain Identified in PCT/US04/23166

From PCT Application No. PCT/US04/23166, many genes were found to be at least three-fold regulated in the spinal nerve ligation (SNL) model of neuropathic pain using the Affymetrix rat U34 GeneChip set for gene expression profiling (see PCT/US04/23166 for details). Included among all the regulated genes were several encoding complement components. Complement components found to be up-regulated were factor H, C1q, C1s, C3, factor B (probesets rc_AI170314_at, rc_AA996499_at, rc_AI177119_at, D88250_at, X71127_at, M29866_s_at, X52477_at, and rc_AI639117_s_at). One complement component, DAF (probeset AF039583_s_at), was found to be down-regulated.

Since multiple components of complement were regulated at least three-fold by SNL, an analysis was conducted to determine if any additional complement components were also regulated but less than the original three-fold cut-off. As described in detail below, bioinformatics were used to identify all probesets in the rat Affymetrix U34 set that encode complement components. Gene expression patterns were then determined across the profiled SNL samples (see PCT/US04/23166 and Table 4 legend for detailed sample descriptions).

### 6.1.1.7. Results: Identifying Nucleic Acid Sequences for Complement Components

The Gene Ontology (GO) project (available on the WorldWideWeb at geneontology.org) is a collaborative effort to develop structured, controlled vocabularies (ontologies) that describe gene products in terms of their associated biological processes, cellular components and molecular functions in a species-independent manner. The use of GO terms by several collaborating databases serves to facilitate uniform queries across them. The controlled vocabularies are structured so that one can query them at different levels: for example, one can use GO to find all the gene products in the mouse genome that are involved in signal transduction, or one can more specifically find all the receptor tyrosine kinases. In order to identify nucleic acid sequences considered to encode for a complement component, the GO database (available on the WorldWideWeb at geneontology.org) was first searched using the search term “complement.” All sequences identified as associated with the GO term “complement” were downloaded to create a “seed” protein sequence database of all complement components curated by the GO project. To identify nucleic acid sequences encoding complement components the seed sequences were used as the query to compare to sequences in the NR database (available on the WorldWideWeb at ncbi.nlm.nih.gov) using the TBLASTN sequence comparison algorithm (Altschul et al., J Mol Biol. 1990, 215:403-10 and Altschul et al., Nucleic Acids Res. 1997, 25:3389-402). The most significant sequence matches are listed in Table 2. Since the GO database is continually curated as sequences are deposited into the public databases, the described method can be used at any time to identify the most complete list of complement component encoding sequences.

TABLE 2
Nucleic Acid Sequences for Complement Components. The GO database
(available on the WorldWideWeb at geneontology.org) was searched for seed sequences
assigned the biological ontology “complement”. The GO seed description for each
retrieved complement component is displayed in Column B. To identify nucleic acid
sequences encoding for each complement component the seed sequence was used as the
query to compare to sequences in the NR database (available on the WorldWideWed at
ncbi.nlm.nih.gov) using the TBLASTN sequence comparison algorithm (Altschul et al., J
Mol Biol. 1990, 215: 403-10 and Altschul et al., Nucleic Acids Res. 1997, 25: 3389-402).
A SEQ ID NO for the most significant sequence match is provided in Column A for each
identified sequence of the given Accession # (Column C). The percent positive identity
(% pos, Column D) over the region of overlap in amino acid sequence (hit length, Column
E), as well as the length of the query GO seed sequence in amino acids (query length,
Column F) are also shown.

| A. | | | | | |
|---|---|---|---|---|---|
| SEQ | | | | | F. |
| ID | | C. | D. | E. hit | query |
| NO: | B. GO seed description | Accession # | % pos | length | length |
| 1 | Complement receptor type 1 precursor | NM_000573.2 | 97.87 | 2019 | 2039 |
| | (C3b/C4b receptor) (CD35 antigen). | | | | |
| 2 | Complement C4 precursor [Contains: C4A | NM_009780.1 | 97.64 | 1738 | 1738 |
| | anaphylatoxin]. | | | | |
| 3 | Complement C5 precursor (Hemolytic | NM_010406.1 | 100 | 1680 | 1680 |
| | complement) [Contains: C5A anaphylatoxin]. | | | | |
| 4 | Complement C3 precursor [Contains: C3a | NM_000064.1 | 100 | 1639 | 1663 |
| | anaphylatoxin]. | | | | |
| 5 | Complement C3 precursor (HSE-MSF) | NM_009778.1 | 97.65 | 1663 | 1663 |
| | [Contains: C3A anaphylatoxin]. | | | | |
| 6 | Complement C3 precursor [Contains: C3A | NM_016994.1 | 97.47 | 1663 | 1663 |
| | anaphylatoxin]. | | | | |
| 7 | Complement C5 precursor [Contains: C5a | M57729.1 | 97.05 | 1662 | 1676 |
| | anaphylatoxin]. | | | | |
| 8 | Complement factor H precursor (Protein | NM_009888.2 | 98.87 | 1234 | 1234 |
| | beta-1-H). | | | | |
| 9 | Complement factor H precursor (H factor 1). | Y00716.1 | 99.03 | 1231 | 1231 |
| 10 | Complement receptor type 2 precursor (Cr2) | M35684.1 | 100 | 1025 | 1025 |
| | (Complement C3d receptor). | | | | |
| 11 | Complement receptor type 2 precursor (Cr2) | M26004.1 | 98.52 | 1013 | 1033 |
| | (Complement C3d receptor) (Epstein-Barr | | | | |
| | virus receptor) (EBV receptor) (CD21 | | | | |
| | antigen). | | | | |
| 12 | Complement component C6 precursor. | NM_000065.1 | 98.5 | 934 | 934 |
| 13 | Complement component C7 precursor. | J03507.1 | 92.41 | 843 | 843 |
| 14 | Complement factor B precursor (EC | S67310.1 | 98.43 | 764 | 764 |
| | 3.4.21.47) (C3/C5 convertase) (Properdin | | | | |
| | factor B) (Glycine-rich beta glycoprotein) | | | | |
| | (GBG) (PBF2). | | | | |
| 15 | Complement C2 precursor (EC 3.4.21.43) | NM_000063.3 | 100 | 752 | 752 |
| | (C3/C5 convertase). | | | | |
| 16 | Complement factor B precursor (EC | NM_008198.1 | 97.9 | 761 | 761 |
| | 3.4.21.47) (C3/C5 convertase). | | | | |
| 17 | Complement C1r component precursor (EC | M14058.1 | 100 | 705 | 705 |
| | 3.4.21.41). | | | | |
| 18 | Complement-activating component of Ra- | D28593.1 | 98.14 | 699 | 699 |
| | reactive factor precursor (EC 3.4.21.—) (Ra- | | | | |
| | reactive factor serine protease p100) (RaRF) | | | | |
| | (Mannan-binding lectin serine protease 1) | | | | |
| | (Mannose-binding protein associated serine | | | | |
| | protease) (MASP-1). | | | | |
| 19 | Complement-activating component of Ra- | NM_008555.1 | 96.73 | 704 | 704 |
| | reactive factor precursor (EC 3.4.21.—) (Ra- | | | | |
| | reactive factor serine protease p100) (RaRF) | | | | |
| | (Mannan-binding lectin serine protease 1). | | | | |
| 20 | Mannan-binding lectin serine protease 2 | Y09926.1 | 98.83 | 686 | 686 |
| | precursor (EC 3.4.21.—) (Mannose-binding | | | | |
| | protein associated serine protease 2) (MASP- | | | | |
| | 2) (MBL-associated serine protease 2). | | | | |
| 21 | Complement C1s component precursor (EC | BC056903.1 | 97.82 | 688 | 688 |
| | 3.4.21.42) (C1 esterase). | | | | |
| 22 | C4b-binding protein alpha chain precursor | BC022312.1 | 100 | 597 | 597 |
| | (C4bp) (Proline-rich protein) (PRP). | | | | |
| 23 | Complement factor I precursor (EC | NM_024157.1 | 98.29 | 586 | 604 |
| | 3.4.21.45) (C3B/C4B inactivator). | | | | |
| 24 | Complement factor H-related protein 5 | NM_030787.1 | 100 | 569 | 569 |
| | precursor (FHR-5). | | | | |
| 25 | Complement component C8 beta chain | NM_000066.1 | 97.97 | 591 | 591 |
| | precursor. | | | | |
| 26 | C4b-binding protein alpha chain precursor | NM_012516.1 | 100 | 558 | 558 |
| | (C4bp). | | | | |
| 27 | Complement component C8 alpha chain | NM_000562.1 | 92.64 | 584 | 584 |
| | precursor. | | | | |
| 28 | Complement component C9 precursor. | BC020721.1 | 96.6 | 559 | 559 |
| 29 | C4b-binding protein precursor (C4bp). | BC012257.1 | 99.79 | 469 | 469 |
| 30 | Properdin (Factor P) (Fragment). | X12905.1 | 100 | 437 | 437 |
| 31 | Plasma protease C1 inhibitor precursor (C1 | NM_009776.1 | 97.82 | 504 | 504 |
| | Inh) (C1 Inh). | | | | |
| 32 | Properdin precursor (Factor P). | NM_002621.1 | 89.98 | 469 | 469 |
| 33 | C3a anaphylatoxin chemotactic receptor | AB065870.1 | 95.44 | 482 | 482 |
| | (C3a-R) (C3AR). | | | | |
| 34 | Clusterin precursor (Complement-associated | BC010514.1 | 94.65 | 449 | 449 |
| | protein SP-40,40) (Complement cytolysis | | | | |
| | inhibitor) (CLI) (NA1/NA2) (Apolipoprotein J) | | | | |
| | (Apo-J) (TRPM-2). | | | | |
| 35 | C3a anaphylatoxin chemotactic receptor | BC003728.1 | 89.31 | 477 | 477 |
| | (C3a-R) (C3AR) (Complement component 3a | | | | |
| | receptor 1). | | | | |
| 36 | Complement decay-accelerating factor, | L41365.1 | 92.87 | 407 | 407 |
| | transmembrane precursor (DAF-TM). | | | | |
| 37 | Complement decay-accelerating factor, GPI- | L41366.1 | 94.87 | 390 | 390 |
| | anchored precursor (DAF-GPI). | | | | |
| 38 | Similar to complement receptor related | BC028945.1 | 86.36 | 440 | 440 |
| | protein. | | | | |
| 38 | Complement receptor related protein. | BC028945.1 | 79.71 | 483 | 483 |
| 39 | Hypothetical Anaphylotoxins. | AK050126.1 | 100 | 352 | 352 |
| 40 | Membrane cofactor protein precursor (CD46 | NM_172351.1 | 88.06 | 377 | 377 |
| | antigen) (Trophoblast leucocyte common | | | | |
| | antigen) (TLX). | | | | |
| 41 | C5a anaphylatoxin chemotactic receptor | NM_007577.1 | 99.71 | 346 | 347 |
| | (C5a-R). | | | | |
| 42 | Complement factor H-related protein 1 | NM_002113.1 | 95.76 | 330 | 330 |
| | precursor (FHR-1) (H factor-like protein 1) (H- | | | | |
| | factor like 1) (H36). | | | | |
| 43 | X/Y protein (Fragment). | M16179.1 | 95.45 | 330 | 330 |
| 44 | Complement decay-accelerating factor | M31516.1 | 87.03 | 347 | 381 |
| | precursor (CD55 antigen). | | | | |
| 45 | Complement component 1, Q subcomponent | NM_007573.1 | 97.12 | 278 | 278 |
| | binding protein, mitochondrial precursor | | | | |
| | (Glycoprotein gC1qBP) (GC1q-R protein). | | | | |
| 46 | Complement factor D precursor (EC | NM_013459.1 | 100 | 259 | 259 |
| | 3.4.21.46) (C3 convertase activator) | | | | |
| | (Properdin factor D) (Adipsin) (28 kDa | | | | |
| | protein, adipocyte). | | | | |
| 47 | C4B-binding protein beta chain precursor. | NM_016995.1 | 100 | 249 | 258 |
| 48 | Complement factor D precursor (EC | S73894.1 | 93.92 | 263 | 263 |
| | 3.4.21.46) (C3 convertase activator) | | | | |
| | (Properdin factor D) (Adipsin) (Endogenous | | | | |
| | vascular elastase). | | | | |
| 49 | C4b-binding protein beta chain precursor. | L11244.1 | 100 | 233 | 252 |
| 50 | Adipsin/complement factor D precursor (EC | NM_001928.2 | 92.49 | 253 | 253 |
| | 3.4.21.46). | | | | |
| 51 | Complement factor D precursor (EC | BC034529.1 | 100 | 232 | 253 |
| | 3.4.21.46) (C3 convertase activator) | | | | |
| | (Properdin factor D) (Adipsin). | | | | |
| 52 | Complement receptor. | NM_013499.1 | 87.16 | 257 | 257 |
| 53 | Complement C1q subcomponent, A chain | BC030153.2 | 91.84 | 245 | 245 |
| | precursor. | | | | |
| 54 | Mannose-binding protein C precursor (MBP- | D11440.1 | 88.93 | 244 | 244 |
| | C) (Mannan-binding protein) (RA-reactive | | | | |
| | factor P28A subunit) (RARF/P28A). | | | | |
| 55 | Complement C1q subcomponent, C chain | X66295.1 | 80.49 | 246 | 246 |
| | precursor. | | | | |
| 56 | Complement C1q subcomponent, B chain | X16874.1 | 77.08 | 253 | 253 |
| | precursor. | | | | |
| 57 | Mannose-binding protein C precursor (MBP- | Y16581.1 | 80.43 | 235 | 248 |
| | C) (MBP1) (Mannan-binding protein) | | | | |
| | (Mannose-binding lectin). | | | | |
| 58 | Complement component C8 gamma chain | NM_000606.1 | 78.71 | 202 | 202 |
| | precursor. | | | | |
| 59 | Mannose-binding protein A precursor (MBP- | AF080507.1 | 79.55 | 220 | 238 |
| | A) (Mannan-binding protein). | | | | |
| 60 | Mannose-binding protein A precursor (MBP- | BC021762.1 | 78.18 | 220 | 239 |
| | A) (Mannan-binding protein) (RA-reactive | | | | |
| | factor polysaccharide-binding component | | | | |
| | P28B polypeptide) (RARF P28B). | | | | |
| 61 | Complement component C8 beta chain | U20194.1 | 100 | 140 | 140 |
| | (Fragment). | | | | |
| 62 | S-100 protein, beta chain. | BC001766.1 | 82.42 | 91 | 91 |
| 63 | Complement C5A anaphylatoxin. | XM_345342.1 | 92.21 | 77 | 76 |
| 64 | Complement C1q subcomponent, C chain | XM_342951.1 | 92.85 | 28 | 28 |
| | (Fragment). | | | | |
| 65 | Complement C1q subcomponent, A chain | XM_216554.2 | 100 | 15 | 15 |
| | (Fragment). | | | | |

### 6.1.1.8. Results: Identifying All Complement Components Profiled in the SNL Model of Neuropathic Pain

In order to identify all complement components represented on the Affymetrix U34 GeneChips, a similar method, BLASTX comparison (Altschul et al., J Mol Biol. 1990, 215:403-10 and Altschul et al., Nucleic Acids Res. 1997, 25:3389-402), was used to query the Affymetrix probeset sequences against the GO database (available on the WorldWideWeb at geneontology.org) for significant sequence matches. Criteria for accepting a match as significant were that the percent positive identity had to be at least 75% and that the hit length ratio (i.e., hit length/subject length) had to be greater than 50%. In some cases probeset reference sequences were re-searched in the non-redundant NR database using the BLASTN algorithm to verify the annotation. The complement components found are reported in Table 3. For each Affymetrix probeset corresponding to an identified complement component, the following information is displayed in Table 3: the GO database annotation (GO seed description, Column C), the percent positive identity when comparing the GO seed sequence for the complement component found with the translated probeset sequence searched (% pos, Column D), the hit length or extent of sequence similarity overlap between subject (GO seed sequence) and query (probeset sequence) in amino acids (hit length, Column E), and the subject length (GO seed sequence) in amino acids (subject length, Column F). In addition, a nucleic acid sequence for each GO seed protein sequence was retrieved by using the TBLASTN algorithm to identify the best sequence match in the NR database. The preferred nucleic acid sequence (and accompanying protein sequence) reported was the one, when identified, from RefSeq (a curated transcript and related protein database maintained by the National Center for Biotechnology Information, Nucleic Acids Res (2001) 29:137-140, available on the WorldWideWeb at ncbi.nlm.nih.gov/RefSeq/) (listed by SEQ ID NO for nucleic acid and protein sequence in Columns H and J, respectively, and by Accession # in Columns G and I, respectively). If a RefSeq sequence was not among the top ten sequence matches (hits), the one with the most significant E-value (a statistic for the significance of the sequence comparison) was chosen.

TABLE 3
Complement components represented by probesets on the Affymetrix GeneChip ® U34. The BLASTX sequence
comparison algorithm was used to compare all Affymetrix U34 probeset sequences to the GO database (available on the
WorldWideWeb at geneontology.org). Any U34 probeset sequence which shared significant sequence identity to a GO seed sequence
assigned “complement” as an ontology was retained (Column B, SEQ ID NO in Column A). The resulting annotation is given by the
GO seed description (Column C). Criteria for significant sequence identity were that the percent positive identity (% pos, Column D)
between the GO seed sequence and the Affymetrix probeset sequence had to be at least 75% and that the region of sequence overlap
had to be greater than 50%. This can be determined by dividing the sequence overlap in the aligned sequences (hit length, Column E)
by the total sequence length of the GO seed (subject length, Column F). In some cases probeset reference sequences were re-searched
in the non-redundant NR database using the BLAST algorithm to verify the annotation. Also shown are SEQ ID NOS for the nucleic
acid and protein sequence for the described complement component (Column H and J, respectively) with the corresponding NR
Accession numbers (Column G and I, respectively).

| A | B | | | | | G | H | I | J |
|---|---|---|---|---|---|---|---|---|---|
| Probeset | | | | | | NR nucleic acid | | NR protein | |
| SEQ | | | | E | F | | SEQ | | SEQ |
| ID | | C | D | hit | subject | | ID | | ID |
| NO: | Probeset | GO seed description | %pos | length | length | Accession # | NO: | Accession # | NO: |
| 66 | X95990exon_s_at | C5a anaphylatoxin chemotactic | 87.03 | 347 | 347 | NM_007577.1 | 97 | NP_031603.1 | 127 |
| | | receptor (C5a-R). | | | | | | | |
| 67 | Z50051_at | C4B-binding protein beta chain | 100 | 558 | 558 | Z50052.1 | 98 | CAA90392.1 | 128 |
| | | precursor. | | | | | | | |
| 68 | U20194_g_at | Complement component C8 beta | 88.21 | 560 | 591 | NM_000066.1 | 99 | NP_000057.1 | 129 |
| | | chain precursor. | | | | | | | |
| 69 | U52948_at | Complement componentC9 | 96.39 | 554 | 554 | NM_057146.1 | 100 | NP_476487.1 | 130 |
| | | precursor. | | | | | | | |
| 70 | X05023_at | Mannose-binding protein A | 86.96 | 207 | 244 | AF080507.1 | 101 | AAC31936.1 | 131 |
| | | precursor (MBP-A) (Mannan- | | | | | | | |
| | | binding protein). | | | | | | | |
| 71 | rc_AI178135_at | Complement component 1, Q | 89.61 | 279 | 278 | NM_007573.1 | 102 | NP_031599.1 | 132 |
| | | subcomponent binding protein, | | | | | | | |
| | | mitochondrial precursor | | | | | | | |
| | | (Glycoprotein gC1qBP) (GC1q-R | | | | | | | |
| | | protein). | | | | | | | |
| 72 | M64733mRNA_s_at | Clusterin precursor | 86.16 | 448 | 449 | NM_203339.1 | 103 | NP_976084.1 | 133 |
| | | (Complement-associated protein | | | | | | | |
| | | SP-40, 40) (Complement cytolysis | | | | | | | |
| | | inhibitor) (CLI) (NA1/NA2) | | | | | | | |
| | | (Apolipoprotein J) (Apo-J) | | | | | | | |
| | | (TRPM-2). | | | | | | | |
| 73 | rc_AI170314_at | Complement factor H precursor | 86.74 | 1237 | 1234 | NM_009888.2 | 104 | NP_034018.1 | 134 |
| | | (Protein beta-1-H). | | | | | | | |
| 74 | rc_AI059560_at | Complement decay-accelerating | 75.76 | 396 | 390 | NM_010016.1 | 105 | NP_034146.1 | 135 |
| | | factor, GPI-anchored precursor | | | | | | | |
| | | (DAF-GPI). | | | | | | | |
| 75 | rc_AA945193_at | Hypothetical Anaphylotoxins. | 89.02 | 173 | 352 | AK050126.1 | 106 | BAC34079.1 | 136 |
| 76 | M92059_s_at | Complement factor D precursor | 82.54 | 252 | 263 | XM_343169.1 | 107 | XP_343170.1 | 137 |
| | | (EC 3.4.21.46) (C3 convertase | | | | | | | |
| | | activator) (Properdin factor D) | | | | | | | |
| | | (Adipsin) (Endogenous vascular | | | | | | | |
| | | elastase). | | | | | | | |
| 77 | rc_AI232490_at | Complement component C7 | 81 | 800 | 843 | NM_000587.2 | 108 | NP_000578.2 | 138 |
| | | precursor. | | | | | | | |
| 78 | rc_AA996499_at | Complement C1q subcomponent, | 93.98 | 83 | 245 | NM_019262.1 | 109 | NP_062135.1 | 139 |
| | | B chain precursor. | | | | | | | |
| 79 | rc_AI177119_at | Complement C1q subcomponent, | 77.64 | 246 | 246 | NM_007574.1 | 110 | NP_031600.1 | 140 |
| | | C chain precursor. | | | | | | | |
| 80 | X52477_at | Complement C3 precursor | 97.47 | 1663 | 1663 | NM_016994.1 | 111 | NP_058690.1 | 141 |
| | | [Contains: C3A anaphylatoxin]. | | | | | | | |
| 81 | rc_AI233300_at | Complement C5 precursor | 94.51 | 346 | 1680 | M35525.1 | 112 | AAA37349.1 | 142 |
| | | (Hemolytic complement) | | | | | | | |
| | | [Contains: C5A anaphylatoxin]. | | | | | | | |
| 82 | D88250_at | Complement C1s component | 82.73 | 689 | 688 | NM_201442.1 | 113 | NP_958850.1 | 143 |
| | | precursor (EC 3.4.21.42) (C1 | | | | | | | |
| | | esterase). | | | | | | | |
| 83 | rc_AA799803_at | Complement C1r component | 90.07 | 453 | 705 | M14058.1 | 114 | AAA51851.1 | 144 |
| | | precursor (EC 3.4.21.41). | | | | | | | |
| 84 | rc_AA800318_at | Plasma protease C1 inhibitor | 81.97 | 488 | 504 | NM_000062.1 | 115 | NP_000053.1 | 145 |
| | | precursor (C1 Inh) (C1 Inh). | | | | | | | |
| 85 | rc_AI178368_s_at | Similar to complement receptor | 74.86 | 354 | 440 | BC028945.1 | 116 | AAH28945.1 | 146 |
| | | related protein. | | | | | | | |
| 86 | rc_AI072392_at | Complement C2 precursor (EC | 91.37 | 742 | 760 | NM_013484.1 | 117 | NP_038512.1 | 147 |
| | | 3.4.21.43) (C3/C5 convertase). | | | | | | | |
| 87 | rc_AI169829_at | Complement-activating | 93.75 | 704 | 704 | NM_008555.1 | 118 | NP_032581.1 | 148 |
| | | component of Ra-reactive factor | | | | | | | |
| | | precursor (EC 3.4.21.—) (Ra- | | | | | | | |
| | | reactive factor serine protease | | | | | | | |
| | | p100) (RaRF) (Mannan-binding | | | | | | | |
| | | lectin serine protease 1). | | | | | | | |
| 88 | rc_AA945094_at | Complement factor I precursor | 98.29 | 586 | 604 | NM_024157.1 | 119 | NP_077071.1 | 149 |
| | | (EC 3.4.21.45) (C3B/C4B | | | | | | | |
| | | inactivator). | | | | | | | |
| 89 | rc_AI045191_at | Complement component C6 | 87.14 | 933 | 934 | NM_000065.1 | 120 | NP_000056.1 | 150 |
| | | precursor. | | | | | | | |
| 90 | rc_AI029040_at | Complement component C8 | 64.71 | 204 | 202 | NM_000606.1 | 121 | NP_000597.1 | 151 |
| | | gamma chain precursor. | | | | | | | |
| 91 | rc_AA996755_at | Mannan-binding lectin serine | 86.92 | 673 | 686 | Y09926.1 | 122 | CAA71059.1 | 152 |
| | | protease 2 precursor (EC 3.4.21—) | | | | | | | |
| | | (Mannose-binding protein | | | | | | | |
| | | associated serine protease 2) | | | | | | | |
| | | (MASP-2) (MBL-associated | | | | | | | |
| | | serine protease 2). | | | | | | | |
| 92 | rc_AI639117_s_at | Complement factor B precursor | 93.82 | 761 | 761 | NM_008198.1 | 123 | NP_032224.1 | 153 |
| | | (EC 3.4.21.47) (C3/C5 | | | | | | | |
| | | convertase). | | | | | | | |
| 93 | rc_AI639534_g_at, | Properdin (Factor P) (Fragment). | 88.4 | 431 | 437 | X12905.1 | 124 | CAA31389.1 | 154 |
| | rc_AI639534_at | | | | | | | | |
| 94 | rc_AI177373_at | Complement receptor type 2 | 78.19 | 1036 | 1025 | M35684.1 | 125 | AAA37448.1 | 155 |
| | | precursor (Cr2) (Complement | | | | | | | |
| | | C3d receptor). | | | | | | | |
| 95 | AB010920_at | Membrane cofactor protein | 64.67 | 317 | 377 | NM_172351.1 | 126 | NP_758861.1 | 156 |
| | | precursor (CD46 antigen) | | | | | | | |
| | | (Trophoblast leucocyte common | | | | | | | |
| | | antigen) (TLX). | | | | | | | |
| 96 | AF039583_s_at | Complement decay-accelerating | 98.95 | 1430 | 1514 | NM_010016.1 | 105 | NP_034146.1 | 135 |
| | | factor, GPI-anchored precursor | | | | | | | |
| | | (DAF-GPI). | | | | | | | |

Finally, from the complete Affymetrix GeneChip data generated for our gene expression profiling of the spinal nerve ligation model, the data was retrieved corresponding to the probesets for complement components listed in Table 3. This data was analyzed and the gene expression summary is given in Table 4.

To compare the expression levels of the complement components of Table 3 in pain and normal states, t-tests were performed on the GeneChip signal data from DRG samples from naïve, sham, and SNL animals. The following t-tests were performed for each probeset comparing the average GeneChip signal from the following: ipsilateral DRG samples from SNL animals with and without GPN treatment versus the contralateral DRG samples from SNL animals with and without GPN treatment (Column C, Table 4); ipsilateral DRG samples from SNL animals with and without GPN treatment versus ipsilateral DRG samples from sham and naïve animals (Column D, Table 4); and ipsilateral DRG samples from sham animals versus ipsilateral DRG from naïve animals (Column E, Table 4). The probability for these t-tests are reported in Columns C, D and E.

In a further comparison as shown in Table 4, ratios comparing the average GeneChip signals from the ipsilateral DRG samples from SNL animals with and without GPN treatment versus the contralateral DRG samples from SNL animals with and without GPN treatment for L4, L5, and L6 were calculated and the results are given in Columns F, G, and H, respectively. In addition, the ratio comparing the average GeneChip signal of ipsilateral sciatic nerve from SNL animals with and without GPN treatment to the average GeneChip signal of ipsilateral sciatic nerve from sham and naïve animals (designated in Table 4 as Nerve) was calculated (Column I). As shown in FIG. 4, the sciatic nerve connects the L4, L5, and L6 of the DRG to the skin and other tissues. GeneChip® signals in the sciatic nerve showing regulation of a gene in a pain versus naïve/sham state can also show that the gene is involved in a pain response.

The maximum GeneChip(® signal observed in all the DRG samples for each probeset is recorded in Column J (designated as Max DRG).

A summary of gene regulation in the DRG and sciatic nerve is shown in Column K of Table 4. Up- or down-regulation in the SNL model when compared to naïve/sham animals is indicated as “up” or “down”, respectively. A probeset is considered to be regulated if p≦0.05 in the t-test (showing that the two values differed significantly) or if the ratio in the DRG or in the sciatic nerve shows at least a 1.5 fold increase or decrease. A probeset is considered to be detected if at least one signal from the DRG samples is greater than 100. Probesets that were not detected and, therefore, could not be assessed for differential expression, are summarized as “not detected”.

In particular, it should be noted that probesets corresponding to the cell-surface expressed complement inhibitor, DAF-GPI (synonymous with DAF), were the only probesets exhibiting down-regulation in DRG during a pain state. DAF, as noted in PCT Application No. PCT/US04/23 166, belongs to transcript class 1, whose characteristic expression pattern is down-regulation by SNL and restricted expression to DRGs. Many known pain genes which are known to be neuronally expressed belong to transcript class 1 (i.e. VR-1, NaN, SNS/PN3/Nav1.8, 5HT3, iGluR5, RGS4, nicotinic acetylcholine receptor, and DREAM) (see PCT Application No. PCT/US04/23166 for details). In contrast, all the other complement components (e.g., C3) in DRG are up-regulated, not apparently regulated, or below the limit of detection.

TABLE 4
Expression profiles of complement components in the SNL model of neuropathic pain using the Affymetrix
GeneChip ® U34. Column A and B give the probeset SEQ ID NO and gene ontology database annotation, respectively. In Column
C, the average GeneChip signal for the ipsilateral (ipsi) DRG in SNL animals with and without GPN treatment was compared to the
average GeneChip signal for the contralateral (contra) DRG in SNL animals with and without GPN treatment using a t-test. In
Column D, the average GeneChip signal for the “injured” ipsilateral DRG in SNL animals with and without GPN treatment was
compared to the average GeneChip signal for the “control” ipsilateral DRG in sham and naive animals using a t-test. In Column E,
the GeneChip signal for the ipsilateral DRG in sham animals was compared to the GeneChip signal for the ipsilateral DRG in naive
animals using a t-test. Ratios comparing the GeneChip signals from the ipsilateral DRGs of SNL animals versus the contralateral
DRGs of SNL animals for L4, L5, and L6 appear in Columns F, G, and H, respectively. In Column I displays the ratio of the average
GeneChip signal for the ipsilateral sciatic nerve in SNL animals with and without GPN treatment versus the average GeneChip signal
for the ipsilateral sciatic nerve in sham and naive animals. Column J displays the maximum GeneChip signal detected among all DRG
samples collected from the SNL model for the probeset indicated. Column K summarizes the apparent regulation in the sciatic nerve
and DRG or states that the complement component mRNA was not detected in any DRG sample within the limits of the assay.

| | | Probability of t-test | | | Ratio of avg GeneChip signal | | | | | |
|---|---|---|---|---|---|---|---|---|---|---|
| | | C | D | | F | G | H | I Nerve | | |
| A | | DRG | DRG ipsi | E | L4 | L5 | L6 | ipsi | | |
| Probeset | | [injured] | [injured] | DRG ipsi | [SNL] | [SNL] | [SNL ] | [injured] | J | K |
| SEQ ID | B | ipsi vs | vs | [sham ] | ipsi vs | ipsi vs | ipsi vs | vs | Max | Regulation |
| NO: | GO seed description | contra | [control] | vs[naïve] | contra | contra | contra | [control] | DRG | Summary |
| 66 | C5a anaphylatoxin chemotactic | 0.313 | 0.097 | 0.025 | 0.990 | 0.7 | 1.2 | 1.3 | 259 | Not |
| | receptor (C5a-R). | | | | | | | | | regulated |
| 67 | C4B-binding protein beta chain | 0.113 | 0.069 | 0.040 | 0.724 | 0.6 | 1.1 | 0.7 | 11 | Not |
| | precursor. | | | | | | | | | detected |
| 68 | Complement component C8 beta | 0.015 | 0.062 | 0.405 | 0.487 | 0.8 | 0.7 | 1.6 | 18 | Not |
| | chain precursor. | | | | | | | | | detected |
| 69 | Complement component C9 | 0.103 | 0.392 | 0.498 | 0.855 | 0.3 | 0.5 | 3.9 | 27 | Not |
| | precursor. | | | | | | | | | detected |
| 70 | Mannose-binding protein A | 0.208 | 0.210 | 0.323 | 1.691 | 0.4 | 0.5 | 1.0 | 17 | Not |
| | precursor (MBP-A) (Mannan- | | | | | | | | | detected |
| | binding protein). | | | | | | | | | |
| 71 | Complement component 1, Q | 0.055 | 0.001 | 0.435 | 0.818 | 1.0 | 0.8 | 0.9 | 658 | Not |
| | subcomponent binding protein, | | | | | | | | | regulated |
| | mitochondrial precursor | | | | | | | | | |
| | (Glycoprotein gC1qBP) (GC1q-R | | | | | | | | | |
| | protein). | | | | | | | | | |
| 72 | Clusterin precursor (Complement- | 0.047 | 0.074 | 0.122 | 1.079 | 1.2 | 1.1 | 2.5 | 5624 | Nerve-up |
| | associated protein SP-40, 40) | | | | | | | | | |
| | (Complement cytolysis inhibitor) | | | | | | | | | |
| | (CLI) (NA1/NA2) (Apolipoprotein J) | | | | | | | | | |
| | (Apo-J) (TRPM-2). | | | | | | | | | |
| 73 | Complement factor H precursor | 0.001 | 0.001 | 0.126 | 2.358 | 3.9 | 9.1 | 1.8 | 370 | DRG and |
| | (Protein beta-1-H). | | | | | | | | | Nerve-up |
| 74 | Complement decay-accelerating | 0.003 | 0.004 | 0.480 | 0.841 | 0.5 | 0.5 | 0.4 | 160 | DRG-down |
| | factor, GPI-anchored precursor | | | | | | | | | |
| | (DAF-GPI). | | | | | | | | | |
| 75 | Hypothetical Anaphylotoxins. | 0.299 | 0.484 | 0.088 | 0.764 | 0.8 | 1.3 | 1.1 | 153 | Not |
| | | | | | | | | | | regulated |
| 76 | Complement factor D precursor (EC | 0.003 | 0.040 | 0.277 | 6.107 | 10.1 | 5.6 | 0.5 | 382 | DRG-up |
| | 3.4.21.46) (C3 convertase | | | | | | | | | Nerve-down |
| | activator) (Properdin factor D) | | | | | | | | | |
| | (Adipsin) (Endogenous vascular | | | | | | | | | |
| | elastase). | | | | | | | | | |
| 77 | Complement component C7 | 0.001 | 0.000 | 0.310 | 1.418 | 2.0 | 2.5 | 2.8 | 112 | DRG and |
| | precursor. | | | | | | | | | Nerve-up |
| 78 | Complement C1q subcomponent, B | 0.003 | 0.004 | 0.012 | 1.599 | 3.9 | 5.3 | 1.5 | 2088 | DRG and |
| | chain precursor. | | | | | | | | | Nerve-up |
| 79 | Complement C1q subcomponent, C | 0.002 | 0.003 | 0.002 | 1.555 | 3.8 | 6.6 | 2.2 | 800 | DRG and |
| | chain precursor. | | | | | | | | | Nerve-up |
| 80 | Complement C3 precursor | 0.002 | 0.001 | 0.094 | 5.393 | 2.8 | 10.8 | 11.1 | 282 | DRG and |
| | [Contains: C3A anaphylatoxin]. | | | | | | | | | Nerve-up |
| 81 | Complement C5 precursor | 0.380 | 0.335 | 0.041 | 2.269 | 0.9 | 1.3 | 0.5 | 37 | Not |
| | (Hemolytic complement) [Contains: | | | | | | | | | detected |
| | C5A anaphylatoxin]. | | | | | | | | | |
| 82 | Complement C1s component | 0.008 | 0.018 | 0.017 | 1.918 | 7.1 | 7.6 | 1.9 | 1358 | DRG and |
| | precursor (EC 3.4.21.42) (C1 | | | | | | | | | Nerve-up |
| | esterase). | | | | | | | | | |
| 83 | Complement C1r component | 0.002 | 0.010 | 0.023 | 1.415 | 3.5 | 3.0 | 1.8 | 1165 | DRG and |
| | precursor (EC 3.4.21.41). | | | | | | | | | Nerve-up |
| 84 | Plasma protease C1 inhibitor | 0.002 | 0.003 | 0.127 | 1.376 | 3.5 | 4.4 | 4.2 | 1101 | DRG and |
| | precursor (C1 Inh) (C1 Inh). | | | | | | | | | Nerve-up |
| 85 | Similar to complement receptor | 0.032 | 0.109 | 0.226 | 0.979 | 1.4 | 2.3 | 1.0 | 1623 | DRG-up |
| | related protein. | | | | | | | | | |
| 86 | Complement C2 precursor (EC | 0.012 | 0.055 | 0.150 | 1.375 | 3.1 | 5.8 | 2.3 | 111 | DRG and |
| | 3.4.21.43) (C3/C5 convertase). | | | | | | | | | Nerve-up |
| 87 | Complement-activating component | 0.100 | 0.066 | 0.074 | 1.248 | 1.8 | 1.3 | 0.9 | 189 | Not |
| | of Ra-reactive factor precursor (EC | | | | | | | | | regulated |
| | 3.4.21.—) (Ra-reactive factor serine | | | | | | | | | |
| | protease p100) (RaRF) (Mannan- | | | | | | | | | |
| | binding lectin serine protease 1). | | | | | | | | | |
| 88 | Complement factor I precursor (EC | 0.332 | 0.055 | 0.419 | 0.788 | 1.0 | 1.1 | 2.7 | 230 | Nerve-up |
| | 3.4.21.45) (C3B/C4B inactivator). | | | | | | | | | |
| 89 | Complement component C6 | 0.222 | 0.292 | 0.208 | 0.888 | 0.8 | 1.0 | 0.9 | 173 | Not |
| | precursor. | | | | | | | | | regulated |
| 90 | Complement component C8 | 0.297 | 0.454 | 0.172 | 1.060 | 1.0 | 1.4 | 1.1 | 144 | Not |
| | gamma chain precursor. | | | | | | | | | regulated |
| 91 | Mannan-binding lectin serine | 0.422 | 0.349 | 0.267 | 0.766 | 0.7 | 1.9 | 2.4 | 47 | Not |
| | protease 2 precursor (EC 3.4.21.—) | | | | | | | | | detected |
| | (Mannose-binding protein | | | | | | | | | |
| | associated serine protease 2) | | | | | | | | | |
| | (MASP-2) (MBL-associated serine | | | | | | | | | |
| | protease 2). | | | | | | | | | |
| 92 | Complement factor B precursor | 0.036 | 0.072 | 0.022 | 1.510 | 3.6 | 3.8 | 1.3 | 157 | DRG-up |
| | (EC 3.4.21.47) (C3/C5 convertase). | | | | | | | | | |
| 93 | Properdin (Factor P) (Fragment). | 0.042 | 0.009 | 0.359 | 0.532 | 4.6 | 3.5 | 1.2 | 96 | DRG-up |
| 93 | Properdin (Factor P) (Fragment). | 0.033 | 0.006 | 0.071 | 0.825 | 1.5 | 2.2 | 1.2 | 236 | DRG-up |
| 94 | Complement receptor type 2 | 0.059 | 0.100 | 0.430 | 0.947 | 0.3 | 0.4 | 0.2 | 6 | Not |
| | precursor (Cr2) (Complement C3d | | | | | | | | | detected |
| | receptor). | | | | | | | | | |
| 95 | Membrane cofactor protein | 0.224 | 0.123 | 0.022 | 1.229 | 0.8 | 2.0 | 0.8 | 31 | Not |
| | precursor (CD46 antigen) | | | | | | | | | detected |
| | (Trophoblast leucocyte common | | | | | | | | | |
| | antigen) (TLX). | | | | | | | | | |
| 96 | Complement decay-accelerating | 0.081 | 0.025 | 0.134 | 0.3 | 0.1 | 0.4 | 0.875 | 1002 | DRG-down |
| | factor, GPI-anchored precursor | | | | | | | | | Nerve-down |
| | (DAF-GPI). | | | | | | | | | |

### 6.1.2. TaqMan® Quantitative Real-Time PCR Analysis

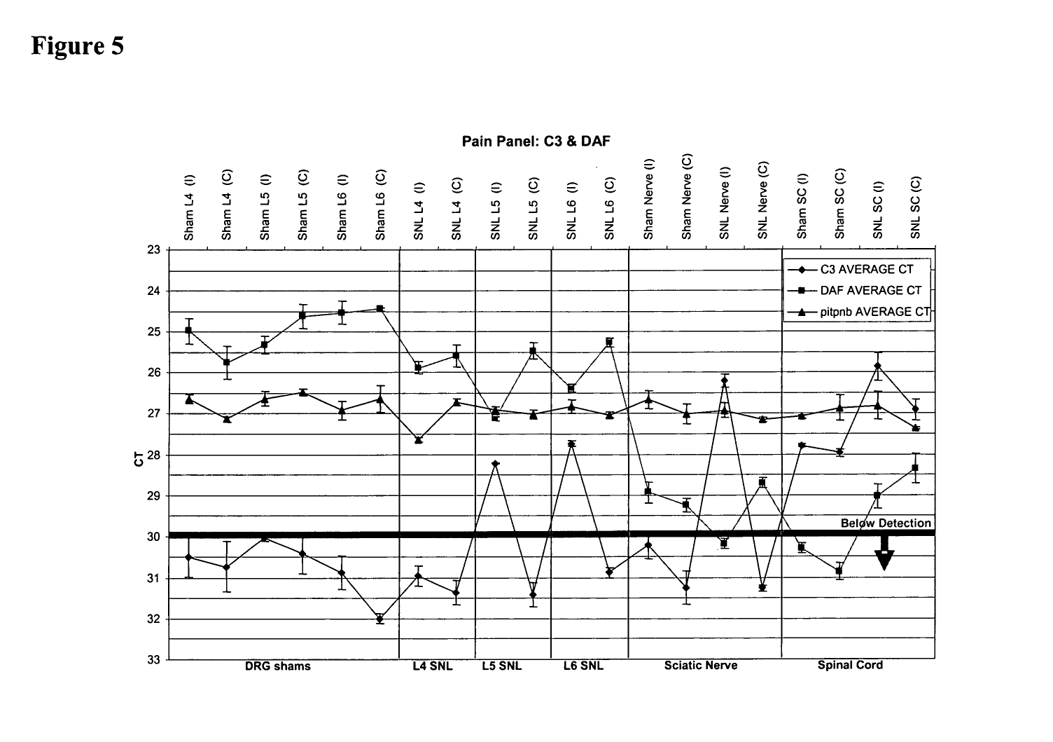

The expression profiles across 20 samples from L4 DRG, L5 DRG, L6 DRG, sciatic nerve, and spinal cord from both sham and SNL animals and from both the ipsi and contra sides were confirmed by TaqMan analysis, as described below for DAF and C3 (FIG. 5). In addition, the Taqman signal for a control gene, pitpnb (phosphatidylinositol transfer protein (beta isoform)), was determined for each of these samples. Results from the control gene showed that this gene was not regulated and that RNA input to the reaction was equal for all samples (FIG. 5).

Total RNA (10 ng, produced as described above) was used to synthesize cDNA with random hexamers using a TaqMan® Reverse Transcription Kit (Applied Biosystems, Foster City, Calif.). Real-time PCR analysis was performed on an Applied Biosystems ABI Prism 7700 Sequence Detection System. Matching primers and fluorescence probes were designed for the gene sequences using Primer Express software from Applied Biosystems. Primer and probe sequences used for DAF and C3 are listed in Table 5.

TABLE 5

List of nucleotide sequences (with nucleotide
sequences shown from 5′ to 3′)

| | Nucleo- | |
|---|---|---|
| SEQ | tide | |
| ID | Descrip- | |
| NO | tion | Sequence |
| 157 | TaqMan 5′ | GTTGTTGGTTCTGTATGCTGTCATC |
| | Primer | |
| | Sequence | |
| | for DAF | |
| 158 | TaqMan 3′ | CCATTCCAGACAACCTCCTTTC |
| | Primer | |
| | Sequence | |
| | for DAF | |
| 159 | TaqMan | CTTGAAGGTGTGCTAGAAATGATAACAAAG |
| | probe for | |
| | DAF | |
| 160 | TaqMan 5′ | CGGTCAAGGTCTACTCCTACTACAATC |
| | Primer | |
| | Sequence | |
| | for C3 | |
| 161 | TaqMan 3′ | CAGCATTCCATCGTCCTTCTC |
| | Primer | |
| | Sequence | |
| | for C3 | |
| 162 | TaqMan | AGGAGTCATGCACCCGGTTCTATCATCC |
| | probe for | |
| | C3 | |
| 163 | In situ | AATTAACCCTCACTAAAGGGGTTGTTGGTTCTGTAT |
| | hybridi- | GCT |
| | zation 5′ | |
| | Primer | |
| | Sequence | |
| | (with T3 | |
| | promoter | |
| | sequence | |
| | in bold) | |
| | for DAF | |
| 164 | In situ | TAATACGACTCACTATAGGGCCATTCCAGACAACC |
| | hybridi- | TCCT |
| | zation 3′ | |
| | Primer | |
| | Sequence | |
| | (with T7 | |
| | promoter | |
| | sequence | |
| | in bold) | |
| | for DAF | |
| 165 | In situ | AATTAACCCTCACTAAAGGGGTTGTTGGTTCTGTAT |
| | hybridi- | GCTGTCATCGTCTTGAAGGTGTGCTAGAAATGATAA |
| | zation | CAAAGCAAGAAGAAAGGAGGTTGTCTGGAATGGCC |
| | probe | CTATAGTGAGTCGTATTA |
| | sequence- | |
| | 1306-1390 | |
| | bp of | |
| | GenBank | |
| | accession | |
| | number | |
| | AF039583 | |
| | (promoter | |
| | sequences | |
| | in bold) | |
| | for DAF | |
| 166 | In situ | AATTAACCCTCACTAAAGGGGATCTCACACTCCGA |
| | hybridi- | AGAA |
| | zation 5′ | |
| | Primer | |
| | Sequence | |
| | (with T3 | |
| | promoter | |
| | sequence | |
| | in bold) | |
| | for C3 | |
| 167 | In situ | TAATACGACTCACTATAGGGATCCGACAGCTCTAT |
| | hybridi- | CGTC |
| | zation 3′ | |
| | Primer | |
| | Sequence | |
| | (with T7 | |
| | promoter | |
| | sequence | |
| | in bold) | |
| | for C3 | |
| 168 | In situ | AATTAACCCTCACTAAAGGGGATCTCACACTCCGA |
| | hybridi- | AGAAGACTGCCTGTCCTTCAAAGTCCACCAGTTCTTT |
| | zation | AACGTGGGACTTATCCAGCCGGGGTCGGTCAAGGTC |
| | probe | TACTCCTACTACAATCTAGAGGAGTCATGCACCCGG |
| | sequence- | TTCTATCATCCGGAGAAGGACGATGGAATGCTGAGC |
| | 201-519 | AAGCTGTGCCACAATGAAATGTGCCGCTGTGCCGAG |
| | bp of | GAGAACTGCTTCATGCATCAGTCACAGGATCAGGTC |
| | Gen Bank | AGCCTGAATGAACGACTAGACAAGGCTTGTGAGCCT |
| | accession | GGAGTGGACTACGTGTACAAGACCAAGCTAACGACG |
| | number | ATAGAGCTGTCGGAT CCCTATAGTGAGTCGTATTA |
| | M29866 | |
| | (promoter | |
| | sequences | |
| | in bold) | |
| | for C3 | |

Both forward and reverse primers were used at 200 nM. In all cases, the final probe concentration was 200 nM. The real-time PCR reaction was performed in a final volume of 25 μl using TaqMan® Universal PCR Master Mix containing AmpliTaq Gold DNA Polymerase, AmpErase UNG, dNTPs (with dUTP), Passive Reference 1, optimized buffer components (Applied Biosystems, Foster City, Calif.) and 5 μl of cDNA template. Three replicates of reverse transcription and real-time PCR for each RNA sample were performed on the same reaction plate. A control lacking a DNA template, and controls using reference genes with stable expressions in all samples in the SNL/GPN study, were included on the same plate to minimize the reaction variability.

In quantitative real-time PCR, exponential amplification of the initial target cDNA is reflected by increasing fluorescence. The amplification cycle at which this measured fluorescence crosses a specified threshold determined by the experimenter to be in the log-linear phase of the amplification is called the cycle threshold or CT value (according to the manual of the ABI Prism 7700 sequence detection system (Applied Biosystems, Foster City, Calif.)). Assuming 100% efficiency of the exponential amplification, CT values between samples can be directly compared with a difference of one CT unit corresponding to a 2-fold difference in expression levels, two CT units to 4-fold, three to 8-fold, and so on. Since CT units are exponential, the apparent fold difference between two samples would be calculated to be 2^{(CTsample1-CTsample2)}.

### 6.1.2.1. Results of TaqMan Analysis

TaqMan data indicates that DAF is down-regulated 3.3-, 3.5-, and 1.6-fold when comparing L5, L6, and the sciatic nerve SNL(ipsi) samples with sham control (ipsi) samples, respectively, as shown in FIG. 5. In contrast, C3 is up-regulated 3.3-, 9.8-, and 16.2-fold when comparing L5, L6 and the sciatic nerve SNL (ipsi) samples with sham control (ipsi) samples, respectively, as shown in FIG. 5. Thus, the above TaqMan data agree with the data generated from GeneChip analysis.

### 6.1.3. In situ Hybridization Analysis

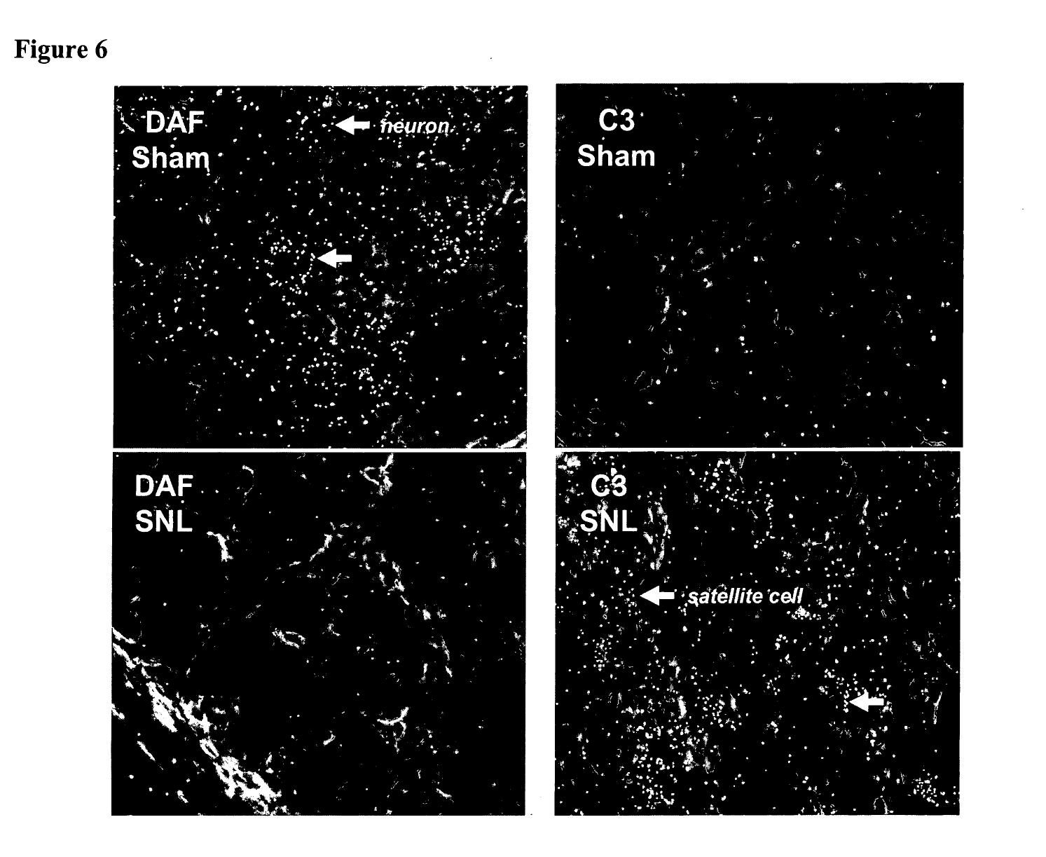

In situ hybridization was used to confirm that DAF was down-regulated and C3 was up-regulated in SNL DRG neurons compared to sham DRG neurons. FIG. 6 shows in situ hybridizations of DRGs from rats subjected to either an SNL or sham surgery. The left and right panels show the presence of DAF and C3, respectively. The top and bottom panels show hybridized DRGs from sham and SNL animals, respectively. In the sham panels, DAF expression is restricted to a subset of small, likely nociceptive neurons (indicated by arrows), whereas C3 expression is not detected. In SNL panels, DAF expression appears to be downregulated in the neurons, whereas C3 is upregulated mostly in the cells surrounding the neurons (satellite cells as indicated by the arrows).

DAF- and C3-specific ³⁵S-UTP labeled antisense RNA probes (SEQ ID NOS:165 and 168) were generated using T7 RNA polymerase from PCR templates. The PCR templates were generated from a rat DRG cDNA library using rat DAF- and C3-specific primers containing T7 and T3 RNA polymerase promoter sequences.

The in situ hybridization protocol was performed according to Frantz et al. (J. Neuroscience 1994, 14: 5725) with the exception of the proteinase K step which was omitted. DRG from Sprague Dawley rats (Taconic, Germantown, N.J.) were dissected and frozen in TBS Tissue Freezing Medium™ (Triangle Biomedical Sciences, Durham, N.C.). Frozen sections (20 μm thick) were fixed with 4% paraformaldehyde onto Fisher Scientific Superfrost glass slides (Pittsburgh, Pa.). Tissue sections were washed with PBS, treated with 0.25% acetic anhydride in 0.1M triethanolamine, and dehydrated using a series of four ethanol washes, (using 50%, 70%, and 2 times 95% ethanol in water).

Sections were incubated with 6×10⁶ cpm/ml of ³⁵S-labeled RNA probe in hybridization buffer (62.5% formamide, 12.5% dextran sulfate, 0.0025% polyvinylpyrolidone, 0.0025% ficoll, 0.0025% bovine serum albumin, 375 mM NaCl, 12.5 mM Tris pH=8, 1.3 mM EDTA, 10 mM dithiothreitol (DTT), 150 μg/ml E. coli tRNA) at 60° C. for 16 hours. Sections were then treated with 50 μg/ml RNAseA in 10 mM Tris/0.5M NaCl and subsequently washed through a series of 4 SSC (0.15 M sodium chloride, 0.15 M sodium citrate) washes containing 1 mM DTT (using 2×SSC buffer, 1×SSC buffer, 0.5×SSC buffer, and 0.1×SSC buffer). A final wash in 0.1×SSC, 1 mM DTT buffer was performed for 30 min at 65° C. Sections were then dehydrated through a series of six ethanol washes (using 50%, 70%, 95% ethanol in water, and 3 times using 100% ethanol), air-dried, and dipped in Kodak NTB2 emulsion (Rochester, N.Y.). Sections were exposed on slides for 2 weeks. Slides were developed using Kodak D19 developer and Rapid Fix (Rochester, N.Y.).

After slides were developed, they were counterstained with hematoxylin (Hematoxylin Stain Gill Formulation #2, Fisher Scientific, Fair Lawn, N.J.) and Eosin-Y (Lerner Laboratories, Pittsburgh, Pa.). Developed slides were first washed in water 3 times for 5 minutes each time and stained in hematoxylin (2 g/L) for 2 minutes. Excess hematoxylin was washed from the sections with water until the water was clear. Slides were then rinsed in 70% ethanol with 0.1% sodium borate for 2 minutes. Slides were then washed in water for 2 minutes, stained with eosin-Y(0.5%) for 2 minutes, washed in water for 2 minutes, and then rinsed through a series of alcohol washes (50%, 70%, 80%, 95%, 100%, and Xylene 2 times) for 1 minute each. Finally, a cover slip was applied using Cytoseal XYL (Richard-Allan Scientific, Kalamazoo, Mich.). As seen in FIG. 6, expression of DAF decreases and expression of C3 increases in the DRGs from SNL animals when compared with DRGs from sham animals.

Thus, in situ data confirms the up-regulation of complement effectors and the down-regulation of complement inhibitors in the DRGs of SNL animals when compared to the DRGs of sham animals.

### 6.1.4. Immunostaining Using Antibodies Against DAF Protein

Tissues used for immunohistochemistry were dissected from rats perfused with 4% paraformaldehyde made in PBS (1× phosphate-buffered saline, Ambion, Austin, Tex.). Tissues were further fixed in 4% paraformaldehyde for 24 hr at 4° C., cryoprotected for 24hr at 4° C. in 40% sucrose made in PBS, and frozen in TBS Tissue Freezing Medium™ (Triangle Biomedical Sciences, Durham, N.C.). Tissue sections (20 μm) were dried on gelatin coated slides, washed in PBS, incubated in 0.3% hydrogen peroxide for 10 min, blocked in 0.6% BSA for 1 hr and incubated overnight at 4° C. in the appropriate dilution of a monoclonal antibody to DAF (gift from Paul Morgan, Cardiff, UK). The sections were further processed by washing in PBS, incubating in the appropriate secondary IgG antibody conjugated to biotin for 1 hr (Jackson ImmunoResearch Laboratories, Inc, West Grove, Pa.) and then visualized using immunoperoxidase staining. Immunoperoxidase staining was done according to protocols included in the Vectastain Elite ABC Kit (PK-6100) and DAB substrate kit for peroxidase (SK-4100) from Vector Laboratories (Burlingame, Calif.). After staining, slides were washed in PBS and a coverslip was applied using Aqua-Mount (Lerner Laboratories, Pittsburgh, Pa.). Mead et al. (J Immunol. 2002, 168:458-65) is a general reference for staining with complement antibodies as described above:

As seen in FIG. 7, DAF protein expression is down regulated in the SNL model compared to sham animals (compare FIG. 7A and FIG. 7B). This result agrees with the results from microchip, TaqMan, and in situ hybridization experiments.

### 6.2. Example 2

### Treating Pain by Inhibition of Complement Using Cobra Venom Factor (CVF)

The present example demonstrates that rats subjected to the SNL model develop chronic neuropathic pain. When treated with CVF to inhibit complement, the chronic pain is alleviated as exhibited by reduced allodynia in treated rats compared to control rats subjected to SNL without subsequent CVF treatment.

### 6.2.1. General Methods: CVF Dosing Experiment

To determine the effect of CVF on complement C3 activity, naïve animals were injected with CVF on days 0, 3, and 6. C3 activity was measured using the hemolysis assay before and after CVF injections as described below.

### 6.2.2. General Methods: Surgery and CVF Injection Experiment

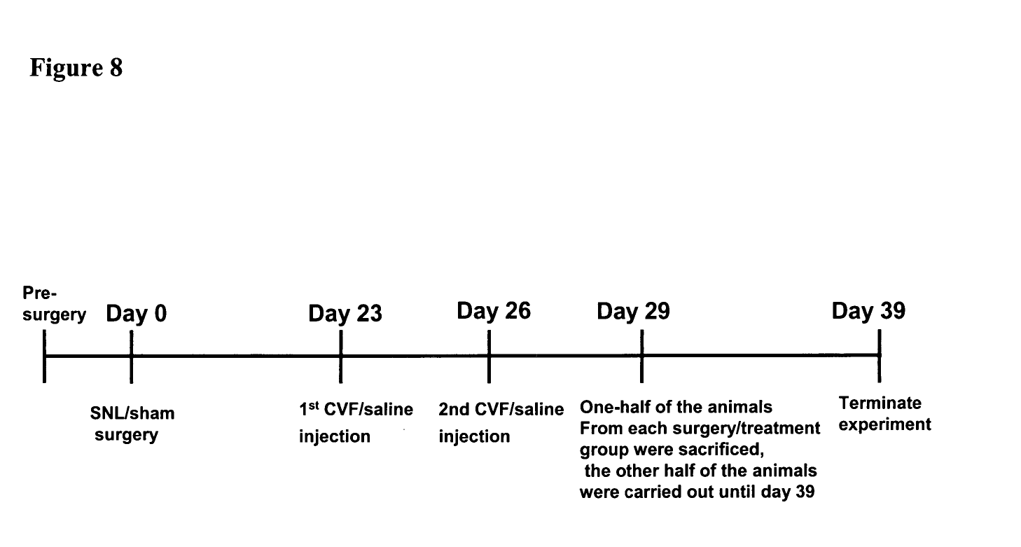

The timeline for the general method of surgery followed by CVF injection is outlined in FIG. 8. Spinal nerve ligation (SNL) was performed on Sprague-Dawley rats as described above in Example 1. On day 0, SNL surgery was performed on 20 rats and sham surgery was performed on 20 rats. At days 23 and 26 post surgery, 10 SNL and 10 sham animals (designated herein as SNL-CVF and Sham-CVF, respectively) were injected (ip) with CVF (350 units/kg). In addition, as controls, 10 SNL and 10 sham animals (designated herein as SNL-saline and Sham-saline, respectively) were given saline injections (ip). Five animals from each group were terminated on day 29. The remaining five animals were terminated on day 39. Pain behavior was measured using the paw pressure test as described in Example 1 above. C3 activity was measured by the hemolysis assay before and after animals received CVF as described below.

### 6.2.3. Method: Hemolysis Assay Including Sensitization of Sheep Erythrocytes (Ea)

The sensitization of sheep erythrocytes and the hemolysis assay to measure C3 activity were performed according to the Quidel Technical Bulletin entitled Measurement of C3 function in non-primate sera by hemolytic assay (Quidel Corporation, Santa Clara, Calif.). References within this protocol are the following: DeSautel and Brode, Laryngoscope 1999, 109:1674-8; Kirshfink, The Complement System 1998, Rother and Till eds, Springer Verlag Berlin Heidelberg, 522-547; Mollnes, Complement and Complement Receptors 1997, Weir ed, 78.1-78.6; Porcel et al., J. Immunol Method. 1993, 157:1-9; Lule et al., Complement 1984, 1:97-102; Mayer, Experimental Immunochemistry, 2nd ed. 1965, Kabat and Mayer eds, Charles C. Thomas, Springfield, 133-240.

Briefly, sheep blood erythrocytes (catalog number CS1113, Colorado Serum Company, Denver, Colo.) were sensitized using Sheep Red Blood Cell Stroma Fractionated Antiserum-Hemolysin (catalog number S1389, Sigma Chemical Company, St. Louis, Mo.) in Gelatin Veronal Buffer (also known as GVB²⁺, catalog number G6514, Sigma Chemical Company, St. Louis, Mo.).

All hemolysis assays were conducted in a total reaction volume of 250 μL and a final concentration of 6.5×10⁷ sensitized erythrocyte cells (Ea)/assay (for preparation of cells, see above). The reaction consists of 12.5 μL of test serum (for preparation of serum, see below) or dilutions of serum in GVB²⁺, 10 μL of Human C3-Depleted Serum (catalog number A508, Quidel Corporation, Santa Clara, Calif.), the appropriate volume of Ea to obtain 6.5×10⁷ cells, and GVB²⁺ to bring the total volume to 250 μL. The reactions were incubated in a 37° C. water bath for 30 minutes, with gentle agitation every 10 minutes. After incubation, the reactions were centrifuged for 10 minutes at 2000 g at 4° C. The supernatant (100 μL) was removed and transferred to a 96-well microplate for analysis of the optical density at A₅₄₀ to measure hemoglobin release (Spectramax 384, Molecular Devices, Sunnyvale, Calif.). Hemoglobin release indicates that cells have been lysed as a result of active C3 in the serum.

For dosing experiments, blood from rats was collected by drawing blood from a jugular vein catheter using a syringe. For the surgery and CVF injection experiments, blood from rats was collected through an intra-orbital eye bleed during the experiment or through a heart puncture after the animal was terminated. Sera used in the hemolysis assay was isolated from the collected blood by incubating the blood for 30 minutes at 37° C. to clot and then separating the blood by centrifugation at 4° C.

To determine the appropriate dilution of sera from animals injected with CVF or saline to be used in the hemolysis assay, sera from animals before CVF injection were serially diluted and tested. The following dilutions using GVB²⁺ buffer were made: 1:3, 1:5, 1:10, 1:30, 1:50, 1:100, 1:300, 1:500, 1:3000, 1:5000, 1:10,000. The hemolysis assay was performed on each of these dilutions and an undiluted sample as described above.

The hemolytic activity from each diluted sample was calculated as % lysis relative to the 100% lysis sample (100% lysis was the A₅₄₀ measurement from Ea incubated in water): (A₅₄₀ sample/A₅₄₀ 100% lysis sample)×100. Theoretical curves were generated using non-linear regression curve fitting analysis in GraphPad Prism version 3.02 using the log₁₀ of the dilutions vs % lysis graph. Based on the Prism analysis, an EC₅₀ was determined for each animal before CVF or control treatments.

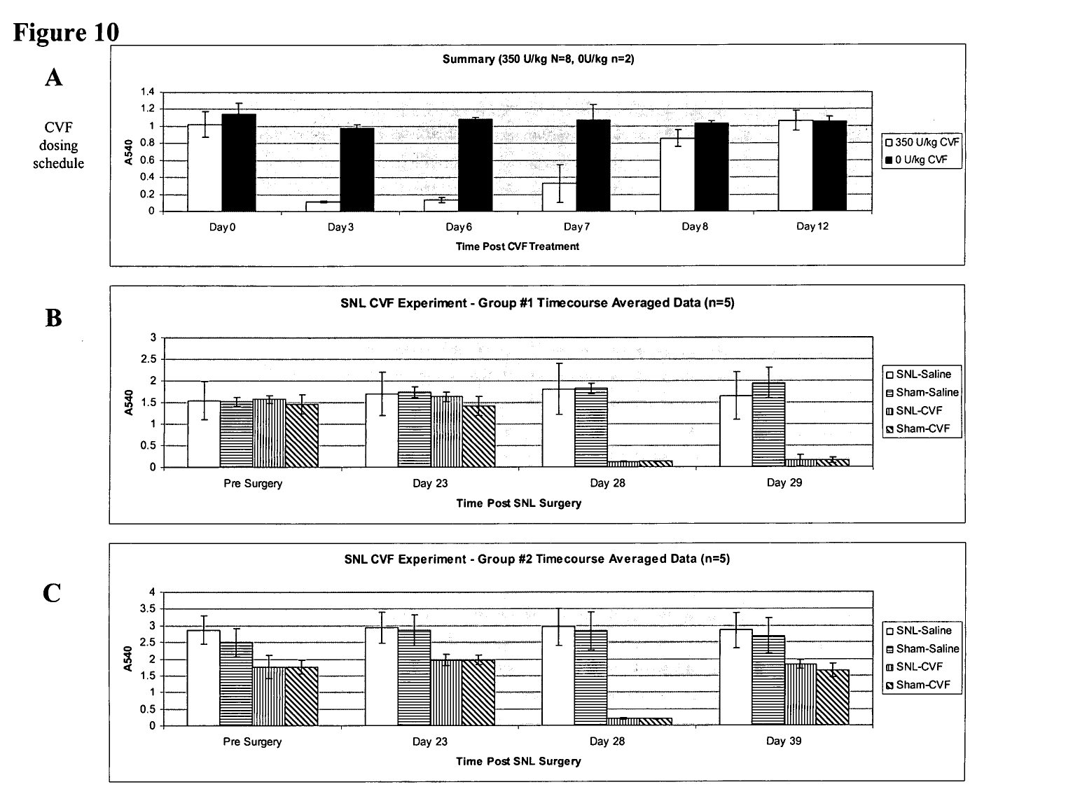

The dilution calculated from the EC₅₀ was then used for the serum samples drawn and prepared at each time point for each animal after CVF or saline treatments. The measured A₅₄₀ values were averaged for each group as shown in FIGS. 10A-C.

### 6.2.4. Results: CVF Dosing Experiment

CVF dosing experiments on naïve animals demonstrate that complement C3 activity levels return to pre-dosing levels by day 8 after animals are injected with CVF on days 0, 3, and 6 (FIG. 10A). C3 activity levels on day 8 of the dosing experiment correspond to day 31 in SNL-CVF experiments (for timeline see FIG. 8). Note that the results of FIG. 10A are consistent with the literature showing that CVF treatment becomes ineffective after a week due to an immune response to the CVF (Morgan and Harris, M61 Immunol. 2003, 40:159-70).

### 6.2.5. Results: Surgery and CVF Injection Experiment

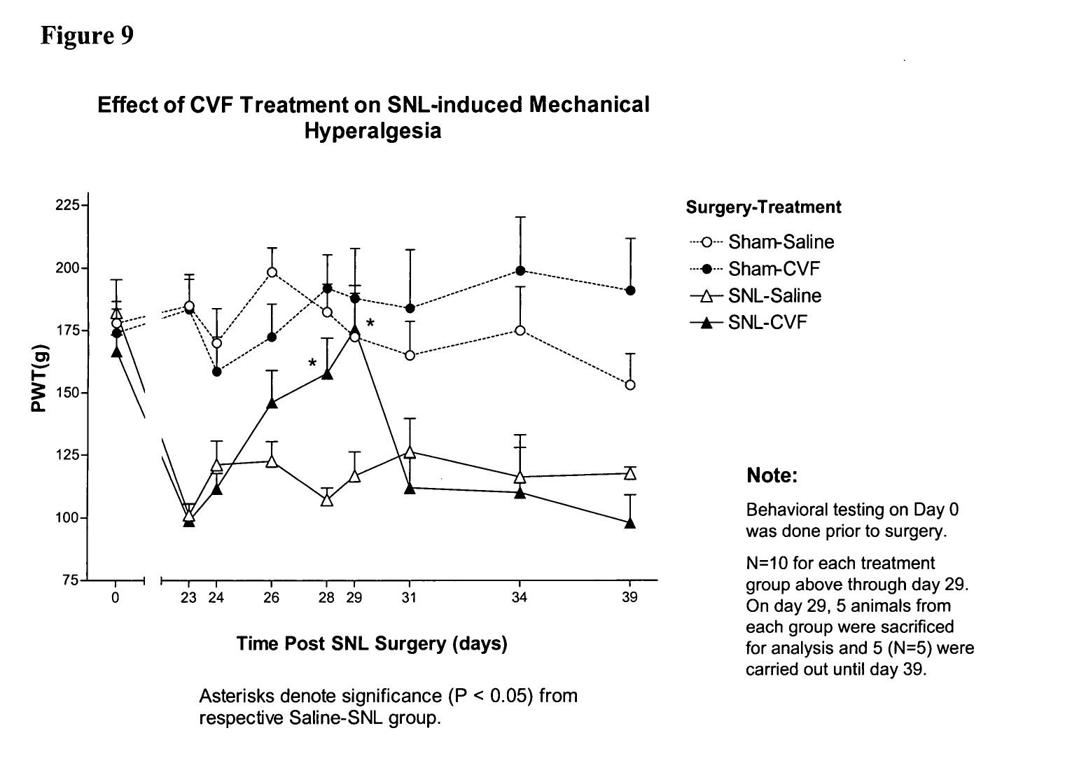

As shown in FIG. 9 in the SNL-CVF experiments, animals that had SNL surgery displayed pain behavior that was significantly different than Sham animals on day 23. By day 29, SNL animals that received CVF showed behavior that was not significantly different than Sham-saline or Sham-CVF animals. By day 31, pain behavior had returned to day 23 levels in SNL-CVF animals. The hemolysis assay was not actually done on day 31, but the timing of the return of pain behavior corresponds to what was seen in the CVF dosing experiment (day 8).

Hemolysis assays done on CVF treated animals in this experiment showed that C3 activity levels were down (below 20%) on days 28 and 29 relative to pre-surgery and day 23 (just before the start of CVF treatment) (FIG. 10B). Hemolysis assays performed on animals on day 39 showed that C3 complement activity levels had returned to pre-surgery and day 23 levels by day 39 (FIG. 10C). Thus, pain threshold results correlate with C3 activity in that a reduction in C3 levels reduces mechanical allodynia of an animal, as indicated by an increased paw withdrawal threshold.

### 6.3. Example 3

### Testing Pain in Animal Model Lacking Complement

The present prophetic example exemplifies a method for comparing the pain thresholds of C3 knockout mice that undergo spinal nerve ligation surgery with the pain thresholds of naïve mice that undergo spinal nerve ligation surgery. This experiment can be used to determine if elimination of C3 affects the pain state of an animal.

### 6.3.1. Experimental Overview for C3 Knockout Mouse: SNL Surgery and Behavioral Testing

C3 Knockout mice from Jackson Laboratory (JAX Research Services, Bar Harbor, Me., Stock Number:003641, Strain Name:B6.129S4-C3tm1Crr/J) can be used to test the effect of complement protein C3 on pain. Spinal Nerve Ligation (SNL) as described below is performed on 10 homozygote C3tm1Crr/J mice and 10 wildtype littermates which are expanded from an earlier cross of heterozygote C3tm1Crr/J mice. Sham surgery is performed on 10 homozygotes C3tm1Crr/J mice and 10 wildtype littermates. Mice are tested for pain behavior (mechanical allodynia) 14 days after surgery using von Frey hairs.

### 6.3.2. Spinal Nerve Ligation in Knockout Mice

Surgery is performed under isoflurane/O₂ anesthesia. Following induction of anesthesia, an incision is made just lateral to the spinal vertebrae from L6 to L3. The L5 transverse process is exposed by blunt dissection and removed with forceps. This process exposes the L5 spinal nerve close to the L5 DRG (within 2-4 mm). The L5 spinal nerve is then isolated and tightly ligated with 7-0 silk suture. After a complete hemostasis is confirmed, the wound (muscle and skin) is sutured using 4-0 Vicryl. Mice are given an injection of Ringer's Lactate solution; the wound is dusted with antibiotic powder; and the mice are returned to their home cages to recover.

### 7. References Cited

Numerous references, including patents, patent applications and various publications, are cited and discussed in the description of this invention. The citation and/or discussion of such references is provided merely to clarify the description of the present invention and is not an admission that any such reference is “prior art” to the invention described here. All references cited and/or discussed in this specification (including references, e.g., to biological sequences or structures in the GenBank, PDB or other public databases) are incorporated herein by reference in their entirety and to the same extent as if each reference was individually incorporated by reference.

1-81. (canceled)

82. A method for treating pain by modulating a biological activity of a complement component in a subject feeling pain, comprising administering to the subject a therapeutically effective amount of a compound that modulates a biological activity of a complement component, with the proviso that the compound is not cobra venom factor (CVF).

83. The method of claim 82, wherein the compound decreases a biological activity of a complement component.

84. The method of claim 83, wherein the complement component is a complement effector.

85. The method of claim 84, wherein the complement effector is C3, C3aR, C5aR, C5, C3 convertase, C5 convertase, Factor D, C1s, MASP-1, MASP-2, MASP-3, Factor B, C1r, or C5b-9.

86. The method of claim 82, wherein the compound inhibits an increase in a biological activity of a complement component.

87. The method of claim 82, wherein the compound increases a biological activity of a complement component.

88. The method of claim 87, wherein the complement component is an endogenous complement inhibitor.

89. The method of claim 88, wherein the complement inhibitor is decay accelerating factor (DAF), Factor H, Factor I, CRRY, CR1, clusterin, CD59, or C1 INH.

90. The method of claim 82, wherein the complement component is active in a pathway selected from the group consisting of the classical pathway, the MB-lectin pathway, the alternative pathway, and the downstream shared pathway.

91. The method of claim 82, wherein the type of pain is neuropathic pain, nociceptive pain, chronic pain, inflammatory pain, pain associated with cancer, or pain associated with rheumatic disease.

92-173. (canceled)


SEQUENCE LISTING (168 sequences)

SEQ ID NO: 1 - DNA, 7465 bp, Homo sapiens
acactctgggcgcggagcacaatgattggtcactcctattttcgctgagcttttcctcttatttcagttttcttcgagatcaaatctggtttgtagatgtgcttggggagaatgggggcctcttctccaagaagcccggagcctgtcgggccgccggcgcccggtctccccttctgctgcggaggatccctgctggcggttgtggtgctgcttgcgctgccggtggcctggggtcaatgcaatgccccagaatggcttccatttgccaggcctaccaacctaactgatgagtttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccagagttaccaagctgctccagggtgtgtcagccgcctccagaaatcctgcatggtgagcataccccaagccatcaggacaacttttcacctgggcaggaagtgttctacagctgtgagcctggctatgacctcagaggggctgcgtctctgcactgcacaccccagggagactggagccctgaagccccgagatgtgcagtgaaatcctgtgatgacttcttgggtcaactccctcatggccgtgtgctatttccacttaatctccagcttggggcaaaggtgtcctttgtctgtgatgaagggtttcgcttaaagggcagttccgttagtcattgtgtcttggttggaatgagaagcctttggaataacagtgttcctgtgtgtgaacatatcttttgtccaaatcctccagctatccttaatgggagacacacaggaactccctctggagatattccctatggaaaagaaatatcttacacatgtgacccccacccagacagagggatgaccttcaacctcattggggagagcaccatccgctgcacaagtgaccctcatgggaatggggtttggagcagccctgcccctcgctgtgaactttctgttcgtgctggtcactgtaaaaccccagagcagtttccatttgccagtcctacgatcccaattaatgactttgagtttccagtcgggacatctttgaattatgaatgccgtcctgggtattttgggaaaatgttctctatctcctgcctagaaaacttggtctggtcaagtgttgaagacaactgtagacgaaaatcatgtggacctccaccagaacccttcaatggaatggtgcatataaacacagatacacagtttggatcaacagttaattattcttgtaatgaagggtttcgactcattggttccccatctactacttgtctcgtctcaggcaataatgtcacatgggataagaaggcacctatttgtgagatcatatcttgtgagccacctccaaccatatccaatggagacttctacagcaacaatagaacatcttttcacaatggaacggtggtaacttaccagtgccacactggaccagatggagaacagctgtttgagcttgtgggagaacggtcaatatattgcaccagcaaagatgatcaagttggtgtttggagcagccctccccctcggtgtatttctactaataaatgcacagctccagaagttgaaaatgcaattagagtaccaggaaacaggagtttcttttccctcactgagatcatcagatttagatgtcagcccgggtttgtcatggtagggtcccacactgtgcagtgccagaccaatggcagatgggggcccaagctgccacactgctccagggtgtgtcagccgcctccagaaatcctgcatggtgagcataccctaagccatcaggacaacttttcacctgggcaggaagtgttctacagctgtgagcccagctatgacctcagaggggctgcgtctctgcactgcacgccccagggagactggagccctgaagcccctagatgtacagtgaaatcctgtgatgacttcctgggccaactccctcatggccgtgtgctacttccacttaatctccagcttggggcaaaggtgtcctttgtttgcgatgaagggttccgattaaaaggcaggtctgctagtcattgtgtcttggctggaatgaaagccctttggaatagcagtgttccagtgtgtgaacaaatcttttgtccaaatcctccagctatccttaatgggagacacacaggaactccctttggagatattccctatggaaaagaaatatcttacgcatgcgacacccacccagacagagggatgaccttcaacctcattggggagagctccatccgctgcacaagtgaccctcaagggaatggggtttggagcagccctgcccctcgctgtgaactttctgttcctgctgcctgcccacatccacccaagatccaaaacgggcattacattggaggacacgtatctctatatcttcctgggatgacaatcagctacacttgtgaccccggctacctgttagtgggaaagggcttcattttctgtacagaccagggaatctggagccaattggatcattattgcaaagaagtaaattgtagcttcccactgtttatgaatggaatctcgaaggagttagaaatgaaaaaagtatatcactatggagattatgtgactttgaagtgtgaagatgggtatactctggaaggcagtccctggagccagtgccaggcggatgacagatgggaccctcctctggccaaatgtacctctcgtgcacatgatgctctcatagttggcactttatctggtacgatcttctttattttactcatcattttcctctcttggataattctaaagcacagaaaaggcaataatgcacatgaaaaccctaaagaagtggctatccatttacattctcaaggaggcagcagcgttcatccccgaactctgcaaacaaatgaagaaaatagcagggtccttccttgacaaagtactatacagctgaagaacatctcgaatacaattttggtgggaaaggagccaattgatttcaacagaatcagatctgagcttcataaagtctttgaagtgacttcacagagacgcagacatgtgcacttgaagatgctgccccttccctggtacctagcaaagctcctgcctctttgtgtgcgtcactgtgaaacccccacccttctgcctcgtgctaaacgcacacagtatctagtcaggggaaaagactgcatttaggagatagaaaatagtttggattacttaaaggaataaggtgttgcctggaatttctggtttgtaaggtggtcactgttcttttttaaaatatttgtaatatggaatgggctcagtaagaagagcttggaaaatgcagaaagttatgaaaaataagtcacttataattatgctacctactgataaccactcctaatattttgattcattttctgcctatcttctttcacatatgtgtttttttacatacgtacttttccccccttagtttgtttccttttattttatagagcagaaccctagtcttttaaacagtttagagtgaaatatatgctatatcagtttttactttctctagggagaaaaattaatttactagaaaggcatgaaatgatcatgggaagagtggttaagactactgaagagaaatatttggaaaataagatttcgatatcttctttttttttgagatggagtctggctctgtctcccaggctggagtgcagtggcgtaatctcggctcactgcaacgtccgcctcccgggttgacaccattttcctgcctcagcctcctgagtagttgggactaccagtagatgggactacaggcacctgccaacacgcccggctaatttttttgtatttttagtagagacggggtttcaccatgttagccaggatggtctggatctcctgacctcgtgatccacccgcctcggcctcccaaagtgctgcgattacaggcatgagccaccgcgcctggccgctttcgatattttctaaactttaattcaaaagcactttgtgctgtgttctatataaaaaacataataaaaattgaaatgaaagaataattgttattataaaagtactagcttacttttgtatggattcagaatatactaaattaactttttaaaacacaacttttaaaaaatgtatcaaaaataataaacgtgttctgatattttta

SEQ ID NO: 2 - DNA, 5373 bp, Mus musculus
cagaagggagcagacagtcagaccagacaggtctgacctttcctgaatcctccagccatgcggctcctctgggggctggcctgggtgttcagcttctgtgcctcatccctgcagaagcccaggttgctcctgttttccccttctgtggttaatttggggacccccctgtcggtgggggtacagctcctggatgcccctccaggacaggaggtaaaaggatcagtgttcctcagaaacccaaagggtggttcctgctccccaaagaaggactttaagctgagctcgggagatgactttgtgctgctcagccttgaggtcccactggaagatgtgaggagctgtggcctctttgacctgcgcagagccccccacatccagctggtagctcagtctccgtggctaaggaacacagctttcaaagccacagagactcagggtgtcaacttgctcttctcttcccgacgaggccacatctttgtgcagaccgatcagcctatctataatccggggcagcgggttcgttatcgggtctttgcactggatcaaaagatgcgcccatccactgatttcctcaccatcacagtggagaactcccatggcctccgtgtactcaagaaggagatatttacttccacatccatcttccaagatgccttcaccattccagacatctcagagcctgggacctggaagatctcagctaggttctcagatggactggagtccaataggagcacccactttgaagtgaagaagtatgtccttcccaacttcgaggtgaagattactccttggaagccatatatcctgatggtgcccagcaacagtgatgaaatccaattagacatccaggccaggtacatctatgggaagcccgtgcagggcgtggcatacacacggtttgcgctcatggatgagcaagggaagaggactttccttcggggcctagagacgcaggccaagttggtggaaggccggacccacatttccatctcaaaggaccagttccaggctgccctggataaaatcaatattggggtcagagacctggaggggctgcgtctctatgctgctacagctgtcatcgagtctccaggaggagagatggaggaggcagaactcacgtcctggcgctttgtatcatctgccttctccttggatctcagccgcactaagaggcatcttgtgcctggggcccacttcctgctgcaggctctggtccaagaaatgtcaggctctgaagcctccaacgttcctgttaaagtctctgccacattggtgtcaggctctgactcccaagtccttgacattcaacagagcaccaatggaattggccaagtcagcatttccttccccatcccaccaactgtcacagaacttcgactcttggtgtctgcgggctccctctacccagccatagccaggctcaccgtgcaagccccaccttcaagaggcactggctttctttctattgagccactagaccctcggtcccctagtgtgggggacacctttatcctaaaccttcaacctgtgggcatccctgcacctaccttctctcattactactacatgatcatctccagaggccagatcatggctatgggtcgggagccccggaagactgtgacctccgtctctgtgttggtggaccatcagctggctccctcgttctactttgtggcttacttctatcaccaaggacacccggtggccaactctctgctcatcaacatccaatccagggactgtgagggcaagctgcaattgaaggtggatggtgccaaggagtatcgtaatgcggacatgatgaagctccgaattcaaactgactccaaagccctggtggcactgggagctgtggacacggctctgtatgctgtgggtggtcggtctcacaaacccctcgacatgagcaaggtctttgaagtaatcaacagctacaatgttggctgtggtcctggaggtggggatgatgcccttcaggtgttccaggatgctggtctggccttttctgatggtgatcgactaactcaaaccagagaggacctgagctgtcccaaggagaagaaaagtcggcaaaagagaaatgttaacttccagaaggctgtcagtgagaagttgggccagtattcttctccagatgccaagcgctgctgccaagacgggatgacgaagctgcccatgaagcgtacctgtgagcagcgggctgcccgtgtgcctcagcaggcctgccgtgagcccttcttgtcctgttgcaagtttgctgaggaccttcgcaggaaccagaccaggagccaggcacaccttgcccgaaacaaccacaacatgctgcaggaggaagacttgatagatgaagacgacattcttgtgcgcacctccttcccagagaactggctctggagagtggaacctgtagacagctccaaactgttgacagtgtggcttcctgattctatgaccacatgggagattcatggtgtgagcctgtccaaaagcaaaggtctgtgtgtagccaagccaactcgtgttcgagtgttcagaaaattccacctgcacctgcgcctgcccatctccatccgccgctttgagcagtttgaattacggcctgttctttacaactatctgaatgatgatgtggctgtgagtgtccatgtgaccccagttgaggggctgtgcctggctggtggtggaatgatggcccagcaggtgacagtgccagcaggttctgcccggcctgtggccttctctgtggtacccacagctgctgccaacgtgcccctgaaggtggtggctcgaggggtttttgatttaggggatgctgtgtctaagattctccaaattgagaaggaaggagccatccacagagaagagttagtctacaacctcgaccccctaaataacctgggtcggactttggagattcctggcagctcggatcccaacatcgtccctgacggagacttcagcagcttagtcagggttacagcctcggaacccttggagactatgggttctgaaggtgccttgtccccaggaggtgtggcctccctcctgaggcttccccagggctgtgcagagcaaaccatgatctatttggctcctaccctgactgcttccaactacctggacaggacagaacagtggagcaaactgtcccctgaaacaaaggaccatgctgtggatctgatccaaaaaggatacatgaggatccagcagtttcggaagaatgatggctcctttggggcttggttacaccgggacagcagcacctggctgactgcctttgtgctgaagattctgagtttggcccaggaacaggtgggcaactccccggagaagctgcaggagacggctagctggctgctggcccagcagctgggtgatggctccttccacgacccatgtccagtcatccacagagcaatgcaggggggcttggtggggtctgacgagacagtggcactgaccgcctttgtggtcattgcccttcaccatgggttggacgtcttccaggatgacgatgcgaagcagctgaagaacagagtggaagcctccatcaccaaggcaaactcattcttggggcagaaggcaagtgctgggctcctgggtgcccatgccgccgccatcacagcctatgcccttacgctgaccaaggcctcggaggacctgcggaatgttgcccacaacagcctgatggccatggctgaggaaacaggggaacacctctactggggcttagtccttggctctcaggacaaagttgtgttgcgccccacagccccccgtagccctacagaacctgtgccccaggccccagccttgtggatcgaaaccacagcctatgccctgctccacctgcttctgcgggagggaaagggaaaaatggctgacaaggctgcatcctggctcacccaccagggaagcttccatggggcattccgcagtacccaggacactgtggtcaccctggatgccctgtctgcctactggatcgcttcacacaccactgaggagaaagcactgaaggtgacgctcagctccatgggccgcaatgggcttaagacccacgggctacacttgaacaaccaccaagtcaagggcctggaggaggagctaaagttctccctgggcagcacaatcagtgtcaaggtggaaggaaacagcaaaggcaccttgaagatccttcgtacctacaatgtcctggacatgaagaacaccacatgccaggaccttcagatagaagtgaaggtcacaggcgctgtggaatacgcatgggatgccaatgaagactacgaagactactatgacatgccagctgcagatgaccccagcgtccccttgcagcctgtcacgcccctgcagctttttgagggtcgtaggagccgccgcaggagggaggcccccaaggtggctgaagagcaggagtccagagttcagtacactgtgtgtatctggcgaaatggcaagctggggctgtctggcatggccatcgcagacatcaccctcctaagtggattccacgccctgagggctgacctggagaagctgacctccctctctgaccgttacgtgagtcactttgagactgacgggccccatgtcctgttgtactttgactcggtccctaccacccgggagtgtgtgggcttcggagcctcgcaggaggtggttgtgggactggtgcagccatccagtgctgtcctgtatgactactacagccctgatcacaagtgctctgtgttttatgctgcacccaccaagagccagctcctggccacactgtgctctggagatgtatgccagtgcgctgaggggaagtgccctcgactgctaaggtcactggagcgaagggtggaggacaaggatggctaccggatgaggttcgcctgctattatccccgagtggagtatggcttcacggttaaggttcttcgagaagatggcagagctgccttccgtctctttgagtccaagatcacccaagtcctgcatttcagaaaggacaccatggcctccataggtcagacccgcaacttcctgagccgggcctcttgccgccttcgtttggagcctaacaaagagtacttgatcatggggatggacggggaaaccagtgacaacaagggagacccccagtacttgctggactcaaatacctggattgaggagatgccttcagaacaaatgtgcaagagcacccgccatcgggcagcctgtttccagctcaaagacttcctgatggagttcagcagccgggggtgccaggtgtgaggccttaggactctggctctctgagctcagctcagggttagggcctcactggattagaggctctgctctacagggtaaataaaagaaaagctttttgac

SEQ ID NO: 3 - DNA, 5403 bp, Mus musculus
gccgctaccagccatgggtctttggggaatactttgtcttttaattttcctggacaaaacttggggacaggaacaaacctacgtcatttcagcacccaaaatcctccgggtcggctcgtctgaaaatgtggtaattcaagtccatggctacactgaagcatttgatgcaactctttctctaaaaagctatcctgacaaaaaagtcaccttctcttcaggctatgttaatttgtccccggaaaacaaattccaaaacgcggcactgttgacactacagcccaatcaagttcctagagaagaaagcccagtctctcacgtgtatctggaagttgtgtcaaaacacttttcaaaatcaaagaaaataccaattacctataacaatggaattctcttcatccatacagacaaacctgtttacacgccggaccagtcagtaaagatcagagtctattctctgggtgacgacttgaagccagccaaacgggagactgtcttaactttcatagaccccgaaggatcagaagttgacattgtagaagaaaatgattacaccggaattatctcttttcctgacttcaagattccatctaatcccaagtatggtgtttggacaattaaagctaactataagaaggattttacaacaactggaactgcatactttgaaattaaagaatatgtcttgccacgattctctgtttcaatagaactagaaagaaccttcattggctataaaaactttaagaactttgaaatcactgtgaaagcaagatatttttataataaagtggtacctgatgctgaagtgtatgccttttttggattgagagaggacataaaagatgaggagaagcagatgatgcacaaagccacacaagccgcaaagttggttgacggagttgctcagatctcttttgattctgaaacagcagttaaagagctgtcctacaacagtctagaagacttaaacaacaagtacctttatattgcagtaacagtcacagaatcttcaggtggattttcagaagaggcagaaatccctggagtcaaatatgtcctctctccctacacactgaatttggtcgctactcctcttttcgtgaagcccgggattccattttccatcaaggcacaggttaaagattcactcgagcaggcggtaggaggggtcccagtaactctgatggcacaaacagtcgatgtgaatcaagagacatctgacttggaaacaaagaggagcatcactcatgacactgatggagtagctgtgtttgtgctgaacctcccatcaaatgtgacggtgctaaagtttgagatcagaactgatgacccagaacttcccgaagaaaatcaagccagcaaagagtacgaagcagttgcgtactcgtctctcagccaaagttacatttacatcgcttggactgaaaactacaagcccatgcttgtgggagaatacctgaatattatggttacccccaagagcccatatatcgacaaaataactcactataattacttgattttatccaaaggcaaaattgtacagtacggcacaagagagaaacttttctcctcaacttatcaaaatataaatattccagtgacacagaacatggttccttcagcacgactcctggtctattacatagtcacaggggagcaaacagcagaattagtggctgacgcagtctggataaatattgaggagaagtgtggcaaccagctccaggtccatctgtctccagatgaatatgtgtattctccaggccaaactgtgtcccttgacatggtgactgaagcagactcatgggtagcactatcagcagtggacagagctgtgtataaagtccagggaaacgccaaaagggccatgcaaagagtctttcaagctttggatgaaaagagtgacctgggctgtggggcaggtggtggccatgacaatgcagatgtattccatctagctgggctcaccttcctcaccaacgcaaacgcagatgactcccattatcgtgatgactcttgtaaagaaattctcaggtcaaagagaaacctgcatctcctaaggcagaaaatagaagaacaagctgctaagtacaaacatagtgtgccaaagaaatgctgctatgacggagcccgagtgaacttctacgaaacctgtgaggagcgagtggcccgggttaccataggccctctctgcatcagggccttcaacgagtgctgtactattgcgaacaagatccgaaaagaaagcccccataaacctgtccaactgggaaggatccacattaagaccctgttaccagtgatgaaggcagatatccgaagctactttccagagagctggctatgggaaattcatcgcgttcccaaaagaaaacagctgcaggtcacgctgcctgactcactaacgacttgggaaattcaaggcattggcatttcagacaatggtatatgtgttgctgatacactcaaggcaaaggtgttcaaagaagtcttcctggagatgaacataccatattctgttgtgcgaggagaacagatccaattgaaaggaactgtttacaactatatgacctcagggacaaagttctgtgttaaaatgtctgctgtggaggggatctgcacttcaggaagctcagctgctagccttcacacctccaggccctccagatgtgtgttccagaggatagagggctcgtccagtcacttggtgaccttcaccctgcttcctctggaaattggccttcactccataaacttctcactagagacctcatttgggaaagacatcttagtaaagacattacgggtagtgccagaaggagtcaagagggaaagctatgccggcgtgattctggaccctaagggaattcgtggtattgttaacagacgaaaggaattcccatacaggatcccattagatttggtccccaagaccaaagttgaaaggattttgagtgtcaaaggactgcttgtaggggagttcttgtccacggttctgagtaaggaaggcatcaacatcctaacccacctccccaagggcagtgcagaggcagagctcatgagcatagctccggtgttctatgttttccactacctggaagcaggaaaccattggaatattttctatcctgatacactgagtaaaagacagagcctggagaaaaaaataaaacaaggggtggtgagcgtcatgtcctacagaaacgctgactattcctacagcatgtggaagggggcgagcgctagtacctggctgacagcttttgctctgagagtgcttggacaggtggccaagtatgtaaaacaggatgaaaactcaatttgtaactctttgctatggctggttgagaagtgtcagctggaaaacggctctttcaaggaaaattcccaatatctaccaataaaattacagggtactttgcctgctgaagcccaagagaaaactttgtatcttacagccttttctgtgattggaattagaaaggcagttgacatatgccccaccatgaaaatccacacagcgctagataaagccgactccttcctgcttgaaaacaccctgccatccaagagcaccttcacactggccattgtagcctatgctctttccctaggagacagaacccacccgaggtttcgtctaattgtgtcggccctgaggaaggaagcttttgttaaaggtgatccgcccatttaccgttactggagagataccctcaaacgtccagacagctctgtgcccagcagcggcacagcaggtatggttgaaaccacagcctatgctttgctcgccagcctgaaactgaaggatatgaattacgccaaccccatcatcaagtggctatctgaagagcagaggtatggaggcggcttttattccacccaggatacgattaatgccatcgagggcctgacagaatattcactcctgttaaaacaaattcatttggatatggacatcaatgtcgcctacaaacacgaaggtgacttccacaagtataaggtgacagagaagcatttcctggggaggccagtggaggtatctctcaatgatgaccttgttgtcagcacaggctacagcagtggcttggccacagtatatgtaaaaactgtggttcacaaaattagtgtctctgaggaattttgcagcttttacttgaaaattgatacccaagatattgaagcatccagccacttcaggctcagtgactctggattcaagcgcataatagcatgtgccagctacaagcccagcaaggaggagtcaacatccgggtcctcccatgcagtaatggatatatcactgccgactggaatcggagcaaacgaggaagatttacgggctcttgtggaaggagtggatcaactactaactgattaccagatcaaagatggccatgtcattctgcaactgaattcgatcccctccagagatttcctctgtgtccggttccggatatttgaacttttccaagttgggtttctgaatcctgctaccttcacggtgtacgagtatcacagaccagataagcagtgcaccatgatttatagcatttctgacaccaggcttcagaaagtctgtgaaggagcagcttgcacatgtgtggaagctgactgtgcgcaactgcaggcagaagtagacctagccatctctgcagactccagaaaagagaaagcctgtaaaccagagactgcatatgcttataaagtcaggatcacatcagccactgaagaaaatgtttttgtcaagtacactgcgactcttctggtcacttacaaaacaggggaagctgctgatgagaattcggaggtcaccttcattaaaaagatgagctgtaccaatgccaacctggtgaaagggaagcagtatttaatcatgggcaaagaggttctgcagatcaaacacaatttcagtttcaagtatatataccctctagattcctccacctggattgaatattggcccacagacacaacgtgtccatcctgtcaagcatttgtagagaatttgaataactttgctgaagacctctttttaaacagctgtgaatgaaaagttctgctgcacgaagattcctcctgcggcggggggattgctcctcctctggcttggaaacctagcctagaatcagatacactttctttagagtaaagcacaagctgatgagttacgactttgtgaaatggatagccttgaggggaggcgaaaacaggtcccccaaggctatcagatgtcagtgccaatagactgaaacaagtctgtaaagttagcagtcaggggtgttggttggggccggaagaagagacccactgaaactgtagccccttatcaaaacatatccttgcttgaaagaaaaataccaaggacagaaaatgccataaaatcttgactttgcactc

SEQ ID NO: 4 - DNA, 5067 bp, Homo sapiens
ctcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacctccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtcagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgccgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcctgaggaggacgaatgccaagacgaagagaaccagaaacaatgccaggacctcggcgccttcaccgagagcatggttgtctttgggtgccccaactgaccacacccccattcc

SEQ ID NO: 5 - DNA, 5087 bp, Mus musculus
gcctctgcccacccctgccccttaccccttcattccttccacctttttccttcactatgggaccagcttcagggtcccagctactagtgctactgctgctgttggccagctccccattagctctggggatccccatgtattccatcattactcccaatgtcctacggctggagagcgaagagaccatcgtactggaggcccacgatgctcagggtgacatcccagtcacagtcactgtgcaagacttcctaaagaggcaagtgctgaccagtgagaagacagtgttgacaggagccagtggacatctgagaagcgtctccatcaagattccagccagtaaggaattcaactcagataaggaggggcacaagtacgtgacagtggtggcaaacttcggggaaacggtggtggagaaagcagtgatggtaagcttccagagtgggtacctcttcatccagacagacaagaccatctacacccctggctccactgtcttatatcggatcttcactgtggacaacaacctactgcccgtgggcaagacagtcgtcatcctcattgagacccccgatggcattcctgtcaagagagacattctgtcttccaacaaccaacacggcatcttgcctttgtcttggaacattcctgaactggtcaacatggggcagtggaagatccgagccttttacgaacatgcgccgaagcagatcttctccgcagagtttgaggtgaaggaatacgtgctgcccagttttgaggtccgggtggagcccacagagacattttattacatcgatgacccaaatggcctggaagtttccatcatagccaagttcctgtacgggaaaaacgtggacgggacagccttcgtgatttttggggtccaggatggcgataagaagatttctctggcccactccctcacgcgcgtagtgattgaggatggtgtgggggatgcagtgctgacccggaaggtgctgatggagggggtacggccttccaacgccgacgccctggtggggaagtccctgtatgtctccgtcactgtcatcctgcactcaggtagtgacatggtagaggcagagcgcagtgggatcccgattgtcacttccccgtaccagatccacttcaccaagacacccaaattcttcaagccagccatgccctttgacctcatggtgttcgtgaccaaccccgatggctctccggccagcaaagtgctggtggtcactcagggatctaatgcaaaggctctcacccaagatgatggcgtggccaagctaagcatcaacacacccaacagccgccaacccctgaccatcacagtccgcaccaagaaggacactctcccagaatcacggcaggccaccaagacaatggaggcccatccctacagcactatgcacaactccaacaactacctacacttgtcagtgtcacgaatggagctcaagccgggggacaacctcaatgtcaacttccacctgcgcacagacccaggccatgaggccaagatccgatactacacctacctggttatgaacaaggggaagctcctgaaggcaggccgccaggttcgggagcctggccaggacctggtggtcttgtccctgcccatcactccagagtttattccttcatttcgcctggtggcttactacaccctgattggagctagtggccagagggaggtggtggctgactctgtgtgggtggatgtgaaggattcctgtattggcacgctggtggtgaagggtgacccaagagataaccatctcgcacctgggcaacaaacgacactcaggattgaaggaaaccagggggcccgagtggggctagtggctgtggacaagggagtgtttgtgctgaacaagaagaacaaactcacacagagcaagatctgggatgtggtagagaaggcagacattggctgcaccccaggcagtgggaagaactatgctggtgtcttcatggatgcaggcctggccttcaagacaagccaaggactgcagactgaacagagagcagatcttgagtgcaccaagccagcagcccgccgccgtcgctcagtacagttgatggaaagaaggatggacaaagctggtcagtacactgacaagggtcttcggaagtgttgtgaggatggtatgcgggatatccctatgagatacagctgccagcgccgggcacgcctcatcacccagggcgagaactgcataaaggccttcatagactgctgcaaccacatcaccaagctgcgtgaacaacacagaagagaccacgtgctgggcctggccaggagtgaattggaggaagacataattccagaagaagatattatctctagaagccacttcccacagagctggttgtggaccatagaagagttgaaagaaccagagaaaaatggaatctctacgaaggtcatgaacatctttctcaaagattccatcaccacctgggagattctggcagtgagcttgtcagacaagaaagggatctgtgtggcagacccctatgagatcagagtgatgcaggacttcttcattgacctgcggctgccctactctgtagtgcgcaacgaacaggtggagatcagagctgtgctcttcaactaccgtgaacagcaggaacttaaggtgagggtggaactgttgcataatccagccttctgcagcatggccaccgccaagaatcgctacttccagaccatcaaaatccctcccaagtcctcggtggctgtaccgtatgtcattgtccccttgaagatcggccaacaagaggtggaggtcaaggctgctgtcttcaatcacttcatcagtgatggtgtcaagaagacactgaaggtcgtgccagaaggaatgagaatcaacaaaactgtggccatccatacactggacccagagaagctcggtcaagggggagtgcagaaggtggatgtgcctgccgcagaccttagcgaccaagtgccagacacagactctgagaccagaattatcctgcaagggagcccggtggttcagatggctgaagatgctgtggacggggagcggctgaaacacctgatcgtgacccccgcaggctgtggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagagaccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagccgtagctctgcaaccacgtttcgcctgctctgggaaaatggcaacctcctgcgatcggaagagaccaagcaaaatgaggccttctctctaacagccaaaggaaaaggccgaggcacattgtcggtggtggcagtgtatcatgccaaactcaaaagcaaagtcacctgcaagaagtttgacctcagggtcagcataagaccagcccctgagacagccaagaagcccgaggaagccaagaataccatgttccttgaaatctgcaccaagtacttgggagatgtggacgccactatgtccatcctggacatctccatgatgactggctttgctccagacacaaaggacctggaactgctggcctctggagtagatagatacatctccaagtacgagatgaacaaagccttctccaacaagaacaccctcatcatctacctagaaaagatttcacacaccgaagaagactgcctgaccttcaaagttcaccagtactttaatgtgggacttatccagcccgggtcggtcaaggtctactcctattacaacctcgaggaatcatgcacccggttctatcatccagagaaggacgatgggatgctcagcaagctgtgccacagtgaaatgtgccggtgtgctgaagagaactgcttcatgcaacagtcacaggagaagatcaacctgaatgtccggctagacaaggcttgtgagcccggagtcgactatgtgtacaagaccgagctaaccaacataaagctgttggatgattttgatgagtacaccatgaccatccagcaggtcatcaagtcaggctcagatgaggtgcaggcagggcagcaacgcaagttcatcagccacatcaagtgcagaaacgccctgaagctgcagaaagggaagaagtacctcatgtggggcctctcctctgacctctggggagaaaagcccaacaccagctacatcattgggaaggacacgtgggtggagcactggcctgaggcagaagaatgccaggatcagaagtaccagaaacagtgcgaagaacttggggcattcacagaatctatggtggtttatggttgtcccaactgactacagcccagccctctaataaagcttcagttgtatttc

SEQ ID NO: 6 - DNA, 5066 bp, Rattus norvegicus
ctaccccttacccctcactccttccacctttgtcctttaccatgggacccacgtcagggtcccagctactagtgctactgctgctgttggccagctccctgctagctctggggagccccatgtactccatcattactcccaatgtcctgcggctggagagtgaagagactttcatactagaggcccatgatgctcagggtgacgtcccagtcactgtcactgtgcaagacttcctaaagaagcaagtgctgaccagtgagaagacagtgttgacaggagccactggacatctgaacagggtcttcatcaagattccagccagtaaggaattcaatgcagataaggggcacaagtacgtgacagtggtggcaaacttcggggcaacagtggtggagaaagcggtgctagtaagctttcagagtggttacctcttcatccagacagacaagaccatctacaccccaggctccactgttttctatcggatcttcactgtggacaacaacctattgcctgtgggcaagacagtcgtcatcgtcattgagaccccggacggcgttcccatcaagagagacattctatcttcccacaaccaatatggcatcttgcctttgtcttggaacattccagaactggtcaacatggggcagtggaagatccgagccttctatgaacatgcaccaaagcagaccttctctgcagagtttgaggtgaaggaatacgtgctgcccagtttcgaagtcctggtggagcctacagagaaattttattacatccatggaccaaagggcctggaagtttccatcacagccagattcctgtatgggaagaacgtggacgggacagctttcgtgatctttggggtccaggatgaggataagaagatttctctggccctgtccctcacccgcgtgctgatcgaggatggttcaggggaggcagtgctcagccgaaaagtgctgatggacggggtacggccctccagcccagaagccctagtggggaagtccctgtacgtctctgtcactgttatcctgcactcaggtagcgacatggtagaggcagagcgcagtgggatcccaattgtcacttccccgtaccagatccacttcaccaagacacccaaattcttcaagccagccatgcctttcgacctcatggtgtttgtgaccaaccctgatggctctccagcccgaagagtgccagtagtcactcagggatccgacgcgcaggctctcacccaggatgacggtgtggccaagctgagcgtcaacacacccaacaaccgccaacccctgactatcacggtaagcaccaagaaggagggtatcccggacgcgcggcaggccaccaggacgatgcaggcccagccctacagcactatgcacaattccaacaactacctgcacttgtcagtgtctcgggtggagctcaagcctggggacaacctcaatgtcaacttccacctgcgcacggacgctggccaagaggccaagatccgatactacacctatctggttatgaacaaggggaagttactgaaggcaggccgtcaggttcgggagcctggccaggacctggtggtcttgtcactgcccatcactccagaatttataccttccttccgcctggtggcttactacaccctgattggagctaatggccaaagggaggtggtggccgactcagtgtgggtggatgtgaaggactcctgtgtaggcacgctggtggtgaaaggtgacccaagagataaccgacagcccgcgcctgggcatcaaacgacactaaggatcgaggggaaccagggggcccgagtggggctagtggctgtggacaagggggtgtttgtgctgaacaagaagaacaaactcacacagagcaagatctgggatgtagtagagaaggcagacattggctgcaccccaggcagtgggaagaactatgcgggtgtcttcatggatgctggcctgaccttcaagacaaaccaaggcctgcagactgatcagagagaagatcctgagtgcgccaagccagctgcccgccgccgtcgctcagtgcagttgatggaaaggaggatggacaaagctggtcagtacaccgacaagggtctgcggaagtgttgtgaggatggcatgcgtgatatccctatgccgtacagctgccagcgccgggctcgcctcatcacccagggcgagagctgcctgaaggccttcatggactgctgcaactatatcaccaagcttcgtgagcagcacagaagagaccatgtgctgggcctggccaggagtgatgtggatgaagacataatcccagaagaagatattatctctagaagccacttcccagagagctggttgtggaccatagaagagttgaaagaaccagagaaaaatggaatctctacgaaggtcatgaacatctttctcaaagattccatcaccacctgggagattctggcagtgagcttgtccgacaagaaagggatttgtgtggcagacccctatgagatcacagtgatgcaggacttcttcattgacctgcgactgccctactctgtggtgcgcaatgaacaggtggagatcagagctgtgctcttcaattaccgtgaacaggagaaacttaaggtaagggtggaactgttgcataacccagccttctgcagcatggccactgccaagaagcggtactaccagaccatcgaaatccctcccaagtcctctgtggctgtgccttatgtcattgtccccttgaagatcggcctccaggaggtggaggtcaaggccgccgtcttcaaccacttcatcagtgatggtgtcaagaagatactgaaggtcgtgccagaaggaatgagagtcaacaaaactgtggctgtccgtacactggatccagaacacctcaatcaagggggagtgcagagggaggatgtgaatgcagcagacctcagtgaccaagtgccagacacagattctgagaccagaattctcctgcaagggaccccggtggctcagatggccgaggacgctgtggacggggagcggctgaaacacctgatcgtgaccccctctggctgtggggagcagaacatgattggcatgacacccacggtcattgcagtacactatctggatcagaccgaacagtgggagaaattcggcctagagaagaggcaagaagctctggagctcatcaagaaagggtacacccagcagctggctttcaaacagcccatctctgcctatgctgccttcaacaaccggcctcccagcacctggctgacagctatgtggtcaaggtctttctctctggctgccaacctcatcgccatcgactctcaggtcctgtgtggggctgtcaaatggctgattctggagaaacagaagccagatggtgtctttcaggaggacggaccagtgattcaccaagaaatgattggtggcttccggaacaccaaggaggcagatgtgtcgcttacagcctttgtcctcatcgcactgcaggaagccagagatatctgtgaggggcaggtcaacagccttcccgggagcatcaacaaggcaggggagtatcttgaagccagttacctgaacctgcagagaccatacacagtagccattgctgggtatgccctggccctgatgaacaaactggaggaaccttacctcaccaagtttctgaacacagccaaagatcggaaccgctgggaggagcctggccagcagctctacaatgtggaggccacctcctacgccctcctggccctgctgctgctgaaagactttgactctgtgcctcctgtggtgcgctggctcaacgacgaaagatactacggaggtggctatggctccacgcaggctaccttcatggtattccaagccttggctcaataccgggcagatgtccctgaccacaaggacttgaacatggatgtgtccctccacctccccagccgcagctccccaactgtgtttcgcctgctatgggaaagtggcagtctcctgagatcagaagagaccaagcagaatgagggcttttctctgacagccaaaggaaaaggccaaggcacactgtcggtggtgacagtgtatcacgccaaagtcaaaggcaaaaccacctgcaagaagtttgacctcagggtcaccataaaaccagcccctgagacagccaagaagccccaggatgccaagagttcgatgatccttgacatctgcaccaggtacttgggagacgtggatgctactatgtccatcctggacatctccatgatgactggctttattccagacacaaacgacctggaactgctgagctctggagtagacagatacatttccaagtatgagatggacaaagccttctccaacaagaacaccctcatcatctacctagaaaagatctcacactccgaagaagactgcctgtccttcaaagtccaccagttctttaacgtgggacttatccagccggggtcggtcaaggtctactcctactacaatctagaggagtcatgcacccggttctatcatccggagaaggacgatggaatgctgagcaagctgtgccacaatgaaatgtgccgctgtgccgaggagaactgcttcatgcatcagtcacaggatcaggtcagcctgaatgaacgactagacaaggcttgtgagcctggagtggactacgtgtacaagaccaagctaacgacgatagagctgtcggatgattttgatgagtacatcatgaccatcgagcaggtcatcaagtcaggctcagatgaggtgcaggcaggtcaggaacgaaggttcatcagccacgtcaagtgcagaaacgccctaaagctgcagaaagggaagcagtacctcatgtggggcctctcctccgacctctggggagaaaagcccaataccagctacatcattgggaaggacacgtgggtggagcactggcccgaggcagaggaacgtcaggatcagaagaaccagaaacagtgcgaagacctcggggcattcacagaaacaatggtggttttcggctgccccaactgaccaccacctccaataaagcttcagttgtatttt

SEQ ID NO: 7 - DNA, 5444 bp, Homo sapiens
ctacctccaaccatgggccttttgggaatactttgttttttaatcttcctggggaaaacctggggacaggagcaaacatatgtcatttcagcaccaaaaatattccgtgttggagcatctgaaaatattgtgattcaagtttatggatacactgaagcatttgatgcaacaatctctattaaaagttatcctgataaaaaatttagttactcctcaggccatgttcatttatcctcagagaataaattccaaaactctgcaatcttaacaatacaaccaaaacaattgcctggaggacaaaacccagtttcttatgtgtatttggaagttgtatcaaagcatttttcaaaatcaaaaagaatgccaataacctatgacaatggatttctcttcattcatacagacaaacctgtttatactccagaccagtcagtaaaagttagagtttattcgttgaatgacgacttgaagccagccaaaagagaaactgtcttaaccttcatagatcctgaaggatcagaagttgacatggtagaagaaattgatcatattggaattatctcttttcctgacttcaagattccgtctaatcctagatatggtatgtggacgatcaaggctaaatataaagaggacttttcaacaactggaaccgcatattttgaagttaaagaatatgtcttgccacatttttctgtctcaatcgagccagaatataatttcattggttacaagaactttaagaattttgaaattactataaaagcaagatatttttataataaagtagtcactgaggctgacgtttatatcacatttggaataagagaagacttaaaagatgatcaaaaagaaatgatgcaaacagcaatgcaaaacacaatgttgataaatggaattgctcaagtcacatttgattctgaaacagcagtcaaagaactgtcatactacagtttagaagatttaaacaacaagtacctttatattgctgtaacagtcatagagtctacaggtggattttctgaagaggcagaaatacctggcatcaaatatgtcctctctccctacaaactgaatttggttgctactcctcttttcctgaagcctgggattccatatcccatcaaggtgcaggttaaagattcgcttgaccagttggtaggaggagtcccagtaatactgaatgcacaaacaattgatgtaaaccaagagacatctgacttggatccaagcaaaagtgtaacacgtgttgatgatggagtagcttcctttgtgcttaatctcccatctggagtgacggtgctggagtttaatgtcaaaactgatgctccagatcttccagaagaaaatcaggccagggaaggttaccgagcaatagcatactcatctctcagccaaagttacctttatattgattggactgataaccataaggctttgctagtgggagaacatctgaatattattgttacccccaaaagcccatatattgacaaaataactcactataattacttgattttatccaagggcaaaattatccattttggcacgagggagaaattttcagatgcatcttatcaaagtataaacattccagtaacacagaacatggttccttcatcccgacttctggtctattatatcgtcacaggagaacagacagcagaattagtgtctgattcagtctggttaaatattgaagaaaaatgtggcaaccagctccaggttcatctgtctcctgatgcagatgcatattctccaggccaaactgtgtctcttaatatggcaactggaatggattcctgggtggcattagcagcagtggacagtgctgtgtatggagtccaaagaggagccaaaaagcccttggaaagagtatttcaattcttagagaagagtgatctgggctgtggggcaggtggtggcctcaacaatgccaatgtgttccacctagctggacttaccttcctcactaatgcaaatgcagatgactcccaagaaaatgatgaaccttgtaaagaaattctcaggccaagaagaacgctgcaaaagaagatagaagaaatagctgctaaatataaacattcagtagtgaagaaatgttgttacgatggagcctgcgttaataatgatgaaacctgtgagcagcgagctgcacggattagtttagggccaagatgcatcaaagctttcactgaatgttgtgtcgtcgcaagccagctccgtgctaatatctctcataaagacatgcaattgggaaggctacacatgaagaccctgttaccagtaagcaagccagaaattcggagttattttccagaaagctggttgtgggaagttcatcttgttcccagaagaaaacagttgcagtttgccctacctgattctctaaccacctgggaaattcaaggcattggcatttcaaacactggtatatgtgttgctgatactgtcaaggcaaaggtgttcaaagatgtcttcctggaaatgaatataccatattctgttgtacgaggagaacagatccaattgaaaggaactgtttacaactataggacttctgggatgcagttctgtgttaaaatgtctgctgtggagggaatctgcacttcggaaagcccagtcattgatcatcagggcacaaagtcctccaaatgtgtgcgccagaaagtagagggctcctccagtcacttggtgacattcactgtgcttcctctggaaattggccttcacaacatcaatttttcactggagacttggtttggaaaagaaatcttagtaaaaacattacgagtggtgccagaaggtgtcaaaagggaaagctattctggtgttactttggatcctaggggtatttatggtaccattagcagacgaaaggagttcccatacaggatacccttagatttggtccccaaaacagaaatcaaaaggattttgagtgtaaaaggactgcttgtaggtgagatcttgtctgcagttctaagtcaggaaggcatcaatatcctaacccacctccccaaagggagtgcagaggcggagctgatgagcgttgtcccagtattctatgtttttcactacctggaaacaggaaatcattggaacatttttcattctgacccattaattgaaaagcagaaactgaagaaaaaattaaaagaagggatgttgagcattatgtcctacagaaatgctgactactcttacagtgtgtggaagggtggaagtgctagcacttggttaacagcttttgctttaagagtacttggacaagtaaataaatacgtagagcagaaccaaaattcaatttgtaattctttattgtggctagttgagaattatcaattagataatggatctttcaaggaaaattcacagtatcaaccaataaaattacagggtaccttgcctgttgaagcccgagagaacagcttatatcttacagcctttactgtgattggaattagaaaggctttcgatatatgccccctggtgaaaatcgacacagctctaattaaagctgacaactttctgcttgaaaatacactgccagcccagagcacctttacattggccatttctgcgtatgctctttccctgggagataaaactcacccacagtttcgttcaattgtttcagctttgaagagagaagctttggttaaaggtaatccacccatttatcgtttttggaaagacaatcttcagcataaagacagctctgtacctaacactggtacggcacgtatggtagaaacaactgcctatgctttactcaccagtctgaacttgaaagatataaattatgttaacccagtcatcaaatggctatcagaagagcagaggtatggaggtggcttttattcaacccaggacaccatcaatgccattgagggcctgacggaatattcactcctggttaaacaactccgcttgagtatggacatcgatgtttcttacaagcataaaggtgccttacataattataaaatgacagacaagaatttccttgggaggccagtagaggtgcttctcaatgatgacctcattgtcagtacaggatttggcagtggcttggctacagtacatgtaacaactgtagttcacaaaaccagtacctctgaggaagtttgcagcttttatttgaaaatcgatactcaggatattgaagcatcccactacagaggctacggaaactctgattacaaacgcatagtagcatgtgccagctacaagcccagcagggaagaatcatcatctggatcctctcatgcggtgatggacatctccttgcctactggaatcagtgcaaatgaagaagacttaaaagcccttgtggaaggggtggatcaactattcactgattaccaaatcaaagatggacatgttattctgcaactgaattcgattccctccagtgatttcctttgtgtacgattccggatatttgaactctttgaagttgggtttctcagtcctgccactttcacagtttacgaataccacagaccagataaacagtgtaccatgttttatagcacttccaatatcaaaattcagaaagtctgtgaaggagccgcgtgcaagtgtgtagaagctgattgtgggcaaatgcaggaagaattggatctgacaatctctgcagagacaagaaaacaaacagcatgtaaaccagagattgcatatgcttataaagttagcatcacatccatcactgtagaaaatgtttttgtcaagtacaaggcaacccttctggatatctacaaaactggggaagctgttgctgagaaagactctgagattaccttcattaaaaaggtaacctgtactaacgctgagctggtaaaaggaagacagtacttaattatgggtaaagaagccctccagataaaatacaatttcagtttcaggtacatctaccctttagattccttgacctggattgaatactggcctagagacacaacatgttcatcgtgtcaagcatttttagctaatttagatgaatttgccgaagatatctttttaaatggatgctaaaattcctgaagttcagctgcatacagtttgcacttatggactcctgttgttgaagttcgtttttttgttttcttctttttttaaacattcatagctggtcttatttgtaaagctcactttacttagaattagtggcacttgcttttattagagaatgatttcaaatgctgtaactttctgaaataacatggccttggagggcatgaagacagatactcctccaaggttattggacaccggaaacaataaattggaacacctcctcaaacctaccactcaggaatgtttgctggggccgaaagaacagtccattgaaagggagtattacaaaaacatggcctttgcttgaaagaaaataccaaggaacaggaaactgatcattaaagcctgagtttgctttc

SEQ ID NO: 8 - DNA, 4252 bp, Mus musculus
aagtctttccctgctgtgaccacagttcatagcagagaggaactggatggtacagcacagatttctcttggagtcagttggtcccagaaagatccaaattatgagactgtcagcaagaattatttggcttatattatggactgtttgtgcagcagaagattgtaaaggtcctcctccaagagaaaattcagaaattctctcaggctcgtggtcagaacaactatatccagaaggcacccaggctacctacaaatgccgccctggataccgaacacttggcactattgtaaaagtatgcaagaatggaaaatgggtggcgtctaacccatccaggatatgtcggaaaaagccttgtgggcatcccggagacacaccctttgggtcctttaggctggcagttggatctcaatttgagtttggtgcaaaggttgtttatacctgtgatgatgggtatcaactattaggtgaaattgattaccgtgaatgtggtgcagatggctggatcaatgatattccactatgtgaagttgtgaagtgtctacctgtgacagaactcgagaatggaagaattgtgagtggtgcagcagaaacagaccaggaatactattttggacaggtggtgcggtttgaatgcaattcaggcttcaagattgaaggacataaggaaattcattgctcagaaaatggcctttggagcaatgaaaagccacgatgtgtggaaattctctgcacaccaccgcgagtggaaaatggagatggtataaatgtgaaaccagtttacaaggagaatgaaagataccactataagtgtaagcatggttatgtgcccaaagaaagaggggatgccgtctgcacaggctctggatggagttctcagcctttctgtgaagaaaagagatgctcacctccttatattctaaatggtatctacacacctcacaggattatacacagaagtgatgatgaaatcagatatgaatgtaattatggcttctatcctgtaactggatcaactgtttcaaagtgtacacccactggctggatccctgttccaagatgtaccttgaaaccatgtgaatttccacaattcaaatatggacgtctgtattatgaagagagcctgagacccaacttcccagtatctataggaaataagtacagctataagtgtgacaacgggttttcaccaccttctgggtattcctgggactaccttcgttgcacagcacaagggtgggagcctgaagtcccatgcgtcaggaaatgtgttttccattatgtggagaatggagactctgcatactgggaaaaagtatatgtgcagggtcagtctttaaaagtccagtgttacaatggctatagtcttcaaaatggtcaagacacaatgacatgtacagagaatggctggtcccctcctcccaaatgcatccgtatcaagacatgttcagcatcagatatacacattgacaatggatttctttctgaatcttcttctatatatgctctaaatagagaaacatcctatagatgtaagcagggatatgtgacaaatactggagaaatatcaggatcaataacttgccttcaaaatggatggtcacctcaaccctcatgcattaagtcttgtgatatgcctgtatttgagaattctataactaagaatactaggacatggtttaagctcaatgacaaattagactatgaatgtctcgttggatttgaaaatgaatataaacataccaaaggctctataacatgtacttattatggatggtctgatacaccctcatgttatgaaagagaatgcagtgttcccactctagaccgaaaactagtcgtttcccccagaaaagaaaaatacagagttggagatttgttggaattctcctgccattcaggacacagagttgggccagattcagtgcaatgctaccactttggatggtctcctggtttccctacatgtaaaggtcaagtagcatcatgtgcaccacctcttgaaattcttaatggggaaattaatggagcaaaaaaagttgaatacagccatggtgaagtggtgaaatatgattgcaaacctagattcctactgaagggacccaataaaatccagtgtgttgatgggaattggacaaccttgcctgtatgtattgaggaggagagaacatgtggagacattcctgaacttgaacatggctctgccaagtgttctgttcctccctaccaccatggagattcagtggagttcatttgtgaagaaaacttcacaatgattggacatgggtcagtttcttgcattagtggaaaatggacccagcttcctaaatgtgttgcaacagaccaactggagaagtgtagagtgctgaagtcaactggcatagaagcaataaaaccaaaattgactgaatttacgcataactccaccatggattacaaatgtagagacaagcaggagtacgaacgctcaatctgtatcaatggaaaatgggatcctgaaccaaactgtacaagcaaaacatcctgccctcctccaccgcagattccaaatacccaagtgattgaaaccaccgtgaaatacttggatggagaaaaattatctgttctttgccaagacaattacctaactcaggactcagaagaaatggtgtgcaaagatggaaggtggcagtcattacctcgctgcattgaaaaaattccatgttcccagccccctacaatagaacatggatctattaatttacccagatcttcagaagaaaggagagattccattgagtccagcagtcatgaacatggaactacattcagctatgtctgtgatgatggtttcaggatacctgaagaaaataggataacctgctacatgggaaaatggagcactccacctcgctgtgttggacttccttgtggacctccaccttcaattcctcttggtactgtttctcttgagctagagagttaccaacatggggaagaggttacataccattgttctacaggctttggaattgatggaccagcatttattatatgcgaaggaggaaagtggtctgacccaccaaaatgcataaaaacggattgtgacgttttacccacagttaaaaatgccataataagaggaaagagcaaaaaatcatataggacaggagaacaagtgacattcagatgtcaatctccttatcaaatgaatggctcagacactgtgacatgtgttaatagtcggtggattggacagccagtatgcaaagataattcctgtgtggatccaccacatgtgccaaatgctactatagtaacaaggaccaagaataaatatctacatggtgacagagtacgttatgaatgtaataaacctttggaactatttgggcaagtggaagtgatgtgtgaaaatgggatatggacagaaaaaccaaagtgccgagactcaacagggaaatgtgggcctcctccacctattgacaatggagacatcacctccttgtcattaccagtatatgaaccattatcatcagttgaatatcaatgccagaagtattatctccttaagggaaagaagacaataacatgtacaaatggaaagtggtctgagccaccaacatgcttacatgcatgtgtaataccagaaaacattatggaatcacacaatataattctcaaatggagacacactgaaaagatttattcccattcaggggaggatattgaatttggatgtaaatatggatattataaagcaagagattcaccgccatttcgtacaaagtgcattaatggcaccatcaattatcccacttgtgtataaaatcataatacatttattagttgattttattgtttagaaaggcacatgcatgtgactaatatactttcaatttgcattgaagtattgtttaactcatgtcttctcataaatataaacatttttgttatatggtgattaacttgtaactttaaaaactattgccaaaatgcaaaagcagtaattcaaaactcctaatctaaaatatgatatgtccaaggacaaactatttcaatcaagaaagtagatgtaagttcttcaacatctgtttctattcagaactttctcagattttcctggataccttttgatgtaaggtcctgatttacagtggataaaggatatattgactgattcttcaaattaatatgatttcccaaagcatgtaacaaccaaactatcatatattatatgactaatgcatacaattaattactatataatactttc

SEQ ID NO: 9 - DNA, 3926 bp, Homo sapiens
aattcttggaagaggagaactggacgttgtgaacagagttagctggtaaatgtcctcttaaaagatccaaaaaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagccaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattagatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagaatcaatcataaagtgcacacctttattcagaactttagtattaaatcagttctcaatttcattttttatgtattgttttactcctttttattcatacgtaaaattttggattaatttgtgaaaatgtaattataagctgagaccggtggctctctt

SEQ ID NO: 10 - DNA, 3220 bp, Mus musculus
atgcttacatggttccttttctatttttcagagatttcttgtgaccctcctcctgaagtcaaaaatgctcggaaaccctattattctcttcccatagttcctggaactgttctgaggtacacttgttcacctagctaccgcctcattggagaaaaggctatcttttgtataagtgaaaatcaagtgcatgccacctgggataaagctcctcctatatgtgaatctgtgaataaaaccatttcttgctcagatcccatagtaccagggggattcatgaataaaggatctaaggcaccattcagacatggtgattctgtgacatttacctgtaaagccaacttcaccatgaaaggaagcaaaactgtctggtgccaggcaaatgaaatgtggggaccaacagctctgccagtctgtgagagtgatttccctctggagtgcccatcacttccaacgattcataatggacaccacacaggacagcatgttgaccagtttgttgctgggttgtctgtgacatacagttgtgaacctggctatttgctcactggaaaaaagacaattaagtgcttatcttcaggagactgggatggtgtcatcccgacatgcaaagaggcccagtgtgaacatccaggaaagtttcccaatgggcaggtaaaggaacctctgagccttcaggttggcacaactgtgtacttctcctgtaatgaagggtaccaattacaaggacaaccctctagtcagtgtgtaattgttgaacagaaagccatctggactaagaagccagtatgtaaagaaattctctgcccaccacctccacctgttcgtaatggaagtcatacaggcagcttttcagaaaatgtaccatatggaagcacagttacctacacctgtgacccaagcccagagaaaggcgtgagcttcactcttattggagagaagactatcaattgtactactggtagtcagaagactgggatctggagtggccctgctccatattgtgtactttcaacttctgcagttctgtgtttacaaccgaagatcaaaagagggcaaatattatctattttgaaagatagttattcatataatgacactgtggcattttcttgtgaacctggcttcaccttgaagggcaacaggagcattcgatgcaatgctcatggcacatgggagccaccggtaccagtgtgtgaaaaaggatgtcaggctcctcctaaaattatcaatgggcaaaaagaagatagttacttgctcaactttgaccctggtacatccataagatatagctgtgaccctggctatttactggtgggagaggacactatacattgcacccctgaggggaagtggacacccattactccccagtgcacagttgcagagtgtaagccagtaggaccacatctctttaagaggcctcagaatcagtttattaggacagctgttaattcttcttgtgatgaagggttccagttaagtgagagtgcttatcaactgtgtcaaggtacaattccttggtttatagaaatccgtctttgtaaagaaatcacctgcccaccacctcctgttatacacaacgggacacatacatggagttcctcagaagatgtcccatatggaactgtggtcacatacatgtgctatcctgggccagaggaaggcgtaaaattcaaactcatcggggagcaaaccatccactgtacaagtgacagcagaggaagaggctcctggagtagccctgctcctctctgtaaactttccctcccagctgtccagtgcacagacgttcatgttgaaaatggagtcaagctcactgacaataaagccccatatttctacaatgatagtgtgatgttcaagtgtgatgatggatatattttgagtggaagcagtcagatccggtgtaaagccaataatacctgggatcctgaaaaaccactttgtaaaaaagaaggatgtgagcctatgagagtacatggccttccagatgattcacatataaaactagtgaaaagaacctgtcaaaatgggtaccagttgactggatatacttatgagaagtgtcaaaatgctgagaatgggacttggtttaaaaagattgaagtttgtacagttattctctgtcaacctccaccaaaaattgcaaatggtggtcacacaggcatgatggcaaagcacttcctatatggaaatgaagtttcttatgaatgtgatgaagggttctatcttttgggagagaaaagtttgcagtgcgtaaatgattctaaaggtcatggctcttggagtggacctccaccacaatgcttacaatcttctcctctaactcattgccccgatccagaagtcaaacatggttacaaactcaataaaactcattctgcattttctcataatgacatagtacattttgtctgcaatcaaggcttcatcatgaacggcagccacttgataaggtgtcatactaataacacatggttaccaggtgtaccaacttgtatcagaaaggcttctttagggtgtcagtctccatccacaatccccaatgggaatcatactggtgggagtatagctcgatttccccctggaatgtcagtcatgtacagttgctaccaaggcttccttatggctggagaggcacgtcttatctgtactcatgagggtacctggagtcaacctccccctttttgcaaagaggtaaactgtagcttccctgaagatacaaatggaatccagaagggatttcaacctgggaaaacctatcgatttggggctactgtgactctggaatgtgaggatgggtataccttggagggaagtccccagagccagtgccaggatgacagccaatggaaccctcccttggctctttgcaaataccgtaggtggtcaactattcctcttatttgtggtatttctgtgggctcagcacttatcattttgatgagtgtcggcttctgtatgatattaaaacacagagaaagcaattattatacaaagacaagacccaaagaaggagctcttcatttagaaacacgagaagtatattctattgatccatataacccagcaagctgatgacatgacaaatcaagatgtagaactctcagctacctcttcagcaccatatctgcttacatgccaccaagctaccctccacgacaataatggactaaacctctgatttgtaagccagccccaattaaatgtttttctctat

SEQ ID NO: 11 - DNA, 3934 bp, Homo sapiens
gccctcccagagctgccggacgctcgcgggtctcggaacgcatcccgccgcgggggcttcggccgtggcatgggcgccgcgggcctgctcggggttttcttggctctcgtcgcaccgggggtcctcgggatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaattttttgcccatcacctccccctattctcaatggaagacatataggcaactcactagcaaatgtctcatatggaagcatagtcacttacacttgtgacccggacccagaggaaggagtgaacttcatccttattggagagagcactctccgttgtacagttgatagtcagaagactgggacctggagtggccctgccccacgctgtgaactttctacttctgcggttcagtgtccacatccccagatcctaagaggccgaatggtatctgggcagaaagatcgatatacctataacgacactgtgatatttgcttgcatgtttggcttcaccttgaagggcagcaagcaaatccgatgcaatgcccaaggcacatgggagccatctgcaccagtctgtgaaaaggaatgccaggcccctcctaacatcctcaatgggcaaaaggaagatagacacatggtccgctttgaccctggaacatctataaaatatagctgtaaccctggctatgtgctggtgggagaagaatccatacagtgtacctctgagggggtgtggacaccccctgtaccccaatgcaaagtggcagcgtgtgaagctacaggaaggcaactcttgacaaaaccccagcaccaatttgttagaccagatgtcaactcttcttgtggtgaagggtacaagttaagtgggagtgtttatcaggagtgtcaaggcacaattccttggtttatggagattcgtctttgtaaagaaatcacctgcccaccaccccctgttatctacaatggggcacacaccgggagttccttagaagattttccatatggaaccacggtcacttacacatgtaaccctgggccagaaagaggagtggaattcagcctcattggagagagcaccatccgttgtacaagcaatgatcaagaaagaggcacctggagtggccctgctcccctatgtaaactttccctccttgctgtccagtgctcacatgtccatattgcaaatggatacaagatatctggcaaggaagccccatatttctacaatgacactgtgacattcaagtgttatagtggatttactttgaagggcagtagtcagattcgttgcaaagctgataacacctgggatcctgaaataccagtttgtgaaaaagaaacatgccagcatgtgagacagagtcttcaagaacttccagctggttcacgtgtggagctagttaatacgtcctgccaagatgggtaccagttgactggacatgcttatcagatgtgtcaagatgctgaaaatggaatttggttcaaaaagattccactttgtaaagttattcactgtcaccctccaccagtgattgtcaatgggaagcacacagggatgatggcagaaaactttctatatggaaatgaagtctcttatgaatgtgaccaaggattctatctcctgggagagaaaaaattgcagtgcagaagtgattctaaaggacatggatcttggagcgggccttccccacagtgcttacgatctcctcctgtgactcgctgccctaatccagaagtcaaacatgggtacaagctcaataaaacacattctgcatattcccacaatgacatagtgtatgttgactgcaatcctggcttcatcatgaatggtagtcgcgtgattaggtgtcatactgataacacatgggtgccaggtgtgccaacttgtatgaaaaaagccttcatagggtgtccacctccgcctaagacccctaacgggaaccatactggtggaaacatagctcgattttctcctggaatgtcaatcctgtacagctgtgaccaaggctacctgctggtgggagaggcactccttctttgcacacatgagggaacctggagccaacctgcccctcattgtaaagaggtaaactgtagctcaccagcagatatggatggaatccagaaagggctggaaccaaggaaaatgtatcagtatggagctgttgtaactctggagtgtgaagatgggtatatgctggaaggcagtccccagagccagtgccaatcggatcaccaatggaaccctcccctggcggtttgcagatcccgttcacttgctcctgtcctttgtggtattgctgcaggtttgatacttcttaccttcttgattgtcattaccttatacgtgatatcaaaacacagagaacgcaattattatacagatacaagccagaaagaagcttttcatttagaagcacgagaagtatattctgttgatccatacaacccagccagctgatcagaagacaaactggtgtgtgcctcattgcttggaattcagcggaatattgattagaaagaaactgctctaatatcagcaagtctctttatatggcctcaagatcaatgaaatgatgtcataagcgatcacttcctatatgcacttattctcaagaagaacatctttatggtaaagatgggagcccagtttcactgccatatactcttcaaggactttctgaagcctcacttatgagatgcctgaagccaggccatggctataaacaattacatggctctaaaaagttttgccctttttaaggaaggcactaaaaagagctgtcctggtatctagacccatcttctttttgaaatcagcatactcaatgttactatctgcttttggttataatgtgtttttaattatctaaagtatgaagcattttctggggttatgatggccttacctttattaggaagtatggttttattttgatagtagcttcctcctctggtggtgttaatcatttcatttttacccttactgtttgagtttctctcacattactgtatatactttgcctttccataatcactcagtgattgcaatttgcacaagtttttttaaattatgggaatcaagatttaatcctagagatttggtgtacaattcaggctttggatgtttctttagcagttttgtgataagttctagttgcttgtaaaatttcacttaataatgtgtacattagtcattcaataaattgtaattgtaaagaaaa

SEQ ID NO: 12 - DNA, 3551 bp, Homo sapiens
ttgccttgtgttagctagcaataagaaaagaagctttgtttggattaacatatataccctcttcattctgcatacctattttttccccaataatttgcagcttaggtccgaggacaccacaaactctgcttaaagggcctggaggctctcaaggcatggccagacgctctgtcttgtacttcatcctgctgaatgctctgatcaacaagggccaagcctgcttctgtgatcactatgcatggactcagtggaccagctgctcaaaaacttgcaattctggaacccagagcagacacagacaaatagtagtagataagtactaccaggaaaacttttgtgaacagatttgcagcaagcaggagactagagaatgtaactggcaaagatgccccatcaactgcctcctgggagattttggaccatggtcagactgtgacccttgtattgaaaaacagtctaaagttagatctgtcttgcgtcccagtcagtttgggggacagccatgcactgagcctctggtagcctttcaaccatgcattccatctaagctctgcaaaattgaagaggctgactgcaagaataaatttcgctgtgacagtggccgctgcattgccagaaagttagaatgcaatggagaaaatgactgtggagacaattcagatgaaagggactgtgggaggacaaaggcagtatgcacacggaagtataatcccatccctagtgtacagttgatgggcaatgggtttcattttctggcaggagagcccagaggagaagtccttgataactctttcactggaggaatatgtaaaactgtcaaaagcagtaggacaagtaatccataccgtgttccggccaatctggaaaatgtcggctttgaggtacaaactgcagaagatgacttgaaaacagatttctacaaggatttaacttctcttggacacaatgaaaatcaacaaggctcattctcaagtcagggggggagctctttcagtgtaccaattttttattcctcaaagagaagtgaaaatatcaaccataattctgccttcaaacaagccattcaagcctctcacaaaaaggattctagttttattaggatccataaagtgatgaaagtcttaaacttcacaacgaaagctaaagatctgcacctttctgatgtctttttgaaagcacttaaccatctgcctctagaatacaactctgctttgtacagccgaatattcgatgactttgggactcattacttcacctctggctccctgggaggcgtgtatgaccttctctatcagtttagcagtgaggaactaaagaactcaggtttaaccgaggaagaagccaaacactgtgtcaggattgaaacaaagaaacgcgttttatttgctaagaaaacaaaagtggaacataggtgcaccaccaacaagctgtcagagaaacatgaaggttcatttatacagggagcagagaaatccatatccctgattcgaggtggaaggagtgaatatggagcagctttggcatgggagaaagggagctctggtctggaggagaagacattttctgagtggttagaatcagtgaaggaaaatcctgctgtgattgactttgagcttgcccccatcgtggacttggtaagaaacatcccctgtgcagtgacaaaacggaacaacctcaggaaagctttgcaagagtatgcagccaagttcgatccttgccagtgtgctccatgccctaataatggccgacccaccctctcagggactgaatgtctgtgtgtgtgtcagagtggcacctatggtgagaactgtgagaaacagtctccagattataaatccaatgcagtagacggacagtggggttgttggtcttcctggagtacctgtgatgctacttataagagatcgagaacccgagaatgcaataatcctgccccccaacgaggagggaaacgctgtgagggggagaagcgacaagaggaagactgcacattttcaatcatggaaaacaatggacaaccatgtatcaatgatgatgaagaaatgaaagaggtcgatcttcctgagatagaagcagattccgggtgtcctcagccagttcctccagaaaatggatttatccggaatgaaaagcaactatacttggttggagaagatgttgaaatttcatgccttactggctttgaaactgttggataccagtacttcagatgcttaccagacgggacctggagacaaggggatgtggaatgccaacggacggagtgcatcaagccagttgtgcaggaagtcctgacaattacaccatttcagagattgtatagaattggtgaatccattgagctaacttgccccaaaggctttgttgttgctgggccatcaaggtacacatgccaggggaattcctggacaccacccatttcaaactctctcacctgtgaaaaagatactctaacaaaattaaaaggccattgtcagctgggacagaaacaatcaggatctgaatgcatttgtatgtctccagaagaagactgtagccatcattcagaagatctctgtgtgtttgacacagactccaacgattactttacttcacccgcttgtaagtttttggctgagaaatgtttaaataatcagcaactccattttctacatattggttcctgccaagacggccgccagttagaatggggtcttgaaaggacaagactttcatccaacagcacaaagaaagaatcctgtggctatgacacctgctatgactgggaaaaatgttcagcctccacttccaaatgtgtctgcctattgcccccacagtgcttcaagggtggaaaccaactctactgtgtcaaaatgggatcatcaacaagtgagaaaacattgaacatctgtgaagtgggaactataagatgtgcaaacaggaagatggaaatactgcatcctggaaagtgtttggcctagcacaattactgctaggcccagcacaatgaacagatttaccatcccgaagaaccaactcctacaaatgagaattcttgcacaaacagcagactggcatgctcaaagttactgacaaaaattattttctgttagtttgagatcattattctcccctgactctcctgtttgggcatgtcttattcagttccagctcatgacgccctgtagcatacccctaggtaccaacttccacagcagtctcgtaaattctcctgttcacattgtacaaaaataatgtgacttctgaggcccttatgtagcctgtgacattaagcattctcacaattagaaataagaataaaacccataattttcttcaatgagttaataaacagaaatctccagaacctctgaaacacattcttgaagcccagctttcatatcttcattcaacaaataatttctgagtgtgtatacaggatgtcaagtactgaccaaagtcctgagaactcggcagataataaaacagacaaaagcctttgccttcatgaagcatacattcattcaggggtagacacacaaaaaatgaaataaacaggtaaaatatgtagc

SEQ ID NO: 13 - DNA, 3890 bp, Homo sapiens
atgaaggtgataagcttattcattttggtgggatttataggagagttccaaagtttttcaagtgcctcctctccagtcaactgccagtgggacttctatgccccttggtcagaatgcaatggctgtaccaagactcagactcgcaggcggtcagttgctgtgtatgggcagtatggaggccagccttgtgttggaaatgcttttgaaacacagtcctgtgaacctacaagaggatgtccaacagaggagggatgtggagagcgtttcaggtgcttttcaggtcagtgcatcagcaaatcattggtttgcaatggggattctgactgtgatgaagacagtgctgatgaagacagatgtgaggactcagaaaggagaccttcctgtgatatcgataaacctcctcctaacatagaacttactggaaatggttacaatgaactcactggccagtttaggaacagagtcatcaataccaaaagttttggtggtcaatgtagaaaggtgtttagtggggatggaaaagatttctacaggctgagtggaaatgtcctgtcctatacattccaggtgaaaataaataatgattttaattatgaattttacaatagtacttggtcttatgtaaaacatacgtcgacagaacacacatcatctagtcggaagcgctccttttttagatcttcatcatcttcttcacgcagttatacttcacataccaatgaaatccataaaggaaagagttaccaactgctggttgttgagaacactgttgaagtggctcagttcattaataacaatccagaatttttacaacttgctgagccattctggaaggagctttcccacctcccctctctgtatgactacagtgcctaccgaagattaatcgaccagtacgggacacattatctgcaatctgggtcgttaggaggagaatacagagttctattttatgtggactcagaaaaattaaaacaaaatgattttaattcagtcgaagaaaagaaatgtaaatcctcaggttggcattttgtcgttaaattttcaagtcatggatgcaaggaactggaaaacgctttaaaagctgcttcaggaacccagaacaatgtattgcgaggagaaccgttcatcagagggggaggtgcaggcttcatatctggccttagttacctagagctggacaatcctgctggaaacaaaaggcgatattctgcctgggcagaatctgtgactaatcttcctcaagtcataaaacaaaagctgacacctttatatgagctggtaaaggaagtaccttgtgcctctgtgaaaaaactatacctgaaatgggctcttgaagagtatctggatgaatttgacccctgtcattgccggccttgtcaaaatggtggtttggctactgttgaggggacccattgtctgtgccattgcaaaccgtacacatttggtgcggcgtgtgagcaaggagtcctcgtagggaatcaagcaggaggggttgatggaggttggagttgctggtcctcttggagcccctgtgtccaagggaagaaaacaagaagccgtgaatgcaataacccacctcccagtgggggtgggagatcctgcgttggagaaacgacagaaagcacacaatgcgaagatgaggagctggagcacttgaggttgcttgaaccacattgctttcctttgtctttggttccaacagaattctgtccatcacctcctgccttgaaagatggatttgttcaagatgaaggtccaatgtttcctgtggggaaaaatgtagtgtacacttgcaatgaaggatactctcttattggaaacccagtggccagatgtggagaagatttacggtggcttgttggggaaatgcattgtcagaaaattgcctgtgttctacctgtactgatggatggcatacagagtcacccccaaaaacctttctacacagttggtgagaaggtgactgtttcctgttcaggtggcatgtccttagaaggtccttcagcatttctctgtggctccagccttaagtggagtcctgagatgaagaatgcccgctgtgtacaaaaagaaaatccgttaacacaggcagtgcctaaatgtcagcgctgggagaaactgcagaattcaagatgtgtttgtaaaatgccctacgaatgtggaccttccttggatgtatgtgctcaagatgagagaagcaaaaggatactgcctctgacagtttgcaagatgcatgttctccactgtcagggtagaaattacacccttactggtagggacagctgtactctgcctgcctcagctgagaaagcttgtggtgcctgcccactgtggggaaaatgtgatgctgagagcagcaaatgtgtctgccgagaagcatcggagtgcgaggaagaagggtttagcatttgtgtggaagtgaacggcaaggagcagacgatgtctgagtgtgaggcgggcgctctgagatgcagagggcagagcatctctgtcaccagcataaggccttgtgctgcggaaacccagtaggctcctggaggccatggtcagcttgcttggaatccagcaggcagctggggctgagtgaaaacatctgcacaactgggcactggacagcttttccttcttctccagtgtctaccttcctcctcaactcccagccatctgtataaacacaatcctttgttctcccaaatctgaatcgaattactcttttgcctcctttttaatgtcagtaaggatatgagcctttgcacaggctggctgcgtgttcttgaaataggtgttaccttctctgggccttggttttttaaaatctgtaaaattagaggattgcactagagaaacttgaatgctccattcaggcctatcattttattaagtatgattgacacagcccatgggccagaacacactctacaaaatgactaggataacagaaagaacgtgatctcctgattagagagggtggttttcctcaatggaaccaaatataaagaggacttgaacaaaaatgacagatacaaactatttctatcctgagtagtaatctcacacttcatcctatagagtcaaccaccacagataggaattccttattctttttttaatttttttaagacagagtctcactttgttgcccaggctggagcgcagtggggtgatctcatctccctgcaacctccgcctcctgggttgaagcgattcttgtgcctcagcttcccaagcagctgggattacaggtgcccgccaccacgcccagctaatttttgcatttttagtagagatgggtttcaccatgttggccatgctcgtctccaactcctgacctcaggtaatccgtctgccttggcctcccaaatgctgggattacagacatgaaccaccacgcctggctggaatacttactcttgtcgggagattgaaccactaaaatgttagagcagaattcattatgctgtggtcacaggggtgtcttgtctgagaacaaatacaattcagtcttctctttggggttttagtatgtgtcaaacataggactggaagtttgcccctgttcttttttcttttgaaagaacatcagttcatgcctgaggcatgagtgactgtgcatttgagatagttttccctattctgtggatacagtcccagagttttcagggagtacacaggtagattagtttgaagcattgaccttttatttattccttatttctctttcatcaaaacaaaacagcagctgtgggaggagaaatgagagggcttaaatgaaatttaaaataagctatattatacaaatactatctctgtattgttctgaccctggtaaatatatttcaaaacttcagatgacaaggattagaacactcattaagatgctattcttc

SEQ ID NO: 14 - DNA, 2447 bp, Homo sapiens
caggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaac

SEQ ID NO: 15 - DNA, 2609 bp, Homo sapiens
ggctctctacctctcgccgcccctagggaggacaccatgggcccactgatggttcttttttgcctgctgttcctgtacccaggtctggcagactcggctccctcctgccctcagaacgtgaatatctcgggtggcaccttcaccctcagccatggctgggctcctgggagccttctcacctactcctgcccccagggcctgtacccatccccagcatcacggctgtgcaagagcagcggacagtggcagaccccaggagccacccggtctctgtctaaggcggtctgcaaacctgtgcgctgtccagcccctgtctcctttgagaatggcatttataccccacggctggggtcctatcccgtgggtggcaatgtgagcttcgagtgtgaggatggcttcatattgcggggctcgcctgtgcgtcagtgtcgccccaacggcatgtgggatggagaaacagctgtgtgtgataatggggctggccactgccccaacccaggcatttcactgggcgcagtgcggacaggcttccgctttggtcatggggacaaggtccgctatcgctgctcctcgaatcttgtgctcacggggtcttcggagcgggagtgccagggcaacggggtctggagtggaacggagcccatctgccgccaaccctactcttatgacttccctgaggacgtggcccctgccctgggcacttccttctcccacatgcttggggccaccaatcccacccagaagacaaaggaaagcctgggccgtaaaatccaaatccagcgctctggtcatctgaacctctacctgctcctggactgttcgcagagtgtgtcggaaaatgactttctcatcttcaaggagagcgcctccctcatggtggacaggatcttcagctttgagatcaatgtgagcgttgccattatcacctttgcctcagagcccaaagtcctcatgtctgtcctgaacgacaactcccgggatatgactgaggtgatcagcagcctggaaaatgccaactataaagatcatgaaaatggaactgggactaacacctatgcagccttaaacagtgtctatctcatgatgaacaaccaaatgcgactcctcggcatggaaacgatggcctggcaggaaatccgacatgccatcatccttctgacagatggaaagtccaatatgggtggctctcccaagacagctgttgaccatatcagagagatcctgaacatcaaccagaagaggaatgactatctggacatctatgccatcggggtgggcaagctggatgtggactggagagaactgaatgagctagggtccaagaaggatggtgagaggcatgccttcattctgcaggacacaaaggctctgcaccaggtctttgaacatatgctggatgtctccaagctcacagacaccatctgcggggtggggaacatgtcagcaaacgcctctgaccaggagaggacaccctggcatgtcactattaagcccaagagccaagagacctgccggggggccctcatctccgaccaatgggtcctgacagcagctcattgcttccgcgatggcaacgaccactccctgtggagggtcaatgtgggagaccccaaatcccagtggggcaaagaattccttattgagaaggcggtgatctccccagggtttgatgtctttgccaaaaagaaccagggaatcctggagttctatggtgatgacatagctctgctgaagctggcccagaaagtaaagatgtccacccatgccaggcccatctgccttccctgcacgatggaggccaatctggctctgcggagacctcaaggcagcacctgtagggaccatgagaatgaactgctgaacaaacagagtgttcctgctcattttgtcgccttgaatgggagcaaactgaacattaaccttaagatgggagtggagtggacaagctgtgccgaggttgtctcccaagaaaaaaccatgttccccaacttgacagatgtcagggaggtggtgacagaccagttcctatgcagtgggacccaggaggatgagagtccctgcaagggagaatctgggggagcagttttccttgagcggagattcaggttttttcaggtgggtctggtgagctggggtctttacaacccctgccttggctctgctgacaaaaactcccgcaaaagggcccctcgtagcaaggtcccgccgccacgagactttcacatcaatctcttccgcatgcagccctggctgaggcagcacctgggggatgtcctgaattttttacccctctagccatggccactgagccctctgctgccctgccagaatctgccgcccctccatcttctacctctgaatggccacccttagaccctgtgatccatcctctctcctagctgagtaaatccgggtctctaggatgccagaggcagcgcacacaagctgggaaatcctcagggctcctaccagcaggactgcctcgctgccccacctcccgctccttggcctgtccccagattccttccctggttgacttgactcatgcttgtttcactttcacatggaatttcccagttatgaaattaataaaaatcaatggtttccac

SEQ ID NO: 16 - DNA, 2443 bp, Homo sapiens
tgtagccagatccagcatttgggtttcagtttggacaggaggtcaaataggcacccagagtgacctggagagggctttgggccactggactctctggtgctttccatgacaatggagagcccccagctctgcctcgtcctcttggtcttaggcttctcctctggaggtgtgagcgcaactccagtgcttgaggcccggccccaagtctcctgctctctggagggagtagagatcaaaggcggctcctttcaacttctccaaggcggtcaggccctggagtacctatgtccctctggcttctacccataccccgtgcagactcgaacctgcagatccacaggctcctggagcgacctgcagacccgagaccaaaagattgtccagaaggcggaatgcagagcaatacgctgcccacgaccgcaggactttgaaaatggggaattctggccccggtcccccttctacaacctgagtgaccagatttcttttcaatgctatgatggttacgttctccggggctctgctaatcgcacctgccaagagaatggccggtgggatgggcaaacagcaatttgtgatgatggagctggatactgtcccaatcccggtattcctattgggacaaggaaggtgggtagccaataccgccttgaagacattgttacttaccactgcagccggggacttgtcctgcgtggctcccagaagcgaaagtgtcaagaaggtggctcatggagtgggacagagccttcctgccaagattccttcatgtatgacagccctcaagaagtggccgaagcattcctatcctccctgacagagaccatcgaaggagccgatgctgaggatgggcacagcccaggagaacagcagaagaggaagattgtcctagacccctcgggctccatgaatatctacctggtgctagatggatcagacagcatcggaagcagcaacttcacaggggctaagcggtgcctcaccaacttgattgagaaggtggcgagttacggggtgaggccacgatatggtctcctgacatatgctacagtccccaaagtgttggtcagagtgtctgatgagaggagtagcgatgccgactgggtcacagagaagctcaaccaaatcagttatgaagaccacaagctgaagtcagggaccaacaccaagagggctctccaggctgtgtatagcatgatgagctgggcaggggatgccccgcctgaaggctggaatagaacccgccatgtcatcatcattatgactgatggcttgcacaacatgggtggaaaccctgtcactgtcattcaggacatccgagccttgctggacatcggcagggatcccaaaaatcccagggaggattacctggatgtgtatgtgtttggggtcgggcctctggtggactccgtgaacatcaatgccttagcttccaaaaaggacaatgagcatcatgtgtttaaagtcaaggatatggaagacctggagaatgttttctaccaaatgattgatgaaaccaaatctctgagtctctgtggcatggtgtgggagcataaaaaaggcaacgattatcataagcaaccatggcaagccaagatctcagtcactcgccctctgaaaggacatgagacctgtatgggggccgtggtgtctgagtacttcgtgctgacagcagcgcactgcttcatggtggatgatcagaaacattccatcaaggtcagcgtggggggtcagaggcgggacctggagattgaagaggtcctgttccaccccaaatacaatattaatgggaaaaaggcagaagggatccctgagttctatgattatgatgtggccctagtcaagctcaagaacaagctcaagtatggccagactctcaggcccatctgtctcccctgcacggagggaaccacacgagccttgaggcttcctcagacagccacctgcaagcagcacaaggaacagttgctccctgtgaaggatgtcaaagctctgtttgtatctgagcaagggaagagcctgactcggaaggaggtgtacatcaagaatggggacaagaaagccagttgtgagagagatgctacaaaggcccaaggctatgagaaggtcaaagatgcctctgaggtggtcactccacggttcctctgcacaggaggggtggatccctatgctgaccccaacacatgcaaaggagattccgggggccctctcattgttcacaagagaagccgcttcattcaagttggtgtgattagctggggagtagtagatgtctgcagagaccagaggcggcaacagctggtaccctcttatgcccgggacttccacatcaacctcttccaggtgctgccctggctaaaggacaagctcaaagatgaggatttgggttttctataaagagcttcctgcagggagagtgtgaggacagattaaagcagttaca

SEQ ID NO: 17 - DNA, 2493 bp, Homo sapiens
ggatcgatttgagtaagagcatagctgtcgggagagcccaggattcaacacgggccttgagaaatgtggctcttgtacctcctggtgccggccctgttctgcagggcaggaggctccattcccatccctcagaagttatttggggaggtgacttcccctctgttccccaagccttaccccaacaactttgaaacaaccactgtgatcacagtccccacgggatacagggtgaagctcgtcttccagcagtttgacctggagccttctgaaggctgcttctatgattatgtcaagatctctgctgataagaaaagcctggggaggttctgtgggcaactgggttctccactgggcaaccccccgggaaagaaggaatttatgtcccaagggaacaagatgctgctgaccttccacacagacttctccaacgaggagaatgggaccatcatgttctacaagggcttcctggcctactaccaagctgtggaccttgatgaatgtgcttcccggagcaaatcaggggaggaggatccccagccccagtgccagcacctgtgtcacaactacgttggaggctacttctgttcctgccgtccaggctatgagcttcaggaagacaggcattcctgccaggctgagtgcagcagcgagctgtacacggaggcatcaggctacatctccagcctggagtaccctcggtcctacccccctgacctgcgctgcaactacagcatccgggtggagcggggcctcaccctgcacctcaagttcctggagccttttgatattgatgaccaccagcaagtacactgcccctatgaccagctacagatctatgccaacgggaagaacattggcgagttctgtgggaagcaaaggccccccgacctcgacaccagcagcaatgctgtggatctgctgttcttcacagatgagtcgggggacagccggggctggaagctgcgctacaccaccgagatcatcaagtgcccccagcccaagaccctagacgagttcaccatcatccagaacctgcagcctcagtaccagttccgtgactacttcattgctacctgcaagcaaggctaccagctcatagaggggaaccaggtgctgcattccttcacagctgtctgccaggatgatggcacgtggcatcgtgccatgcccagatgcaagatcaaggactgtgggcagccccgaaacctgcctaatggtgacttccgttacaccaccacaatgggagtgaacacctacaaggcccgtatccagtactactgccatgagccatattacaagatgcagaccagagctggcagcagggagtctgagcaaggggtgtacacctgcacagcacagggcatttggaagaatgaacagaagggagagaagattcctcggtgcttgccagtgtgtgggaagcccgtgaaccccgtggaacagaggcagcgcataatcggagggcaaaaagccaagatgggcaacttcccctggcaggtgttcaccaacatccacgggcgcgggggcggggccctgctgggcgaccgctggatcctcacagctgcccacaccctgtatcccaaggaacacgaagcgcaaagcaacgcctctttggatgtgttcctgggccacacaaatgtggaagagctcatgaagctaggaaatcaccccatccgcagggtcagcgtccacccggactaccgtcaggatgagtcctacaattttgagggggacatcgccctgctggagctggaaaatagtgtcaccctgggtcccaacctcctccccatctgcctccctgacaacgataccttctacgacctgggcttgatgggctatgtcagtggcttcggggtcatggaggagaagattgctcatgacctcaggtttgtccgtctgcccgtagctaatccacaggcctgtgagaactggctccggggaaagaataggatggatgtgttctctcaaaacatgttctgtgctggacacccatctctaaagcaggacgcctgccagggggatagtgggggcgtttttgcagtaagggacccgaacactgatcgctgggtggccacgggcatcgtgtcctggggcatcgggtgcagcaggggctatggcttctacaccaaagtgctcaactacgtggactggatcaagaaagagatggaggaggaggactgagcccagaattcactaggttcgaatccagagagcagtgtggaaaaaaaaaaaacaaaaaacaactgaccagttgttgataaccactaagagtctctattaaaattactgatgcagaaagaccgtgtgtgaaattctctttcctgtagtcccattgatgtactttacctgaaacaacccaaaggcccctttctttcttctgaggattgcagaggatatagttatcaatctctagttgtcactttcctcttccactttgataccattgggtcattgaatataactttttccaaataaagttttatgagaaatgcc

SEQ ID NO: 18 - DNA, 2787 bp, Homo sapiens
attccggcacagggacacaaacaagctcacccaacaaagccaagctgggaggaccaaggccgggcagccgggagcacccaaggcaggaaaatgaggtggctgcttctctattatgctctgtgcttctccctgtcaaaggcttcagcccacaccgtggagctaaacaatatgtttggccagatccagtcgcctggttatccagactcctatcccagtgattcagaggtgacttggaatatcactgtcccagatgggtttcggatcaagctttacttcatgcacttcaacttggaatcctcctacctttgtgaatatgactatgtgaaggtagaaactgaggaccaggtgctggcaaccttctgtggcagggagaccacagacacagagcagactcccggccaggaggtggtcctctcccctggctccttcatgtccatcactttccggtcagatttctccaatgaggagcgtttcacaggctttgatgcccactacatggctgtggatgtggacgagtgcaaggagagggaggacgaggagctgtcctgtgaccactactgccacaactacattggcggctactactgctcctgccgcttcggctacatcctccacacagacaacaggacctgccgagtggagtgcagtgacaacctcttcactcaaaggactggggtgatcaccagccctgacttcccaaacccttaccccaagagctctgaatgcctgtataccatcgagctggaggagggtttcatggtcaacctgcagtttgaggacatatttgacattcaggaccatcctgaggtgccctgcccctatgactacatcaagatcaaagttggtccaaaagttttggggcctttctgtggagagaaagccccagaacccatcagcacccagagccacagtgtcctgatcctgttccatagtgacaactcggcagagaaccggggctggaggctctcatacagggctgcaggaaatgagtgcccagagctacagcctcctgtccatgggaaaatcgagccctcccaagccaagtatttcttcaaagaccaagtgctcgtcagctgtgacacaggctacaaagtgctgaaggataatgtggagatggacacattccagattgagtgtctgaaggatgggacgtggagtaacaagattcccacctgtaaaattgtagactgtagagccccaggagagctggaacacgggctgatcaccttctctacaaggaacaacctcaccacatacaagtctgagatcaaatactcctgtcaggagccctattacaagatgctcaacaataacacaggtatatatacctgttctgcccaaggagtctggatgaataaagtattggggagaagcctacccacctgccttccagtgtgtgggctccccaagttctcccggaagctgatggccaggatcttcaatggacgcccagcccagaaaggcaccactccctggattgccatgctgtcacacctgaatgggcagcccttctgcggaggctcccttctaggctccagctggatcgtgaccgccgcacactgcctccaccagtcactcgatccgggagatccgaccctacgtgattcagacttgctcagcccttctgacttcaaaatcatcctgggcaagcattggaggctccggtcagatgaaaatgaacagcatctcggcgtcaaacacaccactctccacccccagtatgatcccaacacattcgagaatgacgtggctctggtggagctgttggagagcccagtgctgaatgccttcgtgatgcccatctgtctgcctgagggaccccagcaggaaggagccatggtcatcgtcagcggctgggggaagcagttcttgcaaaggttcccagagaccctgatggagattgaaatcccgattgttgaccacagcacctgccagaaggcttatgccccgctgaagaagaaagtgaccagggacatgatctgtgctggggagaaggaagggggaaaggacgcctgtgcgggtgactctggaggccccatggtgaccctgaatagagaaagaggccagtggtacctggtgggcactgtgtcctggggtgatgactgtgggaagaaggaccgctacggagtatactcttacatccaccacaacaaggactggatccagagggtcaccggagtgaggaactgaatttggctcctcagccccagcaccaccagctgtgggcagtcagtagcagaggacgatcctccgatgaaagcagccatttctcctttccttcctcccatcccccctccttcggcctatccattactgggcaatagagcaggtatcttcaccccctttcactctctttaaagagatggagcaagagagtggtcagaacacaggccgaatccaggctctatcaccttactagtttgcagtgctgggcaggtgacttcatctcttcgaacttcagtttcttcataagatggaaatgctataccttacctacctcgtaaaagtctgatgaggaaaagattaagtaatagatgcatagcacttaacagagtgcatagcatacactgttttcaataaatgcaccttagcagaaggtcgatgtgtctaccaggcagcgaagctctcttacaaacccctgcctgggtcttagcattgatcagtgacacacctctcccctcaaccttgaccatctccatctgcccttaaatgctgtatgcttttttgccaccgtgcaacttgcccaacatcaatcttcaccctcatccctaaaaaagtaaaacagac

SEQ ID NO: 19 - DNA, 5135 bp, Homo sapiens
cacagacaagtctggcagaaggaaccaaagccaggagctcacagagcaggaaaatgaggttcctgtctttctggcggctcctcctctaccacgctctgtgcctcgccctgccggaggtttcagcccataccgtggagctaaacgaaatgtttggtcagatccagtcacctggctatccagattcctatccaagtgactctgaggtgacatggaatattactgtcccggaggggtttcgaatcaagctttacttcatgcacttcaacttggaatcctcctatctttgtgaatacgactatgtgaaggtagaaacagaagaccaggtgctggcaaccttttgtggcagggagaccaccgatactgagcagacccccggccaggaagtggttctttcgcctggcaccttcatgtctgtcactttccggtcagatttctccaatgaggaacgattcacaggcttcgacgcccactacatggctgtagatgtggatgagtgcaaggagagggaagatgaagagctgtcctgtgaccactactgtcacaactacatcggtggctactactgctcctgccgctttggctacatcctccacacagacaacaggacctgccgagtggaatgcagcggcaatctctttacccagaggacaggcacaatcaccagccccgattaccccaacccttatcccaagagctcagaatgttcctataccattgacctggaggaaggcttcatggtcagcctgcagtttgaggacatttttgacattgaagaccatcctgaggtgccctgtccctatgactacattaagattaaagctggttcaaaagtatggggtcccttctgtggagagaaatccccagaaccaatcagcacccagactcacagtgtccagatcctattccgcagcgacaactcaggagagaaccgaggctggaggctctcctacagagcggcaggaaatgagtgcccaaagctacagcctcctgtgtacgggaaaatcgagccctcgcaggccgtgtattccttcaaagaccaagtgctcgtcagctgtgacacaggctacaaagtgctaaaggataacggggtgatggacacattccaaattgagtgtctgaaggacggtgcatggagtaacaagatccccacctgtaaaattgtagactgtggagctcctgcagggctgaaacatgggctagtaaccttctccaccagaaacaacctcaccacatacaaatctgagataaggtactcctgccaacagccctattacaagatgcttcacaataccacaggtgtatatacgtgttctgctcatgggacctggacgaacaaagtgctcaagagaagcctgcccacctgccttccagtgtgtggtgtccccaagttctcccggaagcagatctccaggatcttcaatggccgcccagcccagaagggtaccatgccatggattgccatgctgtcacacctgaacggacaacccttctgtgggggtagccttttaggttccaactgggttttgacagctgctcactgcctccaccagtcacttgatccagaagaaccaaccctacacagctcatacttgctcagcccttctgacttcaaaattatcatgggaaagcactggagacggcgctcagacgaagacgagcagcacctgcatgtaaagcgcaccacgctccacccactgtacaaccccagcacgtttgagaacgaccttggtctggtggaactgtcagagagcccgaggctgaacgactttgtgatgcctgtctgtctgcctgagcagccttccactgaaggaaccatggtcatcgtcagtggctgggggaagcagttcttacagaggtttccagagaacctgatggagattgaaatcccaattgtaaactctgacacctgccaggaggcctataccccattgaagaagaaagtgaccaaggacatgatctgtgccggagaaaaggaaggggggaaagatgcctgtgctggtgactctggaggccctatggtgaccaaagatgcagagagagaccaatggtacctggtgggcgtggtgtcctggggtgaagattgcgggaagaaagatcgctatggagtctattcttacatctatcccaacaaggactggatccagaggatcactggggtgaggaactgagttcgaatcccagcccaacacctgctgtatggtcagtcaccaacagaagatcagtgaatgcaagcaacctttcctccctggtcctcagtcttcactgctcattcctgggtgatactgggattcgttgaaccacctttccctggtctttatagaggcagagtagcaaagcagccaggctggatccaggctccatcactcaaaatttttgtaatgatggacagctgactgcctctttgggtctgttcttcaaccatgagaagcaagtggtcagaccttatctacctcacaaaaccgtgctgaggagaaagttaatacatacatagtacttagcctagtgtttgacctaaactatgttttctaaaaactgtgacttagcaaaaggcgtctgtgtccacgaggcaggtggatggtcccttataaactcttgatagggtcttagggatgattagtgccacccctccaccaccctcagcccttgctttaatctgtccccaaaagtctaagctttttcactaaatgccatcctcctaagcccagccccattccccataaaaatgcaaacaaaatacaattctcagccctatgacgtgaccccagttacacagccagcaatgtcgttcggcacttgagctaagtaccaaatggtaagagaagcgaggctgagaggaaatgggggttgtgaagtcttatcaacccttgtctctcatgggaccctcactacaagtcttttcttcttgttttgaaggtacttatcagccctgacattctagaatccaagggagtcgtgtcccctgtgatgagttagattcagagtaattcaaaagaaaaatgttcattaggttcaaaagacaaaattttcctgctgtccctaaaattcccacagtgatccaccatactcaagtgcagccaaagatcttccccttgctctaaatagagtggctttcctgagccccatcccccttctcgcctctaagcatgggcagcagaaggcaggccctggcaggctcctctctctctctctctctctctctctctctctctctctctctctctctcacacacacacacactcactctctccttctcgctctccgcatcccgctctctgcatcccagctgagcttaggttgccaattctctgattcctgtcgctttgtctcaccaaactgagaacactgtgtttgcataagtttttagaaaccttatccaagacaagatttttgaacaaacagaagcccaaccctgaatttctgtgtatgagaattgttcttcatagaagactttgaccctcgacctgtattgctgctgctagtttccataaaaatctctggtaagtgaggtagacagtgaggaatgagggcttgtgggtataaagcccaaggctccacactcagggacaacactttgccccactacccctctgagcatgtcactctattcctacacgcttgactactattcgaagagatggccgggacccaacaatcagatacttctcaaggaagctgctactctattttagttcctgatgaagacttttgatgcagttttgaaactgctttggaggcaatgcgccctgccccctccacagctcttgctgagcagtctgttatacaggtcatagtgactgctgctgtggcctgctgcagtgagaaacatatgggtcatggcttccagacattcctggtggaactgtgacacacatgtgacttctatatggggatgacccctgacaagtctattttagagaggcatggagatagaaaaaaagcccaattttgtacataatttaaggaggggaacgccaagaatcagcctagagggtgatgaccttcagaaagtgagcatttctgcaagtgaggccaaggaaactcttctaaaaaaacaggagtctgcatccactcagataccaccagcccctcccatagtaatgatatttccagaaaaccagcattcaacatgagaaccaacatctaaacaggcctttctccaaaaatcttcatccagaactaaaatagcgtatttatccttatcagaacaccagcgctttaaaagcttcaggtttcccatgcagataccaacttctggctgggcacaatttattctatttatcctccaaattatgacttcatcttgagaaaaataactaaatataccatggaacttgaaccttgtcctataaatgcctgtgacatgatgtgtactcaaaccattcttactcatggtttgagtaagaatggcccccacaggctcacatatttgtatgcttggtcactagagagtgttgatatttgaaaagatcagatgtcaccgtacaggagtggagtggccttgtggaggaaatgtgccactggaagtgggctttgaggttttcaaaagcccaaaccaggctcaggggctctcttcctgctgcctgtggataaagatgtaggagttttggccaattctccagcaccatgtttgcctgcatgccaccatgctcttgccatgataaaaatgggcaaaacctctgaaactgtaagccagccccatttaaatgcttttttttttttgtaagagttgtgtggtcacagctgtctcttcacagcaatagaacactaagacagaaatctagtttctattatggtccaagaagcctgatcacctaaaactagagacacagaaggaaggtatagccatagagtctatcttgtctaaattcataaccttatgccaaccgactcactaccttcacatccagccagttcctaagcaactctaaaatgtgctgcccataaaaaagcctgtcttccaggcaactgaaatctacctcccgagaaattaatttgtataatgaaagctgtgattttatactgcgagcactggtattagcagtgatgatcatgcctgggattcattagtcaaagaagttgttattcttatgggaaactacacattcgttcaataaacatctgcattgagtcaaag

SEQ ID NO: 20 - DNA, 2485 bp, Homo sapiens
gctggacgggcacaccatgaggctgctgaccctcctgggccttctgtgtggctcggtggccacccccttaggcccgaagtggcctgaacctgtgttcgggcgcctggcatcccccggctttccaggggagtatgccaatgaccaggagcggcgctggaccctgactgcaccccccggctaccgcctgcgcctctacttcacccacttcgacctggagctctcccacctctgcgagtacgacttcgtcaagctgagctcgggggccaaggtgctggccacgctgtgcgggcaggagagcacagacacggagcgggcccctggcaaggacactttctactcgctgggctccagcctggacattaccttccgctccgactactccaacgagaagccgttcacggggttcgaggccttctatgcagccgaggacattgacgagtgccaggtggccccgggagaggcgcccacctgcgaccaccactgccacaaccacctgggcggtttctactgctcctgccgcgcaggctacgtcctgcaccgtaacaagcgcacctgctcagccctgtgctccggccaggtcttcacccagaggtctggggagctcagcagccctgaatacccacggccgtatcccaaactctccagttgcacttacagcatcagcctggaggaggggttcagtgtcattctggactttgtggagtccttcgatgtggagacacaccctgaaaccctgtgtccctacgactttctcaagattcaaacagacagagaagaacatggcccattctgtgggaagacattgccccacaggattgaaacaaaaagcaacacggtgaccatcacctttgtcacagatgaatcaggagaccacacaggctggaagatccactacacgagcacagcgcacgcttgcccttatccgatggcgccacctaatggccacgtttcacctgtgcaagccaaatacatcctgaaagacagcttctccatcttttgcgagactggctatgagcttctgcaaggtcacttgcccctgaaatcctttactgcagtttgtcagaaagatggatcttgggaccggccaatgcccgcgtgcagcattgttgactgtggccctcctgatgatctacccagtggccgagtggagtacatcacaggtcctggagtgaccacctacaaagctgtgattcagtacagctgtgaagagaccttctacacaatgaaagtgaatgatggtaaatatgtgtgtgaggctgatggattctggacgagctccaaaggagaaaaatcactcccagtctgtgagcctgtttgtggactatcagcccgcacaacaggagggcgtatatatggagggcaaaaggcaaaacctggtgattttccttggcaagtcctgatattaggtggaaccacagcagcaggtgcacttttatatgacaactgggtcctaacagctgctcatgccgtctatgagcaaaaacatgatgcatccgccctggacattcgaatgggcaccctgaaaagactatcacctcattatacacaagcctggtctgaagctgtttttatacatgaaggttatactcatgatgctggctttgacaatgacatagcactgattaaattgaataacaaagttgtaatcaatagcaacatcacgcctatttgtctgccaagaaaagaagctgaatcctttatgaggacagatgacattggaactgcatctggatggggattaacccaaaggggttttcttgctagaaatctaatgtatgtcgacataccgattgttgaccatcaaaaatgtactgctgcatatgaaaagccaccctatccaaggggaagtgtaactgctaacatgctttgtgctggcttagaaagtgggggcaaggacagctgcagaggtgacagcggaggggcactggtgtttctagatagtgaaacagagaggtggtttgtgggaggaatagtgtcctggggttccatgaattgtggggaagcaggtcagtatggagtctacacaaaagttattaactatattccctggatcgagaacataattagtgatttttaacttgcgtgtctgcagtcaaggattcttcatttttagaaatgcctgtgaagaccttggcagcgacgtggctcgagaagcattcatcattactgtggacatggcagttgttgctccacccaaaaaaacagactccaggtgaggctgctgtcatttctccacttgccagtttaattccagccttacccattgactcaaggggacataaaccacgagagtgacagtcatctttgcccacccagtgtaatgtcactgctcaaattacatttcattaccttaaaaagccagtctcttttcatactggctgttggcatttctgtaaactgcctgtccatgctctttgtttttaaacttgttcttattgaaaaaaaaaaaaaaaaaaaaaaaaaaaaaaaaaaaaaaaaaaaaaaa

SEQ ID NO: 21 - DNA, 2790 bp, Homo sapiens
ggacagggaggctggccggaggttcctgcagagggagcgtcaaggccctgtgctgctgtccctgggggccagaggggttgcccagcatgcccactggcaggagagagggaactgacccacttgctcctaccagcttctgaaggtgacactgagccccaggtgacgccgcaccaccaaagaaggtgcttgtgtttgtcagacaaatacagccaggcctgccaccccttaggctccaaagtccggaggtgcagaaagccaggaccaagagacaggcagctcaccagggtggacaaatcgccagagatgtggtgcattgtcctgttttcacttttggcatgggtttatgctgagcctaccatgtatggggagatcctgtcccctaactatcctcaggcatatcccagtgaggtagagaaatcttgggacatagaagttcctgaagggtatgggattcacctctacttcacccatctggacattgagctgtcagagaactgtgcgtatgactcagtgcagataatctcaggagacactgaagaagggaggctctgtggacagaggagcagtaacaatccccactctccaattgtggaagagttccaagtcccatacaacaaactccaggtgatctttaagtcagacttttccaatgaagagcgttttacggggtttgctgcatactatgttgccacagacataaatgaatgcacagattttgtagatgtcccttgtagccacttctgcaacaatttcattggtggttacttctgctcctgccccccggaatatttcctccatgatgacatgaagaattgcggagttaattgcagtggggatgtattcactgcactgattggggagattgcaagtcccaattatcccaaaccatatccagagaactcaaggtgtgaataccagatccggttggagaaagggttccaagtggtggtgaccttgcggagagaagattttgatgtggaagcagctgactcagcgggaaactgccttgacagtttagtttttgttgcaggagatcggcaatttggtccttactgtggtcatggattccctgggcctctaaatattgaaaccaagagtaatgctcttgatatcatcttccaaactgatctaacagggcaaaaaaagggctggaaacttcgctatcatggagatccaatgccctgccctaaggaagacactcccaattctgtttgggagcctgcgaaggcaaaatatgtctttagagatgtggtgcagataacctgtctggatgggtttgaagttgtggagggacgtgttggtgcaacatctttctattcgacttgtcaaagcaatggaaagtggagtaattccaaactgaaatgtcaacctgtggactgtggcattcctgaatccattgagaatggtaaagttgaagacccagagagcactttgtttggttctgtcatccgctacacttgtgaggagccatattactacatggaaaatggaggaggtggggagtatcactgtgctggtaacgggagctgggtgaatgaggtgctgggcccggagctgccgaaatgtgttccagtctgtggagtccccagagaaccctttgaagaaaaacagaggataattggaggatccgatgcagatattaaaaacttcccctggcaagtcttctttgacaacccatgggctggtggagcgctcattaatgagtactgggtgctgacggctgctcatgttgtggagggaaacagggagccaacaatgtatgttgggtccacctcagtgcagacctcacggctggcaaaatccaagatgctcactcctgagcatgtgtttattcatccgggatggaagctgctggaagtcccagaaggacgaaccaattttgataatgacattgcactggtgcggctgaaagacccagtgaaaatgggacccaccgtctctcccatctgcctaccaggcacctcttccgactacaacctcatggatggggacctgggactgatctcaggctggggccgaacagagaagagagatcgtgctgttcgcctcaaggcggcaaggttacctgtagctcctttaagaaaatgcaaagaagtgaaagtggagaaacccacagcagatgcagaggcctatgttttcactcctaacatgatctgtgctggaggagagaagggcatggatagctgtaaaggggacagtggtggggcctttgctgtacaggatcccaatgacaagaccaaattctacgcagctggcctggtgtcctgggggccccagtgtgggacctatgggctctacacacgggtaaagaactatgttgactggataatgaagactatgcaggaaaatagcaccccccgtgaggactaatccagatacatcccaccagcctctccaagggtggtgaccaatgcattaccttctgttccttatgatattctcattatttcatcatgactgaaagaagacacgagcgaatgatttaaatagaacttgattgttgagacgccttgctagaggtagagtttgatcatagaattgtgctggtcatacatttgtggtctgactccttggggtcctttccccggagtacctattgtagataacactatgggtggggcactcctttcttgcactattccacagggataccttaattctttgtttcctctttacctgttcaaaattccatttacttgatcattctcagtatccactgtctatgtacaataaaggatgtttataagcaaaaaaaaaaaaaaaaaaaaaaaaaaaaaaaaaaaaaaaaaa

SEQ ID NO: 22 - DNA, 2263 bp, Homo sapiens
tgaaattcagggactctttggtggagcaattaccagtcaacttcagggtattatgataaactctgatctggggaggaaccaggactacatagatcaaggcagttttcttctttgagaaactatcccagatatcatcatagagtcttctgctcttcctcaactaccaaagaaaaacatcagcgaagcagcaggccatgcaccccccaaaaactccatctggggctcttcatagaaaaaggaaaatggcagcctggcccttctccaggctgtggaaagtctctgatccaattctcttccaaatgaccttgatcgctgctctgttgcctgctgttcttggcaattgtggtcctccacccactttatcatttgctgccccgatggatattacgttgactgagacacgcttcaaaactggaactactctgaaatacacctgcctccctggctacgtcagatcccattcaactcagacgcttacctgtaattctgatggcgaatgggtgtataacaccttctgtatctacaaacgatgcagacacccaggagagttacgtaatgggcaagtagagattaagacagatttatcttttggatcacaaatagaattcagctgttcagaaggatttttcttaattggctcaaccactagtcgttgtgaagtccaagatagaggagttggctggagtcatcctctcccacaatgtgaaattgtcaagtgtaagcctcctccagacatcaggaatggaaggcacagcggtgaagaaaatttctacgcatacggcttttctgtcacctacagctgtgacccccgcttctcactcttgggccatgcctccatttcttgcactgtggagaatgaaacaataggtgtttggagaccaagccctcctacctgtgaaaaaatcacctgtcgcaagccagatgtttcacatggggaaatggtctctggatttggacccatctataattacaaagacactattgtgtttaagtgccaaaaaggttttgttctcagaggcagcagtgtaattcattgtgatgctgatagcaaatggaatccttctcctcctgcttgtgagcccaatagttgtattaatttaccagacattccacatgcttcctgggaaacatatcctaggccgacaaaagaggatgtgtatgttgttgggactgtgttaaggtaccgctgtcatcctggctacaaacccactacagatgagcctacgactgtgatttgtcagaaaaatttgagatggaccccataccaaggatgtgaggcgttatgttgccctgaaccaaagctaaataatggtgaaatcactcaacacaggaaaagtcgtcctgccaatcactgtgtttatttctatggagatgagatttcattttcatgtcatgagaccagtaggttttcagctatatgccaaggagatggcacgtggagtccccgaacaccatcatgtggagacatttgcaattttcctcctaaaattgcccatgggcattataaacaatctagttcatacagctttttcaaagaagagattatatatgaatgtgataaaggctacattctggtcggacaggcgaaactctcctgcagttattcacactggtcagctccagcccctcaatgtaaagctctgtgtcggaaaccagaattagtgaatggaaggttgtctgtggataaggatcagtatgttgagcctgaaaatgtcaccatccaatgtgattctggctatggtgtggttggtccccaaagtatcacttgctctgggaacagaacctggtacccagaggtgcccaagtgtgagtgggagacccccgaaggctgtgaacaagtgctcacaggcaaaagactcatgcagtgtctcccaaacccagaggatgtgaaaatggccctggaggtatataagctgtctctggaaattgaacaactggaactacagagagacagcgcaagacaatccactttggataaagaactataatttttctcaaaagaaggaggaaaaggtgtcttgctggcttgcctcttgcaattcaatacagatcagtttagcaaatctactgtcaatttggcagtgatattcatcataataaatatctagaaatgataatttgctaaagtttagtgctttgagattgtgaaattattaatcatcctctgtgtggctcatgtttttgcttttcaacacacaaagcacaaattttttttcgattaaaaatgtatgttaaaaaaaaaaaaaaaaaaaaaaaaaaaaaa

SEQ ID NO: 23 - DNA, 2021 bp, Rattus norvegicus
atgaagctcgctctgcttattctgctactcttgaatcctcacttgagttcttccaagaacacaccagcctcaggtcagcctcaggaggatctggtagagcagaaatgcttactgaaaaactacacgcatcactcctgtgacaaagtcttctgccagccatggcagaaatgtatcgagggaacctgtgcctgcaaactcccttaccagtgcccaaaggccgggaccccggtgtgcgccactaatggaagaggctacccgacatactgtcacctgaagagtttcgaatgtcttcacccggagataaagttctcgaataatggaacatgcacagctgaagaaaagtttaatgtttccttaatttatggaagcacagatacagagggaattgttcaagttaaactcgtggaccaagatgagaaaatgttcatatgtaaaaatagctggagcaccgtggaagccaacgtggcctgcttcgacctcggatttccactgggtgttcgtgacatacaaggaaggtttaatatacctgtaaatcacaaaataaactccaccgaatgcctgcatgtgcgttgccagggagtagagaccagtttggcagagtgtacctttaccaagaagagttcgaaggctccccatggcttggcaggtgtagtgtgctacacacaggatgcagatttcccaacaagtcagtccttccagtgtgtgaatgggaagcgcattcctcaggagaaagcctgtgatggtgtcaacgactgtggagatcaaagtgatgagctgtgttgcaaaggttgccgaggccaagccttcctttgcaagtcgggagtttgcatcccaaaccaacgtaagtgtaacggtgaggtggactgcatcaccggcgaggacgagagtggctgtgaagaagacaaaaagaataaaattcataaaggccttgcacggtcagaccaaggaggagaaactgaaattgagactgaagaaacagaaatgttgactcctgatatggacacagaaagaaaacggataaagtccttattacctaaactatcctgtggagtcaaaagaaatactcacattcgcaggaaaagagtggtcggagggaagccagccgagatgggagattacccatggcaggtggcgattaaggatggagatagaataacctgtgggggcatttatatcggtggctgttggattctgacagctgcacactgtgtcagacccagtagatatcgcaactaccaagtatggacgtctttattagactggctaaagcctaactctcagttggcagttcagggagtgagcagagttgtcgttcatgaaaagtataacggagccacctaccagaatgacatagctttggttgaaatgaaaaaacacccgggcaagaaagaatgtgagctcatcaattctgtccctgcctgtgtcccatggtctccatatctattccaaccgaatgacagatgcatcatttctggatggggtcgagaaaaagataaccaaaaagtctactcactcaggtggggcgaagtcgacctaataggcaactgctcgaggttttacccgggtcgctactatgaaaaagagatgcagtgtgcgggtaccagtgatgggtccattgatgcctgcaaaggagactctggaggccccttggtctgcaaggatgtcaacaatgtcacttatgtttggggcattgtgagctggggagaaaactgtgggaaaccagagttcccaggtgtttacaccagagtggccagctattttgattggattagctactacgtgggaagaccccttgtttctcaatacaatgtctgaagctacgacctccttctttctgcacttcttctttccagggttatactttaattgaaatgaaactgtataattagttctcctcgatgctggcaagaagcaagtcttactggctagttcctaaagtttcttcaaagtttatgccattttagaattctgtcatataatccccaataaatattccagttaagcacacaaaaaaaaaaaaa

SEQ ID NO: 24 - DNA, 2823 bp, Homo sapiens
ggcaggtgcttgttactgttaatgaaagcagatttaaagcaacaccaccatcactggagtatttttagttatatacgattgagactaccaagcatgttgctcttattcagtgtaatcctaatctcatgggtatccactgttgggggagaaggaacactttgtgattttccaaaaatacaccatggatttctgtatgatgaagaagattataaccctttttcccaagttcctacaggggaagttttctattactcctgtgaatataattttgtgtctccttcaaaatccttttggactcgcataacatgcacagaagaaggatggtcaccaacaccgaagtgtctcagaatgtgttcctttccttttgtgaaaaatggtcattctgaatcttcaggactaatacatctggaaggtgatactgtacaaattatttgcaacacaggatacagccttcaaaacaatgagaaaaacatttcgtgtgtagaacggggctggtccactcctcccatatgcagcttcactaaaggagaatgtcatgttccaattttagaagccaatgtagatgctcagccaaaaaaagaaagctacaaagttggagacgtgttgaaattctcctgcagaaaaaatcttataagagttggatcagactcagttcaatgttaccaatttgggtggtcacctaactttccaacatgcaaaggacaagtacgatcatgtggtccacctcctcaactctccaatggtgaagttaaggagataagaaaagaggaatatggacacaatgaagtagtggaatatgattgcaatcctaattttataataaacgggcctaagaaaatacaatgtgtggatggagaatggacaactttacccacttgtgttgaacaagtgaaaacatgtggatacatacctgaactcgagtacggttatgttcagccgtctgtccctccctatcaacatggagtttcagtcgaggtgaattgcagaaatgaatatgcaatgattggaaataacatgattacctgtattaatggaatatggacagagcttcctatgtgtgttgcaacacaccaacttaagaggtgcaaaatagcaggagttaatataaaaacattactcaagctatctgggaaagaatttaatcataattctagaatacgttacagatgttcagacatcttcagatacaggcactcagtctgtataaacgggaaatggaatcctgaagtagactgcacagaaaaaagggaacaattctgcccaccgccacctcagatacctaatgctcagaatatgacaaccacagtgaattatcaggatggagaaaaagtagctgttctctgtaaagaaaactatctacttccagaagcaaaagaaattgtatgtaaagatggacgatggcaatcattaccacgctgtgttgagtctactgcatattgtgggccccctccatctattaacaatggagataccacctcattcccattatcagtatatcctccagggtcaacagtgacgtaccgttgccagtccttctataaactccagggctctgtaactgtaacatgcagaaataaacagtggtcagaaccaccaagatgcctagatccatgtgtggtatctgaagaaaacatgaacaaaaataacatacagttaaaatggagaaacgatggaaaactctatgcaaaaacaggggatgctgttgaattccagtgtaaattcccacataaagcgatgatatcatcaccaccatttcgagcaatctgtcaggaagggaaatttgaatatcctatatgtgaatgaagcaagcataattttcctgaatatattcttcaaacatccatctacgctaaaagtagccattatgtagccaattctgtagttacttcttttattctttcaggtgttgtttaactcagttttatttagaactctggatttttagagctttagaaatttgtaagctgagagaacaatgtttcacttaataggagggtgtcttagtccatattacattgttataacagagtatcacagactggataacttctaaccaatagtttatttgtttcataaatctaaaagctgagaagtccaagatggtggggctgcctctggtgagggtcttctcgaagcatcataatatgctggaaggcatcacaacatggtggaagggatcacgtggcaaaagagcatgtacatgggagtgagagaaaaagagagagagagacagagtggcgggggccggggaggagcgcaaactcatcctttataaagacaccactcctgagataacaatccaatcccatgataatgacattaatccattcaagaagatagagctctcgtgacttaatcaccttctaaagatctcacctgacaacactgttgcattggcagttaagtttccacgtaaactttcggggacacattcaaaccacaggagaaactcaaattgttcctgggcaaatcacaacatggggaattttattcataaatgtccacagaaacagtaaatgttctcgcttcagaacttaattcatctaatccctcctgtttgtctcaaattataggataactttgaaactttctgaattaacgttatttaaaaggaaatgtagatgttattttagtctctatcttcaggttattatcacttaaaaacctgcgaaagctgtcaacttttgtggttgtagcaagtattaataaatatttataaatcctctaatgtaagtctagctacctatccaatactaaataccccttaaagtattaaatgcactatctgctgtaaacggaaaaaaaaaaaaaaaaaaaaaaaaa

SEQ ID NO: 25 - DNA, 1995 bp, Homo sapiens
ctgtggcatctcctgtcacattgggaaatgaagaattccaggacatgggcttggagggcgccggtggagctatttcttctctgtgctgccctgggctgtctcagtttgcctggctccagaggtgaaaggccacattcctttgggtcaaatgcagtcaacaagagctttgctaagagcagacagatgcggagtgtggatgttaccctgatgcccattgattgtgagctgtctagttggtcctcttggaccacatgtgacccctgtcagaagaaaaggtacaggtatgcctacttgctccagccctctcagttccatggggaaccgtgcaacttctctgacaaggaagtcgaagactgtgttaccaacagaccatgcggaagtcaagtgcgatgtgaaggctttgtgtgtgcacagacaggaaggtgtgtaaaccgcagacttctttgcaatggggacaatgactgtggagaccagtcagatgaagcaaactgtagaaggatttataaaaaatgtcagcatgaaatggaccaatactggggaattggcagtctggccagtgggataaatttgttcacaaacagttttgagggcccagttcttgatcacaggtattatgcaggtggatgctccccgcattacatcctgaacacgaggtttaggaagccctacaatgtggaaagctacacgccacagacccaaggcaaatacgaattcatattaaaagagtatgaatcatactcagattttgaacgcaatgtcacagagaaaatggcaagcaagtctggtttcagttttggttttaaaatacctggaatatttgaacttggcatcagtagtcaaagtgatcgaggcaaacactatattaggagaaccaaacgattctctcatactaaaagcgtatttctgcatgcacgctctgaccttgaagtagcacattacaagctgaaacccagaagcctcatgctccattacgagttccttcagagagttaagcggctgcccctggagtacagctacggggaatacagagatctcttccgtgattttgggacccactacatcacagaggctgtgcttgggggcatttatgaatacaccctcgttatgaacaaagaggccatggagagaggagattatactcttaacaacgtccatgcctgtgccaaaaatgattttaaaattggtggtgccattgaagaggtctacgtcagtctgggtgtgtctgtaggcaaatgcagaggtattctgaatgaaataaaagacagaaacaagagggacaccatggtggaggacttggtggtcctggtacgaggaggggcaagtgagcacatcaccaccctggcataccaggagctgccgacggcggacctgatgcaggagtggggagacgctgtgcagtacaacccagccatcatcaaagttaaggtggagcctctgtatgaactagtgacagccacagattttgcctattccagcacagtgaggcagaacatgaagcaggcactggaggagttccagaaggaagttagttcctgccactgtgctccctgccaaggaaatggagtccctgtcctgaaaggatcacgctgtgactgcatctgtcctgttggatcccaaggcctagcctgtgaggtctcctatcggaagaatacccccattgatgggaagtggaattgctggtcaaattggtcttcatgctctggaagacgtaagacaagacaaaggcagtgtaacaatccacctcctcaaaatgggggtagcccctgttcaggccctgcttcagaaacacttgactgctcctagcagatgatacagcagtgggctacatacaatgagagccctgagccctcaagaactcacgccagctcagccctacaccagtttccacctggagttcatgcaagggcaaaaggcagtgccatgcaagctgtttaaaataaagatgttaccttgtaaaatgcaagttgatttaaataaatactgagttaaaggctt

SEQ ID NO: 26 - DNA, 1882 bp, Rattus norvegicus
gggcccttgtctacgttctgcagagcctccggtccaactttgttccaaatgagcctcactgctgctctttgggttgctgtattcggaaaatgtggcccaccacctgatttaccctacgccctgccagcaagtgagatgaaccagacagactttgaaagtcacactaccctgagatacaattgtcgccctggctatagtagagcgagctcaagccagagtctctactgtaaacctctggggaaatggcagattaatatcgcctgcgtcaaaaagtcatgcaggaatccaggagacttacaaaatggaaaggtggaagttaagacagatttcttgtttggatcacagatagaattcagctgctcagagggatatatcttaattggctcatccactagttattgtgagatccaaggcaaaggagtttcctggagtgatcctctcccagaatgtgtaattgccaagtgtgggatgcctccagacatcagcaatgggaagcacaatggtagagaggaagaattcttcacatatcgttcctcagtcacctataagtgtgatcctgacttcacactccttggcaatgcctccattacctgcactgtggtgaacaaaacagtaggtgtttggagcccaagccctcctacctgtgaaagaatcatctgtccttggccaaaagttttgcatggaacaattaattctggattcaagcatacctataaatacaaagactctgtgagatttgtctgccagaaagggtttgtcctcagaggcagcggtgtaatccattgtgaggctgatggcagctggagtcccgtaccagtgtgtgagctcaatagttgcactgatattccagacattcctaatgctgccctgataaccagtcccaggccaagaaaggaagatgtatatccagtgggtactgtgctccgttacatctgtcgtcctggctatgaacctgctacgagacagcccatgactgtgatttgtcagaaagatctcagctggagcatgcttagggggtgtaaggagatatgctgtccagtaccagacccaaagagtgttagagtcattcaacatgaaaaggcacatcctgacaacgactgtacttacttctttggtgacgaagtgtcatacacatgtcaaaatgatataatgcttacagctacttgcaagtcagatggcacctggcatccccggacaccatcatgtcatcagagttgtgattttccgcctgccattgctcacggacgttatacaaaatcttcttcatactacgtcagaactcaggttacatatgaatgtgaagaaggatacagactggttggagaggcaaccatctcctgctggtattcacaatggacaccagcagctccacagtgtaaagctctatgtcggaaaccagagataggaaatggagtactgtctactaataaagatcaatatgtcgaaactgaaaatgtcaccatccaatgtgactcgggctttgtcatgctaggttcccaaagcatcacttgttcggagaatggaacctggtacccaaaggtgtccagatgtgagcaggaggtccctaaagactgtgagcacgtgtttgcaggcaagaagctcatgcaatgtctgccaaattcaaatgacgtgaaaatggccctggaggtctacaagctgactctggagattaaacaattacagctccagatagacaaggcaaagcacgttgaccgggagttatgagcgggtgttctctcaaggaggaagaagtacctcatgggctttctgacttcagtgccaagcagaacgtctgcatttttagcaacctttgtaactttggcaccaatgttcatggtaataaatatctgcttagaataattcattaaagcataatgtaagc

SEQ ID NO: 27 - DNA, 2397 bp, Homo sapiens
ttttttttttcatcctactttgttttattgggcgttgattgttacaggtcccagcctgtagacatcttttactccaatttcctgaatagatagctttattccttcaaggtaatatagtgcggtggcttctggctgagatgtttgctgttgttttcttcatcttgtctttgatgacttgtcagcctggggtaactgcacaggagaaggtgaaccagagagtaagacgggcagctacacccgcagcagttacctgccagctgagcaactggtcagagtggacagattgctttccgtgccaggacaaaaagtaccgacaccggagcctcttgcagccaaacaagtttgggggaaccatctgcagtggtgacatctgggatcaagccagctgctccagttctacaacttgtgtaaggcaagcacagtgtggacaggatttccagtgtaaggagacaggtcgctgcctgaaacgccaccttgtgtgtaatggagaccaggactgccttgatggctctgatgaggacgactgtgaagatgtcagggccattgacgaagactgcagccagtatgaaccaattccaggatcacagaaggcagccttggggtacaatatcctgacccaggaagatgctcagagtgtgtacgatgccagttattatgggggccagtgtgagacggtatacaatggggaatggagggagcttcgatatgactccacctgtgaacgtctctactatggagatgatgagaaatactttcggaaaccctacaactttctgaagtaccactttgaagccctggcagatactggaatctcctcagagttttatgataatgcaaatgaccttctttccaaagttaaaaaagacaagtctgactcatttggagtgaccatcggcataggcccagccggcagccctttattggtgggtgtaggtgtatcccactcacaagacacttcattcttgaacgaattaaacaagtataatgagaagaaattcattttcacaagaatcttcacaaaggtgcagactgcacattttaagatgaggaaggatgacattatgctggatgaaggaatgctgcagtcattaatggagcttccagatcagtacaattatggcatgtatgccaagttcatcaatgactatggcacccattacatcacatctggatccatgggtggcatttatgaatatatcctggtgattgacaaagcaaaaatggaatcccttggtattaccagcagagatatcacgacatgttttggaggctccttgggcattcaatatgaagacaaaataaatgttggtggaggtttatcaggagaccattgtaaaaaatttggaggtggcaaaactgaaagggccaggaaggccatggctgtggaagacattatttctcgggtgcgaggtggcagttctggctggagcggtggcttggcacagaacaggagcaccattacataccgttcctgggggaggtcattaaagtataatcctgttgttatcgattttgagatgcagcctatccacgaggtgctgcggcacacaagcctggggcctctggaggccaagcgccagaacctgcgccgcgccttggaccagtatctgatggaattcaatgcctgccgatgtgggccttgcttcaacaatggggtgcccatcctcgagggcaccagctgcaggtgccagtgccgcctgggtagcttgggtgctgcctgtgagcaaacacagacagaaggagccaaagcagatgggagctggagttgctggagctcctggtctgtatgcagagcaggcatccaggaaaggagaagagagtgtgacaatccagcacctcagaatggaggggcctcgtgtccagggcggaaagtacagacgcaggcttgctgagggcctctggacacaggctggaccagatgctgtggatgtcgacccctgcactgactattggataaagacttctttcaactaagagaagatgcaaatcagcacacttttttctttgttctgccagcttccaggcctaagactaggttttgctgtctacagccaactattctattagttacaaaactcaatcattttattcagcaactggatgttgactgttaactagaagctctgtcctacttacagcactttggatcatcaaaaaaataaagtaaaatagaaaactgagaaaactcaatccatgaccagggagaacttacaggatgttagagacaaaacaagcagacacctgaaacaatcaacgcccaataaaacaaagtaggatgaaaattctcttagttctttgataacaatttgttcactcatagaaacattattaattggtagggtaagcagacactctgaaacaatgagaaaaatactaaaaattgacttgagttatttc

SEQ ID NO: 28 - DNA, 2094 bp, Homo sapiens
gcttgttccctgtcctctggccctttgcaaataaatgccttaccagacctgccctgccaccccactcgcagccacccagcaagagcagcatgtcagcctgccggagctttgcagttgcaatctgcattttagaaataagcatcctcacagcacagtacacgaccagttatgacccagagctaacagaaagcagtggctctgcatcacacatagactgcagaatgagcccctggagtgaatggtcacaatgcgatccttgtctcagacaaatgtttcgttcaagaagcattgaggtctttggacaatttaatgggaaaagatgcaccgacgctgtgggagacagacgacagtgtgtgcccacagagccctgtgaggatgctgaggatgactgcggaaatgactttcaatgcagtacaggcagatgcataaagatgcgacttcggtgtaatggtgacaatgactgcggagacttttcagatgaggatgattgtgaaagtgagccccgtcccccctgcagagacagagtggtagaagagtctgagctggcacgaacagcaggctatgggatcaacattttagggatggatcccctaagcacaccttttgacaatgagttctacaatggactctgtaaccgggatcgggatggaaacactctgacatactaccgaagaccttggaacgtggcttctttgatctatgaaaccaaaggcgagaaaaatttcagaaccgaacattacgaagaacaaattgaagcatttaaaagtatcatccaagagaagacatcaaattttaatgcagctatatctctaaaatttacacccactgaaacaaataaagctgaacaatgttgtgaggaaacagcctcctcaatttctttacatggcaagggtagttttcggttttcatattccaaaaatgaaacttaccaactatttttgtcatattcttcaaagaaggaaaaaatgtttctgcatgtgaaaggagaaattcatctgggaagatttgtaatgagaaatcgcgatgttgtgctcacaacaacttttgtggatgatataaaagctttgccaactacctatgaaaagggagaatattttgcctttttggaaacctatggaactcactacagtagctctgggtctctaggaggactctatgaactaatatatgttttggataaagcttccatgaagcggaaaggtgttgaactaaaagacataaagagatgccttgggtatcatctggatgtatctctggctttctctgaaatctctgttggagctgaatttaataaagatgattgtgtaaagaggggagagggtagagctgtaaacatcaccagtgaaaacctcatagatgatgttgtttcactcataagaggtggaaccagaaaatatgcatttgaactgaaagaaaagcttctccgaggaaccgtgattgatgtgactgactttgtcaactgggcctcttccataaatgatgctcctgttctcattagtcaaaaactgtctcctatatataatctggttccagtgaaaatgaaaaatgcacacctaaagaaacaaaacttggaaagagccattgaagactatatcaatgaatttagtgtaagaaaatgccacacatgccaaaatggaggtacagtgattctaatggatggaaagtgtttgtgtgcctgcccattcaaatttgagggaattgcctgtgaaatcagtaaacaaaaaatttctgaaggattgccagccctagagttccccaatgaaaaatagagctgttggcttctctgagctccagtggaagaagaaaacactagtaccttcagatcctacccctgaagataatcttagctgccaagtaaatagcaacatgcttcatgaaaatcctaccaacctctgaagtctcttctctcttaggtctataattttttttttaatttttcttccttaaactcctgtgatgtttccattttttgttccctaatgagaagtcaacagtgaaatacgccagaactgctttatcccacggaaaatgccaatctcttctaaaaaaaaacaaaattaaactaaaaaaaaaaaaaaaaaaaaaaaaaaaaaa

SEQ ID NO: 29 - DNA, 1779 bp, Mus musculus
gcttgcgtgggtagacttcagaagcttttttgatggagcaagtactggccagctttaggataatatgatcaactctcatcttgggagaaactagtacagtagagatccaagtaccaccacagagtctttctctgctattgcaggtttcctggcagaacaaatgtgtgcaaagcagcagcagaccctgctccccacaagagctgcacatgggaggcttcatagaaacagagatgctgtggcctggcccttttctacgctctgcagagtctctggcccaactttgttccaaatgacctttactgctgctctttgggttgctgtatttggcaaatgtggtccaccacctgctatacccaatgccctgccagcaagtgacgtgaatcggacagacttcgaaagtcacaccaccctgaaatacgaatgcctccctggctatggtaggggaatatcaaggatgatggtttactgcaaaccttcgggagaatgggagatttctgtctcctgtgccaaaaaacattgccgtaatccaggatatttagataatggatatgtaaatggagagactatcacttttggatcacagatagaattcagctgccaggaaggatttatcttagttggctcatccactagttcttgtgaggtccgaggcaaaggagttgcctggagtaatcctttcccagaatgtgtaattgtaaagtgtgggccccctccagacatcagcaatgggaagcacagtggtacagaagacttctacccatataaccatggaatctcctatacctgtgatcccggcttcaggctcgttggcagccccttcataggctgcactgtggtgaacaaaacagtacctgtttggagctcaagccctcctacctgtgaaaagatcatctgttctcagccaaatattttacatggtgtaattgtttctggatataaagctacctatacacacagagactctgttagattggcctgcctgaatgggactgtcctcagaggcagacatgtcatcgagtgtcaaggtaatggcaattggagttctctcccgacttgtgagttcgactgtgatttaccgcctgccattgtcaatggatattatactagtatggtctactctaagataactctggttacatatgaatgtgataaaggatacagactggttggaaaggcaatcatctcctgcagcttttcaaagtggaaagggacagctccacagtgtaaagctctatgtcagaaaccagaggtaggaaatggaacgctgtctgatgagaaagatcagtatgttgagtctgaaaatgtcaccatccaatgtgactctggctttgccatgctgggttcccaaagcatcagttgttcagagagtggaacctggtatccagaggtgcccagatgtgagcaggaggcctctgaagaccttaagcctgcgcttacaggcaacaagaccatgcagtatgtgccaaattcacacgatgtgaaaatggctctggagatctacaagctgactctggaggttgaactactacagctccagatacaaaaggagaaacacactgaagcacactgaccgggagttatgactgtatgttctcttaaggaggaagtagcgtctcattggctttctgacttcagttccaaacagattgcctgcagttaccaacctttgtaatgctggcaccaatgttcatgataataaatatctgcttagaataattcattaattatgttataattaataggtagtaaatctatgaaactatcaaaaaaaaaaaaaaa

SEQ ID NO: 30 - DNA, 1358 bp, Mus musculus
tgtttcacccagtatgaggagtcctctggcaggtgcaaaggcctacttgggagagacatcagggtagaagactgctgtctcaacgctgcctatgccttccaggagcatgatggtggcctctgtcaggcatgcaggtctccacaatggtcagcatggtccttatgggggccctgctcagttacatgttctgaggggtcccagctgcgacacaggcgctgtgtgggcagaggtggtcagtgctctgagaatgtggctcctggaactcttgagtggcagctacaggcctgtgaggaccagccatgctgtccagagatgggtggctggtctgagtggggaccctgggggccttgctctgtcacatgctccaaaggaacccagatccgtcaacgagtatgtgataatcctgctcctaagtgtgggggccactgcccaggagaggcccagcaatcacaggcctgtgacacccagaagacctgccccacacatggggcctgggcatcctggggcccctggagcccccgctcaggatcctgccttggtggtgctcaagaacctaaggagacacgaagccgctcatgttctgcaccagcaccttcacaccagccccctgggaaaccctgctcaggaccagcctatgagcataaggcctgcagtggcctaccaccttgcccagtggctggtggctgggggccatggagccctttgagcccctgctctgtgacttgtggcctgggccagaccctagagcaacggacatgtgatcaccctgcaccccgtcatgggggccccttttgtgctggtgatgccactcggaaccaaatgtgtaacaaagccgtaccttgccctgtaaacggggagtgggaggcctggggaaaatggagtgactgcagccggctgagaatgtccatcaactgtgaaggaaccccaggccagcagtcacgttcaaggagctgtggcgaccgcaaatttaatgggaagccatgtgctggaaaactccaggatattcgacactgctataacatccataactgtatcatgaaaggttcatggtcacagtggagtacctggagtctgtgcacaccaccatgtagtcccaacgccacccgtgtccgccagcgcctctgcacacctttgctccccaagtacccgcctacagtttcaatggttgaaggtcagggtgagaagaatgttaccttctgggggactccacggccactgtgtgaagcgctacaggggcagaagctggtggtggaagagaaacggtcatgtctacatgtgcctgtctgcaaagacccagaagagaagaaaccctaaaatcccttgcttccattctgaccccctgactttctagacccgga

SEQ ID NO: 31 - DNA, 1775 bp, Mus musculus
agtctgtcctgaagctgcctagtgaccaagaacttggaccaggacgcagctgacatcgctgcccagatggcctccaggctgaccccactgaccctcctgctgctgctgctggctggggatagagccttctcagatcccgaagctaccagccacagcacccaggatccactggaggctcaagcgaaaagcagagagagcttccctgaaagagatgactcctggagtcccccagagcctacagtactgccctctacctggccaacaaccagtgtagccatcacaataacaaatgacaccatgggtaaagtagccaacgagtccttcagccagcacagccagccagctgctcagctacccacagattctccaggacagccccctctgaattcttccagccagccctccactgcctcagatcttcccacccaggctactactgaacccttctgcccggagccgcttgctcagtgctctgattcagacagagactcctcagaggcaaagctctcagaggctttgacagatttctctgtgaagctctaccacgccttctcagctaccaagatggctaagaccaacatggccttttccccattcagcattgccagcctcctcacacaggttcttcttggggctggagacagcaccaagagcaacttggagagcatcctttcctaccccaaggattttgcctgtgtccaccaagcactaaagggcttttcatccaaaggtgtcacttctgtgtctcagattttccacagcccagatctggccataagggacacctatgtgaatgcatctcagagcctgtatggaagcagccccagagtcctgggcccagacagtgctgctaacttagaactcatcaacacctgggtggctgagaacaccaaccataagatccgcaagctgctggacagcctgccttctgacacccgcctcgtccttctcaatgctgtctacttgagtgccaagtggaagataacatttgaaccaaaaaagatgatggcgcctttcttctacaaaaactctatgattaaagtgcccatgatgagtagcgtaaagtaccctgtggcccaattcgatgaccatactttgaaggccaaggtgggacagctgcagctctctcacaacctgagctttgtgatcgtggtacccgtgttcccaaagcaccaacttaaagatgtagaaaaggctctcaaccccactgtcttcaaggccatcatgaagaagctggagctgtccaaattcctgcccacttacctgacgatgcctcatataaaagtaaagagcagccaagacatgctgtcagtcatggagaaactggaattctttgacttcacttacgatctcaacctgtgcgggctgaccgaggacccagatcttcaggtgtctgccatgaaacacgagacagtgctggaactgacagagtcaggggtggaagcagctgcagcctctgccatctcctttggccgaagcttacccatctttgaggtgcagcgacctttcctcttcctgctctgggaccagcaacacaggttcccagtcttcatgggtcgagtatatgaccccaggggttgagacaggcttgggtaaacattgtcacccaagcttcagctcctccggttatttccttgccactgcctgcccgagccacttcaagccttaggaactggcagacggaactgtttccatccaccaacccccagggtatcaaccacttttttgcagcttttacggttcaaacctatcaaactctacaaataaaccggaat

SEQ ID NO: 32 - DNA, 1701 bp, Homo sapiens
gagcaccgcacactcacttcaccctggttcaacacccccacgaggttgaccccgtcattatgttagagattatgcattttccacatagggaaactgaggctcaggggtgttaagtgactcacccaaggtcacacggctaggaagttgctgcacgctcctatgctccatttcctctgggagcctatcaacccagataaagcgggacctcctctctggtagaggtgcagggggcagtactcaacatgatcacagagggagcgcaggcccctcgattgttgctgccgccgctgctcctgctgctcaccctgccagccacaggctcagaccccgtgctctgcttcacccagtatgaagaatcctccggcaagtgcaagggcctcctggggggtggtgtcagcgtggaagactgctgtctcaacactgcctttgcctaccagaaacgtagtggtgggctctgtcagccttgcaggtccccacgatggtccctgtggtccacatgggccccctgttcggtgacgtgctctgagggctcccagctgcggtaccggcgctgtgtgggctggaatgggcagtgctctggaaaggtggcacctgggaccctggagtggcagctccaggcctgtgaggaccagcagtgctgtcctgagatgggcggctggtctggctgggggccctgggagccttgctctgtcacctgctccaaagggacccggacccgcaggcgagcctgtaatcaccctgctcccaagtgtgggggccactgcccaggacaggcacaggaatcagaggcctgtgacacccagcaggtctgccccacacacggggcctgggccacctggggcccctggaccccctgctcagcctcctgccacggtggaccccacgaacctaaggagacacgaagccgcaagtgttctgcacctgagccctcccagaaacctcctgggaagccctgcccggggctagcctacgagcagcggaggtgcaccggcctgccaccctgcccagtggctgggggctgggggccttggggccctgtgagcccctgccctgtgacctgtggcctgggccagaccatggaacaacggacgtgcaatcaccctgtgccccagcatgggggccccttctgtgctggcgatgccacccggacccacatctgcaacacagctgtgccctgccctgtggatggggagtgggactcgtggggggagtggagcccctgtatccgacggaacatgaagtccatcagctgtcaagaaatcccgggccagcagtcacgcgggaggacctgcaggggccgcaagtttgacggacatcgatgtgccgggcaacagcaggatatccggcactgctacagcatccagcactgccccttgaaaggatcatggtcagagtggagtacctgggggctgtgcatgcccccctgtggacctaatcctacccgtgcccgccagcgcctctgcacacccttgctccccaagtacccgcccaccgtttccatggtcgaaggtcagggcgagaagaacgtgaccttctgggggagaccgctgccacggtgtgaggagctacaagggcagaagctggtggtggaggagaaacgaccatgtctacacgtgcctgcttgcaaagaccctgaggaagaggaactctaacacttctctcctccactctgagccccctgaccttccaaacctcaataaa

SEQ ID NO: 33 - DNA, 1849 bp, Homo sapiens
atataattgtctatcagagaatgcttttatgtggtcccgtgtgaggtgaaggaaggcaaactaaaacagcgtgaggaccttctggtttcatgatcccacatctttatgtgggaagattagaatcctaagaatatgtatgcattttcaaaaagatactgtttgttttaacatttttttcatctttttgcagaagtttagcaatggcgtctttctctgctgagaccaattcaactgacctactctcacagccatggaatgagcccccagtaattctctccatggtcattctcagccttacttttttactgggattgccaggcaatgggctggtgctgtgggtggctggcctgaagatgcagcggacagtgaacacaatttggttcctccacctcaccttggcggacctcctctgctgcctctccttgcccttctcgctggctcacttggctctccagggacagtggccctacggcaggttcctatgcaagctcatcccctccatcattgtcctcaacatgtttgccagtgtcttcctgcttactgccattagcctggatcgctgtcttgtggtattcaagccaatctggtgtcagaatcatcgcaatgtagggatggcctgctctatctgtggatgtatctgggtggtggcttttgtgatgtgcattcctgtgttcgtgtaccgggaaatcttcactacagacaaccataatagatgtggctacaaatttggtctctccagctcattagattatccagacttttatggagatccactagaaaacaggtctcttgaaaacattgttcagccgcctggagaaatgaatgataggttagatccttcctctttccaaacaaatgatcatccttggacagtccccactgtcttccaacctcaaacatttcaaagaccttctgcagattcactccctaggggttctgctaggttaacaagtcaaaatctgtattctaatgtatttaaacctgctgatgtggtctcacctaaaatccccagtgggtttcctattgaagatcacgaaaccagcccactggataactctgatgcttttctctctactcatttaaagctgttccctagcgcttctagcaattccttctacgagtctgagctaccacaaggtttccaggattattacaatttaggccaattcacagatgacgatcaagtgccaacacccctcgtggcaataacgatcactaggctagtggtgggtttcctgctgccctctgttatcatgatagcctgttacagcttcattgtcttccgaatgcaaaggggccgcttcgccaagtctcagagcaaaacctttcgagtggccgtggtggtggtggctgtctttcttgtctgctggactccataccacatttttggagtcctgtcattgcttactgacccagaaactcccttggggaaaactctgatgtcctgggatcatgtatgcattgctctagcatctgccaatagttgctttaatcccttcctttatgccctcttggggaaagattttaggaagaaagcaaggcagtccattcagggaattctggaggcagccttcagtgaggagctcacacgttccacccactgtccctcaaacaatgtcatttcagaaagaaatagtacaactgtgtgaaaatgtggagcagccaacaagcaggggctcttaggcaatcacatagtgaaagtttataagaggatgaagtgatatggtgagcagcggacttcaaaaactgtcaaagaatcaatccagcggttctcaaacggtacacagactattgacatcagcatcacctagaaacttgttagaaatgcaaattctcaagccgcatccca

SEQ ID NO: 34 - DNA, 1684 bp, Homo sapiens
cgccaggaggagcgcgcgggcacagggtgccgctgaccgaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccacagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcacctacgggggcatctgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctgaaaaaaaaaaaaaaaaaaaaaa

SEQ ID NO: 35 - DNA, 1701 bp, Mus musculus
cttcattttggctggaccagacaggatttgttggtggctcgcagatcatcagtcctggagcctttggattccatctcagtgtgcttgactgagccatggagtctttcgatgctgacaccaattcaactgacctacactcacggcctctgtttcaaccccaagacattgcctccatggtcattcttggtctcacttgtctattgggactgctaggcaatgggctggtgctgtgggtagctggcgtaaagatgaagacgaccgtgaacacagtctggttcctccatctcaccctggccgatttcctctgctgcctctccttgcccttctccttggctcacctgattctccaaggacactggccctatggcttgttcctgtgcaaacttatcccatccatcattattctcaacatgtttgccagtgtcttcctgcttactgccattagcctggaccgatgtctgatagtacataagccaatctggtgccagaatcatcgaaacgtgagaaccgccttcgccatctgtggatgtgtctgggtggtagcctttgtgatgtgtgtgcccgtatttgtataccgtgatctgttcattatggacaatcgcagtatatgtagatataattttgattcctccaggtcatatgattattgggactacgtgtacaaactaagtctaccagaaagcaattctactgataactccactgctcagctaactggacatatgaatgacaggtcagctccttcctctgtacaggcaagggattacttttggacagttaccactgccctccagtcacagccattcctaacatctcctgaagactcattctctctagattcagcaaaccaacaaccccattatggtggaaagcctcctaatgtcctcacagccgccgtacccagcgggtttcctgttgaagatcgtaaatccaatacactgaacgctgacgcttttctctctgctcacacagaacttttccctactgcttctagtggtcatttatacccctatgatttccagggggattatgttgaccaattcacgtatgacaatcatgtgccgacaccgctgatggcaataaccatcacaaggctggtggtgggcttcctggtgccgtttttcatcatggtaatttgttacagcctcatcgtcttcagaatgcgaaaaaccaacttcaccaagtctcggaacaaaacctttcgggtggctgtggctgtggtcactgtcttttttatctgctggactccataccatcttgtcggagtcctgctattgattactgatccagaaagttccttgggggaagctgtgatgtcctgggaccacatgtccattgctttagcatctgccaatagttgcttcaaccctttcctgtatgccctcttggggaaagactttaggaagaaagcaagacagtctataaagggcattctggaagcagccttcagcgaagagctcacgcactctaccaactgtacccaagacaaagcctcttcaaaaagaaacaatatgagtacagatgtgtgaagatgtggccctgggaacctaagcagagttctcaggtgaacagtgatggatgacatgtgagcaggacactttagacaatttggcgactctcagagaaaggtctcttattgacatcagcatcatttgaaaacattaaagatgcaaaatttcaagccccaaaaaaaaaaaaaaa

SEQ ID NO: 36 - DNA, 1332 bp, Mus musculus
cctcaaaacagctccggccaaatcctggcatggtctcttccacctggggctatgatccccgcgcaggcgccggggacttggtcatcaccaccactgctgctggtgctgtcactatcgctgtgctgttgttccaaactgtgtgcggagactgcggcccacctccagacattcctaatgccaggccaatcttgggcagacactccaagtttgctgagcaaagcaaagtggcatactcgtgtaataacggctttaaacaagttccagacaagtcaaacatagttgtctgtcttgaaaatggccaatggtcgagccacgaaacattctgtgagaaatcatgtgatactccagaaagactgagttttgcatccctcaaaaaagagtacttcaacatgaattttttcccagttggtactattgtggaatatgagtgtcggccaggatttcgaaaacaaccttcactctcaggaaaatcaacttgccttgaggatttagtatggtctccagttgctcagttttgtaaaaaaaaatcatgccctaatcctaaagatctggataatggtcacatcaacataccaaccggaatattattcggttcagaaataaacttctcatgcaacccagggtacaggctagtcggtatcacctctattttatgtactattacaggaaatgctgttgattgggatgatgaatttccagtgtgcacagaaatattttgtccagacccaccaaaaatcaacgatggaataatgcgaggggaaagcgattcttataaatatagccaggtggtcatttattcatgtgacaaaggcttcatcctgtttggaaattctaccatatattgtactgtgagcaagtctgatgtaggacaatggagcagtccaccaccccagtgtatagaggaatctaaggtcccaattaagaaaccagtagttaatgttccaagtacaggaatcccctcaacgcctcagaaacccacaacagaaagtgttccaaatccaggagaccaaccaactcctcagaaaccttccacagttaaagttccagcaacccagcatgaacctgataccacgacaagaacatctacagacaaaggagagtctaactcaggtggtgaccgttatatatatggatttgttgctgttattgcaatgattgatagcctaattatagtcaaaactctttggactattttaagtccaaacagaaggtctgactttcaaggaaaggagaggaaggatgtctcaaagtaagaagagagcaagaatccagtctccaccttttcaacctgtttgggtgtgtgctctatgaattaaatggcagcccacatag

SEQ ID NO: 37 - DNA, 1288 bp, Mus musculus
cttctacctggggctatgatccgtgggcgggcgcctaggactcggccatcaccgccgcctccgctgctgccgttgctgtcgctgtctctgttgctgctgtccccaactgtacgcggagactgcggcccacctccagacattcctaatgccaggccaatcttgggcagacactccaagtttgctgagcaaagcaaagtggcatactcgtgtaataacggctttaaacaagttccagacaagtcaaacatagttgtctgtcttgaaaatggccaatggtcgagccacgaaacattctgtgagaaatcatgtgttgctccagaaagactgagttttgcatccctcaaaaaagagtacctcaacatgaattttttcccagttggtactattgtggaatatgagtgtcggccaggatttcgagaacaacctccactcccaggaaaagcaacttgccttgaggatttagtatggtctccagttgctcagttttgtaaaaaaaaatcatgccctaatcctaaagatctggataatggtcacatcaacataccaaccggcatattattcggttcagaaataaacttctcatgcaacccagggtacaggctagtcggtgtctcctctactttctgttctgtcacaggaaatactgttgattgggacgatgagtttccagtgtgcacagaaatacattgtccagagccaccaaaaatcaacaatggaataatgcgaggggaaagtgactcttatacgtatagccaggtggtcacctattcatgtgacaaaggcttcatcctggttggaaatgctagcatttattgtactgtgagcaagtctgatgtaggacaatggagcagtccaccaccccggtgcatagagaaatccaaggtcccaacgaagaaaccaacaattaatgttccaagtacaggaaccccctcaacgcctcagaagcccacaacagaaagtgttccaaatccaggagaccaaccaactcctcagaaaccttccacagttaaagtttcagcaacccagcatgtacctgttaccaagacaacagtacgtcatccaataagaacatctacagacaaaggagagcctaacacaggtggtgaccgttatatatatggacatacatgtttaataaccttgacagttttgcatgtgatgctatcactcattggctacttgacatagccaacgaagagttacgaagaaagtatataaaactactgataatacttctagtttgttagactgtccaagaagaatggatacaataaatttaatagtgtcg

SEQ ID NO: 38 - DNA, 1444 bp, Mus musculus
gtaaaacgttgtttgagaacggtgtgaggggaatggaggtctcttctcggagttcagagcctctggatccggtgtggctccttgtagccttcggccggggaggagtcaagctagaagttttgctgctgttcttgctgccatttactttgggtcactgcccagccccatcacagcttccttctgccaaacctataaatctaactgatgaatccatgtttcccattggaacatatttgttgtatgaatgtctcccaggatatatcaagaggcagttctctatcacctgcaaacaagactcaacctggacgagtgctgaagataagtgtatacgaaaacaatgtaaaactccttcagatcctgagaatggcttggtacatgtacacacaggcattcagtttggatcccgtattaattatacttgtaatcaaggataccgcctcattggttcctcctctgctgtatgtgtcatcactgatcaaagtgttgattgggatactgaggcacctatttgtgagtggattccttgtgagatacccccaggcattcccaatggagatttcttcagttcaaccagagaagactttcattatggaatggtggttacctaccgctgcaacactgatgcgagagggaaggcgctctttaacctggtgggtgagccctccttatactgtaccagcaacgatggtgaaattggagtctggagcggccctcctcctcagtgcattgaactcaacaaatgtactcctcctccctatgttgaaaatgcagtcatgctgtctgagaacagaagcttgttttccttaagggatattgtggagtttagatgtcaccctggctttatcatgaaaggagccagcagtgtgcattgtcagtccctaaacaaatgggagccagagttaccaagctgcttcaagggagtgatatgtcgtctccctcaggagatgagtggattccagaaggggttgggaatgaaaaaagaatattattatggagagaatgtaaccttggaatgtgaggatgggtatactctagaaggcagttctcaaagccagtgccagtctgatggcagctggaatcctcttctggccaaatgtgtatctcgctcaatcagtggtctaattgttggaattttcattgggataatcgtctttattttagtcatcattgttttcatttggatgattctgaagtataaaaaacgcaataccacagatgaaaagtataaagaagtgggtattcatttaaattataaagaagacagctgtgtccgccttcagtctctgctcacaagtcaggagaacagcagtaccactagcccagcacggaattcactcactcaagaagtctcctaaatagcagcaacgtgaaatgagaacatgctctgtctgtatcacttttaaaataaactgtttccttttaaaaaaaaaaaaaaaaaaaaaaa

SEQ ID NO: 39 - DNA, 1648 bp, Mus musculus
gacatgcaaagtagcctcaaagaggtgacagatatggttttgataccgtctcaagctatgggcttttggggaacacttctgtttctgatcttccttgaacaaagttggggacaggaacaaaccagatacatcatttcaaccccaattgtcttccgagttggagctcctgaaaatgttacagtccaagcccacggccatactgaggcatttgacacaactgtctctgtaaaaagttatcctgatgaaaatgttcgttactctttcagcactgttaatttatcaccagaaaataaattccaaaacactgcaatcttaacaattcaggccaaacagttatctgaaggactaaactcattttcaaattcgtatttagaagtcgtgtcaaagcattttgcaaaattagaaatcgtgccaatcatctatgacaatgactctctcttcgttcaaaccgacaagtccgtgtatactccacaacagcctgtaaaggttcgtgtctactctgtgaatgatgacttagagccggccaccagagaaacagtcttaactttcatagatcctgaagggtcccaagttgacacaatagagggaaataatctcaccgggattgcctcttttcctgacttcgagattccttctaatcccaagcacggtagatggacagtcaaggctaagtatagagaagatgcttcaaaaactggaactacatactttgaagttaaagaatacgataaaacttacagaatatctatcatgcccacaattgatctgcaacccgaggtggaaaagcaagaagcacatggcatgtgtcttcatcagccaacagagtgtctgcgacagaagataaacgagcaagcttctacatacaaacatccaatgataaaaaaatgctgttacgatggggccagatataacatacatgagacctgtgtgcagcgagctgcccgtgtgaagataggcccgatctgtgtcaaagccttcactctctgctgtaacatggcacaccagatcctagaaaacagcacctttaagcacatccatctgtcaagtcactacagaagctagcataattgtcctctgagtggctccacccagcaactaagggaaagagatacagaaactcacagccaaacattagatggagctcggggagtcttatggaagagttggggaaagaactgaaggacctgaagaggagaaggaagaccaacagagtcaactaacctggtcccttgggggttcccagagtctgaatcaccaaccaaagaacgaggactgactggacctaggtcccctgcacatatgtgacatatgagcagcttggtctttatatgggtgtcccaacaaactgaagaagctgtccctgaatatgttgcctgcctgtctgcctgcctgcctgcctgcctgcctgcctgcctacctgtggatcctgttcccctaaatggtctgccttgtctggcctcagtggaagaggatgaacctagtcctgcaggggcttgatgtgtgaggggtaatacccagagtggggtacctgcttcacaaaggataaggggagggtggaatggggagaagatctgcatgggggtactggaaggaaaggcagggttgatatgggaatgtaaagtgaataaataaatttaattaaaacacg

SEQ ID NO: 40 - DNA, 3326 bp, Homo sapiens
gctcgggccacgcccacctgtcctgcagcactggatgctttgtgagttggggattgttgcgtcccatatctggacccagaagggacttccctgctcggctggctctcggtttctctgctttcctccggagaaataacagcgtcttccgcgccgcgcatggagcctcccggccgccgcgagtgtccctttccttcctggcgctttcctgggttgcttctggcggccatggtgttgctgctgtactccttctccgatgcctgtgaggagccaccaacatttgaagctatggagctcattggtaaaccaaaaccctactatgagattggtgaacgagtagattataagtgtaaaaaaggatacttctatatacctcctcttgccacccatactatttgtgatcggaatcatacatggctacctgtctcagatgacgcctgttatagagaaacatgtccatatatacgggatcctttaaatggccaagcagtccctgcaaatgggacttacgagtttggttatcagatgcactttatttgtaatgagggttattacttaattggtgaagaaattctatattgtgaacttaaaggatcagtagcaatttggagcggtaagcccccaatatgtgaaaaggttttgtgtacaccacctccaaaaataaaaaatggaaaacacacctttagtgaagtagaagtatttgagtatcttgatgcagtaacttatagttgtgatcctgcacctggaccagatccattttcacttattggagagagcacgatttattgtggtgacaattcagtgtggagtcgtgctgctccagagtgtaaagtggtcaaatgtcgatttccagtagtcgaaaatggaaaacagatatcaggatttggaaaaaaattttactacaaagcaacagttatgtttgaatgcgataagggtttttacctcgatggcagcgacacaattgtctgtgacagtaacagtacttgggatcccccagttccaaagtgtcttaaagtgtcgacttcttccactacaaaatctccagcgtccagtgcctcaggtcctaggcctacttacaagcctccagtctcaaattatccaggatatcctaaacctgaggaaggaatacttgacagtttggatgtttgggtcattgctgtgattgttattgccatagttgttggagttgcagtaatttgtgttgtcccgtacagatatcttcaaaggaggaagaagaaaggcacatacctaactgatgagacccacagagaagtaaaatttacttctctctgagaaggagagatgagagaaaggtttgcttttatcattaaaaggaaagcagatggtggagctgaatatgccacttaccagactaaatcaaccactccagcagagcagagaggctgaatagattccacaacctggtttgccagttcatcttttgactctattaaaatcttcaatagttgttattctgtagtttcactctcatgagtgcaactgtggcttagctaatattgcaatgtggcttgaatgtaggtagcatcctttgatgcttctttgaaacttgtatgaatttgggtatgaacagattgcctgctttcccttaaataacacttagatttattggaccagtcagcacagcatgcctggttgtattaaagcagggatatgctgtattttataaaattggcaaaattagagaaatatagttcacaatgaaattatattttctttgtaaagaaagtggcttgaaatcttttttgttcaaagattaatgccaactcttaagattattctttcaccaactatagaatgtattttatatatcgttcattgtaaaaagcccttaaaaatatgtgtatactactttggctcttgtgcataaaaacaagaacactgaaaattgggaatatgcacaaacttggcttctttaaccaagaatattattggaaaattctctaaaagttaatagggtaaattctctattttttgtaatgtgttcggtgatttcagaaagctagaaagtgtatgtgtggcatttgttttcactttttaaaacatccctaactgatcgaatatatcagtaatttcagaatcagatgcatcctttcataagaagtgagaggactctgacagccataacaggagtgccacttcatggtgcgaagtgaacactgtagtcttgttgttttcccaaagagaactccgtatgttctcttaggttgagtaacccactctgaattctggttacatgtgtttttctctccctccttaaataaagagaggggttaaacatgccctctaaaagtaggtggttttgaagagaataaattcatcagataacctcaagtcacatgagaatcttagtccatttacattgccttggctagtaaaagccatctatgtatatgtcttacctcatctcctaaaaggcagagtacaaagtaagccatgtatctcaggaaggtaacttcattttgtctatttgctgttgattgtaccaagggatggaagaagtaaatatagctcaggtagcactttatactcaggcagatctcagccctctactgagtcccttagccaagcagtttctttcaaagaagccagcaggcgaaaagcagggactgccactgcatttcatatcacactgttaaaagttgtgttttgaaattttatgtttagttgcacaaattgggccaaagaaacattgccttgaggaagatatgattggaaaatcaagagtgtagaagaataaatactgttttactgtccaaagacatgtttatagtgctctgtaaatgttcctttcctttgtagtctctggcaagatgctttaggaagataaaagtttgaggagaacaaacaggaattctgaattaagcacagagttgaagtttatacccgtttcacatgcttttcaagaatgtcgcaattactaagaagcagataatggtgttttttagaaacctaattgaagtatattcaaccaaatactttaatgtataaaataaatattatacaatatacttgtatagcagtttctgcttcacatttgattttttcaaatttaatatttatattagagatctatatatgtataaatatgtattttgtcaaatttgttacttaaatatatagagaccagttttctctggaagtttgtttaaatgacagaagcgtatatgaattcaagaaaatttaagctgcaaaaatgtatttgctataaaatgagaagtctcactgatagaggttctttattgctcattttttaaaaaatggactcttgaaatctgttaaaataaaattgtacatttggagatgtttca

SEQ ID NO: 41 - DNA, 1056 bp, Mus musculus
atggaccccatagataacagcagctttgaaatcaactatgatcactatggaaccatggatcctaacatacctgcggatggcattcacctcccgaagcggcaacctggggatgttgcagcccttatcatctactcggtggtgttcctggtgggagtacccgggaatgccctggtggtgtgggtgacagccttcgagccagacgggccgtcaaacgccatctggtttctgaatctggcggtggccgacctcctctcgtgcttggccatgcctgtcctgttcacgaccgttttaaatcataactactggtactttgatgccaccgcctgtatagtcctgccctcgctcatcctgctcaacatgtacgccagtatcctgctgctggctaccattagtgccgaccgtttcctgctggtgttcaagcccatctggtgtcagaaggtccgcgggactggcctggcatggatggcctgtggagtggcctgggtcttagcattgctcctcaccattccatccttcgtgtaccgggaggcatataaggacttctactcagagcacactgtatgtggtattaactatggtgggggtagcttccccaaagagaaggctgtggccatcctgcggctgatggtgggttttgtgttgcctctgctcactctaaacatctgctacaccttcctcctgctccggacctggagtcgcaaggccacgcgctccaccaagacgctcaaagtggtgatggctgtggtcatctgtttctttatcttctggctgccctatcaggtgaccggggtgatgatagcgtggctgcccccgtcctcgcccaccttgaagagggtggagaagctgaactccctgtgcgtgtccctggcctacatcaactgctgtgttaaccctatcatctacgtcatggctggccagggtttccatggacgactcctaaggtctctccccagcatcatacgaaacgctctctctgaggattcagtgggcagggatagcaagactttcactccgtccacagacgacacctcaacccggaagagtcaggcggtgtag

SEQ ID NO: 42 - DNA, 1266 bp, Homo sapiens
tgttaatgaaagcagattcaaagcaacaccaccaccactgaagtatttttagttatataagattggaactaccaagcatgtggctcctggtcagtgtaattctaatctcacggatatcctctgttgggggagaagcaacattttgtgattttccaaaaataaaccatggaattctatatgatgaagaaaaatataagccattttcccaggttcctacaggggaagttttctattactcctgtgaatataattttgtgtctccttcaaaatcattttggactcgcataacatgcacagaagaaggatggtcaccaacaccaaagtgtctcagactgtgtttctttccttttgtggaaaatggtcattctgaatcttcaggacaaacacatctggaaggtgatactgtgcaaattatttgcaacacaggatacagacttcaaaacaatgagaacaacatttcatgtgtagaacggggctggtccacccctcccaaatgcaggtccactgacacttcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacgggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttatttgagaacaggtgaatcagctgaatttgtgtgtaaacggggatatcgtctttcatcacgttctcacacattgcgaacaacatgttgggatgggaaactggagtatccaacttgtgcaaaaagatagaatcaatcataaaatgcacacctttattcagaactttagtattaaatcagttcttaatttaatttttaagtattgttttactcctttttattcatacgtaaaattttggattaatttgtgaaaatgtaattataagctgagaccggtggctctcttcttaaaagcaccatattaaaacttggaaaactggaaaact

SEQ ID NO: 43 - DNA, 990 bp, Mus musculus
gaattccttttcctgccacagggccttgacgcatactgtaccagcaacgatggtgaaattggagtctggagcggccctcctcctcagtgcattgaactcaacaaatgtactcctcctccctatgttgaaaatgcagtcatgctgtctgagaacagaagcttgttttccttaagggatattgtggagtttagatgtcaccctggctttatcatgaaaggagccagcagtgtgcattgtcagtccctaaacaaatgggagccagagttaccaagctgcttcaagggagtgatatgtcgtctccctcaggagatgagtggattccagaaggggttgggaatgaaaaaagaatattattatggagagaatgtaaccttggaatgtgaggatgggtatactctagaaggcagttctcaagccagtgccatttacctttgggtgagctgcggggaggcctggggaagcacggacacacggttcaccgggaaacccgcggtaaataggctctgcgcagactccaacgctggtctgggctgcctggtgagtgctcagcgcccctttcccatgggtcactgccagccccttccttctgccaaacctataaatctaactgatgaatccatgtttcccattggaacatatttgttgtatgaatgtctcccaggatatatcaagaggcagttctctatcacctgcaaacaagactcaacctggacgagtgctgaagataagtgtatacgaaaacaatgtaaaactccttcagatcctgagaatggcttggtacatgtacacacaggcattgagtttggatcccgtattaattatacttgtaatcaaggataccgcctcattggttcctcctctgctgtatgtgtcatcactgatcaaagtgttgattgggatactgaggcacctatttgtgagtggattccttgtgagatacccccaggcattcccaatggagatttcttcagttcaaccagagaagacttt

SEQ ID NO: 44 - DNA, 2102 bp, Homo sapiens
ccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaacagagcaaggagaaaaaaggcagtcctggaatcacattcttagcacacctacacctcttgaaaatagaacaacttgcagaattgagagtgattcctttcctaaaagtgtaagaaagcatagagatttgttcgtatttagaatgggatcacgaggaaaagagaaggaaagtgatttttttccacaagatctgtaatgttatttccacttataaaggaaataaaaaatgaaaaacattatttggatatcaaaagcaaataaaaacccaattcagtctcttctaagcaaaattgctaaagagagatgaaccacattataaagtaatctttggctgtaaggcattttcatctttccttcgggttggcaaaatattttaaaggtaaaacatgctggtgaaccaggggtgttgatggtgataagggaggaatatagaatgaaagactgaatcttcctttgttgcacaaatagagtttggaaaaagcctgtgaaaggtgtcttctttgacttaatgtctttaaaagtatccagagatactacaatattaacataagaaaagattatatattatttctgaatcgagatgtccatagtcaaatttgtaaatcttattcttttgtaatatttatttatatttatttatgacagtgaacattctgattttacatgtaaaacaagaaaagttgaagaagatatgtgaagaaaaatgtatttttcctaaatagaaataaatgatcccattttttggt

SEQ ID NO: 45 - DNA, 1127 bp, Mus musculus
atcgaattcccgtgtccgccccgcatgctccctctgctgcgttgcgtgccccgctccctcggcgccgcctcgggcctccgaaccgccatcccggcccagccgcttcggcatctcctgcagcccgcgccccggccatgcctccggcccttcggtttgctcagcgtacgggccggctcggctcggcgctctggcctcctgcagcccccggttccctgcgcgtgcggctgtggcgctctgcacacggaaggagacaaggccttcgttgaattcttgactgatgaaattaaggaagaaaagaagatccagaaacacaagtcccttcccaagatgtctggagattgggagctggaggtgaacggcacggaggctaaattattgcgcaaagttgccggagaaaagatcacggtcactttcaacatcaacaacagcatccctccaacatttgatggtgaggaggagccctcacaggggcagaaggctgaagaacaggagccagaacggacatcaactcccaactttgtggttgaagttacaaagactgatggcaagaagacccttgtactggactgtcactatcctgaggatgagattggacacgaagatgaggccgagagtgatattttctctatcaaggaagttagctttcaggccactggtgactctgagtggagggatacaaactatacactcaacacagattccctggactgggccttgtatgaccacctaatggatttccttgcggaccgaggggtggataacacttttgcggatgagttggtggagctcagcacagccctggagcaccaggaatatatcacctttcttgaggacctcaaaagctttgtcaagaaccagtagaactcagagactgcgggccttaatttaaatggcaagctttggccagtgaacaaaagctcccttggcatgagaattatgcttcaaaaatggctgtcatcctaatatatcggggggaagcaagtttaaattactgctgttacacctccattcgctattcctttgggctttttttctctgtacaaatttattatttgtagatttttgtataacatgatgatggacaataaatatgactccaataaaaaaaaaaaaaaaaaaaaaaaaaa

SEQ ID NO: 46 - DNA, 862 bp, Mus musculus
tgcctgctgtcagaatgcacagctccgtgtacttcgtggctctggtgatcctgggagcggctgtatgtgcagcacagccccgaggccggattctgggtggccaggaggccgcagcccatgctcggccctacatggcttccgtgcaagtgaacggcacacacgtgtgcggtggcaccctgctggacgagcagtgggtgctcagtgctgcacactgcatggatggagtgacggatgacgactctgtgcaggtgctcctgggtgcccactccctgtccgcccctgaaccctacaagcgatggtatgatgtgcagagtgtagtgcctcacccgggcagccgacctgacagccttgaggacgacctcattctttttaagctatcccagaatgcctcgttgggtccccacgtgagacccctacccttgcaatacgaggacaaagaagtggaacccggcacgctctgcgacgtggctggttggggtgtggtcacccatgcaggacgcaggcctgatgtcctgcatcaactcagagtgtcaatcatgaaccggacaacctgcaatctgcgcacgtaccatgacggggtagtcaccattaacatgatgtgtgcagagagcaaccgcagggacacttgcaggggagactccggcagccctctagtgtgcggggatgcagtcgaaggtgtggttacgtggggctctcgcgtctgtggcaatggcaaaaagccgggcgtctatacccgagtgtcatcctaccggatgtggatcgaaaacatcacaaatggtaacatgacatcctgaggggacaccagagacacgtggctcagggaaacaagagacacgtggctcacaataaatgcatgcatctg

SEQ ID NO: 47 - DNA, 1091 bp, Rattus norvegicus
attcgcatttctagaaactgggaaatttcttaagattttaattctggcagctctttaattgtctctttgtggttgcaaatccactggatacactgtcttatttctgctattcttctctattacagggtagactttctttttcccatctgttacaggggaaatataattccttagaaggaagttgttttgatctgacgtctttagaggatgcttttgactgatatcagagtttaagtccatcgtgggtcaagtaactggtcaccaaatgctttgtttggttgtgtgctgtctgatatggttgatttctgccttagatgggagctgttcagaaccccctccggtgaacaatagtgtgtttgttggaaaggaaactgaagaacagattctgggaatttacctttgtatcaaaggctaccacttggtgggaaagaagtctttggtctttgatccctcgaaggaatggaattcgaccctccctgagtgcctcctgggccactgtcctgaccctgtactggaaaatggcaagatcaattcttctgggcctgtgaatataagtggcaaaatcatgtttgagtgtaatgatggttacatcctcaagggaagcaattggagccagtgcctagaggaccacacctgggcacctcccttgcccatctgccgaagtagagactgtgaacctcctgagactcctgtccatggctattttgaaggagaaactttcacttcaggatctgtcgttacttattactgtgaagatgggtaccacctagtgggcacacagaaggtgcagtgcagtgatggagagtggagcccgtcctatcctacctgtgagtccatccaggaaccccccaaatcagctgaacagagtgcacttgagaaagctattcttgcctttcaggagagtaaggacctttgcaatgctacagagaactttgtgagacagctaagggaaggtggaataacaatggaagaacttaaatgttctctggagatgaagaaaactaagctgaagtcggatattttactgaactaccatagctaagcagaatggttacagacagacacctatgaataaattgcttctaaaggtg

SEQ ID NO: 48 - DNA, 846 bp, Rattus sp.
atgcacagctccgtgtacctcgtggctctggtggtcctggaggcggctgtatgtgttgcgcagccccgaggtcggattctgggtggccaggaggccatggcccatgctcggccctacatggcttcagtgcaagtgaatggcacgcacgtgtgcggtggcaccctggtggatgagcagtgggtgctgagcgccgcgcactgcatggatggagtgaccaaggatgaggttgtgcaggtgctcctgggtgcccactccctgtccagtcctgaaccctacaagcatttgtatgatgtgcaaagtgtagtgcttcacccgggcagccggcctgacagcgttgaggacgacctcatgctctttaagctctcccacaatgcctcactgggtccccatgtgagacccctgcccttgcaacgcgaggaccgggaggtgaaacccggcacgctctgcgatgtggccggttggggcgtggtcactcatgcgggacgcaggcccgatgtcctgcagcaactgacagtgtcaatcatggaccggaacacctgcaatctgcgcacgtaccatgatggggcaatcaccaagaacatgatgtgtgcagagagcaaccgcagggacacttgcaggggcgactccggcggtcctctggtgtgcggggatgcggtcgaagctgtggttacgtggggatctcgagtctgtggcaaccggagaaagccaggtgtctttacccgcgtggcaacctacgtgccgtggattgaaaacgttctgagtggtaacgtgagtgttaacgtgacggcctgaggggacaccggagaccgtgactcacaataaatgcatgcatctaaaaaaaaaaaa

SEQ ID NO: 49 - DNA, 1157 bp, Homo sapiens
attctgtctttcacatacattgagaccaaaaagaccaagtacctataagaggaccaacccagacgggctgtgacaattacgctgttgcttctgagtgagaagttacaggcccaagaaagggtaatgacagccttagagatacataaaagagacaagcaatttccaaaacaaaaagcaaaggcaaaaagaaaaataaaaaagcaggcctttggagctctcagctttggagtcagttaagaccagttccttgctgggaagccctaactctggagggacagagacaggtgtctgagctgggtgaattccagcctggggagaggactttgatcaccagatgtttttttggtgtgcgtgctgtcttatggttgcgtggcgagtttctgcttcagatgcagagcactgtccagagcttcctccagtggacaatagcatatttgtcgcaaaggaggtggaaggacagattctggggacttacgtttgtatcaagggctaccacctggtaggaaagaagacccttttttgcaatgcctctaaggagtgggataacaccactactgagtgccgcttgggccactgtcctgatcctgtgctggtgaatggagagttcagttcttcagggcctgtgaatgtaagtgacaaaatcacgtttatgtgcaatgaccactacatcctcaagggcagcaatcggagccagtgtctagaggaccacacctgggcacctccctttcccatctgcaaaagtagggactgtgaccctcctgggaatccagttcatggctattttgaaggaaataacttcaccttaggatccaccattagttattactgtgaagacaggtactacttagtgggcgtgcaggagcagcaatgcgttgatggggagtggagcagtgcacttccagtctgcaagttgatccaggaagctcccaaaccagagtgtgagaaggcacttcttgcctttcaggagagtaagaacctctgcgaagccatggagaactttatgcaacaattaaaggaaagtggcatgacaatggaggagctaaaatattctctggagctgaagaaagctgagttgaaggcaaaattgttgtaacactacagctgagcagatgtaatagaaataaacctatgaataaattttcttcttggttctgaaa

SEQ ID NO: 50 - DNA, 1173 bp, Homo sapiens
gtgtctcagccacagcggcttcaccatgcacagctgggagcgcctggcagttctggtcctcctaggagcggccgcctgcgcggcgccgccccgtggtcggatcctgggcggcagagaggccgaggcgcacgcgcggccctacatggcgtcggtgcagctgaacggcgcgcacctgtgcggcggcgtcctggtggcggagcagtgggtgctgagcgcggcgcactgcctggaggacgcggccgacgggaaggtgcaggttctcctgggcgcgcactccctgtcgcagccggagccctccaagcgcctgtacgacgtgctccgcgcagtgccccacccggacagccagcccgacaccatcgaccacgacctcctgctgctacagctgtcggagaaggccacactgggccctgctgtgcgccccctgccctggcagcgcgtggaccgcgacgtggcaccgggaactctctgcgacgtggccggctggggcatagtcaaccacgcgggccgccgcccggacagcctgcagcacgtgctcttgccagtgctggaccgcgccacctgcaaccggcgcacgcaccacgacggcgccatcaccgagcgcttgatgtgcgcggagagcaatcgccgggacagctgcaagggtgactccgggggcccgctggtgtgcgggggcgtgctcgagggcgtggtcacctcgggctcgcgcgtttgcggcaaccgcaagaagcccgggatctacacccgcgtggcgagctatgcggcctggatcgacagcgtcctggcctagggtgccggggcctgaaggtcagggtcacccaagcaacaaagtcccgagcaatgaagtcatccactcctgcatctggttggtctttattgagcacctactatatgcagaaggggaggccgaggtgggaggatcattggatctcaggagttcgagatcagcatgggccacgtagcgcgactccatctctacaaataaataaaaaattagctgggcaattggcgggcatggaggtgggtgcttgtagttccagctactcaggaggctgaggtgggaggatgacttgaacgcaggaggctgaggctgcagtgagttgtgattgcaccactgccctccagcctgggcaacagagtgaaaccttgtctctctctacaaaaaaaaaaaaaaaa

SEQ ID NO: 51 - DNA, 968 bp, Homo sapiens
cgcggcgccgccccgtggtcggatcctgggcggcagagaggccgaggcgcacgcgcggccctacatggcgtcggtgcagctgaacggcgcgcacctgtgcggcggcgtcctggtggcggagcagtgggtgctgagcgcggcgcactgcctggaggacgcggccgacgggaaggtgcaggttctcctgggcgcgcactccctgtcgcagccggagccctccaagcgcctgtacgacgtgctccgcgcagtgccccacccggacagccagcccgacaccatcgaccacgacctcctgctgctacagctgtcggagaaggccacactgggccctgctgtgcgccccctgccctggcagcgcgtggaccgcgacgtggcaccgggaactctctgcgacgtggccggctggggcatagtcaaccacgcgggccgccgcccggacagcctgcagcacgtgctcttgccagtgctggaccgcgccacctgcaaccggcgcacgcaccacgacggcgccatcaccgagcgcttgatgtgcgcggagagcaatcgccgggacagctgcaagggtgactccgggggcccgctggtgtgcgggggcgtgctcgagggcgtggtcacctcgggctcgcgcgtttgcggcaaccgcaagaagcccgggatctacacccgcgtggcgagctatgcggcctggatcgacagcgtcctggcctagggtgccggggcctgaaggtcagggtcacccaagcaacaaagtcccgagcaatgaagtcatccactcctgcatctggttggtctttattgagcacctactatatgcagaaggggaggccgaggtgggaggatcattggatctcaggagttggagatcagcatgggccacgtagcgcgactccatctctacaaataaataaaaattagctgggcaaaaaaaaaaaaaaaaaaaaaaaaaaaaaaaaaaaaaaaaaaaaaaa

SEQ ID NO: 52 - DNA, 1781 bp, Mus musculus
gaattcataggctgccacttcagaatacactcctgctaagttacttagcagagttgatttccccacagatggaaaacccacaaacccaattcgagcacttcacctgtcttggccacatcaaaaccttctcctggccccccgccgccgccacctttaggagtgatgagttctcttcgaagtttagcaaggcgagccttaagcaggccaaggtgatgagctgtggccttgttttctttggagtcgaaggttcctgcaagtggaaacttcctggagctgacctactaggtattgaaccagtttctgcattgctgaatcaatctcccaagggtaattccacagaaatcccaggggcttggagtaaacaagaccgcgcctagcccagctagaggaagttttattccggaacccagcgccatttctgggtgggactgctttctacaccatttgccgtaaaacgttgtttgagaacggtgtgaggggaatggaggtctcttctcggagttcagagcctctggatccggtgtggctccttgtagccttcgcgcggggaggagtcaagctagaagttttgctgctgttcttgctgccatttactttgggtcactgcccagccccatcacagcttccttctgccaaacctataaatctaactgatgaatccatgtttcccattggaacatatttgttgtatgaatgtctcccaggatatatcaagaggcagttctctatcacctgcaaacaagactcaacctggacgagtgctgaagataagtgtatacgaaaacaatgtaaaactccttcagatcctgagaatggcttggtacatgtacacacaggcattgagtttggatcccgtattaattatacttgtaatcaaggataccgcctcattggttcctcctctgctgtatgtgtcatcactgatcaaagtgttgattgggatactgaggacctatttgtgagtggattccttgtgagatacccccaggcattcccaatggagatttcttcagttcaaccagagaagactttcattatggaatggtggttacctaccgctgcaacactgatgcgagagggaaggcgctctttaacctggtgggtgagccctccttatactgtaccagcaacgatggtgaaattggagtctggagcggccctcctcctcagtgcattgaactcaacaaatgtactcctcctccctatgttgaaaatgcagtcatgctgtctgagaacagaagcttgttttccttaagggatattgtggagtttagatgtcaccctggctttatcatgaaaggagccagcagtgtgcattgtcagtccctaaacaaatgggagccagagttaccaagctgcttcaagggagtgatatgtcgtctccctcaggagatgagtggattccagaaggggttgggaatgaaaaaagaatattattatggagagaatgtaaccttggaatgtgaggatgggtatactctagaaggcagttctcaaagccagtgccagtctgatggcagctggaatcctcttctggccaaatgtgtatctcgctcaatcagtggtctaattgttggaattttcattgggataatcgtctttattttagtcatcattgttttcatttggatgattctgaagtataaaaaacgcaataccacagatgaaaagtataaagaagtgggtattcatttaaattataaagaagacagctgtgtccgccttcagtctctgctcacaagtcaggagaacagcagtaccactagcccagcacggaattc

SEQ ID NO: 53 - DNA, 1011 bp, Homo sapiens
ctgggcggagggcaggagcatccagttggagttgacaacaggaggcagaggcatcatggagggtccccggggatggctggtgctctgtgtgctggccatatcgctggcctctatggtgaccgaggacttgtgccgagcaccagacgggaagaaaggggaggcaggaagacctggcagacgggggcggccaggcctcaagggggagcaaggggagccgggggcccctggcatccggacaggcatccaaggccttaaaggagaccagggggaacctgggccctctggaaaccccggcaaggtgggctacccagggcccagcggccccctcggggcccgtggcatcccgggaattaaaggcaccaagggcagcccaggaaacatcaaggaccagccgaggccagccttctccgccattcggcggaaccccccaatggggggcaacgtggtcatcttcgacacggtcatcaccaaccaggaagaaccgtaccagaaccactccggccgattcgtctgcactgtacccggctactactacttcaccttccaggtgctgtcccagtgggaaatctgcctgtccatcgtctcctcctcaaggggccaggtccgacgctccctgggcttctgtgacaccaccaacaaggggctcttccaggtggtgtcagggggcatggtgcttcagctgcagcagggtgaccaggtctgggttgaaaaagaccccaaaaagggtcacatttaccagggctctgaggccgacagcgtcttcagcggcttcctcatcttcccatctgcctgagccagggaaggaccccctcccccacccacctctctggcttccatgctccgcctgtaaaatgggggcgctattgcttcagctgctgaagggagggggctggctctgagagccccaggactggctgccccgtgacacatgctctaagaagctcgtttcttagacctcttcctggaataaacatctgtgtctgtgtctgctgaaaaaaaaaaaaaaaaaaa

SEQ ID NO: 54 - DNA, 1069 bp, Mus musculus
gagcttccttgcctcctgagtctttgctgtgccaaagccctgaaatatcatatctggccatcagacactggtaagttggaggtgtgaacttgtttggctctccctgcctgcagtgacaccagagacctggacaccagtgacctccctcagaagggcgtctcctgcacgtgaggagcatgtccattttcacatccttccttctgctgtgtgtggtgacagtggtttatgcagagaccttaaccgaaggtgttcaaaattcctgccctgtggttacctgcagttctccaggcctgaatggcttcccaggcaaagatggacgtgacggtgccaagggagaaaagggagaaccaggtcaagggctcagaggcttgcaaggccctcctggaaaagtaggacctacaggacccccagggaatccggggttaaaaggagcagtgggaccgaaaggagaccgtggggacagagcagaatttgatactagcgaaattgattcagaaattgcagccctacgatcagagctgagagccctgagaaactgggtgctcttctctctgagtgaaaaagttggaaagaagtattttgtgagcagtgttaaaaagatgagccttgacagagtgaaggccctgtgctccgaattccagggctctgtggccactcccaggaatgctgaggaaaactcggccatccagaaagtggccaaagatattgcctacttgggcatcacagatgtgagggttgaaggcagttttgaggatctgacaggaaacagagtgcgctatactaattggaatgatggggagcccaacaacacgggcgatggggaagactgtgtggtgatcttgggaaatggcaagtggaacgatgtcccctgctctgactcttttttggcaatctgtgaattctctgactgagggtgcttgtttctcagccctccttgattctttagggtactcctgacgtccgcagtttgttctgaaaaataaaatatgggaaaatataaacaattcaacattggttacccaatgcattctcttgtgaaggtgtagaaataaagtgagtttagttttca

SEQ ID NO: 55 - DNA, 1019 bp, Mus musculus
aattccggattaggcctgaagtcccttacaccctcaggatggtcgttggacccagttgccagcctcaatgtggactttgcctgctgctgctgtttcttctggccctaccactcaggagccaggccagcgctggctgctatgggatcccagggatgccaggcatgccgggggcccctgggaaggacgggcatgatggactccaggggcccaagggagagccaggaatcccagccgtccctgggacccaaggacccaagggtcagaagggcgagcctggcatgcctggccaccgtgggaaaaatggccccagggggacctcagggttgccaggggacccaggccccagggggcctccgggggagccaggtgtggagggccgatacaaacagaagcaccagtcggtattcacagtcacccggcagaccacccagtacccagaagccaacgccctcgtcaggttcaactctgtggtcaccaaccctcaggggcattacaacccaagcacagggaagttcacctgtgaagtgccgggcctctactacttcgtctactacacatcgcatacggccaacctgtgcgtgcacctgaacctcaaccttgccagggtggccagcttctgcgaccacatgttcaacagcaagcaggtcagctccggaggagccctcctgcggctccagaggggcgacgaggtgtggctatcagtcaatgactacaatggcatggtgggcatagagggctccaacagcgtcttctctggtttcctactgtttcccgactagaacggcaggctgcttccagcccccaaccacccacctcgctccctctgctttccccatcctcactcagacctcttcctccaagaagtccaccctggttcctgatccatcggccctgtgtctcctcagagtttctctgggaaccacctaatggtattattcctgtggccatttatcaataccttatgagactatttttttgttcaggtggtgagatagagaaataaatggatcaccggaatt

SEQ ID NO: 56 - DNA, 921 bp, Mus musculus
aattccggctgatgaagacacagtggggtgaggtctggacacacctgttactgctgctcctaggttttctccatgtgtcctgggcccaaagcagctgcaccgggccccctggcatccctggcatccctggggtccctggggtccctggctctgatggccaaccaggcactccagggataaagggggagaaagggctccctggactggctggagaccttggtgagtttggagagaaaggggacccagggatccctgggactccaggcaaagttggccctaagggtcccgtcggccctaagggtactccagggccctctggaccccgcggtcccaaaggcgattctggggactacagggctacacagaaagtcgccttctctgccctgaggaccatcaacagccccttgcgaccgaaccaggtcattcgcttcgaaaaggtgatcaccaacgcgaacgagaactatgagccacgcaacggcaagttcacctgcaaggtgcctggcctctactacttcacctatcatgccagctcccggggcaacctgtgtgtgaatctcgtgcgtggccgcgatcgggacagcatgcagaaagtagtcaccttctgtgactatgcccagaacaccttccaagtgaccacaggtggggtagtcttgaagctagagcaagaggaggttgttcacctgcaggccacagacaagaactccctcctgggcattgagggtgccaacagcatcttcactggctttctgcttttccctgacatggatgcgtaatcacggggtcaaattacacctatccaacaccatcttcctgcctccctgccagcaatcctccctggacccctgacatcacccccttgactgcctgaaacccagaccagagccctgtagatgttacagaacgaatgggtcaatcggaatt

SEQ ID NO: 57 - DNA, 1638 bp, Homo sapiens
ttttcaaagggaaacttggaggcttagacctatggggctaggctgctgaggtttcttagggggcaatagctggaagaaagctctgaagaacaaatgaaaggttaatactgagaaatgggaggaggattcaaggcaagttttctaattgccagtggtttttgactcacagaacatggggaattcctgccagaaagtagagaggtatttagcactctgccagggccaacgtagtaagaaatttccagagaaaatgcttacccaggcaagcctgtgtaaaacaccaaggggaagcaaactccagttaattctgggctgggttggtgactaaggttgaggttgatctgaggttgagaccttcctctttggatcaccagctttcagctcagggcctgccaatgagtaaatgatagttaacaggtcctggaggggaatcagctgcccagatacaaagatgggattcaggtggcagatggacccgaagaggacatggagagaaagaggaagctcctacagacacctgggtttccactcattctcattccctaagctaacaggcataagccagctggcaatgcacggtcccatttgttctcactgccacggaaagcatgtttatagtcttccagcagcaacgccaggtgtctaggcacagatgaacccctccttaggatccccactgctcatcatagtgcctacctttgttaaagtactagtcacgcagtgtcacaaggaatgtttacttttccaaatccccagctagaggccagggatgggtcatctatttctatatagcctgcacccagattgtaggacagagggcatgctcggtaaatatgtgttcattaactgagattaaccttccctgagttttctcacaccaaggtgaggaccatgtccctgtttccatcactccctctccttctcctgagtatggtggcagcgtcttactcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctgggtcaccaggaccaaagggccaaaaaggagaccctggaaaaagtccggatggtgatagtagcctggctgcctcagaaagaaaagctctgcaaacagaaatggcacgtatcaaaaagtggctgaccttctctctgggcaaacaagttgggaacaagttcttcctgaccaatggtgaaataatgacctttgaaaaagtgaaggccttgtgtgtcaagttccaggcctctgtggccacccccaggaatgctgcagagaatggagccattcagaatctcatcaaggaggaagccttcctgggcatcactgatgagaagacagaagggcagtttgtggatctgacaggaaatagactgacctacacaaactggaacgagggtgaacccaacaatgctggttctgatgaagattgtgtattgctactgaaaaatggccagtggaatgacgtcccctgctccacctcccatctggccgtctgtgagttccctatctga

SEQ ID NO: 58 - DNA, 857 bp, Homo sapiens
ctgggactttggtggtgctacccttggcctcccagagtcctgccaccctgctgccgccaccatgctgccccctgggactgcgaccctcttgactctgctcctggcagctggctcgctgggccagaagcctcagaggccacgccggcccgcatcccccatcagcaccatccagcccaaggccaattttgatgctcagcagtttgcagggacctggctccttgtggctgtgggctccgcttgccgtttcctgcaggagcagggccaccgggccgaggccaccacactgcatgtggctccccagggcacagccatggctgtcagtaccttccgaaagctggatgggatctgctggcaggtgcgccagctctatggagacacaggggtcctcggccgcttcctgcttcaagcccgaggcgcccgaggggctgtgcacgtggttgtcgctgagaccgactaccagagtttcgctgtcctgtacctggagcgggcggggcagctgtcagtgaagctctacgcccgctcgctccctgtgagcgactcggtcctgagtgggtttgagcagcgggtccaggaggcccacctgactgaggaccagatcttctacttccccaagtacggcttctgcgaggctgcagaccagttccacgtcctggacgaagtgaggaggtgaggccggcacacagctccagtgctgagaagtcagtgccccgagagacgaccccaccagtggggtgcccgctgcctgtcctccgtgaaaccagcctcagatcagggccctgccacccagggcaggggatcttctgccggctgccccagaggacagtgggtggagtggtacctacttattaaatgtctcc

SEQ ID NO: 59 - DNA, 1068 bp, Rattus sp.
gtgtcctcatcagggtcacaaacctgtgaggaaaccctgaagacttgctctgtgatagcctgcggcagagacgggagagatgggcccaaaggggagaagggagaaccaggtcaagggctcaggggcttgcagggccctccagggaaactggggcctccaggaagtgtaggagcccctggaagtcaaggaccaaaaggccaaaaaggggatcgtggagacagcagagccattgaggtgaagctggcaaatatggaggcagagataaacaccctgaagtcaaaactggagctaaccaacaagttgcatgccttctccatgggtaaaaagtctgggaagaagttctttgtgaccaaccatgaaaggatgcccttttccaaagtcaaggccctgtgctcagagctccgaggcactgtggctatccccaggaatgctgaggagaacaaggccatccaagaagtggctaaaacctctgccttcctaggcatcacggacgaggtgactgaaggccaattcatgtatgtgacaggggggaggctcacctacagcaactggaaaaaggatgagcccaatgaccatggctctggggaagactgtgtcactatagtagacaacggtctgtggaatgacatctcctgccaagcttcccacacggctgtctgcgagttcccagcctgaggaaaccagtgcctccatcgtctccttggctctcagtcgcttccaaagaaaattcagttactggtttctcaagtttagtgttaagtgattcttttgatgggagagaatgtatttgcttgtggcatgaggacacgaatagaagctgaccgggaggcaccaggatctggttgagcacggagcaaaggtcacatccattttgctaggaacacagcaagaagtcaactatggaaaacctacaataaatatcccctgcccttttcaccagaggaccaaaggtggtccttatctgtgccagagtggcagctgatctcagccataaaagcacccaattccctttctccatgaattgtcactaagtggtgtcaaacgtgcctgtttgaagtcatcctcagt

SEQ ID NO: 60 - DNA, 894 bp, Mus musculus
ctaccagaagctggactcgagacatagtttctcttccactgctcctttactctaaagaaaccctagtaaggaccatgcttctgcttccattactccctgtccttctgtgtgtggtgagtgtgtcctcatcagggtcacaaacctgtgaggacaccctgaagacttgctctgtgatagcctgtggcagagatgggagagatggacccaaaggggagaagggagaaccaggtcaagggctcaggggcttgcagggccctccagggaaattggggcctccaggaagtgttggaagccctggaagtccaggaccaaaaggccaaaagggggaccatggagacaatagagccattgaggagaagctggcaaatatggaggcagagataaggatcctgaaatcaaaactgcagctaaccaacaagttgcatgccttctcaatgggcaaaaagtctgggaagaagttgtttgtgaccaaccatgagaagatgcccttttccaaagtgaagtctctgtgcacagagctccaaggcactgtggctatccccaggaatgctgaagagaacaaggccattcaagaagtggccacaggcattgccttcctaggcatcacggacgaggcgactgaagggcagttcatgtacgtgacaggggggaggctcacctacagcaactggaaaaaggatgagccaaataaccatggctctggggaagactgtgtcattatattagataatggtttgtggaatgacatttcctgtcaagcttccttcaaggctgtctgcgagttcccagcctgaggaaacgagtgcctccatattctccttgcctcctctctggactctcacttgcttccaaagaaaattcagtacttgtttctcaaaaaaaaaaaaaaaaaaa

SEQ ID NO: 61 - DNA, 599 bp, Rattus norvegicus
ggaaaggggagatgctgtgcagtacatcccagccatcatcaagtctaaggctgagcccctgtatgaacttgtgacagccacagactttgcgtactccagcacagtgaaacagaacatgaagaaggccctagaagaattccagaaggaggtcagctcctgccgctgtgctccgtgcaggaacaatggagtccccatcctgaaagaatcccgctgtgagtgcatctgtcctgtcggtcttcaaggtgtagcctgtgaggttaccaatcggaaagatatccccatagatgggaagtggagttgctggtctgactggtctccatgctctggaggacgcaaaacaagacaaaggcagtgcaacaacccggcacctcagagaggaggcagcccctgctcaggtcctgcttcagaaacactcaactgttaaagggagggaacacagccggcaggtgatcatcagggctctaaccctctcacacttagccaggctttagcacaccagctcccacccagggctaccacaacaaaaagcaatgccactctgccctttaaaggtttagtttcttcagtgcatgttaattccagtaaacagtgggtggagc

SEQ ID NO: 62 - DNA, 759 bp, Homo sapiens
gacccgcagcagagacgacgcctgcagcaaggagaccaggaaggggtgagacaaggaagaggatgtctgagctggagaaggccatggtggccctcatcgacgttttccaccaatattctggaagggagggagacaagcacaagctgaagaaatccgaactcaaggagctcatcaacaatgagctttcccatttcttagaggaaatcaaagagcaggaggttgtggacaaagtcatggaaacactggacaatgatggagacggcgaatgtgacttccaggaattcatggcctttgttgccatggttactactgcctgccacgagttctttgaacatgagtgagattagaaagcagccaaacctttcctgtaacagagacggtcatgcaagaaagcagacagcaagggcttgcagcctagtaggagctgagctttccagccgtgttgtagctaattaggaagcttgatttgctttgtgattgaaaaattgaaaacctctttccaaaggctgttttaacggcctgcatcattctttctgctatattaggcctgtgtgtaagctgactggccccagggactcttgttaacagtaacttaggagtcaggtctcagtgataaagcgtgcaccgtgcagcccgccatggccgtgtagaccctaacccggagggaaccctgactacagaaattaccccggggcacccttaaaacttccactacctttaaaaaacaaagccttatccagcaaaaaaaaaaaaaaaaaa

SEQ ID NO: 63 - DNA, 3579 bp, Rattus norvegicus
atgggcctttggggactactttgccttttaattttcctggacaagacctggggacaggaacaaacctacgtcatttcagcacccaaaatcttccgggtcggatcatccgaaaatgtcgtaattcaagcccatggctacaccgaagcctttgacgcaacgatctctctgaaaagctatcctgacaaaaaagtgacctactcttccggctatgttaacttgtcgccggaaaacaaattccagaactcagcactgttgacattaccgcccaaacaatttcccagagatgaaaacccagtctctcacgtgtatctggaagttgtgtcaatgcacttttcgaaatcgaagaaaataccaataacctatgacaatggatttctcttcatccatacagacaaacctgtttacactccagaccagtcagtaaagattagagtctactctctgagtgacgacttgaagccagccaaacgggagactgtcttaactttcgtagatcccgaaggaacagaagttgacatcgtagaagaaaatgattatactggaattatctcttttcctgacttcaagattccatctaatcccaagtatggtgtatggacaattaaagctaaatataagaaggattttacaacgactggaactgcatactttgaagttaaagaatatgtcttgccccgattctctgtatcaatagaaccagaaagcaacttcattggctataagaactttaagaactttgaaatcactgtgaaagcaagatatttttataataaaatggtccccgatgctgaagtctatatcttttttgggttgagagaggacataaaagaggatgagaaacagatgatgcataaagccatgcaagccgcaacgttggtcaatggagttgctcagatctcttttgactctgaaacagcagtgaaagagctgtcatatgaaagtgggttttcggaagaggcagaaattcctggcatcaaatacgtcctctctccctatacactgaatttggtcgctacccctcttttcctgaagcctgggattccattttccatcaaggtacaggttaaggattcactcgagcagttggtaggaggggtcccagtaactctgatggcacaaacagtcaatgtgaatcaagagacatctgacttggaaccaaagaggagcatcacacactctgctgatggagtggcttcatttgtggtgaacctcccatcagaagtgacatcactgaagtttgaggtcaaaactgatgccccggaacttcccgaagaaaatcaagccagcaaagaatatgaagcagttacatactcatccctcagccagagttacatttacattggctggactgaaaactacaagcccatgcttgtgggagaatatctgaatattatcgtcacccccaagagtccatatattgacaaaataactcactataattacttgattttatccaaaggcaaaattgtacagtatggcacaaaggagaaacttctctattcatcttatcaaaatataaacatcccagtgacacaggacatggttccttcagcgcggctcctggtctattacatagtcacgggggagcagacagcagaattggtggctgacgcagtctggataaacattgaggagaagtgtggcaaccagctccaggtccatctgtctccagataaagacgtgtattctccaggccaaactgtgtcccttgacatggtgactgaagcagactcatgggtggcactatctgcggtggacagcgctgtgtatggagtccggggaaaagccaaaagggccatgcaaagagttaagtgcacttccgaggtgttccaagcttttgatgacaagagtgacctgggctgtggggcaggtggtggccgtgacaatgtagatgtattccatctagctgggctcaccttcctcaccaatgcaaacgcagatgactcccaataccacgatgactcttgtaaggaaattctcaggccaaagagagacctgcagctcctgcatcagaaagtggaagaacaagctgctaaatacaaacaccgtgtgcccaagaaatgctgttatgatggagcccgagaaaacaaatacgaaacctgtgagcagcgagttgcccgggtgaccataggcccacactgcatcagggccttcaacgagtgttgtactattgcggataagatccgaaaagaaagccaccacaaaggcatgctgttgggaaggatccaaataaaggccctgttaccagtgatgaaggcagaaatccgaagctactttccagagagctggctatgggaagttcatcgtgttcccaaaagaaaccagctgcaggttgcactgcctgactcactgacgacctgggaaattcaaggcatcggcatctcagacaatgctgccgaccataaagatgcagtcaactccatgctttctttcacttttgacagtatatgtgttgctgacacactcaaggcaaaggtgttcaaagatgtcttcctggagatgaacataccatattctgttgtacgaggggagcagatccaattgaagggaaccgtttacaattataggacctctgggacaatgccagaagggatcaaaagggaaagctatgctggtgtgactctggaccccaggggagtttatggtattgttaacagacgaaaggaattcccatacaggataccattagatttggtccccaaaaccaacgtcaaaaggattttgagtgtaaaaggactgcttataggggaattcttgtccacggttctgagtaaagaaggcatcgacatcctaacccacctccccaagggcagcgccgaggcagaactcatgagcatagtcccggtgttctacgttttccactacctggaagcaggaaaccattggaatattttccaccctgatacgttagctagaaaacagagcctgcagaaaaaaataaaagaagggctggtgagcgtcatgtcctacagaaacgctgactattcctacagcatgtggaagggagcaagctctagtgcctggctgacagcttttgctctgagagtgcttggacaggtgaacaagtatgtgaaacaagaccaatactcgatctgtaactccttgttatggctgattgagaagtgtcagctggaaaacggatctttcaaggaaaattcccaatatctaccaataaaattacagaaaatctacacagcgctggctaaagctgactccttcctacttgaaaggaccctgccttccaagagcaccttcaccctggccattgtggcctatgctctctccctgggagacagaacccacccgaagtttcgttctattgtgtcagccctgaagagggaagctttggttaaaggagacccgcccatttaccgtttctggagagacactctccaacgtccagacagctcagcacccaacagcggcacagcaggtatggtagaaaccacggcctatgctttgctcaccagcctgaacctgaaggagacgagttatgtcaacccgatcatcaagtggctatctgaggagcagaggtatggaggcggcttttattccacccagaccgtggaaggctcgtag

SEQ ID NO: 64 - DNA, 1227 bp, Rattus norvegicus
gggtggacagaggtgtcactgtggtcagcctggctcagaacctaaaagaaagtgtcaagtcagggaaaatttctggaaaggggatgtgggattagggtgctgtgggggaggggaggggcccctgctctgggcggctcttctctgaccactctgaaacagtgtcttcctgtctgggagaacaggacgtctctctgattaggcctgaagtcccctacatgctcaggatggttgtaggaaccagctgccagccccagcatggactctacctgctgctgctccttctggccctacccctcaggagccaggccaacgctggctgctatgggatcccagggatgccaggcctgccgggaacccctgggaaggatgggcatgatggacttcaggggcccaagggtgagccgggaatcccagccatccctgggacacaaggacccaagggtcagaagggcgagccgggtatgcctggccatcgtgggaaaaacggccccatggggacctctgggtcgccaggggatccaggccccaggggtcctcccggggagccgggtgaggagggtcgatacaaacagaagcatcagtcggtgttcacggtcacccggcagaccgcgcagtacccagcggccaatggccttgtcaagttcaattccgccatcaccaatcctcagggggattacaacacaaacacggggaagttcacctgcaaagtgcccggcctctactacttcgtccaccacacatcccagacggccaacctgtgcgtgcagctgctcctcaacaatgccaaggtgaccagcttctgcgaccacatgtccaacagcaagcaggtcagctcaggaggagtactcctgcggctgcagaggggcgacgaggtgtggctggccgtcaacgactacaacggcatggtgggcactgagggctctgacagcgtcttctctggtttcctactgtttcctgactagaatggcaggctgggtccagcacccggacgcccgcctcgctccctctgctttccccatcctcactcagacctcttccttcaggaagtccaccctggttcctgacccatcagccctctgtctcctcagagtttctctgggaatcactgactggttccattccagtggcagtttatcgagacctttatgagactatttttttttcaggtgggaagagagaaaaataaatagatcactaaataaatatgcctggcccagatttctcact

SEQ ID NO: 65 - DNA, 1000 bp, Rattus norvegicus
atgcccggggtccctgtgtgtctctgcaggaacatcatggagacctctcagggatggctggtggcttgtgtgctggccgtgaccctggtatggacagtggctgaagatgtctgcagagcacccaacgggaaggacggggttgcaggaattcctggccgcccagggaggccgggtctcaaaggagagagaggggagccaggagctgccggcatccggaccggtatccgaggtcttaaaggagacatgggggaatctgggccccctggcaaacccggcaatgtggggttcccagggcccactgggcccctggggaacagcggcccccaagggttgaaaggtgtgaaaggcaatccgggcaatatcagggaccagccccggccagctttctcagctattcggcagaacccaccgacgtatggcaacgtggttgtctttgacaaggtcctcaccaaccaggagaatccataccagaaccgcacaggtcacttcatctgtgcggtgcccggtttctattacttcaccttccaagtgatctccaagtgggacctttgtctgtctatcgtgtcctcctcccggggccagcccaggaattcccttggtttctgtgacaccaacagcaaggggctcttccaggtgttagcagggggcactgtgcttcaattgcaacgaggggacgaggtgtggattgagaaggacccagcaaagggccgcatttaccagggtactgaagccgacagcatcttcagtggattcctcatttttccctcggcctgagctgggtagctgccccacgttctgccatctcctgcactccctgttgcggggccccaccctgcatcccttctctctgtactctgcaaagtgaaggggctggggttttagcactctgggggaggggctggctccgagggcactgaggactgatgtctctctgcacacggcccagtggtttctttaagtactttctggaataaatgaccccatctgtgtctgtggcttg

SEQ ID NO: 66 - DNA, 681 bp, Rattus norvegicus
agcctttgagcgcaaacgtactgtcaacgccatttgtttctgaatctggcggtggccgtcctcctctcgtgcttggcactgcctatcctgtttacgtccattgtaaagcataaccactggcccttcggtgaccaggcctgtatagtcctgccctcgctcattctgctcaacatgtactccagcatccgtctgctggccactattagtgccgaccgtttcctgctggtgttcaaacccatctggtgtcagaagttccgccggcctggcctggcctggatggcttgcggagtaacctgggtcttagcattgctcctcaccattccgtccttcgtgttccggcggatacataaggacccctactcagatagcattctatgtaatattgactatagtaagggtccgttcttcatagagaaggctatagccatcctgcggctgatggtgggtttcgtgttgcctctgctcactcttaacatctgctacaccttcctcctgatccggacctggagtcgcaaggccacgcgctccaccaagacgctcaaagtggtgatggcggtggtcacatgtttctttgtcttctggctaccctaccaggtaacaggggtgatattagcttggctgccccggtcctcatccacttttcagtcagtggagaggctgaactccttgtgcgtttccctg

SEQ ID NO: 67 - DNA, 1882 bp, Rattus norvegicus; identical to SEQ ID NO: 26

SEQ ID NO: 68 - DNA, 599 bp, Rattus norvegicus; identical to SEQ ID NO: 61

SEQ ID NO: 69 - DNA, 2083 bp, Rattus norvegicus
ggttgcaaagaaatgcttctcaggactccagggctgcctaggaggagcggcatggcctcaggcgtgaccatcaccctagccattgcaatctttgccttggagatcaatgcacaggccccagagcccactccccgggaagagccatcagcagacgccctcctaccaatagactgcagaatgagcacatggagtcagtggtcacagtgtgatccttgcctcaaacaaaggtttcgctcaagaagcatggaagtctttggacagtttcagggaaaaagctgtgctgatgctttgggagacagacaacattgtgaacccactcaggagtgtgaagaggtacaggaaaactgtgggaatgactttcagtgtgaaacaggcaggtgcataaagaggaaacttctgtgtaatggtgacaacgactgtggagatttttctgatgagagtgactgtgaaagtgacccgcgcctcccgtgccgtgaccgggtggtagaagaatcggaactgggacgaacagcaggatatgggatcaacatcttagggatggatcccctgggcacgccttttgacaatgagttctacaatggactctgtgaccgggtacgggacggaaacactttgacatactatcgcaaaccttggaacgtagcatttctggcctatgaaaccaaggctgacaaaaatttcagaactgagaattatgaagaacagtttgaaatgttcaaaaccatcgtccgagacaggaccacgagttttaatgctaatttagctctaaaattcacaatcactgaagcacctataaaaaaagttggagttgatgaagtcagcccagaaaaaaactcttcaaagcctaaagactcttctgttgattttcaattttcatatttcaagaaagaaaattttcaacgattgtcatcctacttgtcacagacgaaaaagatgtttctgcacgtgagaggaatgattcaactggggagatttgtcatgaggaatcggggcgttatgctgacgacaactttcctggatgatgtaaaggctttaccagtttcctatgaaaagggcgaatattttgggtttttggagacttatgggactcactacagtagctctgggtccctgggagggctctacgaactgatctatgtcttggataaagcttccatgaaagagaaaggtgttgaactcagcgacgtaaagcggtgtcttgggtttaacctggatgtttctctatatacgcctctacaaactgccttagaaggaccatcattgacagccaatgttaatcacagtgattgcttaaagacaggggatggtaaagtagtaaacatcagccgcgatcacatcatagatgatgttatttcattcataagaggagggaccaggaagcaagcagttctcctgaaagagaagcttctcagaggagccaagacgattgatgtgaacgacttcatcaactgggcctcatccttggatgacgctccagctctcattagtcaaaaactgtcccctatctataatctcattcctttgacaatgaaagatgcatacgcaaagaaacagaatatggaaaaggctattgaagactatgttaatgaattcagtgctagaaagtgctacccatgtcaaaacggaggcacagcaattcttctggatggacagtgcatgtgctcctgcacaatcaagtttaaggggattgcctgcgaaatcagtaaacaaagatagccttcaggaaacaaagcaaaacctggttcacatggaaggtggaaaaaaggacaaaaaaagaagaagagagaggagagagaagagagagagaaaagaaaaaaccccaggactttccaacttagcatcctaccctagagcgaatcctcactgccaagtagaaagcagcttgcttcatggaaatcctaccaacctctgatgtcgtctctgtttcaggtctacagtgcctttctcccctctttaatgcctataatgcttccatttttttttttatccctaatgaagaatcggcagtgagatatgccaggactgccttttcccacaggcaatgccaatctctcgctaataaaacagagttaaattaaaaaca

SEQ ID NO: 70 - DNA, 874 bp, Rattus norvegicus
ggtgaggaccatgtccctgttcacatccttccttctgctctgcgtgctcacggcagtctatgccgagaccttaaccgaaggggctcaaagtagctgccctgtgattgcctgcagttctccgggcctgaacggcttcccaggcaaagatggacacgacggtgccaagggagaaaagggagaaccgggtcaaggcctcagaggcttgcagggccctcctggaaaagtaggacctgcagggcccccagggaatcctgggtcaaaaggagcaacgggaccaaaaggagaccgtggagagagtgtagaatttgatactaccaacattgatttagaaattgcagccctgcgatcggagctgagagctatgagaaagtgggtgctcctttctatgagtgaaaatgttggaaagaagtacttcatgagcagtgttagaaggatgccccttaacagagcgaaggctctgtgctccgaactccagggcactgtggccactcccaggaatgctgaggaaaatagggccatccagaatgtggccaaagatgttgccttcttgggcataacggaccagaggactgaaaacgtttttgaggacctgacaggaaacagagtgcgctacactaactggaatgagggtgagcccaacaatgtgggctctggggaaaactgtgtggtgctcttgacaaatgggaagtggaatgacgttccttgctctgattcttttttggtagtttgtgaattctctgactgagggtgcttgtttctcatccctccttgatacttcagtgtattctataagtccacagtttgttctgaaaatataggcaattcaacattggttacctaattaaactgtaacatttttcagaatagcaaaaaa

SEQ ID NO: 71 - DNA, 578 bp, Rattus sp.
tttggccctcgaggccaagaattcggcacgaggcccttgtactggactgccactatcctgaggacgagatcggacacgaagatgaggccgagagtgacattttctctattaaggaagtgagctttcagaccactggtgactctgagtggagggatacaaactacacactcaacacagactccctggactgggccttgtatgaccacctaatggatttccttgcggaccgaggggtggataacacttttgcagatgagttggtggagctcagcacagccctggagcaccaggaatatatcacctttcttgaggacctcaaaagctttgtcaagagtcagtagaactgtgagactgaaggcctcaatctaaacggccagctctggtgggcgagcaaaagctgccttgacatcacaactatgctttgaaatggctgtcatcctaatatatgggggaaagcaagtttaaattatcgccgttacacctccatttactattcctttgggctcttttcctgtacacatctattatttgtagatttttgtatgacatgatgatgaacaataaatctgacttcatct

SEQ ID NO: 72 - DNA, 1638 bp, Rattus norvegicus
acccgcgtcaccaggaggagcgcactggagccaagccgcagacgggactccagactccaaagaggccacaccatgaagattctcctgctgtgtgtggcactgctgctgacctgggacaatggcatggtcctgggagagcaggagttctctgacaatgagctccaagaactgtccactcaaggaagtaggtatgttaataaggagattcagaacgccgtccagggggtgaagcacataaagaccctcatagaaaaaaccaacgcagagcgcaagtccctgctcaacagtttagaggaagccaagaagaagaaagagggtgctctagatgacaccagggattctgaaatgaagctgaaggctttcccggaagtgtgtaacgagaccatgatggccctctgggaagagtgtaagccctgcctgaagcacacctgcatgaagttctacgcacgcgtctgcaggagcggctcggggctggttggtcgccagctagaggagtttctgaaccagagctcacccttctacttctggatgaacggggaccgcatcgactccctgctggagagtgaccggcagcagagccaagtcctagatgctatgcaggacagcttcactcgggcgtctggcatcatagatacgcttttccaggaccggttcttcacccatgagccccaggacatccaccatttctcccccatgggcttcccacacaagcggcctcatttcttgtaccccaagtcccgcttggtccgcagcctcatgcctctctcccactacgggcctctgagcttccacaacatgttccagcctttctttgatatgatacaccaggctcaacaggccatggacgtccagctccatagcccagctttacagttcccggatgtggatttcttaaaagaaggtgaagatgacccgacagtgtgcaaggagatccgccataactccacaggatgcctgaagatgaagggccagtgtgagaagtgccaagagatcttgtctgtggactgttcgaccaacaatcctgcccaggctaacctgcgccaggagctaaacgactcgctccaggtggctgagaggctgacccagcagtacaacgagctgcttcattccctccagtccaagatgctcaacacctcatccctgctggaacagctgaacgaccagttcacgtgggtgtcccagctggctaacctcacacagggcgatgaccagtaccttcgggtctccacagtgacaacccattcttctgactcagaagtcccctctcgtgtcactgaggtggtggtgaagctgtttgactctgaccccatcacagtggtgttaccagaagaagtctccaaggataaccctaagtttatggacacagtggcagagaaagcgctacaggaataccgcaggaaaagccgcatggaatgagacagaagcatcagttttctatatgtaggagtctcaaggagggaatctcccagctttccgaggttgctgcagacccctagagaactccacatgtctccagcgcctaggcctccaccccagcagcctctccttcctctgggttctgtactctaatgcctgcacttgatgctctcgggaagaactgcttcccccacgcaactaatccaataaagccaccttgcg

SEQ ID NO: 73 - DNA, 631 bp, Rattus sp.
ctcagatggagggaaaatgcaaagatttattcccaatcaggggagaatattgaattcatgtgtaaacctggatatagaaaattcagaggatcacctccgtttcgtacaaagtgcattgagggtcacatcaattatcccacttgtgtataaaatcgctatacaattattagtaaaccttatggatgaacctttgtttagaaatgcacatgtatattactaatacagtttgaatttacatttgaaatattgtttagctcatttcttctaataagtatataaactttttttatatggtggttaatcagtaactttacagactgttgccacaaagcaagaacattgcattcaaaactcctaatccaaaatatgatatgtccaaggacaaactatgtctaagcaagaaaataaatgttagttcttcaatgtctgtttttattcaggacttttcagattttcttggataccttttgttgttaggttctgattcacagtgagtggaagacacactgactctgacttcaaattagtattacttgccaatacataacaaccaaactatcataatatcacaaatgtatacagctaattactgtgtcctacctttgtatcaataaagaaatctaagaaagtt

SEQ ID NO: 74 - DNA, 274 bp, Rattus norvegicus
cggcacgagggccgcatgtgttttttttaaaaaaatttgtactcaacagtgatacccccattttcattcatgaaatgtattagctaagggacacgccctgaaaacaggtactcagtttctactgttacccttactggtatttgtctctctcttagtttatgtttaagccaaagatttcctcagaggtagcccagcaggggcaattgcttttgtacatcttctattttaaatagaaataaatatgttctaagctcatgaaaaaaaaaaaaaaaaa

SEQ ID NO: 75 - DNA, 592 bp, Rattus sp.
ctatcctaataaaaatgttaattactcttcgggcactgttgaattatcaccagaaaataaattccaaaactctgctatcttaacaattcaggcccaagagttgtctgaagaacaaaactggttctcaaatgtgtattcggaagtcgtgtcaaagcatttttcaaaattagaaataatgccaatcgtctatgacaacagctctctctttgttcacactgacaagcctgtgtacactccagaacagcctgtgaaggttgccgtctactcagtggatgatgacctagagcctgtcaccagaacaacagtcttgactttcatagtatttacttcttctcgatcactcgggacactttcctcacagatgtacggactgagaagagcagttctcaggagacagcactctcttggagggctgcctgccttaagaactaactcttcataaatgcttgcctgcagttctgtgtttccatccagcattgaagccacatggctctcctgtccagtgtgggcttctttcaaccccagcttccagaatcgtgggctaaaataagtctgtgttctttaaagcttacctggcaccagatactctttt

SEQ ID NO: 76 - DNA, 832 bp, Rattus norvegicus
gcggccgccgtgtacctcgtggctctggtggtcctggaggcggctgtatgtgttgcgcagccccgaggtcggattctgggtggccaggaggccatggcccatgctcggccctacatggcttcagtgcaagtgaatggcacgcacgtgtgcggtggcaccctggtggatgagcagtgggtgctgagcgccgcgcactgcatggatggagtgaccaaggatgaggttgtgcaggtgctcctgggtgcccactccctgtccagtcctgaaccctacaagcatttgtatgatgtgcaaagtgtagtgcttcacccgggcagccggcctgacagcgttgaggacgacctcatgctctttaagctctcccacaatgcctcactgggtccccatgtgagacccctgcccttgcaacgcgaggaccgggaggtgaaacccggcacgctctgcgatgtggccggttggggcgtggtcactcatgcgggacgcaggcccgatgtcctgcagcaactgacagtgtcaatcatggaccggaacacctgcaatctgcgcacgtaccatgatggggcaatcaccaagaacatgatgtgtgcagagagcaaccgcagggacacttgcaggggcgactccggcggtcctctggtgtgcggggatgcggtcgaagctgtggttacgtggggatctcgagtctgtggcaaccggagaaagccaggtgtctttacccgcgtggcaacctacgtgccgtggattgaaaacgttctgagtggtaacgtgagtgttaacgtgacggcctgaggggacaccggagaccgtgactcacaataaatgcagcggccgc

SEQ ID NO: 77 - DNA, 460 bp, Rattus sp.
atcaaacgcaaacacatgcacacacattgggagaaacagacagatgtaattattttcatctcgaggggtaatgtcacgcttcgccctaatcagccatccccacgtgtaggatctttcttcttgtcagaagatcaaactacttaaatgtcagagcagaattcatcatgctatggtcacagggcaccgtctgccttcttgtctgagaacaaatataattcagtctcttgagggctttcttcagtgtgtgtcaaatataaacctgaaaatcactctgcttttgagagaagtgtcagtttatgctagagctattgagagatgcatatttgagaagaatcttccctaatgtggtcacacccttagatttttttttccagaagcacacaatttgagacattggaatttgctttatttcttattcatctttcattaaacataacaatagcaagaaatccagctgtgg

SEQ ID NO: 78 - DNA, 445 bp, Rattus norvegicus
gctatcgtgtcctcctcccggggccagcccaggaattcccttggtttctgtgacaccaacagcaaggggctcttccaggtgttagcagggggcactgtgcttcaattgcaacgaggggacgaggtgtggattgagaaggacccagcaaagggccgcatttaccagggtactgaagccgacagcatcttcagtggattcctcatttttccctcggcctgagctgggtagctgccccacgttctgccatctcctgcactccctgttgcggggccccaccctgcatcccttctctctgtactctgcaaagtgaaggggctggggttttagcactctgggggaggggctggctccgagggcactgaggactgatgtctctctgcacacggcccagtggtttctttaagtactttctggaataaatgaccccatctgtgtctgtggcttg

SEQ ID NO: 79 - DNA, 568 bp, Rattus norvegicus
tggccttgtcaagttcaattccgccatcaccaatcctcagggggattacaacacaaacacggggaagttcacctgcaaagtgcccggcctctactacttggtccaccacacatcccagacggccaacctgtgcgtgcagttgctcctcaacaatgccaaggtgaccagcttttgcgaccacatgtccaacagcaagcaggtcagctcaggaggagtactcctgcggctgcagaggggcgacaaggtgtggctggccgtcaacgactacaacggcatggtgggcactgagggctctgacagcgtcttctctggtttcctactgtttcctgactagaatggcaggctgggtccagcacccggacgcccgcctcgctccctctgctttccccatcctcactcagacctcttccttcaggaagtccaccctggttcctgacccatcagccctctgtctcctcaaagtttctctgggaatcactgactggttccattccagtggcagtttatcgagacctttatgagactatttttttttcaggtgggaagagagaaaaataaatagatcact

SEQ ID NO: 80 - DNA, 5066 bp, Rattus norvegicus; identical to SEQ ID NO: 6

SEQ ID NO: 81 - DNA, 474 bp, Rattus sp.
caatttcagtttcaagtatatataccctctagattcctccacctggattgaatattggcccacagacacaacgtgtccatcctgccaagcgtttgtagctaatttggacgagttcgctgaagacatctttctaaatggctgtgaaaatgcctgaggaagttctgctgcgtggccttcccgggtactcctgttggtggctcctaggagccaggatcgcttggaaacttagcctagaatcggatacattttctttatagtaaagcgtaagttgaagagttactttgtgaaacaaaatagccttgtggagagccgaaggcaggtcccccaaggctattggacatcagcaccaataagctggaacaagtctgtaacgttagcagccaggggtgtttgttggggccggaagaagagactcactgaaattgtagccccttaggaaaacatggtcttgcttgaaaaaaaaaataccaagga

SEQ ID NO: 82 - DNA, 2908 bp, Rattus norvegicus
ggaggtatcgaggaagagagaacagggaggtggggcggaggttcctcgcagagcctctggagccgcaggggcttcacggcatgaccagaagcaggagaggaggctgacccacttgttcccatcagctcctgaaggtgacactgagccctgggtggcccctcactgccaaagcagtcacctgtatttgtcagataaagacggccagcccggctgccctttacctccaagtcagagatccagagagccatgggcaaatcgccagagatgtggtgctttgtcttcttttctcttttggcatcgttttctgctgagcctaccatgtatggggagatcctgtcccctaattatccccaggcgtaccccaatgaggtcgtgaaaacttgggacatagaagtcccagaggggtttgggattcacctttacttcacccatctggacatggagctgtcagagaactgtgcatacgactcagtgcagataatctcaggaggtatcgaggaagagagactctgtggccagaggtccagcaagagtcccaactcccccactgtagaagagtttcaattcccatacaataggctccaggtggtctttacgtcagacttctccaacgaggaacggtttactggctttgcagcgtattactcagccgtagatgtaaatgaatgcacagactttacagatgtcccttgcagccacttctgcaataacttcattggtggatacttctgctcctgccccccagaatacttcctccacgatgacatgaggacttgtggggtcaactgtagtggggatgtattcactgccttgattggggagatcgcaagtcccaattatcccaacccatacccggagaactcaaggtgtgaataccagattcggctgcaggagggcttccgactggtgttgactatccggagagaagattttgatgtggaaccagcggactcagaggggaactgccacgacagtttgacttttgctgcaaaaaaccaacagtttggtccttactgtggcaatggattccctggacctctaactattaaaacccagagcaatactcttgatattgtctttcaaactgacctaacggggcaaaataaaggctggaagcttcgttaccatggagatcccatcccctgtcccaaagaaatcagtgctaattctatctgggagcccgaaaaggcaaaatacgtgttcaaagatgtcgtgaagataacctgtgtggatggattcgaagttgtggagggaaatgttggctcaacatcattctattccacttgtcaaagcaacggacagtggagcaattccaggctagagtgtcaacctgtggactgtggtgttccagaacccattgagaatggtaaagttgaagacccagaagacactgtattcggctccgtcatccactacacgtgcgaagagccatattactacatggaacaggaagaaggcggagagtatcactgtgctgctaatgggagctgggtgaatgaccagctgggtgtcgagcttccaaaatgtattccagtctgtggagtacccaccgagccctttaaagtacagcagaggatatttggaggatactctacaaagattcaaagttttccttggcaggtctactttgagtccccccgaggtggcggggctcttatcgatgagtactgggtgctgacggccgctcacgttgtggagggaaactctgacccagtgatgtatgtcgggtccacacttctgaaaatagagcggttgagaaatgcccagaggctcatcactgaacgtgtgattattcatcccagctggaaacaagaggacgacctgaatacacggacaaattttgacaatgacattgccctggtgcagctcaaagaccctgtgaaaatgggacccactgttgcccccatctgcctgccagaaaccttctcagactacaacccctcagaggttgacctggggctgatctctgggtggggccgaacagagattagaaccaatgttattcaactcagaggggcgaagttacccataacatctttagaaaagtgccagcaggtgaaagtggaaaacccgaaagcgaggtcaaacgactatgttttcactgacaacatgatctgtgctggggaaaagggtgtggacagctgtgaaggtgacagcggaggggcttttgctctgccggtccccaatgtcaaggaccccaaattctatgtggctggcctggtgtcctgggggaaaaagtgtgggacctatgggatctacacaaaggtaaagaactacgtggactggatcctgaaaactatgcaggagaatagtgggcccaagaaggactgatccgtagtaacaacacccctccaggactagcaaggtcatttttctcagatcctgggacggtcccattatttcaaaatgatggagagagggtgtgggagcatggttaacgttgaacatgattgtcaagaagcctgcttggaggcagagttgatcactgagccgtgttggttattcagttgctattgctaacaacatgcggaagcctttctgtcttgcttcatcccacagggatatcttaaacgatttccccctcatttaacccgcttgaaatccttattgcttacagtaaagcatgtttccaatctggttctggctgctcgagagcccagaaggagagggaaatttgagggtattttgtcatggaattcaggcatcgacaggttgtctgaaacactatgcagtcagggaacacagccttttttctaagtgagatttacccaatagctggaagtcagaattgactaccttagctttcctttgtgagttgtttcaatatgttccctagaaattagttttcttataatcctcctttgtatcatacaatgtaatgacttaataaaagagaaattgaacattg

SEQ ID NO: 83 - DNA, 522 bp, Rattus sp.
gagaccttttatgacaaagaccttatgggttatgtcagcggcttcgggataacagaagataaaatagcttttaatctcaggtttgtccgtctgcccatagccgatcgagaggcatgccagaggtggctccggacgaaaaacagtaatgatgtattttctcaaaatatgttctgttctggggacccaactctcaagcatgacgcctgccagggggacagtgggggtgtttttgcagtcagggaccgcagtcgtgatatctgggtggctacaggcatcgtatcctggggcattgggtgtggtgagggatacggcttttacaccaagttactgaattatgttgactggatcaagaaagagattggagatgaaaactgaagccagcattcattgggttagaatccagtgtgtagtatattaaaaaaaaaaaagtatctaaccaatttttgataagcactacgtttctcatattaaaatcacggaggcagacagtgtatagaataaattctcttttactataatctc

SEQ ID NO: 84 - DNA, 560 bp, Rattus sp.
tttcagcccacttacgtgatgatgcctcgcattaaagtaaagagcatccaagacatgctgtcaatcatggagaaactggaattctttgacttcacttacgatctcaacctgtgtgggctgactgaggacccagatcttcaggtgtcttccatgaaacacgagacggtgttggaactgacagagacaggtgtggaagcagccgcagcctccaccatctccgtggcccgaaacttactcatctttgaggtgcagcagcctttcctcttcctgctctgggaccagcgacacaagttcccagtcttcatgggccgtgtatatgaccccagggcctgagacaggcaggagtcaggcttgagtaagcactgccacccaagcttcagctcatccagctatttcctcgccactgcctgccctagccacttccagccttaggaactggcagaaggaacagtttccacccaccaacccccatggtattgacatgctctctaaaccactgttttgcagcttttatggttcaagcttaacagagtctacaaataaaacttgcagacactttc

SEQ ID NO: 85 - DNA, 444 bp, Rattus sp.
tagtcaatgtgttcttgatggagtggaaagcatttggaatagcagcgttcctgtgtgtgaccaagtgatatgtaaactccctcagaatatgagtggattccagaaagggttgcaaatgaataaagattattactatggagataatgtagccttggaatgtgaggatgggtatactctagaaggcagttctcagagccagtgccagtccgatgccagctgggatcctcctctgcccaaatgtgtctctcgctcaaacagtggtctaatagctggaattttcattgggataatcgtccttattttattcatcattttttcctactggatgattatgaagtttaaaaaacgtaccagtagcccagcacggaattcactcactcaagaagtctaatagccgccatgtacagtgatgtctctgaaacgcaaaacatttcctgtctgtat

SEQ ID NO: 86 - DNA, 317 bp, Rattus norvegicus
ccttgcacagtgggggccaacatggctctgcggagatccccaggtagtacctgtaaagaccatgagacagaacttctgtcacagcagaaggttcctgcgcatttcgtagctctgagtgttcttgctcccaggtacataaatcctggaacgcagagacaaaagctaggaatccccagggttgtgccagaaccactgtgccctcccgagaagcaagtgtctctctagcgtggatggcccgtgctccttttgctttcacacagaatttcccagttatgaaattaataaaaatcaatgatttccaaaaaaaaaaaaaaaaa

SEQ ID NO: 87 - DNA, 464 bp, Rattus sp.
accccttggtgttctgggggtcaaggttgagggacagagtctcactcacttagaagagatagagagccctgagtcttgggttctgtcacgaactcccccttcagctgcccatatgacctttgccatcattttctcctgcaaacctcagtctgctcatctggaaggtgagatcagaagatccctacgctctcaacgggcacctccctgattgctagcatcggccgtggccctcacaggctgagtgctcactgtcccaggaatgtccgtcagacagaagcagctggaaaggcctggagagggaatgtggagggaactgggaactggggtcatcaaatcacactccgatatccatgaatggcacatataaatgatactatattttgtatatttaatgtcacgggctccgctactctacaagggcctctgcactccttttctcttgtcaggggtctacagcggcaata

SEQ ID NO: 88 - DNA, 597 bp, Rattus sp.
  misc_feature (12)..(12): n is a, c, g, or t
caattctgtccntgcctgtgtcccatggtctccatatctattccaaccgaatgacagatgcatcatttctggatggggtcgagaaaaagataaccaaaaagtctactcactcaggtggggcgaagtcgacctaataggcaactgctcgaggttttacccgggtcgctactatgaaaaagagatgcagtgtgcgggtaccagtgatgggtccattgatgcctgcaaaggagactctggaggccccttggtctgcaaggatgtcaacaatgtcacttatgtttggggcattgtgagctggggagaaaactgtgggaaaccagagttcccaggtgtttacaccagagtggccagctattttgattggattagctactacgtgggaagaccccttgtttctcaatacaatgtctgaagctacgacctccttctttctgcacttcttctttccagggttatactttaattgaaatgaaactgtataattagttctcctcgatgctggcaagaagcaagtcttactggctagttcctaaagtttcttcaaagtttatgccattttagaattctgtcatataatccccaataaatattccagtt

SEQ ID NO: 89 - DNA, 481 bp, Rattus norvegicus
taggagactatggaacatggtcagactgtgacccttgtattagaaaacaggttaaagttagatctgttctgcgcccaagtcagtttgggggacaaccatgcacagagcccttggtgacctttcaaccatgtgtcccatctgagctctgcaaaattgaagagactgattgcaagaataaattcctctgtgacagtggcaaatctgcaactgcagctgccggaatccaggtcaacagcgttatgtccacagccactgtttaaagactggaaggacaagatggttcagcatgctgatgagcaaccagagttcattccttgagagtcacataatagaaagagaaccaaatcctataaattgtcttccggtctcttgaacctcaaccccatcccctgtggctgctatgccatcacccctccaataaaagctaattaagttctgcaataaaattaaaagttaaaacatccaaaataaaaaaaaaaaa

SEQ ID NO: 90 - DNA, 401 bp, Rattus norvegicus
gggccagtacatgtggttgttgctgagaccgactaccagagctttgccatcctgtatctggagcaggcgaggaggctgtctgtgaaactatacacccgcacactgcctgtgagtgattctgccctgaatgcgtttgaggagcgagtcaggggagccaacctgacagaagaccagatctttttctttcccaagtatggtttctgtgagactgcagaccaattccacatcctgaatgagatgccgaagtgaaatgaggccatctagtagagaagctatggactcccctcagagagttctgagcatcctgaatacaatcttggagtctttcctgtctctccccagaagcaccccttcatacggctctgttgtcctgagacttttcagtaaacatcagacttggc

SEQ ID NO: 91 - DNA, 388 bp, Rattus norvegicus
atgacactttatactcactgggtcccagcgtaaaggtcaccttccactccgattactccaatgagaagccattcacaggaattgaggccttctatgcagcggaggatgtggatgaatgcagaacatccttgggagactcagtcccttgtgaccattattgccacaactacttgggcggatactactgctcctgccgagtgggctacattatgcaccagaacaagcatacctgctcagagcagagcctataacttccatcgatccagcgtgcccaccccagtgagtcagacaccagtcaatacatgcccacacgactcagctctgtcaggctggagtggctcccagcacccattcacctttcccagacaataaacctgcctccacttcc

SEQ ID NO: 92 - DNA, 398 bp, Rattus norvegicus
ggggaaaagaaagccagttgtgagagagatgctacaaaggcccaaggctatgagaaggtcaaagttgcctctgaggtggtcacccccaggttcctgtgcaccggaggggtagatccctatgctgaccccaacacatgcaaaggagactccgggggccctctcattgttcacaagagaagccgcttcattcaagttggtgtgatcagctggggagtagtggatgtctgcaaagacccgaggcggcaacagttggtgccctcctatgcccgggacttccacatcaatctcttccaggtgctgccctggctaaaggagaagctcaaagacgaggacttgggtttcttataaggagcttcctgctgggagggtgagggcagattaaagcagctccaatccaa

SEQ ID NO: 93 - DNA, 453 bp, Rattus norvegicus
ccaggccagcagtcacgttcaaggagctgtggtggccgcaaatttgatgggcagccatgtactgggaaactccaagatattcgacactgctatgacatccataactgtgtcttgaaaggttcatggtcacagtggagtacctggggtctgtgcacaccaccatgtggacccaaccccacccgtgtccgtcagcgactttgcacacctttgctccccaagtactcgcctacagtttccatggttgaaggtcagggtgagaagaatgttaccttctgggggatcccacggccactgtgtgaggtgctacaggggcagaagctggtggtggaagagaaacggccatgtctacatgtgccttcctgcagagatccagaagagaagaaaccctaaaatcccttgcctccattttgaccccctgaccttctaaacctcaataaaccagcctttttagaa

SEQ ID NO: 94 - DNA, 499 bp, Rattus sp.
gccagtgccaggattaaaaccaatggaaccctccgctggctatctgcaaaaaccggtcaactgttcctcttgtttctggtatttctgcaggctcagcatttatcattttgattagtgtcatcttctttataatattaaaatccagagagcgcaattattatacaaaaacaaggcccaaagatggagctcttcatttagaaacgcgagaagtatatgctgttgatccatacaacccagcaagttgatcacatgacaagttggtatataatgaaattgaagtacagttacttatacatagtcaagatttgctttaatattttctcatagacttttaaagcacagtcttaggtaaatattgttctatttcagtctttatatccaggcacaacattattaacttatctactgatgattccaactgattccaactgatgatcttagttaaaaacatttgtctgttcagtaaatatgtaacaaatgtaattaataaactgattac

SEQ ID NO: 95 - DNA, 1192 bp, Rattus norvegicus
agtctggtaacatgacagcggcgcctctcacgccagacccaacgcatccccgtcgcagaaggaagagctacactttcttctccctgggcatttacgctgaggcccttctgtttctgctgtctagtttatctgatgcctgtgaaccaccaccaccatttgaagctatggaactcaaggataagcctaaaccccattatgcgattggagagataatagaatatacgtgtaaaaaaggatacctatatctgtctccatacccaatgactgctatctgtcagccaaatcacacatgggtccctatttcagatcatggttgtattaaagttcaatgtactatgttacaggacccttcgtttggcaaagtacactacatagatggtagattttcatggggtgctcgagttaaatatacttgtatgaatggttattacatggttggtatgtcagttctacagtgtgagcttaatggcaacggtgatgcattctggaatggccatcccccaagttgtaaaaaagtctattgtttaccacctccaaaaataaaaaatggaacacacacctttactgatataaaagtattcaaataccatgaagcagtaatttacagttgtgatcctaacccagggccagataagttttcccttgttggaccgagcatgctattctgtgctggccataacacctggagtagcgaccctccggagtgtaaagtggtaaaatgtccatttccagtgctacaaaatggaagacagatatcaagaactgaaaaaaaattttcctaccaagcactagtgctgtttcagtgtttggagggattttacatggagggcagtagcatggtggtctgtggtgctaagagctcttgggagccctctatcccacaatgtcttaaaggtcctaagcctcattctaccaagcctccagtttacagtgaatcaggatatcctagtccccgtgaaggaatatttggccaagaattcgatgcatggatcattgctttgattgttgttacttcagttgttggagttattgtaatttgtctcatcatactcaggtgttctgagtacaggaagaaatgaaatgtatctgcagcaagatgaaaaatcccacgtgtggaagtcattactgttccatttttgaaaactggttcttcaagtctgcaaaagcaaaattatatatttgcaggagcttc

SEQ ID NO: 96 - DNA, 1514 bp, Rattus norvegicus
atgatccgcacgcgggcgccggggactcgggctccgccgccgccgccgctgctgctgtcgctgtcgctgctgctgctgctgctgtccccgactgtaagcggagactgcggcccacctccagacattcctaatgccacgccaaactcgggtaaacacaccaagtttgcccaccaaagccaagtgacatattcgtgtaataagggcttcaaacaaattccagacaagccaaacacagtggtctgccttgaaaatgaccaatggtctaactacgaaactttctgtaataaaacctgtagtgctccactgagactgaattatgcatccctcaaaaaggagtatcgtaacatgaattttttcccaattggcaccactgtggaatttgagtgtcggccaggatttcaaaaagtaacttcacgcacaggaaaatcaacttgccttgaggaattagtatggtctccagttgttgaattttgtaaaaaaagagcatgccctaatcctagagaactgaaaaatggtcacatcaacataccaactgacatattattcggatcagaaataatcttctcatgcgacaaagggtacaagctggtcggtgtaacttctacattctgttctattacggggaatgctgtggactgggatgacgtatttccagtgtgcacaacaattttttgtccagacccaccaaaaatcaataatggaagaattcgagaggaaagcgactcctacgcatatagacagtcggtgacctattcctgtgaccaaggcttcattctggttggaaattcttccgttcattgtactctgaacggtgaagtaggagaatggagtagtgcaccacccaagtgcatagagagacacaaggtcccaactaagaccccaccagaagttgatattccaagtacaatgcctcatgaatccaccttaattaattttccaagtacaagagccccactgtctcataaacccactacagttaaagttccaggtacaagagtcaagccaacgtctcacaagcctactgaagttaaagttccagcaacacagcatgtacctgtttccaagacaacggtacgtcatccaacgagaacatctaaagacagaggagagtctaattcaggtggtgaccattttatatatggacatacatgtttaataaccatgacaattttgcatgtgatgctcttactcactggctagttgacgtagccaacgaagagttaagaagaaagtatatacaaccactgagaatacttctagtttgttagaatgtccaggaagaatggatacaataaatttaatagtgttgttggttctgtatgctgtcatcgtcttgaaggtgtgctagaaatgataacaaagcaagaagaaaggaggttgtctggaatggtcttttcaatgcatataagtctcttgaaaatatttcaaaactagacaaattcacaatgttgtgtagtatcttttctaaaaaaaagtgtgggaaagtgtagaaaatttttcaggggagagggaaa

SEQ ID NO: 97 - DNA, 1056 bp, Mus musculus; identical to SEQ ID NO: 41

SEQ ID NO: 98 - DNA, 1091 bp, Rattus norvegicus; identical to SEQ ID NO: 47

SEQ ID NO: 99 - DNA, 1995 bp, Homo sapiens; identical to SEQ ID NO: 25

SEQ ID NO: 100 - DNA, 2083 bp, Rattus norvegicus; identical to SEQ ID NO: 69

SEQ ID NO: 101 - DNA, 1068 bp, Rattus sp.; identical to SEQ ID NO: 59

SEQ ID NO: 102 - DNA, 1127 bp, Mus musculus; identical to SEQ ID NO: 45

SEQ ID NO: 103 - DNA, 2979 bp, Homo sapiens
gggcagcctgctgtcggcttagaggggatgggcagtgtggagggcctggcagagcaagaggactcatccttccaaagggactttctctgggaagcctgctcctcgggccactgcgaaccctctctactctccgaagggaattgtccttcctggcttccactacttccacccctgaatgcacaggcagcccggcccaagtctcccactagggatgcagatggattcggtgtgaagggctggctgctgttgcctccggctcttgaaagtcaagttcagaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccatagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcacctacgggggcatctgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctgatcgcttggagggtcctctttttatgttgagttgctgcttcccggcatgccttcattttgctatggggggcaggcaggggggatggaaaataagtagaaacaaaaaagcagtggctaagatggtatagggactgtcataccagtgaagaataaaagggtgaagaataaaagggatatgatgacaaggttgatccacttcaagaattgcttgctttcaggaagagagatgtgtttcaacaagccaactaaaatatattgctgcaaatggaagcttttctgttctattataaaactgtcgatgtattctgaccaaggtgcgacaatctcctaaaggaatacactgaaagttaaggagaagaatcagtaagtgtaaggtgtacttggtattataatgcataattgatgttttcgttatgaaaacatttggtgcccagaagtccaaattatcagttttatttgtaagagctattgcttttgcagcggttttatttgtaaaagctgttgatttcgagttgtaagagctcagcatcccaggggcatcttcttgactgtggcatttcctgtccaccgccggtttatatgatcttcatacctttccctggaccacaggcgtttctcggcttttagtctgaaccatagctgggctgcagtaccctacgctgccagcaggtggccatgactacccgtggtaccaatctcagtcttaaagctcaggcttttcgttcattaacattctctgatagaattctggtcatcagatgtactgcaatggaacaaaactcatctggctgcatcccaggtgtgtagcaaagtccacatgtaaatttatagcttagaatattcttaagtcactgtcccttgtctctctttgaagttataaacaacaaacttaaagcttagcttatgtccaaggtaagtattttagcatggctgtcaaggaaattcagagtaaagtcagtgtgattcacttaatgatatacattaattagaattatggggtcagaggtatttgcttaagtgatcataattgtaaagtatatgtcacattgtcacattaatgtcaaaaaaaaaaaaaaaaaa

SEQ ID NO: 104 - DNA, 4252 bp, Mus musculus; identical to SEQ ID NO: 8

SEQ ID NO: 105 - DNA, 1288 bp, Mus musculus; identical to SEQ ID NO: 37

SEQ ID NO: 106 - DNA, 1648 bp, Mus musculus; identical to SEQ ID NO: 39

SEQ ID NO: 107 - DNA, 827 bp, Rattus norvegicus
atgcacagctccgtgtacctcgtggctctggtggtcctggaggcggctgtatgtgttgcgcagccccgaggtcggattctgggtggccaggaggccatggcccatgctcggccctacatggcttcagtgcaagtgaatggcacgcacgtgtgcggtggcaccctggtggatgagcagtgggtgctgagcgccgcgcactgcatggatggagtgaccaaggatgaggttgtgcaggtgctcctgggtgcccactccctgtccagtcctgaaccctacaagcatttgtatgatgtgcaaagtgtagtgcttcacccgggcagccggcctgacagcgttgaggacgacctcatgctctttaagctctcccacaatgcctcactgggtccccatgtgagacccctgcccttgcaacgcgaggaccgggaggtgaaacccggcacgctctgcgatgtggccggttggggcgtggtcactcatgcgggacgcaggcccgatgtcctgcagcaactgacagtgtcaatcatggaccggaacacctgcaatctgcgcacgtaccatgatagggcaatcaccaagaacatgatgtgtgcagagagcaaccgcagggacacttgcaggggcgactccggcggtcctctggtgtgcggggatgcggtcgaagctgtggttacgtggggatctcgagtctgtggcaaccggagaaagccaggtgtctttacccgcgtggcaacctacgtgccgtggattgaaaacgttctgagtggtaacgtgagtgttaacgtgacggcctgaggggacaccggagaccgtgactcacaataaatgca

SEQ ID NO: 108 - DNA, 4034 bp, Homo sapiens
agggagaggcagagaggcaggcagcctgctgggctcttcctgctgttgaaaacttacccggcccttacagaggaaatcttcctcctctcttctgccctgaatgttttcccaaacatgaaggtgataagcttattcattttggtgggatttataggagagttccaaagtttttcaagtgcctcctctccagtcaactgccagtgggacttctatgccccttggtcagaatgcaatggctgtaccaagactcagactcgcaggcggtcagttgctgtgtatgggcagtatggaggccagccttgtgttggaaatgcttttgaaacacagtcctgtgaacctacaagaggatgtccaacagaggagggatgtggagagcgtttcaggtgcttttcaggtcagtgcatcagcaaatcattggtttgcaatggggattctgactgtgatgaagacagtgctgatgaagacagatgtgaggactcagaaaggagaccttcctgtgatatcgataaacctcctcctaacatagaacttactggaaatggttacaatgaactcactggccagtttaggaacagagtcatcaataccaaaagttttggtggtcaatgtagaaaggtgtttagtggggatggaaaagatttctacaggctgagtggaaatgtcctgtcctatacattccaggtgaaaataaataatgattttaattatgaattttacaatagtacttggtcttatgtaaaacatacgtcgacagaacacacatcatctagtcggaagcgctccttttttagatcttcatcatcttcttcacgcagttatacttcacataccaatgaaatccataaaggaaagagttaccaactgctggttgttgagaacactgttgaagtggctcagttcattaataacaatccagaatttttacaacttgctgagccattctggaaggagctttcccacctcccctctctgtatgactacagtgcctaccgaagattaatcgaccagtacgggacacattatctgcaatctgggtcgttaggaggagaatacagagttctattttatgtggactcagaaaaattaaaacaaaatgattttaattcagtcgaagaaaagaaatgtaaatcctcaggttggcattttgtcgttaaattttcaagtcatggatgcaaggaactggaaaacgctttaaaagctgcttcaggaacccagaacaatgtattgcgaggagaaccgttcatcagagggggaggtgcaggcttcatatctggccttagttacctagagctggacaatcctgctggaaacaaaaggcgatattctgcctgggcagaatctgtgactaatcttcctcaagtcataaaacaaaagctgacacctttatatgagctggtaaaggaagtaccttgtgcctctgtgaaaaaactatacctgaaatgggctcttgaagagtatctggatgaatttgacccctgtcattgccggccttgtcaaaatggtggtttggctactgttgaggggacccattgtctgtgccattgcaaaccgtacacatttggtgcggcgtgtgagcaaggagtcctcgtagggaatcaagcaggaggggttgatggaggttggagttgctggtcctcttggagcccctgtgtccaagggaagaaaacaagaagccgtgaatgcaataacccacctcccagtgggggtgggagatcctgcgttggagaaacgacagaaagcacacaatgcgaagatgaggagctggagcacttgaggttgcttgaaccacattgctttcctttgtctttggttccaacagaattctgtccatcacctcctgccttgaaagatggatttgttcaagatgaaggtacaatgtttcctgtggggaaaaatgtagtgtacacttgcaatgaaggatactctcttattggaaacccagtggccagatgtggagaagatttacggtggcttgttggggaaatgcattgtcagaaaattgcctgtgttctacctgtactgatggatggcatacagagtcacccccaaaaacctttctacacagttggtgagaaggtgactgtttcctgttcaggtggcatgtccttagaaggtccttcagcatttctctgtggctccagccttaagtggagtcctgagatgaagaatgcccgctgtgtacaaaaagaaaatccgttaacacaggcagtgcctaaatgtcagcgctgggagaaactgcagaattcaagatgtgtttgtaaaatgccctacgaatgtggaccttccttggatgtatgtgctcaagatgagagaagcaaaaggatactgcctctgacagtttgcaagatgcatgttctccactgtcagggtagaaattacacccttactggtagggacagctgtactctgcctgcctcagctgagaaagcttgtggtgcctgcccactgtggggaaaatgtgatgctgagagcagcaaatgtgtctgccgagaagcatcggagtgcgaggaagaagggtttagcatttgtgtggaagtgaacggcaaggagcagacgatgtctgagtgtgaggcgggcgctctgagatgcagagggcagagcatctctgtcaccagcataaggccttgtgctgcggaaacccagtaggctcctggaggccctggtcagcttgcttggaatccagcaggcagctggggctgagtgaaaacatctgcacaactgggcactggacagcttttccttcttctccagtgtctaccttcctcctcaactcccagccatctgtataaacacaatcctttgttctcccaaatctgaatcgaattactcttttgcctcctttttaatgtcagtaaggatatgagcctttgcacaggctggctgcgtgttcttgaaataggtgttaccttctctgggccttggttttttaaaatctgtaaaattagaggattgcactagagaaacttgaatgctccattcaggcctatcattttattaagtatgattgacacagcccatgggccagaacacactctacaaaatgactaggataacagaaagaacgtgatctcctgattagagagggtggttttcctcaatggaaccaaatataaagaggacttgaacaaaaatgacagatacaaactatttctatcctgagtagtaatctcacacttcatcctatagagtcaaccaccacagataggaattccttattctttttttaatttttttaagacagagtctcactttgttgcccaggctggagcgcagtggggtgatctcatctccctgcaacctccgcctcctgggttcaagcgattcttgtgcctcagcttcccaagcagctgggattacaggtgcccgccaccacgcccagctaatttttgcatttttagtagagatggggtttcaccatgttggccacgctcgtctccaactcctgacctcaggtaatccgcctgccttggcctcccaaagtgctgggattacagacatgaaccaccacgcctggctggaatacttactcttgtcgggagattgaaccactaaaatgttagagcagaattcattatgctgtggtcacaggggtgtcttgtctgagaacaaatacaattcagtcttctctttggggttttagtatgtgtcaaacataggactggaagtttgcccctgttcttttttcttttgaaagaacatcagttcatgcctgaggcatgagtgactgtgcatttgagaatagttttccctattctgtggatacagtcccagagttttcagggagtacacaggtagattagtttgaagcattgaccttttatttattccttatttctctttcatcaaaacaaaacagcagctgtgggaggagaaatgagagggcttaaatgaaatttaaaataagctatattatacaaatactatctctgtattgttctgaccctggtaaatatatttcaaaacttcagatgacaaggattagaacactcattaaagatgctattcttcagaaaaaaaaaaaaaaaaaaaaaaaa

SEQ ID NO: 109 - DNA, 1136 bp, Rattus norvegicus
gggcttcttggacgtttttgggaggggacagcaagggaaggtccttctgcctctagggacccagacttccgctttctgcagacagcagcaggctctgggctctgggaatccactgctgtctggcctagaagcatcatagaacacgaggattccatacacaggaggcccctgaagctgagctgagctgatgaagacacagtggagtgagatcttgacacccctgttgctgctgctcctgggtttgctccatgtctcctgggcccaaagcagctgtactgggtcccctggcatccctggggtccctggcatccctggggtccctggctctgatggcaaaccaggcactccagggataaaaggagagaaagggctccccggactggctggagaccatggtgagttaggagagaaaggggatgcagggatccctgggatcccaggcaaagttggccccaagggtcccgtcggccctaagggtgctccaggcccccctggaccccgcggtcccaaaggtggctctggagactacaaggctacccagaaagtagccttctctgccctgaggacggtcaacagcgccctgcgaccaaaccaggccattcgcttcgaaaaggtgatcaccaatgttaatgataactacgagccgcgcagtggcaagttcacctgcaaggtacctggcctctactacttcacctaccacgccagttcccgcgggaatctgtgtgtgaacatcgtgcgcggccgcgaccgagaccgcatgcagaaagttctcaccttctgcgactatgcccaaaacaccttccaggtcaccacgggtggggtagtcttgaagctggagcaggaagaggttgttcacctgcaggccacagacaagaactccctgctgggcgtcgagggagccaatagcatcttcactggctttctgcttttccctgacatggatgtatgatcacggggtcaaatcactcctatccaaaacctcctccctgccagtaatcctccctggaccccagacactgccctttgactgcccaaagccctgaccagagccctgtagatgttacagaatgggtaaataaactcttcaaggccaagaaaaaaaaaaaaaaaaaaaaaaaaaaaaaaaaaaaaaaccc

SEQ ID NO: 110 - DNA, 1019 bp, Mus musculus; identical to SEQ ID NO: 55

SEQ ID NO: 111 - DNA, 5066 bp, Rattus norvegicus; identical to SEQ ID NO: 6

SEQ ID NO: 112 - DNA, 5403 bp, Mus musculus; identical to SEQ ID NO: 3

SEQ ID NO: 113 - DNA, 2813 bp, Homo sapiens
agggaatcttatgaacagaaccaggacagggaggctggccggaggttcctgcagagggagcgtcaaggccctgtgctgctgtccctgggggccagaggggttgcccagcatgcccactggcaggagagagggaactgacccacttgctcctaccagcttctgaaggtgacactgagccccaggtgacgccgcaccaccaaagaaggtgcttgtgtttgtcagacaaatacagccaggcctgccaccccttaggctccaaagtccggaggtgcagaaagccaggaccaagagacaggcagctcaccagggtggacaaatcgccagagatgtggtgcattgtcctgttttcacttttggcatgggtttatgctgagcctaccatgtatggggagatcctgtcccctaactatcctcaggcatatcccagtgaggtagagaaatcttgggacatagaagttcctgaagggtatgggattcacctctacttcacccatctggacattgagctgtcagagaactgtgcgtatgactcagtgcagataatctcaggagacactgaagaagggaggctctgtggacagaggagcagtaacaatccccactctccaattgtggaagagttccaagtcccatacaacaaactccaggtgatctttaagtcagacttttccaatgaagagcgttttacggggtttgctgcatactatgttgccacagacataaatgaatgcacagattttgtagatgtcccttgtagccacttctgcaacaatttcattggtggttacttctgctcctgccccccggaatatttcctccatgatgacatgaagaattgcggagttaattgcagtggggatgtattcactgcactgattggggagattgcaagtcccaattatcccaaaccatatccagagaactcaaggtgtgaataccagatccggttggagaaagggttccaagtggtggtgaccttgcggagagaagattttgatgtggaagcagctgactcagcgggaaactgccttgacagtttagtttttgttgcaggagatcggcaatttggtccttactgtggtcatggattccctgggcctctaaatattgaaaccaagagtaatgctcttgatatcatcttccaaactgatctaacagggcaaaaaaagggctggaaacttcgctatcatggagatccaatgccctgccctaaggaagacactcccaattctgtttgggagcctgcgaaggcaaaatatgtctttagagatgtggtgcagataacctgtctggatgggtttgaagttgtggagggacgtgttggtgcaacatctttctattcgacttgtcaaagcaatggaaagtggagtaattccaaactgaaatgtcaacctgtggactgtggcattcctgaatccattgagaatggtaaagttgaagacccagagagcactttgtttggttctgtcatccgctacacttgtgaggagccatattactacatggaaaatggaggaggtggggagtatcactgtgctggtaacgggagctgggtgaatgaggtgctgggcccggagctgccgaaatgtgttccagtctgtggagtccccagagaaccctttgaagaaaaacagaggataattggaggatccgatgcagatattaaaaacttcccctggcaagtcttctttgacaacccatgggctggtggagcgctcattaatgagtactgggtgctgacggctgctcatgttgtggagggaaacagggagccaacaatgtatgttgggtccacctcagtgcagacctcacggctggcaaaatccaagatgctcactcctgagcatgtgtttattcatccgggatggaagctgctggaagtcccagaaggacgaaccaattttgataatgacattgcactggtgcggctgaaagacccagtgaaaatgggacccaccgtctctcccatctgcctaccaggcacctcttccgactacaacctcatggatggggacctgggactgatctcaggctggggccgaacagagaagagagatcgtgctgttcgcctcaaggcggcaaggttacctgtagctcctttaagaaaatgcaaagaagtgaaagtggagaaacccacagcagatgcagaggcctatgttttcactcctaacatgatctgtgctggaggagagaagggcatggatagctgtaaaggggacagtggtggggcctttgctgtacaggatcccaatgacaagaccaaattctacgcagctggcctggtgtcctgggggccccagtgtgggacctatgggctctacacacgggtaaagaactatgttgactggataatgaagactatgcaggaaaatagcaccccccgtgaggactaatccagatacatcccaccagcctctccaagggtggtgaccaatgcattaccttctgttccttatgatattctcattatttcatcatgactgaaagaagacacgagcgaatgatttaaatagaacttgattgttgagacgccttgctagaggtagagtttgatcatagaattgtgctggtcatacatttgtggtctgactccttggggtcctttccccggagtacctattgtagataacactatgggtggggcactcctttcttgcactattccacagggataccttaattctttgtttcctctttacctgttcaaaattccatttacttgatcattctcagtatccactgtctatgtacaataaaggatgtttataagcaaaaaaaaaaaaaaaaaaaaaaaaaaaaaaaaaaaaaaaaaa

SEQ ID NO: 114 - DNA, 2493 bp, Homo sapiens; identical to SEQ ID NO: 17

SEQ ID NO: 115 - DNA, 1826 bp, Homo sapiens
agtctgcactggagctgcctggtgaccagaagtttggagtccgctgacgtcgccgcccagatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctgggcagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagacctgcaggatcaggttagggcgagcgctacctctccagcctcagctctcagttgcagccctgctgctgcctgcctggacttgcccctgccacctcctgcctcaggtgtccgctatccaccaaaagggctcctgagggtctgggcaagggacctgcttctattagcccttctccatggccctgccatgctctccaaaccactttttgcagctttctctagttcaagttcaccagactctataaataaaacctgacagaccat

SEQ ID NO: 116 - DNA, 1444 bp, Mus musculus; identical to SEQ ID NO: 38

SEQ ID NO: 117 - DNA, 2587 bp, Mus musculus
gtcagatgacttttcctctcccctagctctccgttctactgtcctggagaccatggcccctctgctggctctcttctacctgctgcagctgggcccaggcctggctgctctcttctgcaaccagaatgtcaatatcaccggtggtaatttcaccctcagccatggctgggcccctgggagcctcctcatctactcctgccccctgggcaggtacccgtccccagcctggaggaaatgtcagagcaacggacagtggctgacaccaaggtctagctcacatcacaccctgcgatcctctcggatggttaaagcagtctgcaaaccggttcgatgcctagctccttcatcctttgaaaatggcatctatttccctcggctggtgtcctaccctgtgggtagcaacgtgagctttgagtgtgacgaagacttcaccttgcggggctcacctgtgcggtactgtcgccccaacggcctgtgggatggagagacggctgtgtgtgacaatggggctagccactgccccaaccctggcatctcagtgggcacagctcggacaggcttgaactttgaccttggggacaaggtcaggtaccgctgctcctcctcaaatatggtattgactggctctgcagagcgggagtgtcagagcaatggagtgtggagtgggtcggaacccatttgccgacagccttactcttacgacttccctgaggatgtagcatctgccctagacacctccctcaccaacctgcttggagccaccaatcccacccagaaccttctgacaaaaagtttgggccgtaagatcataatccagcgctcgggtcacctgaacctctatttgctgcttgatgcttctcagagtgtgacagaaaaagactttgacatcttcaagaagagtgccgaactcatggtggagaggatcttcagctttgaggtaaatgtcacggtagctatcatcacctttgcctctcagcccaaaaccatcatgtcgatcctgagtgagagatcccaggatgtgacggaggtgatcaccagtctggactctgccagctacaaagatcacgaaaatgccactggcgctaacacttatgaggttctcatccgcgtttactccatgatgcaaacgcagatggatcgcctgggcatggagacctctgcctggaaggaaatccgtcacaccatcatccttctgactgacggaaagtccaacatgggtgactctcccaagaaagcagtcaccagaatcagagagctcctgagcatcgaacagaacagagatgactacctggacatctatgctattggggtgggcaagctggatgtggactggaaagaactgaatgagctgggttccaagaaggatggcgagaggcatgccttcatcttgcaggatgcaaaggccttgcaacagatctttgagcacatgttggatgtctctaagctcacagataccatctgtggggtggggaacatgtccgccaatgcctctgaccaggagaggacaccttggcaagtcacctttaagcccaagagcaaggaaacttgccagggatcactcatctctgatcagtgggtgctgacagcagctcactgcttccatgacattcagatggaggaccaccacctgtggagggtcaatgtaggtgatcccacctctcagcatggcaaagaatttcttgtggaggacgtgataattgccccagggtttaatgtccatgcaaagcggaagcagggcatctcagagttctatgctgatgacattgccttgctgaagctatctcggaaagtgaaaatgtccacccatgccagacccatctgccttccttgcactgtgggagccaacatggctctgcggagatccccaggtagtacctgtaaagatcatgagacagaacttctgtcacagcagaaagttcctgcacattttgtagctttgaatgggaacagactcaacatcaacctcaggacaggacctgagtggacaaggtgtatccaggctgtctcccaaaacaaaaacatcttccccagcttgacaaacgttagcgaggtggtgacagaccagttcctatgcagtgggatggaggaggaagatgacaatccttgcaaaggagaatctgggggagccgttttccttggacggagatacaggttcttccaggtgggcctggtgagttggggtctttttgacccttgtcatggttcctccaacaaaaacttgcgcaagaaacctccacgtggtgttctgccaagggacttccacattagtcttttccgcctgcagccctggctgaggcagcacctggatggtgtcctggactttctgccactttaacatggtcactgactcctttattaggtctgaacttcctgtctaatacctctgagcgttctcactcctggatacataaatcttgggccccgggagcccagagacagtggcaaaagctagggaatccccaggattgtgccgggacctctgtgccacctgtgaagcaagtctctctctagtgtggatgacccgtgctccttttgctttcacacggaatttcccagttatgtaattaataaaaatcaatgatttcca

SEQ ID NO: 118 - DNA, 5135 bp, Mus musculus; identical to SEQ ID NO: 19

SEQ ID NO: 119 - DNA, 2021 bp, Rattus norvegicus; identical to SEQ ID NO: 23

SEQ ID NO: 120 - DNA, 3551 bp, Homo sapiens; identical to SEQ ID NO: 12

SEQ ID NO: 121 - DNA, 857 bp, Homo sapiens; identical to SEQ ID NO: 58

SEQ ID NO: 122 - DNA, 2455 bp, Homo sapiens
gctggacgggcacaccatgaggctgctgaccctcctgggccttctgtgtggctcggtggccacccccttaggcccgaagtggcctgaacctgtgttcgggcgcctggcatcccccggctttccaggggagtatgccaatgaccaggagcggcgctggaccctgactgcaccccccggctaccgcctgcgcctctacttcacccacttcgacctggagctctcccacctctgcgagtacgacttcgtcaagctgagctcgggggccaaggtgctggccacgctgtgcgggcaggagagcacagacacggagcgggcccctggcaaggacactttctactcgctgggctccagcctggacattaccttccgctccgactactccaacgagaagccgttcacggggttcgaggccttctatgcagccgaggacattgacgagtgccaggtggccccgggagaggcgcccacctgcgaccaccactgccacaaccacctgggcggtttctactgctcctgccgcgcaggctacgtcctgcaccgtaacaagcgcacctgctcagccctgtgctccggccaggtcttcacccagaggtctggggagctcagcagccctgaatacccacggccgtatcccaaactctccagttgcacttacagcatcagcctggaggaggggttcagtgtcattctggactttgtggagtccttcgatgtggagacacaccctgaaaccctgtgtccctacgactttctcaagattcaaacagacagagaagaacatggcccattctgtgggaagacattgccccacaggattgaaacaaaaagcaacacggtgaccatcacctttgtcacagatgaatcaggagaccacacaggctggaagatccactacacgagcacagcgcacgcttgcccttatccgatggcgccacctaatggccacgtttcacctgtgcaagccaaatacatcctgaaagacagcttctccatcttttgcgagactggctatgagcttctgcaaggtcacttgcccctgaaatcctttactgcagtttgtcagaaagatggatcttgggaccggccaatgcccgcgtgcagcattgttgactgtggccctcctgatgatctacccagtggccgagtggagtacatcacaggtcctggagtgaccacctacaaagctgtgattcagtacagctgtgaagagaccttctacacaatgaaagtgaatgatggtaaatatgtgtgtgaggctgatggattctggacgagctccaaaggagaaaaatcactcccagtctgtgagcctgtttgtggactatcagcccgcacaacaggagggcgtatatatggagggcaaaaggcaaaacctggtgattttccttggcaagtcctgatattaggtggaaccacagcagcaggtgcacttttatatgacaactgggtcctaacagctgctcatgccgtctatgagcaaaaacatgatgcatccgccctggacattcgaatgggcaccctgaaaagactatcacctcattatacacaagcctggtctgaagctgtttttatacatgaaggttatactcatgatgctggctttgacaatgacatagcactgattaaattgaataacaaagttgtaatcaatagcaacatcacgcctatttgtctgccaagaaaagaagctgaatcctttatgaggacagatgacattggaactgcatctggatggggattaacccaaaggggttttcttgctagaaatctaatgtatgtcgacataccgattgttgaccatcaaaaatgtactgctgcatatgaaaagccaccctatccaaggggaagtgtaactgctaacatgctttgtgctggcttagaaagtgggggcaaggacagctgcagaggtgacagcggaggggcactggtgtttctagatagtgaaacagagaggtggtttgtgggaggaatagtgtcctggggttccatgaattgtggggaagcaggtcagtatggagtctacacaaaagttattaactatattccctggatcgagaacataattagtgatttttaacttgcgtgtctgcagtcaaggattcttcatttttagaaatgcctgtgaagaccttggcagcgacgtggctcgagaagcattcatcattactgtggacatggcagttgttgctccacccaaaaaaacagactccaggtgaggctgctgtcatttctccacttgccagtttaattccagccttacccattgactcaaggggacataaaccacgagagtgacagtcatctttgcccacccagtgtaatgtcactgctcaaattacatttcattaccttaaaaagccagtctcttttcatactggctgttggcatttctgtaaactgcctgtccatgctctttgtttttaaacttgttcttattgaaaaaaaaaaaaaaaaa

SEQ ID NO: 123 - DNA, 2443 bp, Mus musculus; identical to SEQ ID NO: 16

SEQ ID NO: 124 - DNA, 1358 bp, Mus musculus; identical to SEQ ID NO: 30

SEQ ID NO: 125 - DNA, 3220 bp, Mus musculus; identical to SEQ ID NO: 10

SEQ ID NO: 126 - DNA, 3326 bp, Homo sapiens; identical to SEQ ID NO: 40

SEQ ID NO: 127 - protein, 351 aa, Mus musculus
MDPIDNSSFEINYDHYGTMDPNIPADGIHLPKRQPGDVAALIIYSVVFLVGVPGNALVVWVTAFEPDGPSNAIWFLNLAVADLLSCLAMPVLFTTVLNHNYWYFDATACIVLPSLILLNMYASILLLATISADRFLLVFKPIWCQKVRGTGLAWMACGVAWVLALLLTIPSFVYREAYKDFYSEHTVCGINYGGGSFPKEKAVAILRLMVGFVLPLLTLNICYTFLLLRTWSRKATRSTKTLKVVMAVVICFFIFWLPYQVTGVMIAWLPPSSPTLKRVEKLNSLCVSLAYINCCVNPIIYVMAGQGFHGRLLRSLPSIIRNALSEDSVGRDSKTFTPSTDDTSTRKSQAV

SEQ ID NO: 128 - protein, 258 aa, Rattus norvegicus
MLCLVVCCLIWLISALDGSCSEPPPVNNSVFVGKETEEQILGIYLCIKGYHLVGKKSLVFDPSKEWNSTLPECLLGHCPDPVLENGKINSSGPVNISGKIMFECNDGYILKGSNWSQCLEDHTWAPPLPICRSRDCEPPETPVHGYFEGETFTSGSVVTYYCEDGYHLVGTQKVQCSDGEWSPSYPTCESIQEPPKSAEQSALEKAILAFQESKDLCNATENFVRQLREGGITMEELKCSLEMKKTKLKSDILLNYHS

SEQ ID NO: 129 - protein, 591 aa, Homo sapiens
MKNSRTWAWRAPVELFLLCAALGCLSLPGSRGERPHSFGSNAVNKSFAKSRQMRSVDVTLMPIDCELSSWSSWTTCDPCQKKRYRYAYLLQPSQFHGEPCNFSDKEVEDCVTNRPCGSQVRCEGFVCAQTGRCVNRRLLCNGDNDCGDQSDEANCRRIYKKCQHEMDQYWGIGSLASGINLFTNSFEGPVLDHRYYAGGCSPHYILNTRFRKPYNVESYTPQTQGKYEFILKEYESYSDFERNVTEKMASKSGFSFGFKIPGIFELGISSQSDRGKHYIRRTKRFSHTKSVFLHARSDLEVAHYKLKPRSLMLHYEFLQRVKRLPLEYSYGEYRDLFRDFGTHYITEAVLGGIYEYTLVMNKEAMERGDYTLNNVHACAKNDFKIGGAIEEVYVSLGVSVGKCRGILNEIKDRNKRDTMVEDLVVLVRGGASEHITTLAYQELPTADLMQEWGDAVQYNPAIIKVKVEPLYELVTATDFAYSSTVRQNMKQALEEFQKEVSSCHCAPCQGNGVPVLKGSRCDCICPVGSQGLACEVSYRKNTPIDGKWNCWSNWSSCSGRRKTRQRQCNNPPPQNGGSPCSGPASETLDCS

SEQ ID NO: 130 - protein, 567 aa, Rattus norvegicus
MLLRTPGLPRRSGMASGVTITLAIAIFALEINAQAPEPTPREEPSADALLPIDCRMSTWSQWSQCDPCLKQRFRSRSMEVFGQFQGKSCADALGDRQHCEPTQECEEVQENCGNDFQCETGRCIKRKLLCNGDNDCGDFSDESDCESDPRLPCRDRVVEESELGRTAGYGINILGMDPLGTPFDNEFYNGLCDRVRDGNTLTYYRKPWNVAFLAYETKADKNFRTENYEEQFEMFKTIVRDRTTSFNANLALKFTITEAPIKKVGVDEVSPEKNSSKPKDSSVDFQFSYFKKENFQRLSSYLSQTKKMFLHVRGMIQLGRFVMRNRGVMLTTTFLDDVKALPVSYEKGEYFGFLETYGTHYSSSGSLGGLYELIYVLDKASMKEKGVELSDVKRCLGFNLDVSLYTPLQTALEGPSLTANVNHSDCLKTGDGKVVNISRDHIIDDVISFIRGGTRKQAVLLKEKLLRGAKTIDVNDFINWASSLDDAPALISQKLSPIYNLIPLTMKDAYAKKQNMEKAIEDYVNEFSARKCYPCQNGGTAILLDGQCMCSCTIKFKGIACEISKQR

SEQ ID NO: 131 - protein, 224 aa, Rattus sp.
VSSSGSQTCEETLKTCSVIACGRDGRDGPKGEKGEPGQGLRGLQGPPGKLGPPGSVGAPGSQGPKGQKGDRGDSRAIEVKLANMEAEINTLKSKLELTNKLHAFSMGKKSGKKFFVTNHERMPFSKVKALCSELRGTVAIPRNAEENKAIQEVAKTSAFLGITDEVTEGQFMYVTGGRLTYSNWKKDEPNDHGSGEDCVTIVDNGLWNDISCQASHTAVCEFPA

SEQ ID NO: 132 - protein, 278 aa, Mus musculus
MLPLLRCVPRSLGAASGLRTAIPAQPLRHLLQPAPRPCLRPFGLLSVRAGSARRSGLLQPPVPCACGCGALHTEGDKAFVEFLTDEIKEEKKIQKHKSLPKMSGDWELEVNGTEAKLLRKVAGEKITVTFNINNSIPPTFDGEEEPSQGQKAEEQEPERTSTPNFVVEVTKTDGKKTLVLDCHYPEDEIGHEDEAESDIFSIKEVSFQATGDSEWRDTNYTLNTDSLDWALYDHLMDFLADRGVDNTFADELVELSTALEHQEYITFLEDLKSFVKNQ

SEQ ID NO: 133 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 134 - protein, 1234 aa, Mus musculus
MRLSARIIWLILWTVCAAEDCKGPPPRENSEILSGSWSEQLYPEGTQATYKCRPGYRTLGTIVKVCKNGKWVASNPSRICRKKPCGHPGDTPFGSFRLAVGSQFEFGAKVVYTCDDGYQLLGEIDYRECGADGWINDIPLCEVVKCLPVTELENGRIVSGAAETDQEYYFGQVVRFECNSGFKIEGHKEIHCSENGLWSNEKPRCVEILCTPPRVENGDGINVKPVYKENERYHYKCKHGYVPKERGDAVCTGSGWSSQPFCEEKRCSPPYILNGIYTPHRIIHRSDDEIRYECNYGFYPVTGSTVSKCTPTGWIPVPRCTLKPCEFPQFKYGRLYYEESLRPNFPVSIGNKYSYKCDNGFSPPSGYSWDYLRCTAQGWEPEVPCVRKCVFHYVENGDSAYWEKVYVQGQSLKVQCYNGYSLQNGQDTMTCTENGWSPPPKCIRIKTCSASDIHIDNGFLSESSSIYALNRETSYRCKQGYVTNTGEISGSITCLQNGWSPQPSCIKSCDMPVFENSITKNTRTWFKLNDKLDYECLVGFENEYKHTKGSITCTYYGWSDTPSCYERECSVPTLDRKLVVSPRKEKYRVGDLLEFSCHSGHRVGPDSVQCYHFGWSPGFPTCKGQVASCAPPLEILNGEINGAKKVEYSHGEVVKYDCKPRFLLKGPNKIQCVDGNWTTLPVCIEEERTCGDIPELEHGSAKCSVPPYHHGDSVEFICEENFTMIGHGSVSCISGKWTQLPKCVATDQLEKCRVLKSTGIEAIKPKLTEFTHNSTMDYKCRDKQEYERSICINGKWDPEPNCTSKTSCPPPPQIPNTQVIETTVKYLDGEKLSVLCQDNYLTQDSEEMVCKDGRWQSLPRCIEKIPCSQPPTIEHGSINLPRSSEERRDSIESSSHEHGTTFSYVCDDGFRIPEENRITCYMGKWSTPPRCVGLPCGPPPSIPLGTVSLELESYQHGEEVTYHCSTGFGIDGPAFIICEGGKWSDPPKCIKTDCDVLPTVKNAIIRGKSKKSYRTGEQVTFRCQSPYQMNGSDTVTCVNSRWIGQPVCKDNSCVDPPHVPNATIVTRTKNKYLHGDRVRYECNKPLELFGQVEVMCENGIWTEKPKCRDSTGKCGPPPPIDNGDITSLSLPVYEPLSSVEYQCQKYYLLKGKKTITCTNGKWSEPPTCLHACVIPENIMESHNIILKWRHTEKIYSHSGEDIEFGCKYGYYKARDSPPFRTKCINGTINYPTCV

SEQ ID NO: 135 - protein, 390 aa, Mus musculus
MIRGRAPRTRPSPPPPLLPLLSLSLLLLSPTVRGDCGPPPDIPNARPILGRHSKFAEQSKVAYSCNNGFKQVPDKSNIVVCLENGQWSSHETFCEKSCVAPERLSFASLKKEYLNMNFFPVGTIVEYECRPGFREQPPLPGKATCLEDLVWSPVAQFCKKKSCPNPKDLDNGHINIPTGILFGSEINFSCNPGYRLVGVSSTFCSVTGNTVDWDDEFPVCTEIHCPEPPKINNGIMRGESDSYTYSQVVTYSCDKGFILVGNASIYCTVSKSDVGQWSSPPPRCIEKSKVPTKKPTINVPSTGTPSTPQKPTTESVPNPGDQPTPQKPSTVKVSATQHVPVTKTTVRHPIRTSTDKGEPNTGGDRYIYGHTCLITLTVLHVMLSLIGYLT

SEQ ID NO: 136 - protein, 352 aa, Mus musculus
MQSSLKEVTDMVLIPSQAMGFWGTLLFLIFLEQSWGQEQTRYIISTPIVFRVGAPENVTVQAHGHTEAFDTTVSVKSYPDENVRYSFSTVNLSPENKFQNTAILTIQAKQLSEGLNSFSNSYLEVVSKHFAKLEIVPIIYDNDSLFVQTDKSVYTPQQPVKVRVYSVNDDLEPATRETVLTFIDPEGSQVDTIEGNNLTGIASFPDFEIPSNPKHGRWTVKAKYREDASKTGTTYFEVKEYDKTYRISIMPTIDLQPEVEKQEAHGMCLHQPTECLRQKINEQASTYKHPMIKKCCYDGARYNIHETCVQRAARVKIGPICVKAFTLCCNMAHQILENSTFKHIHLSSHYRS

SEQ ID NO: 137 - protein, 263 aa, Rattus norvegicus
MHSSVYLVALVVLEAAVCVAQPRGRILGGQEAMAHARPYMASVQVNGTHVCGGTLVDEQWVLSAAHCMDGVTKDEVVQVLLGAHSLSSPEPYKHLYDVQSVVLHPGSRPDSVEDDLMLFKLSHNASLGPHVRPLPLQREDREVKPGTLCDVAGWGVVTHAGRRPDVLQQLTVSIMDRNTCNLRTYHDRAITKNMMCAESNRRDTCRGDSGGPLVCGDAVEAVVTWGSRVCGNRRKPGVFTRVATYVPWIENVLSGNVSVNVTA

SEQ ID NO: 138 - protein, 843 aa, Homo sapiens
MKVISLFILVGFIGEFQSFSSASSPVNCQWDFYAPWSECNGCTKTQTRRRSVAVYGQYGGQPCVGNAFETQSCEPTRGCPTEEGCGERFRCFSGQCISKSLVCNGDSDCDEDSADEDRCEDSERRPSCDIDKPPPNIELTGNGYNELTGQFRNRVINTKSFGGQCRKVFSGDGKDFYRLSGNVLSYTFQVKINNDFNYEFYNSTWSYVKHTSTEHTSSSRKRSFFRSSSSSSRSYTSHTNEIHKGKSYQLLVVENTVEVAQFINNNPEFLQLAEPFWKELSHLPSLYDYSAYRRLIDQYGTHYLQSGSLGGEYRVLFYVDSEKLKQNDFNSVEEKKCKSSGWHFVVKFSSHGCKELENALKAASGTQNNVLRGEPFIRGGGAGFISGLSYLELDNPAGNKRRYSAWAESVTNLPQVIKQKLTPLYELVKEVPCASVKKLYLKWALEEYLDEFDPCHCRPCQNGGLATVEGTHCLCHCKPYTFGAACEQGVLVGNQAGGVDGGWSCWSSWSPCVQGKKTRSRECNNPPPSGGGRSCVGETTESTQCEDEELEHLRLLEPHCFPLSLVPTEFCPSPPALKDGFVQDEGTMFPVGKNVVYTCNEGYSLIGNPVARCGEDLRWLVGEMHCQKIACVLPVLMDGIQSHPQKPFYTVGEKVTVSCSGGMSLEGPSAFLCGSSLKWSPEMKNARCVQKENPLTQAVPKCQRWEKLQNSRCVCKMPYECGPSLDVCAQDERSKRILPLTVCKMHVLHCQGRNYTLTGRDSCTLPASAEKACGACPLWGKCDAESSKCVCREASECEEEGFSICVEVNGKEQTMSECEAGALRCRGQSISVTSIRPCAAETQ

SEQ ID NO: 139 - protein, 253 aa, Rattus norvegicus
MKTQWSEILTPLLLLLLGLLHVSWAQSSCTGSPGIPGVPGIPGVPGSDGKPGTPGIKGEKGLPGLAGDHGELGEKGDAGIPGIPGKVGPKGPVGPKGAPGPPGPRGPKGGSGDYKATQKVAFSALRTVNSALRPNQAIRFEKVITNVNDNYEPRSGKFTCKVPGLYYFTYHASSRGNLCVNIVRGRDRDRMQKVLTFCDYAQNTFQVTTGGVVLKLEQEEVVHLQATDKNSLLGVEGANSIFTGFLLFPDMDV

SEQ ID NO: 140 - protein, 246 aa, Mus musculus
MVVGPSCQPQCGLCLLLLFLLALPLRSQASAGCYGIPGMPGMPGAPGKDGHDGLQGPKGEPGIPAVPGTQGPKGQKGEPGMPGHRGKNGPRGTSGLPGDPGPRGPPGEPGVEGRYKQKHQSVFTVTRQTTQYPEANALVRFNSVVTNPQGHYNPSTGKFTCEVPGLYYFVYYTSHTANLCVHLNLNLARVASFCDHMFNSKQVSSGGALLRLQRGDEVWLSVNDYNGMVGIEGSNSVFSGFLLFPD

SEQ ID NO: 141 - protein, 1663 aa, Rattus norvegicus
MGPTSGSQLLVLLLLLASSLLALGSPMYSIITPNVLRLESEETFILEAHDAQGDVPVTVTVQDFLKKQVLTSEKTVLTGATGHLNRVFIKIPASKEFNADKGHKYVTVVANFGATVVEKAVLVSFQSGYLFIQTDKTIYTPGSTVFYRIFTVDNNLLPVGKTVVIVIETPDGVPIKRDILSSHNQYGILPLSWNIPELVNMGQWKIRAFYEHAPKQTFSAEFEVKEYVLPSFEVLVEPTEKFYYIHGPKGLEVSITARFLYGKNVDGTAFVIFGVQDEDKKISLALSLTRVLIEDGSGEAVLSRKVLMDGVRPSSPEALVGKSLYVSVTVILHSGSDMVEAERSGIPIVTSPYQIHFTKTPKFFKPAMPFDLMVFVTNPDGSPARRVPVVTQGSDAQALTQDDGVAKLSVNTPNNRQPLTITVSTKKEGIPDARQATRTMQAQPYSTMHNSNNYLHLSVSRVELKPGDNLNVNFHLRTDAGQEAKIRYYTYLVMNKGKLLKAGRQVREPGQDLVVLSLPITPEFIPSFRLVAYYTLIGANGQREVVADSVWVDVKDSCVGTLVVKGDPRDNRQPAPGHQTTLRIEGNQGARVGLVAVDKGVFVLNKKNKLTQSKIWDVVEKADIGCTPGSGKNYAGVFMDAGLTFKTNQGLQTDQREDPECAKPAARRRRSVQLMERRMDKAGQYTDKGLRKCCEDGMRDIPMPYSCQRRARLITQGESCLKAFMDCCNYITKLREQHRRDHVLGLARSDVDEDIIPEEDIISRSHFPESWLWTIEELKEPEKNGISTKVMNIFLKDSITTWEILAVSLSDKKGICVADPYEITVMQDFFIDLRLPYSVVRNEQVEIRAVLFNYREQEKLKVRVELLHNPAFCSMATAKKRYYQTIEIPPKSSVAVPYVIVPLKIGLQEVEVKAAVFNHFISDGVKKILKVVPEGMRVNKTVAVRTLDPEHLNQGGVQREDVNAADLSDQVPDTDSETRILLQGTPVAQMAEDAVDGERLKHLIVTPSGCGEQNMIGMTPTVIAVHYLDQTEQWEKFGLEKRQEALELIKKGYTQQLAFKQPISAYAAFNNRPPSTWLTAMWSRSFSLAANLIAIDSQVLCGAVKWLILEKQKPDGVFQEDGPVIHQEMIGGFRNTKEADVSLTAFVLIALQEARDICEGQVNSLPGSINKAGEYLEASYLNLQRPYTVAIAGYALALMNKLEEPYLTKFLNTAKDRNRWEEPGQQLYNVEATSYALLALLLLKDFDSVPPVVRWLNDERYYGGGYGSTQATFMVFQALAQYRADVPDHKDLNMDVSLHLPSRSSPTVFRLLWESGSLLRSEETKQNEGFSLTAKGKGQGTLSVVTVYHAKVKGKTTCKKFDLRVTIKPAPETAKKPQDAKSSMILDICTRYLGDVDATMSILDISMMTGFIPDTNDLELLSSGVDRYISKYEMDKAFSNKNTLIIYLEKISHSEEDCLSFKVHQFFNVGLIQPGSVKVYSYYNLEESCTRFYHPEKDDGMLSKLCHNEMCRCAEENCFMHQSQDQVSLNERLDKACEPGVDYVYKTKLTTIELSDDFDEYIMTIEQVIKSGSDEVQAGQERRFISHVKCRNALKLQKGKQYLMWGLSSDLWGEKPNTSYIIGKDTWVEHWPEAEERQDQKNQKQCEDLGAFTETMVVFGCPN

SEQ ID NO: 142 - protein, 1680 aa, Mus musculus
MGLWGILCLLIFLDKTWGQEQTYVISAPKILRVGSSENVVIQVHGYTEAFDATLSLKSYPDKKVTFSSGYVNLSPENKFQNAALLTLQPNQVPREESPVSHVYLEVVSKHFSKSKKIPITYNNGILFIHTDKPVYTPDQSVKIRVYSLGDDLKPAKRETVLTFIDPEGSEVDIVEENDYTGIISFPDFKIPSNPKYGVWTIKANYKKDFTTTGTAYFEIKEYVLPRFSVSIELERTFIGYKNFKNFEITVKARYFYNKVVPDAEVYAFFGLREDIKDEEKQMMHKATQAAKLVDGVAQISFDSETAVKELSYNSLEDLNNKYLYIAVTVTESSGGFSEEAEIPGVKYVLSPYTLNLVATPLFVKPGIPFSIKAQVKDSLEQAVGGVPVTLMAQTVDVNQETSDLETKRSITHDTDGVAVFVLNLPSNVTVLKFEIRTDDPELPEENQASKEYEAVAYSSLSQSYIYIAWTENYKPMLVGEYLNIMVTPKSPYIDKITHYNYLILSKGKIVQYGTREKLFSSTYQNINIPVTQNMVPSARLLVYYIVTGEQTAELVADAVWINIEEKCGNQLQVHLSPDEYVYSPGQTVSLDMVTEADSWVALSAVDRAVYKVQGNAKRAMQRVFQALDEKSDLGCGAGGGHDNADVFHLAGLTFLTNANADDSHYRDDSCKEILRSKRNLHLLRQKIEEQAAKYKHSVPKKCCYDGARVNFYETCEERVARVTIGPLCIRAFNECCTIANKIRKESPHKPVQLGRIHIKTLLPVMKADIRSYFPESWLWEIHRVPKRKQLQVTLPDSLTTWEIQGIGISDNGICVADTLKAKVFKEVFLEMNIPYSVVRGEQIQLKGTVYNYMTSGTKFCVKMSAVEGICTSGSSAASLHTSRPSRCVFQRIEGSSSHLVTFTLLPLEIGLHSINFSLETSFGKDILVKTLRVVPEGVKRESYAGVILDPKGIRGIVNRRKEFPYRIPLDLVPKTKVERILSVKGLLVGEFLSTVLSKEGINILTHLPKGSAEAELMSIAPVFYVFHYLEAGNHWNIFYPDTLSKRQSLEKKIKQGVVSVMSYRNADYSYSMWKGASASTWLTAFALRVLGQVAKYVKQDENSICNSLLWLVEKCQLENGSFKENSQYLPIKLQGTLPAEAQEKTLYLTAFSVIGIRKAVDICPTMKIHTALDKADSFLLENTLPSKSTFTLAIVAYALSLGDRTHPRFRLIVSALRKEAFVKGDPPIYRYWRDTLKRPDSSVPSSGTAGMVETTAYALLASLKLKDMNYANPIIKWLSEEQRYGGGFYSTQDTINAIEGLTEYSLLLKQIHLDMDINVAYKHEGDFHKYKVTEKHFLGRPVEVSLNDDLVVSTGYSSGLATVYVKTVVHKISVSEEFCSFYLKIDTQDIEASSHFRLSDSGFKRIIACASYKPSKEESTSGSSHAVMDISLPTGIGANEEDLRALVEGVDQLLTDYQIKDGHVILQLNSIPSRDFLCVRFRIFELFQVGFLNPATFTVYEYHRPDKQCTMIYSISDTRLQKVCEGAACTCVEADCAQLQAEVDLAISADSRKEKACKPETAYAYKVRITSATEENVFVKYTATLLVTYKTGEAADENSEVTFIKKMSCTNANLVKGKQYLIMGKEVLQIKHNFSFKYIYPLDSSTWIEYWPTDTTCPSCQAFVENLNNFAEDLFLNSCE

SEQ ID NO: 143 - protein, 688 aa, Homo sapiens
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 144 - protein, 705 aa, Homo sapiens
MWLLYLLVPALFCRAGGSIPIPQKLFGEVTSPLFPKPYPNNFETTTVITVPTGYRVKLVFQQFDLEPSEGCFYDYVKISADKKSLGRFCGQLGSPLGNPPGKKEFMSQGNKMLLTFHTDFSNEENGTIMFYKGFLAYYQAVDLDECASRSKSGEEDPQPQCQHLCHNYVGGYFCSCRPGYELQEDRHSCQAECSSELYTEASGYISSLEYPRSYPPDLRCNYSIRVERGLTLHLKFLEPFDIDDHQQVHCPYDQLQIYANGKNIGEFCGKQRPPDLDTSSNAVDLLFFTDESGDSRGWKLRYTTEIIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 145 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGQNTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 146 - protein, 440 aa, Mus musculus
MEVSSRSSEPLDPVWLLVAFGRGGVKLEVLLLFLLPFTLGHCPAPSQLPSAKPINLTDESMFPIGTYLLYECLPGYIKRQFSITCKQDSTWTSAEDKCIRKQCKTPSDPENGLVHVHTGIQFGSRINYTCNQGYRLIGSSSAVCVITDQSVDWDTEAPICEWIPCEIPPGIPNGDFFSSTREDFHYGMVVTYRCNTDARGKALFNLVGEPSLYCTSNDGEIGVWSGPPPQCIELNKCTPPPYVENAVMLSENRSLFSLRDIVEFRCHPGFIMKGASSVHCQSLNKWEPELPSCFKGVICRLPQEMSGFQKGLGMKKEYYYGENVTLECEDGYTLEGSSQSQCQSDGSWNPLLAKCVSRSISGLIVGIFIGIIVFILVIIVFIWMILKYKKRNTTDEKYKEVGIHLNYKEDSCVRLQSLLTSQENSSTTSPARNSLTQEVS

SEQ ID NO: 147 - protein, 760 aa, Mus musculus
MAPLLALFYLLQLGPGLAALFCNQNVNITGGNFTLSHGWAPGSLLIYSCPLGRYPSPAWRKCQSNGQWLTPRSSSHHTLRSSRMVKAVCKPVRCLAPSSFENGIYFPRLVSYPVGSNVSFECDEDFTLRGSPVRYCRPNGLWDGETAVCDNGASHCPNPGISVGTARTGLNFDLGDKVRYRCSSSNMVLTGSAERECQSNGVWSGSEPICRQPYSYDFPEDVASALDTSLTNLLGATNPTQNLLTKSLGRKIIIQRSGHLNLYLLLDASQSVTEKDFDIFKKSAELMVERIFSFEVNVTVAIITFASQPKTIMSILSERSQDVTEVITSLDSASYKDHENATGANTYEVLIRVYSMMQTQMDRLGMETSAWKEIRHTIILLTDGKSNMGDSPKKAVTRIRELLSIEQNRDDYLDIYAIGVGKLDVDWKELNELGSKKDGERHAFILQDAKALQQIFEHMLDVSKLTDTICGVGNMSANASDQERTPWQVTFKPKSKETCQGSLISDQWVLTAAHCFHDIQMEDHHLWRVNVGDPTSQHGKEFLVEDVIIAPGFNVHAKRKQGISEFYADDIALLKLSRKVKMSTHARPICLPCTVGANMALRRSPGSTCKDHETELLSQQKVPAHFVALNGNRLNINLRTGPEWTRCIQAVSQNKNIFPSLTNVSEVVTDQFLCSGMEEEDDNPCKGESGGAVFLGRRYRFFQVGLVSWGLFDPCHGSSNKNLRKKPPRGVLPRDFHISLFRLQPWLRQHLDGVLDFLPL

SEQ ID NO: 148 - protein, 704 aa, Mus musculus
MRFLSFWRLLLYHALCLALPEVSAHTVELNEMFGQIQSPGYPDSYPSDSEVTWNITVPEGFRIKLYFMHFNLESSYLCEYDYVKVETEDQVLATFCGRETTDTEQTPGQEVVLSPGTFMSVTFRSDFSNEERFTGFDAHYMAVDVDECKEREDEELSCDHYCHNYIGGYYCSCRFGYILHTDNRTCRVECSGNLFTQRTGTITSPDYPNPYPKSSECSYTIDLEEGFMVSLQFEDIFDIEDHPEVPCPYDYIKIKAGSKVWGPFCGEKSPEPISTQTHSVQILFRSDNSGENRGWRLSYRAAGNECPKLQPPVYGKIEPSQAVYSFKDQVLVSCDTGYKVLKDNGVMDTFQIECLKDGAWSNKIPTCKIVDCGAPAGLKHGLVTFSTRNNLTTYKSEIRYSCQQPYYKMLHNTTGVYTCSAHGTWTNKVLKRSLPTCLPVCGVPKFSRKQISRIFNGRPAQKGTMPWIAMLSHLNGQPFCGGSLLGSNWVLTAAHCLHQSLDPEEPTLHSSYLLSPSDFKIIMGKHWRRRSDEDEQHLHVKRTTLHPLYNPSTFENDLGLVELSESPRLNDFVMPVCLPEQPSTEGTMVIVSGWGKQFLQRFPENLMEIEIPIVNSDTCQEAYTPLKKKVTKDMICAGEKEGGKDACAGDSGGPMVTKDAERDQWYLVGVVSWGEDCGKKDRYGVYSYIYPNKDWIQRITGVRN

SEQ ID NO: 149 - protein, 604 aa, Rattus norvegicus
MKLALLILLLLNPHLSSSKNTPASGQPQEDLVEQKCLLKNYTHHSCDKVFCQPWQKCIEGTCACKLPYQCPKAGTPVCATNGRGYPTYCHLKSFECLHPEIKFSNNGTCTAEEKFNVSLIYGSTDTEGIVQVKLVDQDEKMFICKNSWSTVEANVACFDLGFPLGVRDIQGRFNIPVNHKINSTECLHVRCQGVETSLAECTFTKKSSKAPHGLAGVVCYTQDADFPTSQSFQCVNGKRIPQEKACDGVNDCGDQSDELCCKGCRGQAFLCKSGVCIPNQRKCNGEVDCITGEDESGCEEDKKNKIHKGLARSDQGGETEIETEETEMLTPDMDTERKRIKSLLPKLSCGVKRNTHIRRKRVVGGKPAEMGDYPWQVAIKDGDRITCGGIYIGGCWILTAAHCVRPSRYRNYQVWTSLLDWLKPNSQLAVQGVSRVVVHEKYNGATYQNDIALVEMKKHPGKKECELINSVPACVPWSPYLFQPNDRCIISGWGREKDNQKVYSLRWGEVDLIGNCSRFYPGRYYEKEMQCAGTSDGSIDACKGDSGGPLVCKDVNNVTYVWGIVSWGENCGKPEFPGVYTRVASYFDWISYYVGRPLVSQYNV

SEQ ID NO: 150 - protein, 934 aa, Homo sapiens
MARRSVLYFILLNALINKGQACFCDHYAWTQWTSCSKTCNSGTQSRHRQIVVDKYYQENFCEQICSKQETRECNWQRCPINCLLGDFGPWSDCDPCIEKQSKVRSVLRPSQFGGQPCTEPLVAFQPCIPSKLCKIEEADCKNKFRCDSGRCIARKLECNGENDCGDNSDERDCGRTKAVCTRKYNPIPSVQLMGNGFHFLAGEPRGEVLDNSFTGGICKTVKSSRTSNPYRVPANLENVGFEVQTAEDDLKTDFYKDLTSLGHNENQQGSFSSQGGSSFSVPIFYSSKRSENINHNSAFKQAIQASHKKDSSFIRIHKVMKVLNFTTKAKDLHLSDVFLKALNHLPLEYNSALYSRIFDDFGTHYFTSGSLGGVYDLLYQFSSEELKNSGLTEEEAKHCVRIETKKRVLFAKKTKVEHRCTTNKLSEKHEGSFIQGAEKSISLIRGGRSEYGAALAWEKGSSGLEEKTFSEWLESVKENPAVIDFELAPIVDLVRNIPCAVTKRNNLRKALQEYAAKFDPCQCAPCPNNGRPTLSGTECLCVCQSGTYGENCEKQSPDYKSNAVDGQWGCWSSWSTCDATYKRSRTRECNNPAPQRGGKRCEGEKRQEEDCTFSIMENNGQPCINDDEEMKEVDLPEIEADSGCPQPVPPENGFIRNEKQLYLVGEDVEISCLTGFETVGYQYFRCLPDGTWRQGDVECQRTECIKPVVQEVLTITPFQRLYRIGESIELTCPKGFVVAGPSRYTCQGNSWTPPISNSLTCEKDTLTKLKGHCQLGQKQSGSECICMSPEEDCSHHSEDLCVFDTDSNDYFTSPACKFLAEKCLNNQQLHFLHIGSCQDGRQLEWGLERTRLSSNSTKKESCGYDTCYDWEKCSASTSKCVCLLPPQCFKGGNQLYCVKMGSSTSEKTLNICEVGTIRCANRKMEILHPGKCLA

SEQ ID NO: 151 - protein, 202 aa, Homo sapiens
MLPPGTATLLTLLLAAGSLGQKPQRPRRPASPISTIQPKANFDAQQFAGTWLLVAVGSACRFLQEQGHRAEATTLHVAPQGTAMAVSTFRKLDGICWQVRQLYGDTGVLGRFLLQARGARGAVHVVVAETDYQSFAVLYLERAGQLSVKLYARSLPVSDSVLSGFEQRVQEAHLTEDQIFYFPKYGFCEAADQFHVLDEVRR

SEQ ID NO: 152 - protein, 686 aa, Homo sapiens [sequence omitted]

SEQ ID NO: 153 - protein, 761 aa, Mus musculus
MESPQLCLVLLVLGFSSGGVSATPVLEARPQVSCSLEGVEIKGGSFQLLQGGQALEYLCPSGFYPYPVQTRTCRSTGSWSDLQTRDQKIVQKAECRAIRCPRPQDFENGEFWPRSPFYNLSDQISFQCYDGYVLRGSANRTCQENGRWDGQTAICDDGAGYCPNPGIPIGTRKVGSQYRLEDIVTYHCSRGLVLRGSQKRKCQEGGSWSGTEPSCQDSFMYDSPQEVAEAFLSSLTETIEGADAEDGHSPGEQQKRKIVLDPSGSMNIYLVLDGSDSIGSSNFTGAKRCLTNLIEKVASYGVRPRYGLLTYATVPKVLVRVSDERSSDADWVTEKLNQISYEDHKLKSGTNTKRALQAVYSMMSWAGDAPPEGWNRTRHVIIIMTDGLHNMGGNPVTVIQDIRALLDIGRDPKNPREDYLDVYVFGVGPLVDSVNINALASKKDNEHHVFKVKDMEDLENVFYQMIDETKSLSLCGMVWEHKKGNDYHKQPWQAKISVTRPLKGHETCMGAVVSEYFVLTAAHCFMVDDQKHSIKVSVGGQRRDLEIEEVLFHPKYNINGKKAEGIPEFYDYDVALVKLKNKLKYGQTLRPICLPCTEGTTRALRLPQTATCKQHKEQLLPVKDVKALFVSEQGKSLTRKEVYIKNGDKKASCERDATKAQGYEKVKDASEVVTPRFLCTGGVDPYADPNTCKGDSGGPLIVHKRSRFIQVGVISWGVVDVCRDQRRQQLVPSYARDFHINLFQVLPWLKDKLKDEDLGFL

SEQ ID NO: 154 - protein, 437 aa, Mus musculus
CFTQYEESSGRCKGLLGRDIRVEDCCLNAAYAFQEHDGGLCQACRSPQWSAWSLWGPCSVTCSEGSQLRHRRCVGRGGQCSENVAPGTLEWQLQACEDQPCCPEMGGWSEWGPWGPCSVTCSKGTQIRQRVCDNPAPKCGGHCPGEAQQSQACDTQKTCPTHGAWASWGPWSPRSGSCLGGAQEPKETRSRSCSAPAPSHQPPGKPCSGPAYEHKACSGLPPCPVAGGWGPWSPLSPCSVTCGLGQTLEQRTCDHPAPRHGGPFCAGDATRNQMCNKAVPCPVNGEWEAWGKWSDCSRLRMSINCEGTPGQQSRSRSCGDRKFNGKPCAGKLQDIRHCYNIHNCIMKGSWSQWSTWSLCTPPCSPNATRVRQRLCTPLLPKYPPTVSMVEGQGEKNVTFWGTPRPLCEALQGQKLVVEEKRSCLHVPVCKDPEEKKP

SEQ ID NO: 155 - protein, 1025 aa, Mus musculus
MLTWFLFYFSEISCDPPPEVKNARKPYYSLPIVPGTVLRYTCSPSYRLIGEKAIFCISENQVHATWDKAPPICESVNKTISCSDPIVPGGFMNKGSKAPFRHGDSVTFTCKANFTMKGSKTVWCQANEMWGPTALPVCESDFPLECPSLPTIHNGHHTGQHVDQFVAGLSVTYSCEPGYLLTGKKTIKCLSSGDWDGVIPTCKEAQCEHPGKFPNGQVKEPLSLQVGTTVYFSCNEGYQLQGQPSSQCVIVEQKAIWTKKPVCKEILCPPPPPVRNGSHTGSFSENVPYGSTVTYTCDPSPEKGVSFTLIGEKTINCTTGSQKTGIWSGPAPYCVLSTSAVLCLQPKIKRGQILSILKDSYSYNDTVAFSCEPGFTLKGNRSIRCNAHGTWEPPVPVCEKGCQAPPKIINGQKEDSYLLNFDPGTSIRYSCDPGYLLVGEDTIHCTPEGKWTPITPQCTVAECKPVGPHLFKRPQNQFIRTAVNSSCDEGFQLSESAYQLCQGTIPWFIEIRLCKEITCPPPPVIHNGTHTWSSSEDVPYGTVVTYMCYPGPEEGVKFKLIGEQTIHCTSDSRGRGSWSSPAPLCKLSLPAVQCTDVHVENGVKLTDNKAPYFYNDSVMFKCDDGYILSGSSQIRCKANNTWDPEKPLCKKEGCEPMRVHGLPDDSHIKLVKRTCQNGYQLTGYTYEKCQNAENGTWFKKIEVCTVILCQPPPKIANGGHTGMMAKHFLYGNEVSYECDEGFYLLGEKSLQCVNDSKGHGSWSGPPPQCLQSSPLTHCPDPEVKHGYKLNKTHSAFSHNDIVHFVCNQGFIMNGSHLIRCHTNNTWLPGVPTCIRKASLGCQSPSTIPNGNHTGGSIARFPPGMSVMYSCYQGFLMAGEARLICTHEGTWSQPPPFCKEVNCSFPEDTNGIQKGFQPGKTYRFGATVTLECEDGYTLEGSPQSQCQDDSQWNPPLALCKYRRWSTIPLICGISVGSALIILMSVGFCMILKHRESNYYTKTRPKEGALHLETREVYSIDPYNPAS

SEQ ID NO: 156 - protein, 377 aa, Homo sapiens
MEPPGRRECPFPSWRFPGLLLAAMVLLLYSFSDACEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVSTSSTTKSPASSASGPRPTYKPPVSNYPGYPKPEEGILDSLDVWVIAVIVIAIVVGVAVICVVPYRYLQRRKKKGTYLTDETHREVKFTSL

SEQ ID NO: 157 - DNA, 25 bp, artificial
  primer
gttgttggttctgtatgctgtcatc

SEQ ID NO: 158 - DNA, 22 bp, artificial
  primer
ccattccagacaacctcctttc

SEQ ID NO: 159 - DNA, 30 bp, artificial
  probe
cttgaaggtgtgctagaaatgataacaaag

SEQ ID NO: 160 - DNA, 27 bp, artificial
  primer
cggtcaaggtctactcctactacaatc

SEQ ID NO: 161 - DNA, 21 bp, artificial
  primer
cagcattccatcgtccttctc

SEQ ID NO: 162 - DNA, 28 bp, artificial
  probe
aggagtcatgcacccggttctatcatcc

SEQ ID NO: 163 - DNA, 39 bp, artificial
  primer
aattaaccctcactaaaggggttgttggttctgtatgct

SEQ ID NO: 164 - DNA, 39 bp, artificial
  primer
taatacgactcactatagggccattccagacaacctcct

SEQ ID NO: 165 - DNA, 125 bp, artificial
  probe
aattaaccctcactaaaggggttgttggttctgtatgctgtcatcgtcttgaaggtgtgctagaaatgataacaaagcaagaagaaaggaggttgtctggaatggccctatagtgagtcgtatta

SEQ ID NO: 166 - DNA, 39 bp, artificial
  primer
aattaaccctcactaaaggggatctcacactccgaagaa

SEQ ID NO: 167 - DNA, 39 bp, artificial
  primer
taatacgactcactatagggatccgacagctctatcgtc

SEQ ID NO: 168 - DNA, 359 bp, artificial
  primer
aattaaccctcactaaaggggatctcacactccgaagaagactgcctgtccttcaaagtccaccagttctttaacgtgggacttatccagccggggtcggtcaaggtctactcctactacaatctagaggagtcatgcacccggttctatcatccggagaaggacgatggaatgctgagcaagctgtgccacaatgaaatgtgccgctgtgccgaggagaactgcttcatgcatcagtcacaggatcaggtcagcctgaatgaacgactagacaaggcttgtgagcctggagtggactacgtgtacaagaccaagctaacgacgatagagctgtcggatccctatagtgagtcgtatta